(19) United States
(12) Patent Application Publication
(10) Pub. No.: US 2014/0303092 A1
(43) Pub. Date: Oct. 9, 2014

(54) HEXADECYLOXYPROPYL CIDOFOVIR FOR THE TREATMENT OF DOUBLE-STRANDED DNA VIRUS INFECTION

(71) Applicant: CHIMERIX, INC., Durham, NC (US)

(72) Inventors: George R. Painter, Chapel Hill, NC (US); Ernest Randall Lanier, Chapel Hill, NC (US); Merrick R. Almond, Apex, NC (US); Dorothy Margolskee, Voorhees, NJ (US); Gwendolyn Powell Painter, Chapel Hill, NC (US)

(73) Assignee: CHIMERIX, INC, Durham, NC (US)

(21) Appl. No.: 14/354,481

(22) PCT Filed: Oct. 26, 2012
(86) PCT No.: PCT/US12/62177

§ 371 Date: Apr. 25, 2014

Related U.S. Application Data
(60) Provisional application No. 61/551,626, filed on Oct. 26, 2011.
(60) Provisional application No. 61/639,764, filed on Apr. 27, 2012.
(60) Provisional application No. 61/684,524, filed on Aug. 17, 2012.
(60) Provisional application No. 61/696,524, filed on Sep. 4, 2012.

(51) Int. Cl. C07F 9/6512; A61K 45/06; A61K 31/675

(52) U.S. Cl. CPC C07F 9/65121; A61K 31/675; A61K 45/06
USPC 514/20.5; 514/86

(57) ABSTRACT

The present application provides methods and compositions for treatment or prevention of dsDNA virus infection in post-hematopoietic cell transplant (HCT or HSCT) patients.

### RELATED APPLICATIONS

This application claims the benefit of U.S. Provisional Application No. 61/551,626, filed Oct. 26, 2011; U.S. Provisional Application No. 61/639,764, filed Apr. 27, 2012; U.S. Provisional Application No. 61/684,524, filed Aug. 17, 2012; and U.S. Provisional Application No. 61/696,524, filed Sep. 4, 2012, each of which is incorporated by reference herein in its entirety.

### FIELD OF THE INVENTION

The present invention concerns methods of treating diseases associated with cytomegalovirus with a prodrug of cidofovir.

### BACKGROUND OF THE INVENTION

Cidofovir is taken up by pinocytosis and requires intravenous infusion that can result in nephrotoxicity. The lipid analogue, hexadecyloxypropyl-cidofovir (HDP-CDV), is orally bioavailable and no nephrotoxicity has been detected in preclinical toxicity studies or human trials. HDP-CDV is under development as an active IND drug (Bidanset D J, et al. Oral Activity of Ether Lipid Ester Prodrugs of Cidofovir Against Experimental Human Cytomegalovirus Infection. J Infect Dis (2004) 190(3):499-50). HDP-CDV (1-O-hexadecyloxypropyl cidofovir, HDP-cidofovir) is a lipid conjugate of cidofovir. Mechanistically, the lipid moiety dictates the drug's pharmacokinetic properties in target organs, while the antiviral activity is contained within the nucleotide residue. Compared to cidofovir, which is taken up into cells by inefficient processes, the conjugate is designed to act like lysophosphatidylcholine (LPC) utilizing natural lipid uptake pathways to achieve high intracellular concentrations. Once inside target cells, the lipid side chain of HDP-CDV is cleaved, presumably by phospholipase C, to yield free cidofovir. Conversion of cidofovir to the active antiviral agent, cidofovir-PP (cidofovir diphosphate), occurs via a two-step phosphorylation process catalyzed by intracellular anabolic kinases. Cidofovir-PP exerts its antiviral effects intracellularly by acting as a potent alternate substrate inhibitor of viral DNA synthesis.

Improving drug bioavailability is an established goal in the medical arts. It is important in pharmacology that a drug has sufficient bioavailability for its therapeutic purpose. The sequence of events for an oral composition includes absorption through the various mucosal surfaces, distribution via the blood stream to various tissues, biotransformation in the liver and other tissues, action at the target site, and elimination of drug or metabolites in urine or bile. Bioavailability can be reduced by poor absorption from the gastrointestinal tract, hepatic first-pass effect, or degradation of the drug prior to reaching the circulatory system.

There is a challenge to maximizing the effectiveness of the lipid prodrug or derivative in the body due to metabolic or other undesired actions on the drugs in vivo. This has been a particular problem with lipid derivatives, given the body's elaborate and complex mechanisms for degrading and synthesizing lipids.

There is a need for methods and compositions to treat viral infections with improved lipophilic compounds while minimizing the impact of drug metabolism and interactions on the therapeutic agent.

### SUMMARY OF THE INVENTION

The present application provides methods and compositions for treatment or prevention of dsDNA virus infection in post-hematopoietic cell transplant (HCT or HSCT) patients.

The embodiments of the current invention also provides a method of treatment, prevention, or delaying on-set of cytomegalovirus (CMV) infection or CMV infection associated disease or disorder, by orally administering to a subject a pharmaceutical composition of a therapeutically effective dose of a compound of formula:

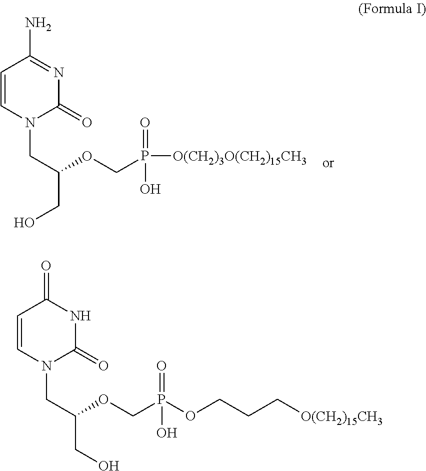

or a pharmaceutically acceptable salt thereof, where the subject may be a post-hematopoietic stem cell transplant (HSCT) subject and may be CMV seropositive before transplantation.

In one embodiment, the current invention provides a method of treating, preventing, or delaying time-to-onset of CMV infection and/or CMV infection associated disease or disorder in a HSCT recipient with once a week (QW) with about 200 mg or twice a week (BIW) about 100 mg of the compound of formula:

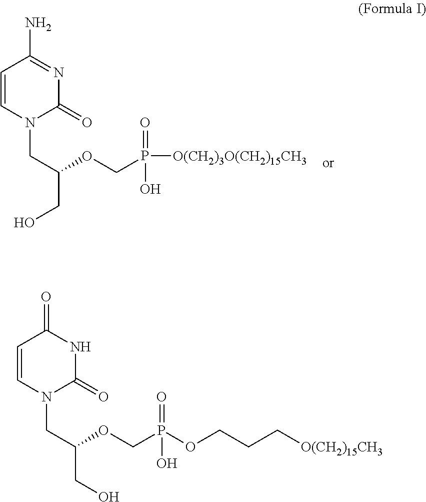

In some embodiments, the subject may be treated with about 100 mg of the compound(s) of the current invention, BIW. In additional embodiments, the subject may be treated once a week (QW) with about 150 mg or about 200 mg, or twice a week (BIW) with about 75 mg or about 100 mg of the compound.

The current embodiments also provides a method of prophylactic treatment, prevention, or delaying on-set of cytomegalovirus (CMV) infection or CMV infection related disease or disorder by orally administering to a subject a pharmaceutical composition comprising a therapeutically effective dose of a compound of formula:

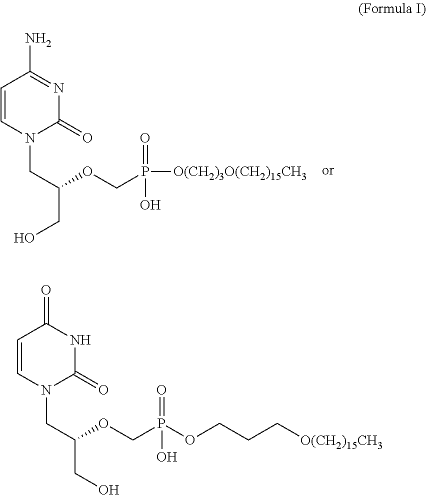

or a pharmaceutically acceptable salt thereof, where the subject may be a post-hematopoietic stem cell transplant (HSCT) subject and may be CMV seronegative before transplantation.

In some embodiments, a CMV seronegative HSCT recipient may be administered QW or BIW about 100 mg of the compounds of the current invention or a pharmaceutically acceptable composition thereof. The CMV seronegative HSCT recipient may be administered QW with about 150 mg or about 200 mg, or BIW with about 75 mg or about 100 mg of the compound of the current invention or a pharmaceutically acceptable composition thereof.

In one embodiment, pharmaceutically acceptable compositions are provided that include an antiviral lipid-containing compound, or salt, ester or prodrug thereof, and one or more bioavailability enhancing compounds. The compositions may be administered to a host in need thereof in an effective amount for the treatment or prophylaxis of a host infected with a virus, such as an adenovirus.

In one embodiment, a method of treating, preventing, or time-to-onset of a viral infection and/or viral infection associated disease or disorder, e.g., CMV infection, is provided, the method comprising administering an effective amount of antiviral lipid-containing compound, or salt, ester or prodrug thereof, and one or more bioavailability enhancing compounds to a host in need thereof. The compositions may be administered in an effective amount for the treatment or prophylaxis of a host infected with a virus, such as an adenovirus, optionally in combination with a pharmaceutically acceptable carrier. The compounds or compositions are administered, e.g., orally or parenterally.

In one embodiment, a method of treating, preventing, or delaying time-to-onset of a viral infection and/or viral infection associated disease or disorder (e.g., a cytomegalovirus infection, Epstein-Barr virus infection, herpes simplex virus infection, human herpes virus 6 infection, vaccinia virus infection, molluscum contagiosum virus infection) is provided, the method comprising administering an effective amount of antiviral lipid-containing compound, or salt, ester or prodrug thereof, and one or more bioavailability enhancing compounds to a host in need thereof. The compounds or compositions are administered, e.g., orally or parenterally.

In one embodiment, a method of treating a viral infection, e.g., a double stranded DNA (dsDNA) viral infection, is provided, the method comprising administering an effective amount of antiviral lipid-containing compound, or salt, ester or prodrug thereof, and one or more bioavailability enhancing compounds to a host in need thereof. The compositions may be administered in an effective amount for the treatment or prophylaxis of a host infected with a virus, such as a dsDNA virus, optionally in combination with a pharmaceutically acceptable carrier. The compounds or compositions are administered, e.g., orally or parenterally.

In one embodiment, a method of preventing a viral infection, e.g., CMV or an adenovirus infection, is provided, the method comprising administering an effective amount of a prodrug of an anti-viral nucleoside containing a lipid group, or salt, ester or prodrug thereof, and one or more bioavailability enhancing compounds to a host in need thereof, wherein the bioavailability enhancer in one embodiment is an agent that reduces the degradation of the lipid group. The compositions may be administered in combination or alternation in an effective amount for the prophylaxis of a host susceptible to infection with a virus, such as an orthopox virus or an adenovirus, optionally in combination with a pharmaceutically acceptable carrier. The compounds or compositions are administered, e.g., orally or parenterally.

In one embodiment, pharmaceutical compositions are provided that may include an amount of bioavailability enhancer effective to improve the bioavailability of the antiviral lipid-containing compound in comparison to that when the compound is administered alone. In another embodiment, the enhancer is administered sequentially or together with the antiviral lipid-containing compound in an amount effective to enhance the bioavailability of the antiviral compound in comparison to that when the antiviral compound is administered without the enhancer.

In one embodiment, the antiviral compound is cidofovir, adefovir, or cyclic cidofovir, optionally covalently linked to a lipid, or linked to an alkylglycerol, alkylpropanediol, 1-S-alkylthioglycerol, alkoxyalkanol or alkylethanediol. The enhancer may be, for example without being limited to those disclosed herein, an imidazole antifungal, e.g., ketoconazole or troleandomycin; a macrolide, such as erythromycin; a calcium channel blocker, such as nifedipine; or a steroid, such as gestodene. Optionally, the compound may be an inhibitor of cytochrome P450 3A (CYP3A), such as naringenin, found in grapefruit.

In one particular embodiment, a composition is provided that includes a cidofovir lipid prodrug and a bioavailability enhancer, administered to a subject in an effective amount for the treatment of a viral infection, such as a CMV infection. In one embodiment, the nucleoside prodrug is an alkoxyalkyl ester of cidofovir, such as an alkoxyalkanol of cidofovir (HDP-cidofovir or HDP-CDV). For example, the compound may have the structure:

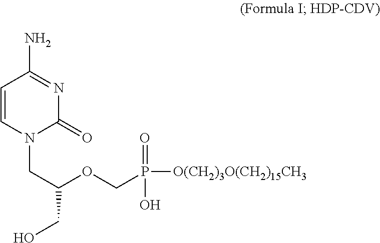

HDP-CDV is a broad spectrum lipid acyclic nucleoside phosphonate that may be converted intracellularly into the active antiviral cidofovir diphosphate (CDV-PP), which may have a long intracellular half-life of ˜6.5 days.

In one embodiment, the nucleoside prodrug of the current invention is obtained by deamination or hydrolysis of an alkoxyalkyl ester of cidofovir, such as an alkoxyalkanol of cidofovir (HDP-cidofovir or HDP-CDV). For example, the compound derived by deamination or hydrolysis of HDP-CDV may have the following structure:

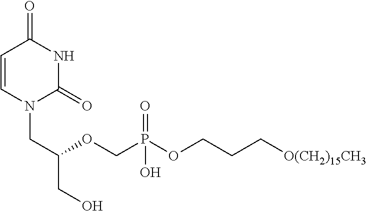

### BRIEF DESCRIPTION OF THE DRAWINGS

FIG. 1 illustrates time from first dose to CMV DNAemia (≧100 copies/mL) during treatment, for subjects who were CMV negative at baseline [modified CMV negative, mITT population].

### DETAILED DESCRIPTION OF THE INVENTION

Methods are provided for improving the bioavailability of a lipid containing prodrug, wherein the prodrug is administered in combination or alternation with a bioavailability enhancer. Also provided are pharmaceutically acceptable compositions, comprising a lipid containing prodrug and a bioavailability enhancer. The prodrug in one embodiment is an antiviral lipid-containing compound, such as cidofovir linked to a lipid.

In some embodiments, about 200 mg of the compound(s) of the current invention may be administered once a week (QW) or about 100 mg of the compound(s) twice a week (BIW) to a subject. The subject may be treated QW with about 150 mg or about 200 mg, or BIW with about 75 mg or about 100 mg of the compound. In yet other embodiments, a subject may be treated with about 50-99 mg, 101-149 mg, 151-199 mg, 201-250 mg, or >251 mg dose without resulting in significant adverse effects (AEs). In some embodiments, the dose in mg may vary within one week, two weeks, or during the entire treatment period.

The current invention provides a method of preventing a disease or disorder in a subject at risk of virus infection reactivation, by orally administering to the subject a pharmaceutical composition of a therapeutically effective dose of a compound of formula:

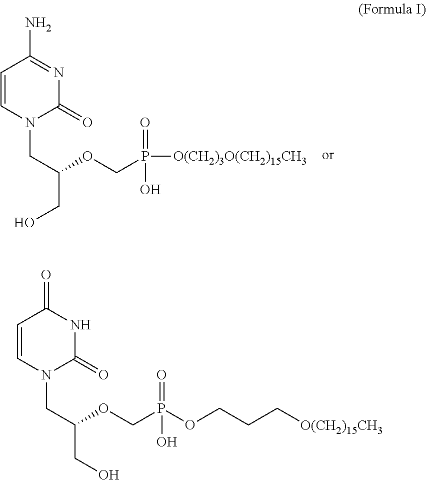

or a pharmaceutically acceptable salt thereof, wherein the virus at risk of reactivation may be CMV.

In one embodiment, the subject at risk of virus infection reactivation may be stem cell transplant (e.g., HSCT) or renal transplant recipients. In an embodiment, the subject may be a post-hematopoietic stem cell transplant (HSCT) subject. In yet other embodiments, the subject may be islet cell transplant recipient, bone marrow transplant recipient, endothelial cell transplant recipient, epidermal cell transplant recipient, myoblast transplant recipient, muscle derived stem cell recipient, and/or neural stem cell transplant recipient.

In yet other embodiments, the subject may be islet cell transplant recipient, bone marrow transplant recipient, endothelial cell transplant recipient, epidermal cell transplant recipient, myoblast transplant recipient and/or neural stem cell transplant recipient.

In yet another embodiment, the method of the current invention prevents hematuria or renal impairment in a post-HSCT subject. The prevention of hematuria or renal impairment in post-HSCT patient may be associated with prevention of viral reactivation in the subject. In one embodiment, the prevention of virus infection reactivation prevents hematuria or renal impairment in said subject.

The embodiments of the current invention also provides a method of treatment, prevention, or delaying on-set of cytomegalovirus (CMV) infection or CMV infection associated disease or disorder, by orally administering to a subject a pharmaceutical composition of a therapeutically effective dose of a compound of formula:

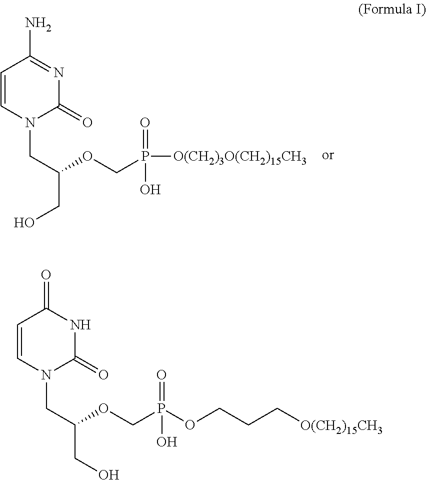

or a pharmaceutically acceptable salt thereof, where the subject is a post-hematopoietic stem cell transplant (HSCT) subject and is CMV seropositive before transplantation. In yet other embodiments, the subject may be islet cell transplant recipient, bone marrow transplant recipient, endothelial cell transplant recipient, epidermal cell transplant recipient, myoblast transplant recipient, muscle derived stem cell recipient, and/or neural stem cell transplant recipient.

In one embodiment, the current invention provides a method of treating or preventing CMV infection and/or delaying time-to-onset of a disease or disorder associated with CMV infection in a HSCT recipient with once a week (QW) or twice a week (BIW) about 100 mg of the compound of formula:

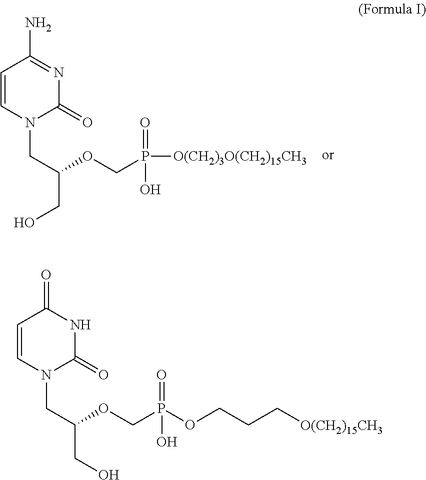

In some embodiments, about 200 mg of the compound(s) of the current invention may be administered once a week (QW) or with about 100 mg twice a week (BIW) to a subject for treating CMV infection. In additional embodiments, the subject may be treated QW with about 150 mg or about 200 mg, or BIW with about 75 mg or about 100 mg of the compound. In yet other embodiments, a subject may be treated with about 50-99 mg, 101-149 mg, 151-199 mg, 201-250 mg, or >251 mg dose without resulting in significant adverse effects (AEs). In some embodiments, the dose in mg may vary within one week, two weeks, or during the entire treatment period.

The current embodiments also provides a method of prophylactic treatment, prevention and/or delaying time-to-onset of a disease or disorder associated with CMV infection by orally administering to a subject a pharmaceutical composition comprising a therapeutically effective dose of a compound of formula:

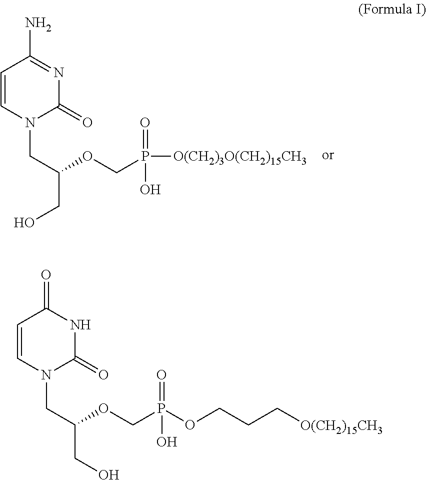

or a pharmaceutically acceptable salt thereof, where the subject is a post-hematopoietic stem cell transplant (HSCT) subject and is CMV seronegative before transplantation. In yet other embodiments, the subject may be islet cell transplant recipient, bone marrow transplant recipient, endothelial cell transplant recipient, epidermal cell transplant recipient, myoblast transplant recipient, muscle derived stem cell recipient, and/or neural stem cell transplant recipient.

In some embodiments, a CMV seronegative HSCT recipient may be treated QW with about 200 mg or BIW with about 100 mg of the compounds of the current invention or a pharmaceutically acceptable composition thereof. The CMV seronegative HSCT recipient may be treated QW with about 150 mg or about 200 mg, or BIW with about 75 mg or about 100 mg of the compounds of the current invention or a pharmaceutically acceptable composition thereof.

In some embodiments, about 100 mg of the compound(s) of the current invention may be administered BIW to a CMV seronegative subject. In additional embodiments, the subject may be treated QW with about 150 mg or about 200 mg, or BIW with about 75 mg or about 100 mg of the compounds. In yet other embodiments, a subject may be treated with about 50-99 mg, 101-149 mg, 151-199 mg, 201-250 mg, or >251 mg dose without resulting in significant adverse effects (AEs). In some embodiments, the dose in mg may vary within one week, two weeks, or during the entire treatment period.

In one particular embodiment, a composition may be provided that includes a cidofovir lipid prodrug and a bioavailability enhancer, administered to a subject in an effective amount for the treatment of a viral infection, such as a CMV infection. In one embodiment, the nucleoside prodrug may be an alkoxyalkyl ester of cidofovir, such as an alkoxyalkanol of cidofovir (HDP-cidofovir or HDP-CDV). For example, the compound may have the structure:

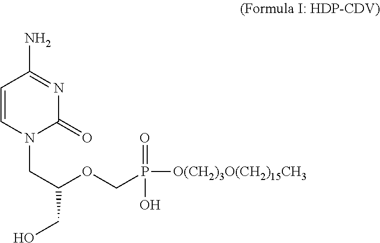

HDP-CDV is a broad spectrum lipid acyclic nucleoside phosphonate that may be converted intracellularly into the active antiviral cidofovir diphosphate (CDV-PP), which may have a long intracellular half-life of ˜6.5 days.

In one embodiment, the nucleoside prodrug of the current invention may be obtained by deamination or hydrolysis of HDP-CDV alkoxyalkyl ester of cidofovir, such as an alkoxyalkanol of cidofovir (HDP-cidofovir or HDP-CDV). For example, the compound derived by deamination or hydrolysis of HDP-CDV may have the structure:

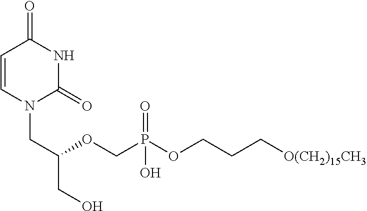

The embodiments of the current invention provide reduction of CMV DNAemia, or treatment of CMV disease or CMV disease progression in post-HSCT subjects. In certain embodiment, the invention provides a trend toward lower use of antivirals for CMV preemptive therapy in subjects who may have received 100 mg or higher dose of the compound(s) of the current invention versus placebo and/or 40 mg of the compound.

In some embodiments, higher doses of the compound(s) (about 200 mg QW, about 100 mg BIW, and about 200 mg BIW) may be superior to placebo in reducing the proportion of subjects reaching a composite endpoint of initiation of anti-viral therapy or viral disease or viral dsDNAemia of >1000 copies/mL. In one embodiment, the invention provides a trend in subjects who may have received about 100 mg QW of the compound(s) versus placebo in preventing the viral disease and/or infection outcome, and the about 40 mg QW dose was ineffective. The virus infection and/or virus associated disease or disorder may be CMV or CMV related disease or disorder. The compound of the invention may be of the formula:

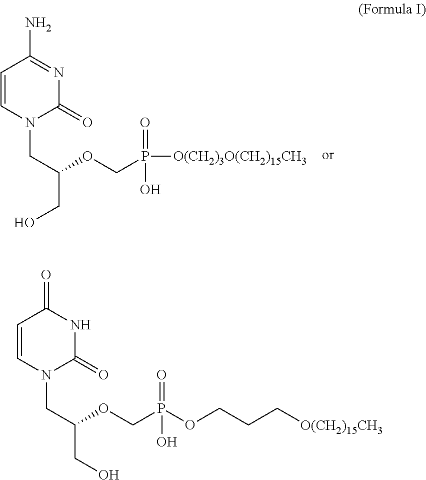

In one embodiment, CMV DNAemia prior to dosing was evaluated in pooled HDP-CDV treatment groups, which showed that HDP-CMV achieved a statistically significant reduction of CMV DNAemia in treated groups compared to the placebo group. The analysis was adjusted for the presence or absence of CMV DNAemia prior to dosing (p=0.041).

In one embodiment of the current invention, the proportion of subjects who received about 100 mg BIW of HDP-CDV and developed CMV disease or CMV DNAemia at the end of treatment was 10% versus 37.3% for placebo-treated subjects (p=0.001; TABLE 2).

In an embodiment of the current invention, except for a dose of 40 mg QW, other HDP-CDV doses and dose regimens, of the current invention, may have better antiviral activity when compared to placebo. Activity of the compound may increase with dose and/or dose frequency.

In subjects CMV negative at baseline, the pooled HDP-CDV group or each dose regimen of 100 mg QW or greater may be superior compared to placebo. See TABLE 5. In a particular embodiment, no subjects may developed CMV DNAemia>1000 copies/mL, compared to placebo-treated subjects, when treated with about 100 and/or about 200 mg BIW. See TABLE 6.

In another embodiment of the current invention, pooled HDP-CDV may be numerically superior to placebo (22% versus 31%), as was the 100 mg BIW group (14%) (TABLE 7). The proportion of subjects reaching the composite endpoint of initiation of anti-CMV therapy or CMV disease or CMV DNAemia>1,000 copies/mL. Higher doses of HDP-CDV (≧200 mg QW) may be superior to placebo in reducing the incidence of this composite endpoint (less than 15% for these doses versus 38.3% for placebo; p≦0.05 for 200 mg QW and 200 mg BIW; p<0.01 for 100 mg BIW). See TABLE 8.

The embodiments of the current invention provide that a trend may be apparent in subjects who receive HDP-CDV about 100 mg QW versus placebo in preventing viral infection and/or end-organ disease or impairment outcome (21.7% versus 38.3%). In an embodiment about 40 mg QW dose may be inactive. The embodiments of the current invention provide evidence for a dose-dependent response to treatment.

In one embodiment, the current invention provides that about 100 mg BIW dose regimen may result in a lower frequency and/or lower overall levels of CMV DNAemia when visually compared to either placebo or about 200 mg QW, suggesting that BIW dosing may have an impact on CMV suppression over and above the total weekly administered dose.

The embodiments of the current invention provide that no UL97 and/or UL54 mutations, for drug resistance, may be detected in subjects enrolled on active drug in the studies of the current application. In specific embodiments, the current invention provides that no mutation in UL54 and UL97 genes may be observed in patients receiving dosing regimen of about ≧100 mg QW.

The embodiments of the current invention provide that there may be no indication of nephrotoxicity or myelotoxicity associated with HDP-CDV, regardless of dose and dosing frequency. HDP-CDV dose regimens of about 40 mg QW and about 100 mg QW may have tolerability profiles similar to placebo in terms of AEs and laboratory abnormalities. A dose-related increase in ALT may be associated with HDP-CDV therapy.

In additional embodiments, the current invention provides that the safety profile of HDP-CDV about 200 mg per week may be acceptable in the context of the benefit derived from the prevention of CMV reactivation as compared to the safety profile of preemptive therapy which is the current standard of care.

In some embodiments, the compound of the current invention may be administered orally with food. Food may be consumed before, concurrently, or after administration of the compound or a pharmaceutically acceptable salt and/or composition thereof. Food may be consumed about 30 min., 60 min., 90 min., 120 min., 150 min., 180 min., 210 min., 240 min., or >270 min. before or after the administration of the compound.

The embodiments of the current invention also provide that once treatment is completed subjects treated with HDP-CDV at active antiviral doses may be less likely to need preemptive (i.e., initiation of treatment based on the detection of viral replication during regular monitoring) and/or other interventions.

In some embodiments, the current invention provides substantial reduction of CMV viral load (“VL”) in highly immunosuppressed HCT subjects with refractory CMV or intolerance to other antivirals. Subjects may have life-threatening or serious disease or condition caused by infection with a dsDNA virus (for e.g., CMV), and may have life-expectancy of about 2 weeks or more and with a commitment to continuation of supportive care for about 4 weeks or more. Some subjects may have no available comparable or satisfactory therapeutic alternative.

In some embodiments, subjects may receive either a weight-based or fixed dose or HDP-CDV. Subjects may be pediatric (12 years or less) and may receive about 4 mg/kg total weekly dose (may not exceed about 200 mg), as either 2 mg/kg twice-weekly (BIW) or about 4 mg/kg once-weekly (QW). Adults and adolescent (13-17 years old) subjects may receive about 200 mg (not to exceed 4 mg/kg) total weekly dose, as either 100 mg BIW or 200 mg QW. BIW doses may be administered at alternating 3- and 4-day intervals and QW dose on the same day each week.

The embodiments of the current also provides that subjects may be treated for an initial period of up to 3 months until either resolution or stabilization of their clinical disease or for 4 weeks following resolution of viral DNAemia, depending on the disease under treatment. Treatment may be extended for up to an additional 3 months depending on the subject's clinical response.

In yet other embodiments of the current invention the virologic responses of subjects with no CMV disease may be measured. Subjects may have no CMV disease and may have had their last on-treatment CMV DNAemia value about ≦1,000 copies/mL. The CMV DNAemia values for most subjects may be reduced to the lower limit of quantitation (“LLOQ”) (100 copies/mL or 2 log₁₀ copies/mL) of the CMV qPCR assay. One subject may have preexisting Ganciclovir (“GCV”) resistance (“GCV-R”) mutations at H520Q and A594P.

In one embodiment a transient virologic response may be observed in subject(s) being treated with the compounds of the current invention. Subjects showing transient virologic response may have a preexisting GCV-R mutation at L595F, and may have developed CMV disease on-treatment, suggesting the development of resistance to GCV. In another embodiment, subject(s) with a preexisting GCV+FOS resistance mutation at V781I and CMV colitis at baseline may achieve CMV DNAemia values of <LLOQ, before eventually succumbing to GVHD.

In another embodiment of the current invention, subject(s) may complete about 6-month period of HDP-CDV treatment. Subject's CMV DNAemia may decline at the end of treatment (last on-treatment value may be about 1,000 copies/mL) and may be ≦LLOQ at the +1 month posttreatment follow-up (“FU”) visit. In one embodiment, subjects being treated may have preexisting resistance mutations.

In additional embodiments, the current invention provides the time to onset of viral DNAemia (for e.g., CMV DNAemia) may be delayed in subjects, who may be negative for the viral DNA at baseline and received about 100 mg QW, about 200 mg BIW, and about 100 mg BIW of the compound(s) of the current invention, as compared to placebo. The time to onset may be delayed by several days, for e.g., 2-10 days, 3-11 days, 4-12 days, 5-13 days, 6-14 days, 7-15 days, 8-16 days, 9-17 days, 10-18 days, 11-19 days, 12-20 days, 13-21 days, 14-22 days, 15-23 days, 16-24 days, 17-25 days, or >25 days.

In additional embodiments, the current invention provides prevention of varicella-zoster virus (“VZV”) spread. Subjects may be treated with an effective dose of HDP-CDV or the deaminated or hydrolyzed compound of HDP-CDV to prevent the spread of VZV. In some embodiments, the compounds of the current invention, for e.g., HDP-CDV, may prevent VZV spread by about 2-10 folds, 11-19 folds, or 20-25 folds. In one embodiment, the compounds of the current invention may prevent VZV spread by about 10 fold.

In certain embodiments, the compound of the current invention, for e.g., HDP-CDV, may prevent zoster infections, which may otherwise be difficult to treat with other drug(s) or therapeutic means. The compounds of the current invention may prevent post-herpetic neuralgia.

The compounds of the current invention may be used to treat and/or prevent neuropathic pain caused by a disease or symptom associated with a viral infection, including, for example, an inflammatory disorder, a neoplastic tumor, an acquired immune deficiency syndrome (AIDS), shingles, and/or other herpes infection.

Types of neuropathic pain for treatment with the compounds of the current invention include “Neuralgia.” A neuralgia is a pain that radiates along the course of one or more specific nerves usually without any demonstrable pathological change in the nerve structure. Neuralgia is most common in elderly persons, but it may occur at any age. According to the current invention, neuralgia may include, without limitation, a trigeminal neuralgia, a post-herpetic neuralgia, a post-herpetic neuralgia, a glossopharyngeal neuralgia, a sciatica and an atypical facial pain.

Neuralgia is pain in the distribution of a nerve or nerves. Examples are trigeminal neuralgia (“TN”), atypical facial pain, and post-herpetic neuralgia (caused by shingles or herpes). The affected nerves are responsible for sensing touch, temperature and pressure in the facial area from the jaw to the forehead. The disorder generally causes short episodes of excruciating pain, usually for less than two minutes and on only one side of the face. The pain can be described in a variety of ways such as “stabbing,” “sharp,” “like lightning,” “burning,” and even “itchy.” In the atypical form of TN, the pain can also present as severe or merely aching and last for extended periods. The pain associated with TN is recognized as one the most excruciating pains that can be experienced.

According to the current invention, compounds may be used for treating or ameliorating Neuralgia, which may occur after infections such as shingles—caused by the varicella-zoster virus, a type of herpesvirus. This neuralgia in the subject population may produce a constant burning pain after the shingles rash has healed. The pain may be worsened by movement of or contact with the affected area. Not all of those diagnosed with shingles in the current subject population may go on to experience post-herpetic neuralgia, which may be more painful than shingles. The pain and sensitivity in the subject population may last for months or even years. The pain may be in the form of an intolerable sensitivity to any touch, including light touch. Post-herpetic neuralgia, which may be treated or ameliorated with the compounds of the current invention, may occur anywhere on the body—especially at the location of the shingles rash—including the face.

The compounds of the current invention may be administered in conjunction with Granulocyte Colony-Stimulating Factor (G-CSF) and/or blood or blood product transfusion. The subjects receiving both HDP-CDV or a derivative and/or salts thereof, may be require less blood or blood product transfusion, compared to the subjects receiving placebo. The subjects receiving both HDP-CDV, for example, and G-CSF and/or blood or blood product transfusion may be effectively treated for pre-emptive and/or prophylactic treatment of CMV. Subjects may receive any other growth factors including, without being limited to the examples herein, Adrenomedullin (AM), Angiopoietin (Ang), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha (TGF-α), Transforming growth factor beta (TGF-β), Tumor necrosis factor-alpha (TNF-α), Vascular endothelial growth factor (VEGF), Wnt Signaling Pathway, and/or placental growth factor (PlGF).

### Prodrug Compounds

Prodrugs of a variety of compounds may be used in the methods and compositions disclosed herein. In particular, the prodrug may be one that includes a hydrocarbon chain, for example, a C4-C30, or a C8-22 hydrocarbon chain. The drug can be any of a variety of drugs, such as a variety of anticancer or antiviral compounds.

In one embodiment the prodrug is a prodrug of a nucleoside including phosphonates and phosphates. In a particular embodiment, the prodrug is antiviral lipid-containing nucleoside, such as an anti-orthopox agent.

The prodrug in one embodiment is the prodrug of an antiviral compound. The prodrug is, for example, cidofovir, adefovir, or cyclic cidofovir, e.g., covalently linked to a lipid, such as an alkylglycerol, alkylpropanediol, 1-S-alkylthioglycerol, alkoxyalkanol or alkylethanediol, or a lipid containing a C₈₋₃₀ alkyl alkenyl or alkynyl. As used herein, where a compound is “covalently linked to a lipid” the compound may include a linker between the compound and the lipid group. The lipid group is e.g., a C₈₋₃₀ alkyl, alkenyl or alkynyl.

In one embodiment, the antiviral prodrug is cidofovir, e.g., covalently linked to a lipid.

In one embodiment, the antiviral prodrug has the structure:

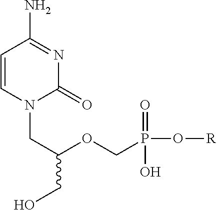

wherein R is H; optionally substituted alkyl, e.g., C₁₋₃₀ alkyl; alkenyl, e.g., C₂₋₃₀ alkenyl; or alkynyl, e.g., C₂₋₃₀ alkynyl; acyl; mono- or di-phosphate; alkylglycerol, alkylpropanediol, 1-S-alkylthioglycerol, alkoxyalkanol or alkylethanediol. In one embodiment R is an alkoxyalkanol. For example, R is —(CH₂)ₘ—O—(CH₂)ₙ—CH₃ wherein, e.g., m is 1-5 and n is 1-25; or m is 2-4 and n is 10-25.

In another embodiment, the antiviral prodrug compound has the following structure:

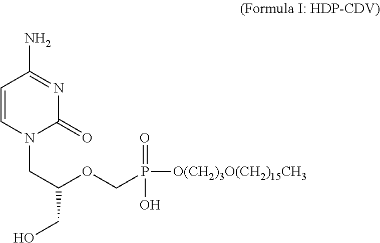

HDP-CDV is an orally administered lipid conjugate of the synthetic nucleotide analog CDV, which is absorbed in the small intestine and delivered to target organs throughout the body. Inside the cell, HDP-CDV is cleaved to release CDV, presumably by phospholipases, which is converted to the active antiviral agent, CDV-diphosphate (CDV-PP), by intracellular anabolic kinases. This active metabolite of HDP-CDV has been shown to have a long intracellular half-life of approximately 6.5 days.

HDP-cidofovir (HDP-CDV) has broad spectrum in vitro antiviral activity against all dsDNA viruses of importance to immunocompromised patients, including transplant recipients and has not been associated with either myelosuppression or nephrotoxicity in extensive clinical trials. For comparison with HDP-cidofovir, the in vitro antiviral activity of relevant, currently available nucleoside/nucleotide analogs against AdV, CMV, EBV, herpes simplex virus 1 (HSV-1), VZV and human herpesvirus 6 (HHV-6), assessed in cell culture experiments, is presented in TABLE 1.

Cell culture experiments have demonstrated>100 to 1000-fold higher potency in comparison to cidofovir, ganciclovir and other nucleoside analogs against dsDNA viruses of interest, including cytomegalovirus (CMV) and adenovirus (AdV).

TABLE 1
Comparison of in vitro Antiviral Activity of
Nucleoside/Nucleotide Analogs

| | EC50s (μM) | | | | |
|---|---|---|---|---|---|
| | HDP- | | | | |
| Virus | CDV | Cidofovir | Ganciclovir | Maribavir | Acyclovir |
| CMV | 0.001 | 0.4 | 3.8 | 0.31 | >200 |
| VZV | 0.0004 | 0.5 | 1.3 | No Activity | 3.6 |
| HHV-6(B) | 0.007 | 5.4 | 5.9 | ND | 100 |
| AdV | 0.02 | 1.3 | 4.5-33 | ND | >100 |
| EBV | 0.04 | >108 | 0.9 | 0.63 | 8.5 |
| HSV-1 | 0.06 | 5.5 | 0.007 | No Activity | 2.5 |

In one embodiment, the antiviral prodrug is adefovir, e.g., covalently linked to a lipid group.

Prodrugs of other compounds also may be used including prodrugs of the following agents: analgesic; anesthetic; anorectic; anti-adrenergic; anti-allergic; anti-anginal; anti-anxiety; anti-arthritic; anti-asthmatic; anti-atherosclerotic; antibacterial; anticoagulant; anticonvulsant; antidepressant; antidiabetic; antidiarrheal; antidiuretic; anti-estrogen; antifibrinolytic; antifungal; antiglaucoma agent; antihistamine; anti-infective; anti-inflammatory; antikeratinizing agent; antimalarial; antimicrobial; antimigraine; antimitotic; antimycotic, antinauseant, antineoplastic, antineutropenic, antiobessional agent; antiparasitic; antiparkinsonian; antiperistaltic, antipneumocystic; antiproliferative; liver disorder treatment; psychotropic; serotonin inhibitor; serotonin receptor antagonist; steroid; stimulant; suppressant; thyroid hormone; thyroid inhibitor; thyromimetic; tranquilizer; agent for treatment of amyotrophic lateral sclerosis; agent for treatment of cerebral ischemia; agent for treatment of Paget's disease; agent for treatment of unstable angina; uricosuric; vasoconstrictor; vasodilator; vulnerary; or a wound healing agent.

### Methods of Treatment

Methods of treating, preventing, or ameliorating disorders such as viral infections are provided herein. In practicing the methods, effective amounts of a prodrug, e.g. of an anti-viral compound, in particular, an antiviral lipid-containing compound and an enhancer, sequentially or in combination, are administered. The compounds may be administered in any desired manner, e.g., via oral, rectal, nasal, topical (including buccal and sublingual), vaginal, or parenteral (including subcutaneous, intramuscular, subcutaneous, intravenous, intradermal, intraocular, intratracheal, intracisternal, intraperitoneal, and epidural) administration. The compounds may be administered in combination or alternation by the same or different route of administration.

In certain embodiments, the viral infections that can be treated include influenza; pestiviruses such as bovine viral diarrhea virus (BVDV), classic swine fever virus (CSFV, also known as hog cholera virus), and Border disease virus of sheep (BDV); flaviviruses like dengue hemorrhagic fever virus (DHF or DENV), yellow fever virus (YFV), West Nile virus (WNV), shock syndrome and Japanese encephalitis virus; hepatitis B and C virus; cytomegalovirus (CMV); herpes infections, such as those caused by Varicella zoster virus, Herpes simplex virus types 1 & 2, human herpes virus 6, Epstein-Barr virus, Herpes type 6 (HHV-6) and type 8 (HHV-8); Varicella zoster virus infections such as shingles or chicken pox; Epstein Barr virus infections, including, but not limited to infectious mononucleosis/glandular; retroviral infections including, but not limited to SIV, HIV-1 and HIV-2; Ebola virus; adenovirus and papilloma virus.

In further embodiments, the anti-viral compounds and the enhancer are administered in an effective amount for the treatment or prophylaxis of viral infections resulting from orthopox viruses, such as variola major and minor, vaccinia, molluscum contagiosum, orf (eethyma contagiosum) smallpox, cowpox, camelpox, mousepox, rabbitpox, and monkeypox.

In one embodiment, a therapeutically effective dosage to treat such an orthopox infection should produce a serum concentration of anti-viral agent of about 0.1 ng/ml to about 50-100.mu.g/ml. The pharmaceutical compositions, in another embodiment, should provide a dosage of from about 0.001 mg to about 2000 mg of compound per kilogram of body weight per day. Pharmaceutical dosage unit forms are prepared, e.g., to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The amount of the enhancer can be selected using methods known in the art to enhance the bioavailability of the anti-viral agent. Any amount can be used that provides an desired response. The dosages may range, in a non-limiting example, from 0.001 mg to about 2000 mg of compound per kilogram of body weight per day, e.g. 0.01 to 500 mg/kg, or e.g., 0.1-10 mg/kg.

### Combination Therapy

The compounds and compositions provided herein may also be used in combination, and alternatively, in combination with other active ingredients. In certain embodiments, the compounds may be administered in combination, or sequentially, with another therapeutic agent. Such other therapeutic agents include those known for treatment, prevention, or amelioration of one or more symptoms associated with viral infections. The compounds and/or composition provided herein may be used in combination with one or more of: midazolam, cyclosporin A, tacrolimus, azoles, ganciclovir (CYTOVENE®, GCV), valganciclovir (VALCYTE®, vGCV), foscavir, cidofovir, second-line anti-CMV drugs, foscarnet (FOSCAVIR®, FOS), intravenously administered (IV) cidofovir (CDV, VISTIDE®), filgrastim (NEUPOGEN®), pegfilgrastim (NEULASTA®), corticosteroids such as budesonide, beclomethasone, and broad-spectrum CYP inhibitor aminobenzotriazole (ABT).

It should be understood that any suitable combination of the compounds provided herein with one or more of the above-mentioned compounds and optionally one or more further pharmacologically active substances are considered to be within the scope of the present disclosure. In another embodiment, the compound provided herein is administered prior to or subsequent to the one or more additional active ingredients. In one embodiment, two or more of the antiviral agents disclosed herein are administered serially or in combination.

### Pharmaceutical Compositions

Pharmaceutical carriers suitable for administration of the compounds provided herein include any such carriers known to those skilled in the art to be suitable for the particular mode of administration. The compounds may be formulated as the sole pharmaceutically active ingredient in the composition or may be combined with other active ingredients.

Compositions comprising the compounds disclosed herein may be suitable for oral, rectal, nasal, topical (including buccal and sublingual), vaginal, or parenteral (including subcutaneous, intramuscular, subcutaneous, intravenous, intradermal, intraocular, intratracheal, intracistemal, intraperitoneal, and epidural) administration.

The compositions may conveniently be presented in unit dosage form and may be prepared by conventional pharmaceutical techniques. Such techniques include the step of bringing into association one or more compositions provided herein and one or more pharmaceutical carriers or excipients.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof may be mixed with one or more suitable pharmaceutical carriers. The compounds may be derivatized as the corresponding salts, esters, enol ethers or esters, acetals, ketals, orthoesters, hemiacetals, hemiketals, acids, bases, solvates, hydrates or prodrugs prior to formulation. The concentrations of the compounds in the compositions are effective for delivery of an amount, upon administration, that treats, prevents, or ameliorates one or more of the symptoms of the target disease or disorder. In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

Compositions suitable for oral administration may be presented as discrete units such as, but not limited to, tablets, caplets, pills or dragees capsules, or cachets, each containing a predetermined amount of one or more of the compositions; as a powder or granules; as a solution or a suspension in an aqueous liquid or a non-aqueous liquid; or as an oil-in-water liquid emulsion or a water-in-oil emulsion or as a bolus, etc.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents, preservatives, flavoring agents, and the like, for example, acetate, sodium citrate, cyclodextrine derivatives, sorbitan monolaurate, triethanolamine sodium acetate, triethanolamine oleate, and other such agents. Methods of preparing such dosage forms are known, or will be apparent, to those skilled in this art; for example, see Remington's Pharmaceutical Sciences, Mack Publishing Company, Easton, Pa., 15th Edition, 1975.

Compositions of the present invention suitable for topical administration in the mouth include for example, lozenges, having the ingredients in a flavored basis, usually sucrose and acacia or tragacanth; pastilles, having one or more of the compositions of the present invention in an inert basis such as gelatin and glycerin, or sucrose and acacia; and mouthwashes, having one or more of the compositions of the present invention administered in a suitable liquid carrier.

The tablets, pills, capsules, troches and the like can contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laurel ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

Compositions suitable for topical administration to the skin may be presented as ointments, creams, gels, and pastes, having one or more of the compositions administered in a pharmaceutical acceptable carrier.

Compositions for rectal administration may be presented as a suppository with a suitable base comprising, for example, cocoa butter or a salicylate.

Compositions suitable for nasal administration, when the carrier is a solid, include a coarse powder having a particle size, for example, in the range of 20 to 500 microns which is administered in the manner in which snuff is taken, (i.e., by rapid inhalation through the nasal passage from a container of the powder held close up to the nose). When the carrier is a liquid (for example, a nasal spray or as nasal drops), one or more of the compositions can be admixed in an aqueous or oily solution, and inhaled or sprayed into the nasal passage.

Compositions suitable for vaginal administration may be presented as pessaries, tampons, creams, gels, pastes, foams or spray formulations containing one or more of the compositions and appropriate carriers.

Compositions suitable for parenteral administration include aqueous and non-aqueous sterile injection solutions which may contain anti-oxidants, buffers, bacteriostats, and solutes which render the formulation isotonic with the blood of the intended recipient; and aqueous and non-aqueous sterile suspensions which may include suspending agents and thickening agents. The compositions may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules, and tablets of the kind previously described above.

Pharmaceutical organic or inorganic solid or liquid carrier media suitable for enteral or parenteral administration can be used to fabricate the compositions. Gelatin, lactose, starch, magnesium stearate, talc, vegetable and animal fats and oils, gum, polyalkylene glycol, water, or other known carriers may all be suitable as carrier media.

Compositions may be used as the active ingredient in combination with one or more pharmaceutically acceptable carrier mediums and/or excipients. As used herein, “pharmaceutically acceptable carrier” includes any and all carriers, solvents, diluents, or other liquid vehicles, dispersion or suspension aids, surface active agents, isotonic agents, thickening or emulsifying agents, preservatives, solid binders, lubricants, adjuvants, vehicles, delivery systems, disintegrants, absorbents, preservatives, surfactants, colorants, flavorants, or sweeteners and the like, as suited to the particular dosage form desired.

Additionally, the compositions may be combined with pharmaceutically acceptable excipients, and, optionally, sustained-release matrices, such as biodegradable polymers, to form therapeutic compositions. A “pharmaceutically acceptable excipient” includes a non-toxic solid, semi-solid or liquid filler, diluent, encapsulating material or formulation auxiliary of any type.

It will be understood, however, that the total daily usage of the compositions will be decided by the attending physician within the scope of sound medical judgment. The specific therapeutically effective dose level for any particular host will depend upon a variety of factors, including for example, the disorder being treated and the severity of the disorder; activity of the specific composition employed; the specific composition employed, the age, body weight, general health, sex and diet of the patient; the time of administration; route of administration; rate of excretion of the specific compound employed; the duration of the treatment; drugs used in combination or co-incidental with the specific composition employed; and like factors well known in the medical arts. For example, it is well within the skill of the art to start doses of the composition at levels lower than those required to achieve the desired therapeutic effect and to gradually increase the dosage until the desired effect is achieved.

Compositions are preferably formulated in dosage unit form for ease of administration and uniformity of dosage. “Dosage unit form” as used herein refers to a physically discrete unit of the composition appropriate for the host to be treated. Each dosage should contain the quantity of composition calculated to produce the desired therapeutic affect either as such, or in association with the selected pharmaceutical carrier medium.

Exemplary unit dosage formulations are those containing a daily dose or unit, daily sub-dose, or an appropriate fraction thereof, of the administered ingredient. The dosage will depend on host factors such as weight, age, surface area, metabolism, tissue distribution, absorption rate and excretion rate. Exemplary systemic dosages for all of the herein described conditions are those ranging from 0.01 mg/kg to 2000 mg/kg of body weight per day as a single daily dose or divided daily doses. Typical dosages for topical application are those ranging from 0.001 to 100% by weight of the active compound.

The therapeutically effective dose level will depend on many factors as noted above. In addition, it is well within the skill of the art to start doses of the composition at relatively low levels, and increase the dosage until the desired effect is achieved.

Compositions comprising a compound disclosed herein may be used with a sustained-release matrix, which can be made of materials, usually polymers, which are degradable by enzymatic or acid-based hydrolysis or by dissolution. Once inserted into the body, the matrix is acted upon by enzymes and body fluids. A sustained-release matrix for example is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (copolymers of lactic acid and glycolic acid).

The compounds may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The liposome can contain, in addition to one or more compositions of the present invention, stabilizers, preservatives, excipients, and the like. Examples of lipids are the phospholipids and the phosphatidyl cholines (lecithins), both natural and synthetic. Methods to form liposomes are known in the art.

The compounds may be formulated as aerosols for application, such as by inhalation. These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

Compositions comprising the compounds disclosed herein may be used in combination with other compositions and/or procedures for the treatment of the conditions described above.

### Clinical Studies

The clinical study of the current invention (HDP-CDV-201) was a 9-11 week randomized, placebo-controlled, double-blind, dose-escalation study (40 mg weekly [QW], 100 mg QW, 200 mg QW, 200 mg twice-weekly [BIW], and 100 mg BIW) of HDP-CDV for the prevention of CMV infection post-HCT. Treatment was initiated at the time of engraftment and continued until Week 13 post-HCT. HDP-CDV, at various doses, was active and well tolerated in the prevention CMV infection or disease.

### DEFINITIONS

The term “alkyl,” as used herein, unless otherwise specified, includes a saturated straight, branched, or cyclic, primary, secondary, or tertiary hydrocarbon, of, e.g., C₁₋₃₀ or C₁₋₂₂, and specifically includes methyl, ethyl, propyl, isopropyl, cyclopropyl, butyl, isobutyl, secbutyl, t-butyl, pentyl, cyclopentyl, isopentyl, neopentyl, hexyl, isohexyl, cyclohexyl, cyclohexylmethyl, heptyl, cycloheptyl, octyl, cyclo-octyl, dodecyl, tridecyl, pentadecyl, icosyl, hemicosyl, and decosyl. The alkyl group may be optionally substituted with, e.g., halogen (fluoro, chloro, bromo or iodo), hydroxyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., Protective Groups in Organic Synthesis, John Wiley and Sons, Second Edition, 1991, hereby incorporated by reference.

The term “lower alkyl”, as used herein, and unless otherwise specified, includes a C₁ to C₄ saturated straight, branched, or if appropriate, a cyclic (for example, cyclopropyl) alkyl group, which is optionally substituted.

Whenever a range of carbon atoms is referred to, it includes independently and separately every member of the range. As a nonlimiting example, the term “C₁-C₁₀ alkyl” is considered to include, independently, each member of the group, such that, for example, C₁-C₁₀ alkyl includes straight, branched and where appropriate cyclic C₁, C₂, C₃, C₄, C₅, C₆, C₇, C₈, C₉ and C₁₀ alkyl functionalities.

The term “protected” as used herein and unless otherwise defined includes a group that is added to an atom such as an oxygen, nitrogen, or phosphorus atom to prevent its further reaction or for other purposes. A wide variety of oxygen and nitrogen protecting groups are known to those skilled in the art of organic synthesis.

The term “halo”, as used herein, specifically includes to chloro, bromo, iodo, and fluoro.

The term “alkenyl” includes a straight, branched, or cyclic hydrocarbon of, for example, C₂₋₁₀₀, or C₂₋₂₂ with at least one double bond. Examples include, but are not limited to, vinyl, allyl, and methyl-vinyl. The alkenyl group can be optionally substituted in the same manner as described above for the alkyl groups.

The term “alkynyl” includes, for example, a C₂₋₁₀₀ or C₂₋₂₂ straight or branched hydrocarbon with at least one triple bond. The alkynyl group can be optionally substituted in the same manner as described above for the alkyl groups.

The term “alkoxy” includes a moiety of the structure —O-alkyl.

The term “acyl” includes a group of the formula R′C(O), wherein R′ is a straight, branched, or cyclic, substituted or unsubstituted alkyl or aryl.

As used herein, “aryl” includes aromatic groups having in the range of 6 up to 14 carbon atoms and “substituted aryl” refers to aryl groups further bearing one or more substituents as set forth above.

As used herein, “heteroaryl” includes aromatic groups containing one or more heteroatoms (e.g., N, O, S, or the like) as part of the ring structure, and having in the range of 3 up to 14 carbon atoms and “substituted heteroaryl” refers to heteroaryl groups further bearing one or more substituents as set forth above.

As used herein, the term “bond” or “valence bond” includes a linkage between atoms consisting of an electron pair.

The term “host”, as used herein, unless otherwise specified, includes mammals (e.g., cats, dogs, horses, mice, monkeys, etc.), humans, or other organisms in need of treatment. The host is for example, a human or an animal, including without limitation, primates, including macaques, baboons, as wells as chimpanzee, gorilla, and orangutan, ruminants, including sheep, goats, deer, and cattle, for example, cows, steers, bulls, and oxen; swine, including pigs; and poultry including chickens, turkeys, ducks, or geese.

The term “pharmaceutically acceptable salt” as used herein, unless otherwise specified, includes those salts which are, within the scope of sound medical judgment, suitable for use in contact with the tissues of hosts without undue toxicity, irritation, allergic response and the like, and are commensurate with a reasonable benefit/risk ratio and effective for their intended use. The salts can be prepared in situ during the final isolation and purification of one or more compounds of the composition, or separately by reacting the free base function with a suitable organic acid. Non-pharmaceutically acceptable acids and bases also find use herein, as for example, in the synthesis and/or purification of the compounds of interest. Nonlimiting examples of such salts are (a) acid addition salts formed with inorganic salts (for example hydrochloric acid, hydrobromic acid, sulfuric acid, phosphoric acid, nitric acid, and the like), and salts formed with organic salts such as acetic acid, oxalic acid, tartaric acid, succinic acid, ascorbic acid, benzoic acid, tannic acid, and the like; (b) base addition salts formed with metal cations such as zinc, calcium, magnesium, aluminum, sodium, potassium, copper, nickel and the like; (c) combinations of (a) and (b). Also included as “pharmaceutically acceptable salts” are amine salts.

The term “pharmaceutically acceptable esters” as used herein, unless otherwise specified, includes those esters of one or more compounds, which are, within the scope of sound medical judgment, suitable for use in contact with the tissues of hosts without undue toxicity, irritation, allergic response and the like, are commensurate with a reasonable benefit/risk ratio, and are effective for their intended use.

The term “pharmaceutically acceptable prodrug” includes a compound that is metabolized, for example, hydrolyzed or oxidized, in the host to form an active compound. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound.

The term “enantiomerically enriched”, as used herein, refers to a compound that is a mixture of enantiomers in which one enantiomer is present in excess, and preferably present to the extent of 95% or more, and more preferably 98% or more, including 100%.

The term “effective amount” includes an amount required for prevention, treatment, or amelioration of one or more of the symptoms of diseases or disorders provided herein.

It is to be understood that the compounds disclosed herein may contain chiral centers. Such chiral centers may be of either the (R) or (S) configuration, or may be a mixture thereof. Thus, the compounds provided herein may be enantiomerically pure, or be stereoisomeric or diastereomeric mixtures. It is understood that the disclosure of a compound herein encompasses any racemic, optically active, polymorphic, or steroisomeric form, or mixtures thereof, which preferably possesses the useful properties described herein, it being well known in the art how to prepare optically active forms and how to determine activity using the standard tests described herein, or using other similar tests which are well known in the art.

The invention will be further understood from the following non-limiting examples.

### EXAMPLES

### Example 1

Summary:

Clinical Studies of HDP-CDV were performed and described in detail in the following Examples. For example, HDP-CDV-201 is a placebo-controlled, dose-escalating trial in HSCT CMV (R+) recipients, evaluating the ability of HDP-CDV to prevent or control CMV infection was carried out. Five cohorts were established in which participants or subjects received either placebo or the HDP-CDV orally, in doses ranging from 40 mg weekly (QW) to 200 mg twice weekly (BIW). Subjects who were post-HSCT were enrolled at the time of engraftment and randomized to HDP-CDV or placebo (3 to 1 ratio) and received blinded therapy until approximately 100 day post-transplantation. HDP-CDV doses were 40 mg QW, 100 mg QW, 200 mg QW, 200 mg BIW and 100 mg BIW. Escalation to the next dose was decided by the data monitoring committee after review of the safety data from the previous Cohort. Subjects who developed CMV disease or CMV infection requiring pre-emptive therapy with local standard of care were discontinued from blinded therapy and followed for 4 weeks. Subjects who completed treatment with blinded therapy were followed for 8 weeks post-therapy.

Summary of Dosage and Adverse Events:

The results showed that at HDP-CDV doses of 200 mg BIW resulted in diarrhea. Thus, dose of 200 mg BIW was considered dose limiting. In contrast, lower doses of HDP-CDV were generally well tolerated with no indication of myelotoxicity or nephrotoxicity.

### Prevention of CMV Infection in Allogeneic HSCT Recipients

A randomized, double-blind, placebo-controlled study was performed, comparing two dosing regimens of the compound of formula I in the prevention of CMV infection in adult allogeneic HSCT recipients who were CMV (R+) prior to transplant. The primary endpoint was proposed as the occurrence of clinically important CMV infection, defined as the occurrence of CMV disease; initiation of anti-CMV specific therapy based on the treating physician's judgment of the patient's clinical status and documented CMV DNAemia; or the confirmed occurrence of CMV DNAemia>1,000 copies/mL (increasing on a second measurement to at least 2,000 copies/mL).

Over 700 subjects received HDP-CDV. Phase I studies included evaluations of safety, tolerability and pharmacokinetics (PK) in healthy volunteers and those with hepatic impairment; drug metabolism in healthy subjects; and food effects on PK. In addition to 210 patients were treated in emergency INDs.

Phase 2 studies were conducted in hematopoietic stem cell transplant (HSCT) recipients. These trials included a study, which enrolled over 200 subjects with various life-threatening dsDNA viral infections. Another phase 2 evaluated once weekly (QW) or twice weekly (BIW) regimens for the treatment of adenovirus infection in 17 pediatric or adult recipients. Another study evaluated CMV infection in 230 subjects. A Phase 2 placebo-controlled, dose-escalation trial, designed to assess the ability of HDP-CDV to prevent or control CMV infection following HSCT and identify a dose for further evaluation was conducted.

A placebo-controlled, dose-escalating trial in HSCT CMV (R+) recipients, evaluated the ability of HDP-CDV to prevent or control CMV infection. Subjects in five Cohorts received either placebo or oral HDP-CDV, in doses ranging from 40 mg weekly (QW) to 200 mg BIW.

The Efficacy Analyses and Results for the CMV Endpoints

The primary endpoint was defined as the incidence of CMV disease at any time during treatment or CMV DNAemia≧200 copies/mL at the end of treatment. All subjects who received at least one dose of drug/placebo and had at least one efficacy evaluation post baseline were included in the primary analysis, regardless of their CMV DNAemia status (negative or positive) at baseline (mITT population). Results for the pooled HDP-CDV Cohorts and for each Cohort separately were analyzed versus that for pooled placebo.

CMV DNAemia prior to dosing was evaluated in pooled HDP-CDV treatment groups, which showed that HDP-CMV achieved a statistically significant reduction of CMV DNAemia in treated groups compared to the placebo group. The analysis was adjusted for the presence or absence of CMV DNAemia prior to dosing (p=0.041).

In the largest Cohort of the Study (Cohort 4A, 100 mg BIW), the proportion of subjects who developed CMV disease or CMV DNAemia at the end of treatment was 10% versus 37.3% for placebo-treated subjects (p=0.001; TABLE 2).

TABLE 2
Rates of CMV Disease or End of Treatment CMV Infection (≧200 copies/ml)
[mITT Population (Missing CMV Values = Infection)]

| | | | | | | P-value (Relative to Placebo) | | |
|---|---|---|---|---|---|---|---|---|
| Treatment Group | | Infection | | Difference ^{[1]} | | Fisher's | | |
| Primary Analysis | N | n | Rate (%) | Result (%) | 95% C.I. | Exact | CMH1^{[2]} | CMH3^{[3]} |
| All HDP-CDV | 171 | 43 | 25.1% | −12.1% | (−26.1, 1.8) | 0.093 | 0.057 | 0.041 |
| 40 mg QW | 25 | 13 | 52.0% | 14.7% | (−8.4, 37.9) | 0.234 | 0.283 | 0.298 |
| 100 mg QW | 27 | 6 | 22.2% | −15.1% | (−35.0, 4.9) | 0.218 | 0.149 | 0.216 |
| 200 mg QW | 39 | 12 | 30.8% | −6.5% | (−25.5, 12.5) | 0.525 | 0.421 | 0.365 |
| 200 mg BIW | 30 | 7 | 23.3% | −14.0% | (−33.5, 5.6) | 0.235 | 0.178 | 0.109 |
| 100 mg BIW | 50 | 5 | 10.0% | −27.3% | (−42.2, −12.4) | 0.002 | 0.002 | 0.001 |
| Pooled Placebo | 59 | 22 | 37.3% | | | | | |

^{[1]} Difference is HDP-CDV minus placebo. C.I. = Confidence intervals calculated using binomial distribution.
^{[2]}Cochran-Mantel-Haenszel (CMH) test adjusted for CMV randomization strata.
^{[3]}Cochran-Mantel-Haenszel (CMH) test adjusted for CMV modified strata.

In the pre-specified subgroup analysis of subjects who were CMV negative at baseline, pooled HDP-CDV was superior to placebo treatment (15.8% versus 29.8% incidence; p=0.052), as was the HDP-CDV 100 mg BIW dose (incidence of 4.9% versus 29.8% versus placebo; p=0.002) (TABLE 3).

TABLE 3
Rates of CMV Disease or End of Treatment CMV Infection [mITT
Population (Missing CMV Values = Infection)]

| Infection | Treatment | | Infection | | Difference^{[1]} | | |
|---|---|---|---|---|---|---|---|
| Outcome | Group | N | N | Rate (%) | Result (%) | 95% C.I. | P-value ^{[2]} |
| aGVHD Pre-defined | All HDP-CDV | 162 | 41 | 25.3% | −6.8% | (−21.0, 7.5) | 0.374 |
| Randomization | 40 mg QW | 23 | 12 | 52.2% | 20.1% | (−3.9, 44.1) | 0.125 |
| Strata: Negative | 100 mg QW | 25 | 6 | 24.0% | −8.1% | (−29.0, 12.9) | 0.597 |
| | 200 mg QW | 39 | 12 | 30.8% | −1.3% | (−20.5, 17.9) | 1.000 |
| | 200 mg BIW | 29 | 7 | 24.1% | −7.9% | (−27.9, 12.1) | 0.612 |
| | 100 mg BIW | 46 | 4 | 8.7% | −23.4% | (−38.4, −8.4) | 0.006 |
| | Pooled Placebo | 53 | 17 | 32.1% | | | |
| CMV Modified | All HDP-CDV | 133 | 21 | 15.8% | −14.0% | (−28.5, 0.5) | 0.052 |
| Strata: Negative | 40 mg QW | 18 | 7 | 38.9% | 9.1% | (−16.9, 35.1) | 0.558 |
| | 100 mg QW* | 23 | 3 | 13.0% | −16.7% | (−35.7, 2.2) | 0.150 |
| | 200 mg QW | 29 | 6 | 20.7% | −9.1% | (−28.8, 10.6) | 0.433 |
| | 200 mg BIW* | 22 | 3 | 13.6% | −16.2% | (−35.6, 3.3) | 0.231 |
| | 100 mg BIW* | 41 | 2 | 4.9% | −24.9% | (−39.6, −10.3) | 0.002 |
| | Pooled Placebo | 47 | 14 | 29.8% | | | |

^{[1]}Difference is HDP-CDV minus placebo. C.I. = Confidence intervals calculated using binomial distribution.
^{[2]} Fisher's exact test relative to placebo
*Treatment groups that met the exploratory endpoint of 50% less than placebo but not greater than 25% overall.

With the exception of the 40 mg QW dose, all other HDP-CDV doses and dose regimens demonstrated antiviral activity when compared to placebo. Depending upon the analysis, activity increased with dose and/or dose frequency.

### HDP-CDV Versus Pooled Placebo Analysis

In subjects initially CMV viremia negative at baseline, the incidence of detectable CMV DNAemia of ≧100 copies/mL (limit of PCR detection), occurring at any time during treatment was measured.

Pooled HDP-CDV versus placebo was active (p=0.003), as shown for analyses for each dose regimen of 100 mg QW or higher (TABLE 4).

TABLE 4
Incidence and Time from First Dose to Onset of CMV DNAemia (≧100 copies/ml) During the
Treatment Period for Subjects who were Plasma CMV DNA Negative at Baseline (mITT Population)

| | | Cohort 1 | Cohort 2 | Cohort 3 | Cohort 4 | Cohort 4A |
|---|---|---|---|---|---|---|
| | Pooled | HDP-CDV | HDP-CDV | HDP-CDV | HDP-CDV | HDP-CDV |
| | Placebo | 40 mg QW | 100 mg QW | 200 mg QW | 200 mg BIW | 100 mg BIW |
| | N = 59 | N = 25 | N = 27 | N = 39 | N = 30 | N = 50 |
| CMV DNA Negative at | 49 | 19 | 23 | 30 | 23 | 42 |
| Baseline | | | | | | |
| Developed CMV DNAemia | 24 (49.0%) | 10 (52.6%) | 5 (21.7%) | 7 (23.3%) | 4 (17.4%) | 10 (23.8.%) |
| (≧100 copies/mL) N(%)^{[1]} | | | | | | |
| Fisher's Exact test p value | | 1.0 | 0.039 | 0.034 | 0.018 | 0.028 |
| relative to placebo | | | | | | |
| Time to CMV DNAemia | | | | | | |
| (Days)^{[2]} | | | | | | |
| Mean | 15.5 | 17.8 | 38.8 | 22.6 | 24.8 | 28.8 |
| Median | 13.5 | 14.5 | 30.0 | 15.0 | 25.5 | 20.0 |
| Median Survival Time (Days) | 37.0 | 51.0 | NA | NA | NA | NA |
| Log-Rank p-value^{[3]} | N/A | 0.864 | 0.011 | 0.045 | 0.020 | 0.009 |

^{[1]}Number of subjects with CMV DNA negative at baseline who subsequently developed CMV DNAemia during the treatment period. Subjects who did not have any events by the end of treatment were considered censored at the end of treatment/study.
^{[2]}Censored values are not included.
^{[3]}Results based on Kaplan-Meier (KM) method.

The incidence of CMV DNAemia>1,000 copies/mL during treatment was measured. Pooled HDP-CDV doses versus placebo were active in the mITT analysis of all subjects (p<0.001), as was each regimen of 200 mg QW and higher (TABLE 5). In subjects CMV negative at baseline, the pooled HDP-CDV group or each dose regimen of 100 mg QW or greater was superior to placebo (p<0.05).

TABLE 5
Rates of CMV DNAemia ≧1000 copies/mL During the Treatment
Period (mITT Population) Post Hoc Analysis (mITT population)

| | | | | | | | P-value (Relative to Placebo) | | |
|---|---|---|---|---|---|---|---|---|---|
| DNAemia | Treatment | | DNAemia | | Difference^{[1]} | | Fisher's | | |
| Outcome | Group | N | n | Rate (%) | Result (%) | 95% C.I. | Exact | CMH1^{[2]} | CMH3^{[3]} |

Primary Analysis

| | | | | | | | | |
|---|---|---|---|---|---|---|---|---|
| All HDP-CDV | 171 | 29 | 17.0% | −25.4% | −39.2, −11.6 | <0.001 | <0.001 | <0.001 |
| 40 mg QW | 25 | 10 | 40.0% | −2.4% | −25.3, 20.6 | 1.000 | 0.665 | 0.528 |
| 100 mg QW | 27 | 6 | 22.2% | −20.2% | −40.3, 0.0 | 0.092 | 0.056 | 0.086 |
| 200 mg QW | 39 | 7 | 17.9% | −24.4% | −41.9, −7.0 | 0.015 | 0.004 | 0.003 |
| 200 mg BIW | 30 | 2 | 6.7% | −35.7% | −51.2, −20.3 | <0.001 | <0.001 | <0.001 |
| 100 mg BIW | 50 | 4 | 8.0% | −34.4% | −49.1, −19.7 | <0.001 | <0.001 | <0.001 |
| Pooled Placebo | 59 | 25 | 42.4% | | | | | |

Exploratory Analysis, Met Endpoint^{[4]}

| | | | | | | | | |
|---|---|---|---|---|---|---|---|---|
| All HDP-CDV | 119 | 13 | 10.9% | −23.2% | −38.8, −7.7 | 0.001 | <0.001 | <0.001 |
| Pooled Placebo | 41 | 14 | 34.1% | | | | | |

^{[1]}Difference is HDP-CDV minus placebo. C.I. = Confidence intervals calculated using binomial distribution.
^{[2]}Cochran-Mantel-Haenszel (CMH) test adjusted for CMV randomization strata.
^{[3]}Cochran-Mantel-Haenszel (CMH) test adjusted for CMV modified strata.
^{[4]}All Cohorts with HDP-CDV failure ≦25% and <50% of failure rate in combined placebo group.

Of particular note, no subjects (0%) developed CMV DNAemia>1000 copies/mL, compared to 31.9% in the pooled placebo-treated subjects, in the 100 and 200 mg BIW groups (TABLE 6).

TABLE 6
Rates of CMV DNAemia ≧1,000 copies/ml During the Treatment
Period (mITT Population) Post Hoc Analysis (CMV DNAemia levels
of at least 1,000 copies/mL) CMV Modified Strata: Negative

| | | DNAemia | | Difference^{[1]} | | |
|---|---|---|---|---|---|---|
| | | | Rate | Result | | P- |
| Treatment Group | N | N | (%) | (%) | 95% C.I. | value^{[2]} |

Primary Analysis

| | | | | | | |
|---|---|---|---|---|---|---|
| All HDP-CDV | 133 | 8 | 6.0% | −25.9% | −39.8, −12.0 | <0.001 |
| 40 mg QW | 18 | 4 | 22.2% | −9.7% | −33.1, 13.7 | 0.550 |
| 100 mg QW | 23 | 2 | 8.7% | −23.2% | −40.8, −5.6 | 0.040 |
| 200 mg QW | 29 | 2 | 6.9% | −25.0% | −41.2, −8.8 | 0.012 |
| 200 mg BIW | 22 | 0 | 0.0% | −31.9% | −45.2, −18.6 | 0.002 |
| 100 mg BIW | 41 | 0 | 0.0% | −31.9% | −45.2, −18.6 | <0.001 |
| Pooled Placebo | 47 | 15 | 31.9% | | | |

^{[1]}Difference is HDP-CDV minus placebo. C.I. = Confidence intervals calculated using binomial distribution.
^{[2]}Fisher's exact test relative to placebo.

The Incidence of CMV Disease or Initiation of Anti-CMV Preemptive Therapy with an Excluded Medication (e.g., Ganciclovir or Foscarnet), Analyzed for all Subjects (mITT Population)

Pooled HDP-CDV was numerically superior to placebo (22% versus 31%), as was the 100 mg BIW group (14%) (TABLE 7). The proportion of subjects reaching the composite endpoint of initiation of anti-CMV therapy or CMV disease or CMV DNAemia>1,000 copies/mL. Higher doses of HDP-CDV (≧200 mg QW) were superior to placebo in reducing the incidence of this composite endpoint (less than 15% for these doses versus 38.3% for placebo; p≦0.05 for 200 mg QW and 200 mg BIW; p<0.01 for 100 mg BIW). (TABLE 8).

TABLE 7
Incidence and Time from First Dose Date to Initiation of Antiviral
Therapy to Treat CMV Infection and/or Disease (mITT Population)

| | | Cohort 1 | Cohort 2 | Cohort 3 | Cohort 4 | Cohort 5 |
|---|---|---|---|---|---|---|
| | Pooled | HDP-CDV | HDP-CDV | HDP-CDV | HDP-CDV | HDP-CDV |
| | Placebo | 40 mg QW | 100 mg QW | 200 mg QW | 200 mg BIW | 100 mg BIW |
| | N = 59 | N = 25 | N = 27 | N = 39 | N = 30 | N = 50 |
| Subjects Receiving Antiviral | 18 (30.5%) | 11 (44.0%) | 7 (25.9%) | 7 (17.9%) | 5 (16.7%) | 7 (14.0%) |
| Therapy and/or had CMV | | | | | | |
| disease^{[1]} | | | | | | |
| Subjects without a CMV | 41 (69.5%) | 14 (56.0%) | 20 (74.1%) | 32 (82.1%) | 25 (83.3%) | 43 (86.0%) |
| event^{[2]} | | | | | | |
| Fisher's exact p-value | | 0.316 | 0.80 | 0.236 | 0.204 | 0.066 |
| Time to Antiviral Therapy | | | | | | |
| (Days)^{a} | | | | | | |
| Mean | 26.8 | 27.1 | 41.9 | 38.1 | 21.6 | 23.1 |
| Median | 22.5 | 29.0 | 41.0 | 40.0 | 15.0 | 23.0 |
| Log-Rank p-value^{[3]} | NA | 0.225 | 0.455 | 0.231 | 0.298 | 0.060 |

^{a}Censored values are not included.
^{[1]}The denominator is the number of mITT subjects.
^{[2]}Subjects who did not have antiviral therapy to treat CMV infection and/or disease were considered censored at the time of discontinuation from the study.
^{[3]}Results are based on Kaplan-Meier (KM) method.

TABLE 8
Incidence of Emergence of CMV Disease, Plasma CMV ≧1,000 copies/mL OR Initiation of
Excluded CMV Medication (Subjects in the mITT Population, Modified CMV Negative Stratum).

| | | Cohort 1 | Cohort 2 | Cohort 3 | Cohort 4 | Cohort 4A |
|---|---|---|---|---|---|---|
| | Pooled | HDP-CDV | HDP-CDV | HDP-CDV | HDP-CDV | HDP-CDV |
| | Placebo | 40 mg QW | 100 mg QW | 200 mg QW | 200 mg BIW | 100 mg BIW |
| | N = 47 | N = 18 | N = 23 | N = 29 | N = 22 | N = 41 |
| Subjects Who Had CMV- | 18 (38.3%) | 5 (27.8%) | 5 (21.7%) | 4 (13.8%) | 3 (13.6%) | 5 (12.2%) |
| related Event^{[1]} | | | | | | |
| Subjects without a CMV- | 29 (61.7%) | 13 (72.2%) | 18 (78.3%) | 25 (86.2%) | 20 (86.4%) | 36 (87.8%) |
| related event | | | | | | |
| Fisher's exact p-value | | 0.565 | 0.188 | 0.036 | 0.050 | 0.007 |

^{[1]}The denominator is the number of mITT subjects; events included CMV Disease, CMV DNAemia ≧1,000 c/mL or initiation of anti-CMV treatment.

There was a trend in subjects who received HDP-CDV 100 mg QW versus placebo in preventing this outcome (21.7% versus 38.3%) and the 40 mg QW dose was inactive, providing evidence for a dose response.

Smooth line scatter plots of individual subject data from the 200 mg QW, 100 mg BIW and placebo groups (CMV DNAemia copies/mL over time) shows that the 100 mg BIW dose regimen resulted in a lower frequency and/or lower overall levels of CMV DNAemia when visually compared to either placebo or 200 mg QW, suggesting that BIW dosing may have an impact on CMV suppression over and above the total weekly administered dose.

CMV from subjects who developed virologic failure during active HDP-CDV treatment were analyzed for genotypic changes in the UL54 and UL97 genes (n=30 from Cohorts with dosing regimens of ≧100 mg QW). No mutations known to confer resistance to antivirals directed at CMV were detected.

### Pharmacokinetics of HDP-CDV

The pharmacokinetics of HDP-CDV (oral solution) were investigated in a dose-escalation study in healthy volunteers (Study HDP-CDV-102) in which single doses of up to 2 mg/kg and multiple doses (3 doses, each separated by 6 days) of up to 1 mg/kg were administered. The maximum plasma concentration (Cₘₐₓ) and systemic exposure area under the curve (AUC) for both HDP-CDV and CDV increased approximately in proportion to dose. The Cₘₐₓ for HDP-CDV occurred about 2 to 3 hours after administration while that for CDV occurred about 9 to 15 hours after administration. At the 1 mg/kg dose level, the half-life of elimination (t½ elim) for HDP-CDV was 27 hours and that for CDV was 65 hours. After 3 doses of HDP-CDV (Days 0, 6, and 12) there was negligible accumulation of HDP-CDV or CDV on Day 12; the PK profiles of HDP-CDV and CDV on Day 12 were not different from Day 0. CDV was detectable in pre-dose plasma samples on Days 6 and 12 indicating a long half-life. HDP-CDV was not quantifiable in the urine, but was eliminated in urine slowly as its metabolites including CDV.

Oral administration of HDP-CDV as a tablet resulted in systemic exposures that were about 13% lower than exposures following administration of the same dose as an oral solution. Peak plasma concentrations (Cₘₐₓ) were reduced 48% and systemic exposure (AUC_{inf}) was reduced 28% when HDP-CDV tablets were given to subjects following a high fat meal as compared to fasted. For this reason, in Study HDP-CDV-201, HDP-CDV was administered to fasted subjects. Subsequently, anecdotal reports have indicated that gastrointestinal symptoms during HDP-CDV therapy may be mitigated by administration of HDP-CDV with food. For this reason, in future studies, HDP-CDV may be administered with a food whenever possible.

### Safety and Tolerability Profile of HDP-CDV

Data from Study HDP-CDV-201 also provided characterization of the safety and tolerability profile of HDP-CDV when administered for the prevention or control of CMV infection in high risk (R⁺) post-HSCT adult subjects.

There was no indication of nephrotoxicity or myelotoxicity associated with HDP-CDV, regardless of dose and dosing frequency. HDP-CDV dose regimens of 40 mg QW and 100 mg QW had tolerability profiles similar to placebo in terms of AEs and laboratory abnormalities. A dose-related increase in ALT was associated with HDP-CDV therapy. ALT increases typically resolved after completion of HDP-CDV therapy and do not appear to be of toxicologic importance, based upon the preclinical and clinical safety profiles to date. Few clinical hepatobiliary AEs were reported in association with treatment with HDP-CDV and most were mild or moderate in intensity. No case of drug induced liver injury (DILI) clearly attributable to HDP-CDV was noted during the course of the study.

Diarrhea, frequently associated with other gastrointestinal symptoms, was dose limiting in this study and a dose of 200 mg BIW is not considered tolerable in this subject population. In contrast, with respect to diarrhea, QW HDP-CDV doses in the range studied (40 to 200 mg QW) were sufficiently well tolerated. When comparing Cohorts 3 (200 mg QW) and 4a (100 mg BIW), the frequency and severity of diarrhea appeared to be somewhat related to dose as adjusted for weight, using a 3 mg/kg cut off. Events of diarrhea (often reported as GVHD of the intestine) were more frequent and more severe, compared to placebo, in subjects who received HDP-CDV 100 mg BIW in the fasted state.

These events infrequently led to permanent discontinuation of HDP-CDV. In addition, approximately one-third of the subjects in the 100 mg BIW Cohort interrupted HDP-CDV due to an adverse event; the majority were able to resume dosing. Therefore diarrhea appears to have been a manageable AE in this patient population. An increased frequency and severity of apparent GVHD of the intestine, but not of the skin or the liver, was noted in subjects receiving HDP-CDV BIW.

Analyses of these findings suggest that this increased reporting of GVHD was prompted by the occurrence of GI symptoms commonly ascribed to a presumptive diagnosis of GVHD, but were in fact due to a HDP-CDV-related diarrheal event.

In summary, the safety profile of HDP-CDV 200 mg per week appears acceptable in the context of the benefit derived from the prevention of CMV reactivation as compared to the safety profile of preemptive therapy which is the current standard of care.

Taking into account completed and planned studies listed in TABLE 9, more than 950 adult subjects are exposed to at least one dose of HDP-CDV in both controlled and uncontrolled studies, including nearly 600 adult subjects enrolled in randomized, placebo controlled studies (TABLE 10). Of these, over 500 subjects received doses of at least 150 mg/wk in controlled clinical trials at the conclusion of the development program.

TABLE 9 displays the studies that comprise the safety database at the time of completion of Phase 3 study.

TABLE 9
Description of Clinical Safety Studies

| | | | | | |
|---|---|---|---|---|---|
| | Study Status | | | | |
| | (Expected | | Total Enrollment | | Study |
| Study ID | Completion) | Study Design | (Planned/Actual) | Dose and Schedule | Population |

Phase 1 and Clinical Pharmacology Studies

| | | | | | |
|---|---|---|---|---|---|
| HDP-CDV- | Completed | This was a Phase 1, | Overall: | SAD part: | Healthy subjects |
| 102 | | dose-escalation, First- | N = 84 total | 0.025, 0.05, 0.1, | Age: 18-55 years |
| | | Time-in-Human study | N = 56 HDP-CDV | 0.2, 0.4, 0.6, 1, 1.5 | |
| | | of the safety, | N = 28 placebo | and 2 mg/kg | |
| | | tolerability and PK of | SAD part: | MAD part: | |
| | | HDP-CDV in healthy | N = 54 total | 0.1, 0.2, 0.4, 0.6 | |
| | | subjects. A solution | N = 36 HDP-CDV | and 1.0 mg/kg | |
| | | formulation of HDP- | N = 18 placebo | (Subjects in the | |
| | | CDV was administered | MAD part: | MAD Cohorts | |
| | | to subjects in a total of | N = 30 total | received a total of | |
| | | 9 single ascending dose | N = 20 HDP-CDV | 3 doses of HDP- | |
| | | (SAD) Cohorts and 5 | N = 10 placebo | CDV, one dose | |
| | | multiple ascending | | every 6 days) | |
| | | dose (MAD) Cohorts. | | | |
| | | Each Cohort enrolled | | | |
| | | 6 subjects randomized | | | |
| | | 2:1 (active:placebo). | | | |
| HDP-CDV- | Completed | This was a Phase 1, | N = 24 | Each subject | Healthy subjects |
| 103 | | 3-way crossover study | | received 3 single | Age: 18-55 years |
| | | to evaluate the | | doses of HDP-CDV | |
| | | comparative | | in crossover | |
| | | bioavailability of the | | fashion (each | |
| | | HDP-CDV tablet and | | separated by a 14- | |
| | | solution formulations | | day washout | |
| | | and the effect of food | | interval): | |
| | | as a high-fat meal on | | 40 mg solution | |
| | | the bioavailability of | | (fasted); | |
| | | the HDP-CDV tablet | | 40 mg tablet (fed); | |
| | | formulation in healthy | | 40 mg tablet | |
| | | subjects | | (fasted) | |
| HDP-CDV- | Completed; | This was an open- | Overall: | Single 200 mg dose | Male and female |
| 106 | CSR in | label, nonrandomized, | N = 25 | of HDP-CDV | subjects with |
| | preparation | multi-center, sequential | Cohort 1: | (fasted) | moderate and |
| | | group, safety, | N = 17 (9 MHI, | | severe hepatic |
| | | tolerability and PK | 8 HC) | | impairment |
| | | study of HDP-CDV | Cohort 2: | | (based on Child- |
| | | after single dose | N = 8 | | Pugh-Turcotte |
| | | administration in | | | Class B and C, |
| | | subjects with moderate | | | respectively) and |
| | | hepatic impairment | | | healthy control |
| | | (MHI) who were | | | subjects with |
| | | matched with healthy | | | normal hepatic |
| | | control (HC) subjects | | | function. |
| | | with normal hepatic | | | The |
| | | function and in | | | demographics |
| | | subjects with severe | | | (age [±5 years], |
| | | hepatic impairment | | | body mass index |
| | | (SHI). The safety, | | | [±15%] and |
| | | tolerability and PK | | | gender) of HC |
| | | data from Cohort 1 | | | subjects were |
| | | (MHI and HC) subjects | | | matched as |
| | | was reviewed prior to | | | closely as |
| | | initiating dosing in SHI | | | possible with |
| | | subjects in Cohort 2. | | | those of MHI |
| | | | | | subjects. |
| | | | | | Age: 18-65 years |
| HDP-CDV- | In Progress | This was a Phase 1, | N = 6 | Single dose of 200 mg | Healthy male |
| 112 | (Expected | single-center, single | | ¹⁴C-HDP-CDV | subjects |
| | Study | oral dose, open-label, | | (fasted) | Age: 18-55 years |
| | Completion: | radiolabeled study of | | | |
| | 2Q2012) | ¹⁴C-HDP-CDV to | | | |
| | | assess the excretion | | | |
| | | pathways and | | | |
| | | metabolite profile of | | | |
| | | HDP-CDV in fasted | | | |
| | | healthy human | | | |
| | | subjects. | | | |
| HDP-CDV- | Protocol | A Phase 1, double- | N = 52 (Planned) | Subjects receive | Healthy subjects |
| 108 | developed | blind, randomized, | | each of the | Age: 18-45 years |
| | (Expected | 4-way crossover study | | following 4 single- | |
| | Study | to define the ECG | | dose treatments: | |
| | Completion: | effects of HDP-CDV | | Placebo | |
| | 3Q2012) | administered using a | | 400-mg | |
| | | clinical and a | | moxifloxacin | |
| | | supratherapeutic dose | | 200-mg HDP-CDV | |
| | | compared to placebo | | 350-mg HDP-CDV | |
| | | and moxifloxacin in | | | |
| | | healthy subjects | | | |
| HDP-CDV- | Protocol | This is a Phase 1, | N = 20 (Planned) | Subjects receive | Healthy subjects |
| 113 | Development | open-label, | | two 1 mg IV doses | Age: 18-55 years |
| | (Expected | randomized, 2-way | | of MDZ on | |
| | Study | crossover study to | | 2 consecutive days | |
| | Completion: | evaluate the effect of | | and two 2.5 mg PO | |
| | 2Q2012) | HDP-CDV co- | | doses of MDZ on | |
| | | administration on the | | 2 consecutive days. | |
| | | safety and PK of single | | The second IV dose | |
| | | oral (PO) and | | and second PO | |
| | | intravenous (IV) doses | | dose of MDZ is co- | |
| | | of midazolam (MDZ) | | administered with | |
| | | in healthy subjects | | 200-mg HDP- | |
| | | | | CDV. There is a | |
| | | | | washout interval | |
| | | | | (dose-to-dose) of | |
| | | | | ≧14 days between | |
| | | | | the HDP-CDV | |
| | | | | doses. | |

Controlled Clinical Studies

| | | | | | |
|---|---|---|---|---|---|
| HDP-CDV- | Completed; | This was a Phase 2, | N = 32 per Cohort | Cohort 1: | Allogeneic stem |
| 201 | CSR in | multicenter, | (Planned) | 40 mg HDP-CDV | cell transplant |
| Viral target: | preparation | randomized, double- | Randomized 3:1 | or placebo QW | recipients who |
| CMV | | blind, placebo- | active (A): placebo | Cohort 2: | were CMV |
| | | controlled, dose- | (P) | 100 mg HDP-CDV | seropositive (R+) |
| | | escalation study of | Actual Enrollment: | or placebo QW | at the time of |
| | | HDP-CDV in | Cohort 1: N = 40 | Cohort 3; | transplant |
| | | allogeneic stem cell | (30A:10P) | 200 mg HDP-CDV | Males and |
| | | transplant recipients. | Cohort 2: N = 39 | or placebo QW | females |
| | | Dosing was initiated | (29A:10P) | Cohort 4: | Age: ≧18 years |
| | | immediately following | Cohort 3: N = 53 | 200 mg HDP-CDV | |
| | | engraftment (between | (39A:14P) | or placebo BIW | |
| | | Days 14 to | Cohort 4: N = 40 | Cohort 4A: | |
| | | 30[+5] post-transplant) | (30A:10P) | 100 mg HDP-CDV | |
| | | and continued through | Cohort 4A: N = 67 | or placebo BIW | |
| | | Week 13 post- | (50A:17P) | | |
| | | transplant | | | |
| HDP-CDV- | Enrolling | This is a Phase 2, | Overall N = 48 | Adult subjects are | Pediatric and |
| 202 | (Expected | multicenter, | (Planned) | randomized to | adult recipients |
| Viral target: | Completion: | randomized, placebo- | Randomization | receive 100 mg | of an allogeneic |
| AdV | 4Q2012) | controlled preemptive | ratio is 1:1 for | BIW or 200 mg | HSCT with |
| | | therapy study in HSCT | dosing frequency | QW of HDP-CDV | detectable AdV |
| | | recipients who have | (QW:BIW) and 2:1 | or placebo for | DNA in their |
| | | asymptomatic AdV | for study treatment | 6-12 weeks. | plasma by PCR |
| | | DNAemia. | (HDP- | Subjects in open- | Males and |
| | | Assignment to dosing | CDV:placebo). | label treatment | females |
| | | frequency (i.e., QW | N = 14 (actual, as of | receive 100 mg | Age: ≦75 years |
| | | versus BIW) is | 12Mar2012) | HDP-CDV BIW | |
| | | unblinded, while | | for up to 12 weeks. | |
| | | treatment assignment | | Pediatric subjects | |
| | | (i.e., HDP-CDV versus | | (i.e., <18 years old) | |
| | | placebo) is double- | | are randomized to | |
| | | blinded. | | receive 4 mg/kg | |
| | | Any subject who is | | QW or 2 mg/kg | |
| | | considered a treatment | | BIW of HDP-CDV | |
| | | failure with respect to | | or placebo (not to | |
| | | the primary endpoint is | | exceed 200 mg/ | |
| | | offered open-label | | week) for 6-12 | |
| | | treatment with HDP- | | weeks. Subjects in | |
| | | CDV. | | open label | |
| | | | | treatment receive | |
| | | | | 2 mg/kg HDP-CDV | |
| | | | | BIW (not to exceed | |
| | | | | 200 mg/week) for | |
| | | | | up to 12 weeks. | |
| HDP-CDV- | Protocol | This is a Phase 3, | ~540 subjects | Subjects are | Allogeneic stem |
| 301 | Development | multicenter, | (2 active to | randomized to | cell transplant |
| Viral target: | (Expected | randomized, double- | 1 placebo) | receive HDP-CDV | recipients who |
| CMV | Completion: | blind, placebo- | | 200 mg QW (not to | were CMV |
| | 4Q2014) | controlled study of the | | exceed 3 mg/kg) or | seropositive (R+) |
| | | safety, tolerability, and | | HDP-CDV 75 mg | at the time of |
| | | ability of HDP-CDV to | | BIW or placebo | transplant, CMV |
| | | prevent clinically | | | dsDNAemia |
| | | significant CMV | | | negative at |
| | | infection or disease in | | | screening |
| | | R+ hematopoietic stem | | | Males and |
| | | cell transplant | | | females |
| | | recipients. | | | Age: ≧18 years |

Uncontrolled Clinical Studies

| | | | | | |
|---|---|---|---|---|---|
| HDP-CDV- | Enrollment | This is a multi-center, | N = 200 (planned) | Adults and | Male and female |
| 350 | complete | open-label, expanded | N = 207 (actual, as | adolescent subjects | children and |
| Viral target: | (Expected | access study of safety | of 12MAR2012) | receive 100 mg | adults with |
| various | Completion: | and antiviral activity of | | BIW or 200 mg | serious or life- |
| dsDNA viruses | 4Q2012) | HDP-CDV in subjects | | QW of HDP-CDV | threatening |
| (including | | with serious or life- | | (not to exceed | disease or |
| CMV and | | threatening disease or | | 4 mg/kg/week) for | condition caused |
| AdV) | | condition caused by | | up to 6 months. | by infection with |
| | | infection with a | | Pediatric subjects | a dsDNA virus. |
| | | dsDNA virus | | (i.e., ≦12 years old) | |
| | | | | receive 4 mg/kg | |
| | | | | QW or 2 mg/kg | |
| | | | | BIW of HDP-CDV | |
| | | | | (not to exceed | |
| | | | | 200 mg/week) for | |
| | | | | up to 6 months. | |
| | | | | Treatment may be | |
| | | | | extended after | |
| | | | | 6 months at | |
| | | | | Agency's discretion | |
| | | | | on a case-by-case | |
| | | | | basis. | |
| HDP-CDV- | Enrolling | Subset of subjects | N = 30 (planned) | Adults and | Male and female |
| 350 CDV-PP | (Expected | participating in HDP- | N = 5 (actual, as of | adolescent subjects | children and |
| PK Substudy | Completion | CDV-350, at selected | 12MAR2012) | receive 100 mg | adults with |
| Viral target: | 4Q2012) | investigative sites is | | BIW or 200 mg | serious or life- |
| various | | enrolled in this | | QW of HDP-CDV | threatening |
| dsDNA viruses | | substudy which | | (not to exceed | disease or |
| (including | | measures intracellular | | 4 mg/kg/week) for | condition caused |
| CMV and | | levels of cidofovir- | | up to 6 months. | by infection with |
| AdV) | | diphosphate (CDV-PP) | | Pediatric subjects | a dsDNA virus. |
| | | in peripheral blood | | (i.e., ≦12 years old) | |
| | | mononuclear cells. | | receive 4 mg/kg | |
| | | Subjects must weigh | | QW or 2 mg/kg | |
| | | ≧60 kg. | | BIW of HDP-CDV | |
| | | | | (not to exceed | |
| | | | | 200 mg/week) for | |
| | | | | up to 6 months. | |
| | | | | Treatment may be | |
| | | | | extended after | |
| | | | | 6 months at | |
| | | | | Agency's discretion | |
| | | | | on a case-by-case | |
| | | | | basis. | |
| EINDs and | No new EINDs | A separate EIND | N = 250 (Projected) | Adults and | Male and female |
| foreign | are currently | requesting use of HDP- | N = 214 (as of | adolescent subjects | children and |
| equivalents | planned. | CDV was prepared for | 13FEB2012) | receive 100 mg | adults with |
| Viral target: | | each patient by the | | BIW or 200 mg | serious or life- |
| various | | responsible | | QW of HDP-CDV | threatening |
| dsDNA viruses | | investigator. | | (not to exceed | disease or |
| (including | | Individualized | | 4 mg/kg/week) for | condition caused |
| CMV and | | protocols have been | | up to 6 months. | by infection with |
| AdV) | | developed for each | | Pediatric subjects | a dsDNA virus. |
| | | patient to allow | | (i.e., ≦12 years old) | Age: at the |
| | | flexibility in treating | | receive 4 mg/kg | Division's |
| | | patients with different | | QW or 2 mg/kg | discretion |
| | | medical histories and | | BIW of HDP-CDV | |
| | | disease states. As | | (not to exceed | |
| | | such, each EIND | | 200 mg/week) for | |
| | | protocol has been | | up to 6 months. | |
| | | unique but contains | | Treatment may be | |
| | | some standard safety | | extended after | |
| | | assessments as well as | | 6 months at | |
| | | virological and plasma | | Agency's discretion | |
| | | PK sampling | | on a case-by-case | |
| | | schedules. | | basis. | |

TABLE 10
HDP-CDV Development Program Overall
Estimated Extent of Exposure

| | | | Multiple Doses | | | |
|---|---|---|---|---|---|---|
| | | Sin- | Uncontrolled Studies | | Controlled Studies | |
| | | gle | <150 | ≧150 | <150 | ≧150 |
| | Total | dose | mg/week | mg/week | mg/week | mg/week |
| Adults | 983 | 146 | NA | 250 | 114 | 479 |
| Children | 165 | 0 | 20 | 113 | 0 | 32 |
| Total | 1148 | 146 | NA | 363 | 114 | 511 |

### Overall Protocol of the Phase 1 and 2 Trials for Prevention of CMV Infection

The data from the trials showed that doses of HDP-CDV of 100 mg QW or higher demonstrated antiviral activity in the prevention of clinically meaningful CMV infection and disease. Furthermore, the data showed that that BIW dosing may provide additional benefit in the extent of viral suppression; and that doses up to 200 mg per week were sufficiently well tolerated, with diarrhea as the most prominent dose limiting adverse event.

### Protocol Synopsis

To explore the incidence and time to CMV events, a Kaplan-Meier “time-to” analysis was conducted using CMV disease and/or discontinuation due to initiation of alternative anti-CMV therapy. This endpoint assessed the emergence of CMV disease or CMV DNAemia requiring initiation of antiviral therapy, comparing HDP-CDV- and placebo-treated subjects. As shown in TABLE 11, a total of 55 subjects, distributed across all treatment groups, were discontinued due to CMV events.

The following table presents a top-level summary of the design for Study HDP-CDV-201.

TABLE 11
Study HDP-CDV-201: Study Synopsis

| | |
|---|---|
| Title: | A multicenter, randomized, double-blind, placebo-controlled, dose-escalation study of the |
| | safety, tolerability and ability of HDP-CDV to prevent or control CMV infection in R+ |
| | hematopoietic stem cell transplant (HSCT) recipients. |
| | Phase 2 |
| Study Population: | Allogeneic stem cell transplant recipients who were CMV seropositive (R+) at the time of transplant |
| | and who satisfied the following entry criteria were enrolled into the study. |
| | Key Inclusion Criteria: |
| | 1. Age ≧18 years, males or females. |
| | 2. Allogeneic HSCT recipients who were CMV seropositive before transplantation (i.e., R+ |
| | patients). |
| | 3. Recipients who were between 14 and 30 (+5 days) postqualifying transplant. |
| | 4. Recipients who had evidence of engraftment before randomization and receiving their first dose |
| | of study drug. Recipients must have engrafted on or before Day-30 post-transplant to be eligible for |
| | enrollment. |
| | Key Exclusion Criteria: |
| | 1. Recipients for whom the current, predose clinical course of CMV infection suggested that the |
| | investigator would not be able to withhold treatment for CMV for a minimum of 5, but preferably 7 |
| | days following the subject's first dose of study drug. |
| | 2. Recipients who had received any of the following: |
| | GCV, vGCV, foscarnet or CDV within 14 days prior to enrollment; |
| | any anti-CMV therapy following transplantation (including Cytogam ®), |
| | any CMV vaccine, |
| | any investigational drug with antiviral activity against dsDNA viruses within 14 days prior to |
| | enrollment, [Note: An investigational drug was defined as a drug that was not approved for any |
| | indication by the FDA.] |
| | any other investigational drug (i.e., those without any “anti-dsDNA virus” activity; for example, |
| | anti-influenza compounds) within 14 days prior to enrollment without the prior written consent of the |
| | medical monitor. |
| | 3. Patients receiving high dose ACV (>2000 mg total oral daily dose or >5 mg/kg IV three times |
| | daily) or vACV (Valtrex; >3000 mg total daily dose) at the time of dosing. |
| | 4. Patients with active CMV disease diagnosed within 6 months prior to enrollment; patients with |
| | CMV DNAemia requiring intervention with antiviral therapy at the time of enrollment. |
| | 5. Patients with hepatic dysfunction as evidenced by ALT or AST >5 × ULN or direct bilirubin |
| | >2.5 × ULN. |
| | 6. Patients with Grade 3 or 4 GVHD of the GI tract; patients with any GI disease that would, in the |
| | judgment of the investigator, preclude the patient from taking or absorbing oral medications. |
| Total Randomized | Cohort 1 (40 mg HDP-CDV/placebo QW (QW)); 40 subjects randomized (data censored from 7 |
| (Cohorts 1-4A) | subjects enrolled at site 024) |
| | Cohort 2 (100 mg HDP-CDV/placebo QW); 39 subjects randomized (data censored from 2 subjects |
| | enrolled at site 024) |
| | Cohort 3 (200 mg HDP-CDV/placebo QW); 53 subjects randomized |
| | Cohort 4 (200 mg HDP-CDV/placebo BIW (BIW) initially, reduced to 200 mg QW); 40 subjects |
| | randomized |
| | Cohort 4A (100 mg HDP-CDV/placebo BIW); 67 subjects randomized |
| Overall Study Design | This was a multicenter, randomized, double-blind, placebo-controlled, dose-escalation study, initially |
| | designed to have two parts. Part I was a randomized, double-blind, placebo-controlled, dose- |
| | escalation study of multiple doses of HDP-CDV in HSCT transplant recipients. A minimum of |
| | 32 HSCT recipients randomized 3:1 (24 HDP-CDV:8 placebo) were to be enrolled into each dose- |
| | escalating Cohort. |
| | All subjects randomized in this study were to be stratified by 2 criteria: |
| | Presence or absence of aGVHD requiring systemic treatment or therapy with steroids |
| | (≧1 mg/kg) assessed close to the time of randomization (i.e., as close as possible to the time of |
| | randomization but not more than 7 days earlier). |
| | Presence or absence of CMV DNAemia (>100 copies/mL) determined within the 7 |
| | days prior to dosing. |
| | In Part II, one of the dose levels administered in Part I was to be chosen to test against placebo to |
| | evaluate the statistical significance of HDP-CDV as a therapy for preventing and/or controlling CMV |
| | infection or preventing CMV disease. [Note: Part II of the study was not conducted]. |
| Duration of Treatment | Subjects were treated beginning 14-30 (+5) days post-transplant and continuing through Week 13 |
| | (Days 84-90) post-transplant. As a result, subjects received 9, 10 or 11 weeks of treatment. |
| Primary Objectives | To determine the safety and tolerability of HDP-CDV in hematopoietic stem cell |
| (Part I, Dose | transplant (HSCT) recipients |
| Escalation Cohorts) | To determine the ability of HDP-CDV to prevent or control CMV infection in R+ |
| | HSCT recipients during the treatment period |
| | To select the dose of HDP-CDV to be used in Part II based on safety and efficacy |
| Secondary Objectives | In subjects who were plasma CMV DNA negative at baseline, to compare the incidence and |
| | time to onset of CMV DNAemia between HDP-CDV treated and placebo subjects and between HDP- |
| | CDV treatment groups during the treatment and subsequent 8 week follow-up period |
| | In subjects who were plasma CMV DNA positive at baseline, to compare the change from |
| | baseline in CMV DNAemia between HDP-CDV treated and placebo subjects and between HDP- |
| | CDV treatment groups during the treatment period and subsequent 8 week follow-up period |
| | To compare all-cause subject drop-out rate and/or time to discontinuation from the study |
| | between HDP-CDV-treated subjects versus placebo-treated subjects and between HDP-CDV |
| | treatment groups |
| | To compare incidence, severity, and progression of GVHD between HDP-CDV-treated subjects |
| | and placebo-treated subjects and between HDP-CDV treatment groups during the treatment period |
| | To monitor trough levels of HDP-CDV and CDV throughout the treatment period |
| | To compare the incidence and time to onset of CMV disease between HDP-CDV treated and |
| | placebo subjects and between HDP-CDV treatment groups during the treatment and subsequent |
| | 8 week follow-up period |
| | To monitor for development of adenovirus (AdV) disease |
| | To monitor for development of Epstein Barr virus (EBV) associated syndromes |
| Subject | Subjects who experienced any one of the following criteria after dosing were discontinued from the |
| Discontinuation | study: 1) treatment with an excluded medication*; 2) emergence of CMV disease as evidenced by |
| Criteria: | pneumonia, gastrointestinal disease, or other organ pathology; 3) persistent neutropenia without an |
| | alternative explanation; 4) decrease in GFR to <30 mL/min that is study drug-related or persistent, |
| | 5) pregnancy. |
| | * Note that all approved treatments for CMV infection were excluded, so subjects who developed |
| | CMV DNAemia requiring treatment per site standard of care were discontinued from the study and |
| | treated. |
| Outcome Measures of | Safety endpoints include clinical assessments and laboratory values, AEs (and SAEs), changes from |
| Primary Endpoints: | baseline in laboratory values, vital signs, ECGs, and renal function |
| | Efficacy endpoint is CMV DNAemia >200 copies/mL at the conclusion of treatment or diagnosis of |
| | CMV disease during the treatment period |
| Outcome Measures of | Emergence or increase in plasma CMV DNA levels in stratification subgroups, as |
| Secondary Endpoints: | available |
| | Occurrence of CMV disease; type and severity of disease, and time to onset |
| | Subject drop-out and/or discontinuation rate |
| | Incidence, severity, and progression of GVHD |
| | Trough levels of HDP-CDV, CDV, and other selected metabolites |

### Critical Design and Analysis Considerations: Design Considerations

Key design issues to be considered when reviewing the data are described below:

Screening and Baseline:

Patients were screened for viral load during a visit after transplant or (in the vast majority of cases) conducted a combined screening/predose visit after transplant prior to randomization. Baseline values were assessed immediately before dosing on the first dose day (FDD).

Randomization and Replacement of Subjects:

Subjects were randomized by contacting the Integrated Voice/Web Response System (IV/WRS). Subjects who were randomized but not dosed were considered screen failures and were replaced in the study at the next opportunity, i.e., during the first randomization request made following notification that a randomized subject needed to be replaced. The replacement subject was randomized to the same treatment as the subject being replaced, regardless of site or stratification parameters for the replacement subject. The system was designed in this manner since enrollment was halted in each Cohort when the specified number of subjects was enrolled, regardless of whether or not all individual randomization-stratification blocks were complete. The number for the replacement subjects were identified by having a “5” in the 100s decimal place (e.g., 1513 replaced subject 1013).

For this study, subjects were enrolled when they received their first dose of study drug (as opposed to when they were randomized). Thus, “randomized” does not equal “enrolled” in this study; however, “enrolled” equals “dosed”.

Duration of Therapy:

In the first version of the protocol, the duration of treatment was set at 12 weeks for all subjects; dosing was initiated on “Day 0”; and extended post-treatment follow-up visits were not included. However, this resulted in confusion at the sites, since standard practice at transplant centers is for the Day of Transplant to be designated as “Day 0”; the length of follow-up for each patient is then based on that Day 0. To better minor clinical practice and avoid confusion, the protocol was amended to designate the first day of dosing as “FDD” and the duration of therapy was adjusted to be completed by Day 100 (post-transplant), the day most subjects are returned to the care of their local oncologist. Day 100 post-transplant is also considered to be the end of the period when subjects are at highest risk of CMV infection. The result of this schedule adjustment was that subjects completed study treatment after 9, 10, or 11 weeks, depending on when treatment was initiated.

Discontinuation of Therapy:

Discontinuation criteria as specified in the protocol included: Treatment with an excluded medication (note: all approved treatments for CMV infection were excluded so subjects who developed CMV DNAemia requiring treatment per site standard of care had to be discontinued from the study and treated); emergence of CMV disease as evidenced by pneumonia, gastrointestinal disease, or other organ pathology; persistent neutropenia without an alternative explanation; decrease in GFR to <30 mL/min that was study drug-related or persistent; or pregnancy.

Emergence of CMV infection (CMV DNAemia) up to a specified viral load was not, per se, a discontinuation criterion. Sites were instructed to discontinue subjects requiring treatment for CMV based on standard of care at their site.

### Considerations for the Above Analysis

Statistical Definitions:

The primary efficacy measure for this study was treatment failure, defined as the occurrence of CMV infection, defined as CMV DNAemia>200 copies/mL at the End of Treatment or diagnosis of CMV disease at any time during the treatment period. For the analysis, the following definitions apply:

First Dose Day (FDD):

The first dose day was the first/initial day study medication was given.

Baseline:

The Baseline was defined as the last non-missing assessment prior to, or at the same time as, the first dose of study medication (including Screening, Pre-dose, (or combined Screening/Pre-dose), CMV for stratification, and FDD).

End of Treatment:

For subjects who completed the treatment phase of the study, the End of Treatment was defined as the latest assessment from date of the last dose of study drug and up to 7 days for QW dosing, or the date of the last dose of study drug and up to 4 days for BIW dosing. For subjects who discontinued during the treatment phase of the study, the End of Treatment was defined as the date of the last dose of study drug.

Treatment Period:

The treatment period was defined as the period between FDD and End of Treatment (inclusive).

Time to Event/Onset:

The time to event was defined as the period between FDD and date of first occurrence of the event of interest (inclusive). The events included onset of CMV DNAemia, onset of CMV disease, and discontinuation because of excluded anti-CMV medications. Time to event/onset was derived as (the event/onset date—FDD)+1. Subjects who did not have any event/onset by the end of the study were considered censored at the last study visit.

Discontinuation Date:

The discontinuation date was the date the investigator made the decision to discontinue the subject from randomized treatment (or from continuing in the post-treatment follow-up if the subject completed randomized therapy).

Randomization Stratum:

Two randomization variables were used in this study: CMV viremia and acute GVHD. These randomization strata were defined as follows: (1) CMV: CMV positive was defined as CMV DNA in plasma≧100 copies/mL (i.e., below the limit of detection at the central laboratory) and CMV negative was defined as CMV DNA in plasma<100 copies/mL (i.e., any undetectable value) measured within 7 days prior to dosing; (2) aGVHD: Presence of acute GVHD (aGVHD) was defined as requiring systemic treatment or therapy with steroids (≧1 mg/kg) within 7 days prior to dosing. Subject data relative to randomization strata were entered by the site during the randomization call and used by the system to assign treatment.

Modified Strata for CMV:

As detailed in the Statistical Analysis Plan, a modified CMV stratum was defined prior to conducting the statistical analysis based upon polymerase chain reaction (PCR) results from the central laboratory. If any positive CMV DNA value in plasma was collected before the first dose of study drug (including Screening, Pre-dose, (combined Screening/Pre-dose), CMV for stratification, and FDD), then the subject was placed into the modified CMV positive (≧100 copies/mL) stratum, otherwise the subject was in the modified CMV negative stratum.

Estimation of Placebo CMV Infection Rate:

The estimation of the placebo infection rate for this study was based on existing literature describing the rate of CMV infection in subjects at risk post-HSCT. (Winston D J, et al. Maribavir prophylaxis for prevention of cytomegalovirus infection in allogeneic stem cell transplant recipients: a multicenter, randomized, double-blind, placebo-controlled, dose-ranging study. Blood 2008 June 1; 111(11):5403-10; Boeckh M et al., Randomized, placebo-controlled, double-blind study of a cytomegalovirus-specific monoclonal antibody (MSL-109) for prevention of cytomegalovirus infection after allogeneic hematopoietic stem cell transplantation. Biol Blood Marrow Transplant. 2001; 7(6):343-51; Goodrich J M, et al. Ganciclovir Prophylaxis to Prevent Cytomegalovirus Disease after Allogeneic Marrow Transplant. Ann Intern Med 1993; 118:173-8). Based on these data a rate of CMV infection in the placebo arm was expected to be approximately 50%.

Variables that affect the rate of CMV viremia in subjects enrolled into clinical trials include the time of enrollment into the study relative to the date of transplantation and the analytical approach to measurement of virus in blood including the laboratory's limit of detection; use of whole blood or plasma; and PCR measured DNA copies or PP65 antigen assay for example.

Pharmacokinetics Analysis Considerations:

Plasma samples were collected during the study to assess weekly trough concentrations of HDP-CDV and CDV. The vast majority of weekly trough HDP-CDV concentrations were near or below the lower limit of quantification for the assay. There was no evidence for increasing weekly trough CDV plasma concentrations; therefore, plasma concentrations are not presented in this document. These data are included in the Study HDP-CDV-201 Clinical Study Report.

### Populations Analyzed

Populations analyzed were defined as listed below:

The Intent-To-Treat (ITT) population was defined as all randomized subjects who took at least one dose of study treatment (HDP-CDV or placebo), since subjects who withdrew before the first administration of study drug were replaced. In the event that a subject was incorrectly dosed, the subjects were classified based on the randomization schedule, not what was actually taken. (This population was to be used for the primary efficacy analysis in Part II; Part II of the study was not conducted).

The modified Intent-to-Treat (mITT) population included all randomized subjects who took at least one dose of study treatment and who had at least one efficacy evaluation following baseline. This population was used to summarize efficacy outcomes based on the treatment actually received, in cases in which a subject was treated contrary to the randomization schedule.

The Per Protocol (PP) population included all mITT subjects who completed the study and did not have any major protocol deviations. More specifically, subjects who completed all protocol-specified treatment visits or met the primary efficacy endpoint were considered evaluable subjects and constituted the per protocol population. This population was used to summarize primary efficacy outcomes based on the treatment actually received, in cases where a subject was treated contrary to the randomization schedule.

The Safety population (SAFETY) included all randomized subjects who took at least one dose of study treatment. This population was used to summarize safety outcomes based on the treatment actually received, in cases where a subject was treated contrary to the randomization schedule.

For this study, the ITT, mITT, and SAFETY populations were identical populations.

### Changes Made During Study

During the course of the dose escalation study, several changes were made in reaction to emerging data.

After completion of Cohort 1, it was noted that the rate of CMV disease as a reason for discontinuation was unusually high as compared to historical controls; upon review, some of these subjects had viremia but no proven disease.

After the first three Cohorts of the study were un-blinded, it was noted that Investigators were reporting AEs of GVHD inconsistently, with some using multiple specific terms (i.e., acute GVHD of the intestine and acute GVHD of the liver) and some other using very broad terms (i.e., GVHD). To facilitate interpretation of the data and starting with Cohort 4, the Investigators were ask to report on the AE page either acute or chronic GVHD only and to enter organ involvement in the GVHD staging and grading module.

Due to an excess of SAEs associated with AEs of diarrhea during the dosing of Cohort 4, enrollment in this Cohort was discontinued. The dose was reduced in these subjects still on study drug to 200 mg QW from 200 mg BIW.

A Randomized, Placebo-Controlled, Phase 2 Study, Assessing the Overall Safety, Tolerability and Ability of HDP-CDV to Prevent and/or Control CMV Infection in Post-Engraftment HSCT Recipients

The study included two parts: a dose-escalation phase (Part I) designed to define a range of potentially active doses of HDP-CDV and a confirmatory phase (Part II) designed to confirm the antiviral activity of the dose selected in Part I compared to placebo. Due to positive antiviral effects seen following the interim analyses and increased subject numbers enrolled in Cohort 4a, Part II was not performed.

The efficacy results are presented in detail below, including protocol prescribed analyses and additional exploratory evaluations to help define the antiviral activity of HDP-CDV in adult CMV seropositive (R+) HSCT recipients at risk for clinically significant CMV infection or disease.

### Primary Objectives

The primary efficacy objective of this study was to determine the ability of HDP-CDV to prevent or control CMV infection in R+HSCT recipients during the treatment period.

The specific primary objectives for Part I were: to determine the safety and tolerability of HDP-CDV in R+HSCT recipients; to determine the ability of HDP-CDV to prevent or control CMV infection in R+HSCT recipients during the treatment period; and to select the dose of HDP-CDV to be used in Part II based on safety and efficacy.

The primary objectives for Part II were: to compare the safety and tolerability of HDP-CDV to that of placebo in HSCT recipients; and to compare the activity of HDP-CDV, at the dose selected based upon the results from Part I, to that of placebo in terms of prevention or control of CMV infection in R+HSCT recipients during the treatment period. The overall primary analysis for the study was to be measured by the difference in CMV infection rates of all HDP-CDV treated subjects who received the dose given to subjects in Part II compared to the placebo rate. Based on the results from interim analyses and enrollment into Cohorts 3 and 4a, Part II was not conducted.

### Endpoints

The primary efficacy endpoint for the study was a comparison between HDP-CDV- and placebo-treated subjects of treatment failures, defined as the occurrence of CMV infection (CMV DNAemia>200 copies/mL obtained at the end of treatment with study drug) OR the diagnosis of CMV disease at any point during the treatment phase. The CMV infection/disease rate was calculated as the number of subjects with the outcome of CMV infection/disease (by definition) divided by the number of subjects within each treatment group. Since this was a prevention/preemptive therapy trial, patients with known CMV infection requiring imminent treatment with an anti-CMV agent were not eligible for enrollment. The analysis population for Part I was the mITT population; a missing value was considered as failure and a sensitivity analysis was conducted using “last observation carried forward” (LOCF).

Efficacy for the dose-escalation Cohorts in Part I was to be assessed in several ways. First, pairwise comparisons of the differences in infection rates were to be performed for all HDP-CDV treated subjects combined and for each individual HDP-CDV dose compared to the pooled placebo using a Fisher's exact test. As supportive analyses, Cochran-Mantel-Haenszel (CMH) tests adjusted for stratification factors were also performed, including a Breslow-Day test for homogeneity of odds ratios across the levels of the stratification factor. In addition, similar pairwise comparisons were to be carried out for an exploratory analysis in which all of the HDP-CDV-treated subjects from the dose-escalation Cohorts satisfying the CMV disease or viremia endpoint of 50% less than the placebo rate but not greater than 25% overall were compared to all placebo subjects from those Cohorts. The analysis for this endpoint was conducted for each efficacy assessment and labeled “Exploratory Analysis” at the bottom of each relevant SAS efficacy table. Additional subgroup analyses were to be carried out for the individual strata levels (presence of aGVHD requiring treatment and modified strata for CMV) for exploring treatment differences using a Fisher's exact test. Results of these comparisons are described below.

### Sample Size Calculations

To estimate the sample size, each of the dose-escalation Cohorts was powered to detect a difference in CMV infection/disease rates between HDP-CDV-treated subjects and historical infection rates for untreated patients, in order to identify a dose for comparison to placebo in Part II. A limited sample size was selected in order to limit exposure of study subjects to potentially ineffective doses of HDP-CDV. Specifically, the initial sample size calculation for each Cohort in Part I was based on identifying a confidence interval around an acceptable clinical rate of success for the HDP-CDV group alone. Based on literature review, the incidence of emergent CMV infection following allogeneic HSCT in R+ subjects ranged from approximately 45% to 70%. (Boeckh M et al. Cytomegalovirus pp 65 Antigenemia-Guided Early Treatment with Ganciclovir Versus Ganciclovir at Engraftment after Allogeneic Marrow Transplantation: A Randomized Double-Blind Study. Blood 1996 November 15; 88(10):4063-71). Reducing the incidence of infection by 50% was considered a clinical success.

Based upon the published historical rates and assuming that an acceptable overall CMV infection rate would be 25%, but not more than 35%, in HDP-CDV treated subjects (defined as CMV DNAemia>200 copies/mL at the end of treatment or the diagnosis of CMV disease), a sample size of 21 subjects randomized to HDP-CDV would yield an 85% confidence interval around the 25% infection rate that would exclude a rate greater than 35%. Assuming a dropout rate of 15%, at least 24 subjects were to be randomized to receive HDP-CDV in each Cohort to ensure that sufficient evaluable subjects were available.

Subjects were randomized 3:1 (HDP-CDV: placebo) in each dose-escalation Cohort, with at least 8 placebo subjects enrolled per Cohort, for a total of 32 subjects per Cohort. Per protocol, subjects who had signed the informed consent prior to confirmed enrollment (i.e., dosing) of the 32^{nd} subject into each Cohort were allowed to enroll into the study, either into the ongoing Cohort if the subsequent Cohort had not yet been opened, or into the next Cohort if DSMB approval had been received and the Cohort was open for enrollment. Since Cohort 4a was the last Cohort in the study, this practice resulted in significant over-enrollment into that Cohort (50 drug- and 17 placebo-treated). Placebo subjects from all Cohorts were pooled for efficacy and safety analyses.

### Results: Analyses of the Primary Endpoint

Analysis of the mITT Population

The primary endpoint was the rate of failure to prevent CMV infection as defined by either CMV disease occurring at any time during the treatment period OR the presence of CMV DNAemia>200 copies/mL at the end of treatment. Results from the primary analysis are shown in TABLE 2, which includes a detailed listing of the CMV event rates of all HDP-CDV subjects combined; each HDP-CDV Cohort; and the pooled placebo group.

The first pre-specified analysis compared the CMV infection rates (as defined by the primary endpoint) of the combined HDP-CDV treatment groups to that for the pooled placebo group. The overall infection rate in the pooled placebo group was 37.3%, which was lower than the anticipated 50% rate based upon historical reports in the literature.

The infection rates among all HDP-CDV subjects combined was 25.1% (p=0.09 compared to pooled placebo). Within the mITT population, there were 50 subjects who were CMV positive at baseline (38 HDP-CDV- and 12 placebo-treated; 21.7% of subjects enrolled) and 180 who were CMV negative (133 HDP-CDV- and 47 placebo-treated; 78.2% of total enrolled). Using a Cochran-Mantel-Haenszel (CHM) test, when the pooled data from all HDP-CDV doses were adjusted for the screening-dose-defined CMV randomization strata, the result was marginally significant (p=0.057); when adjusted by the modified CMV strata (which took into account the actual CMV status of each subject on the first day of dosing), the difference in the combined CMV infection endpoint (CMV disease at any time or CMV DNAemia>200 copies/mL at end of treatment) was statistically significant (p=0.041).

The pre-specified primary endpoint analyses also included the comparison of the CMV event rates of the individual HDP-CDV treatment groups by Cohort to that for the pooled placebo group. When individual Cohorts were analyzed, treatment with the 100 mg BIW dose met the primary endpoint of the study as it was significantly superior to the pooled placebo in preventing CMV disease/end of treatment infection (event rate of 10% vs. 37%, p=0.002). The dose of 40 mg QW (event rate of 52%) was not different from placebo (Cohort 1 placebo, 50%; Pooled placebo, 37%), while the event rates of the other three doses, 100 mg QW (22%), 200 mg QW (31%), and 200 mg BIW (23%) were comparable, but numerically lower than that for placebo-treated subjects. Event rates for the individual placebo groups ranged from a low of 29% (Cohort 3) to a high of 50% (Cohort 1) (data not shown); the variability in placebo CMV disease/infection rates across Cohorts was likely a reflection of the small individual Cohort sample sizes.

An exploratory analysis related to the primary endpoint was defined per protocol and compared doses of HDP-CDV to a predefined clinically relevant endpoint of 50% reduction versus placebo but not greater than a 25% overall infection rate (CMV disease at any time or DNAemia>200 copies/ml at end of treatment). This exploratory analysis allowed for pooling of results from HDP-CDV dose Cohorts that satisfied the endpoint in an effort to more accurately assess statistical significance in given small sample sizes. In this analysis, only the 100 mg BIW dose met the exploratory endpoint and was statistically superior to the respective Cohort 4a placebo response (p=0.008).

The primary endpoint comparing differences in CMV disease/end of treatment infection rates for all HDP-CDV treated subjects combined versus pooled placebo was significantly different when adjusted for CMV modified strata. This adjustment is particularly relevant in planning for subsequent studies focused upon the prevention of CMV reactivation in HSCT subjects who are CMV viremia negative at baseline. When the full complement of subjects were included (CMV positive and negative at baseline), the difference between HDP-CDV and placebo treatment trended towards significance using a Fisher's exact text. When each Cohort was analyzed separately, the 100 mg BIW treated subjects demonstrated a statistically significant difference in infection rates compared to placebo.

### Analysis by Stratification Groups

Subjects were stratified at randomization by two parameters which are considered to be significant predictors of development of CMV disease post-transplant: detectable CMV DNAemia and aGVHD requiring systemic therapy.

Results of the primary analysis for the aGVHD and CMV positive strata are summarized here. Few subjects were stratified into either the aGVHD (N=15) or CMV (N=50) positive strata. In the aGVHD positive strata, for all Cohorts combined, HDP-CDV treatment resulted in a lower CMV disease/infection rate, with 2 of 9 subjects (22.2%) achieving this endpoint versus 5 of 6 subjects (83.3%) in the pooled placebo group (p=0.041). Furthermore, results from the four Cohorts with doses≧100 mg QW met the exploratory endpoint of 50% less than placebo but not greater than 25%. Although the combined exploratory analysis group was not significantly different from placebo, only 1 out of 7 subjects developed CMV disease/infection (14.3%) versus 4 out of 5 placebo subjects (80%) (p=0.07).

In the CMV modified positive stratum, there were 38 subjects treated with various doses of HDP-CDV versus 12 with placebo. While the primary analysis was not statistically significant for the pooled data or any Cohort, in the 100 mg BIW Cohort, 3 of 9 subjects (33%) developed CMV events (disease at any time or >200 copies at end of treatment) compared to 8 of 12 (66.7%) pooled placebo subjects.

The majority of subjects in Study HDP-CDV-201 were enrolled into the negative strata, as expected based on this early enrollment, relative to transplantation, for either aGVHD (N=215) or CMV DNAemia (N=180). Results of the analyses for the combined CMV infection endpoint for each of these negative strata are shown below in TABLE 3. (See above).

The results demonstrate that subjects in the 100 mg BIW Cohort, stratified to either the aGVHD or CMV negative stratum, met the primary endpoint of the study in that the CMV disease/infection rates for either HDP-CDV-treatment subgroup was significantly better than that for placebo (9% and 5% compared to 32% and 30% for aGVHD and CMV, respectively). Analysis of pooled HDP-CDV treated subjects versus pooled placebo treatment was not significant for the aGVHD negative stratum (p>0.05) and was of borderline significance for the modified CMV negative stratum (p=0.052). Consistent with the previous results, a dose of 40 mg QW was not effective, and results from the three middle dose groups trended together.

Each Cohort of HDP-CDV-treated subjects was also assessed to determine if it satisfied the exploratory dose-escalation endpoint of achieving a CMV disease/infection rate that was 50% less than that for placebo but not greater than 25% overall; for this assessment, placebo results from the individual Cohorts were used. For the CMV negative stratum, three HDP-CDV treatment groups (100 mg QW, 200 mg BIW, and 100 mg BIW; shown with an asterisk above) satisfied this endpoint, yielding a pooled sample size of 86 subjects of which 8 (9%) failed compared to 10 (31%) out of 32 placebo subjects (p=0.007).

Conclusions from Primary Endpoint and Stratification Analyses

As noted above, the ability of HDP-CDV to prevent CMV disease or end of treatment CMV infection in R+HSCT recipients during the treatment period was demonstrated in the mITT population when the difference between the pooled HDP-CDV Cohorts and placebo was adjusted for CMV modified strata (p=0.041). In addition, there was a significant difference in the 100 mg BIW HDP-CDV (the largest Cohort with n=50 subjects) compared to pooled placebo for the mITT population and for the modified CMV negative stratum. Furthermore, for subjects who were CMV DNA negative prior to dosing, the exploratory endpoint of a greater than 50% reduction in the CMV disease/infection rate was significantly different for the pooled Cohorts 100 mg QW, 200 mg BIW, and 100 mg BIW compared to pooled placebo (p=0.007). For subjects who were aGVHD positive prior to dosing, a clinically relevant reduction in the emergence of CMV disease/infection was seen, although the number of subjects was small.

### Secondary Objective of CMV Infection Study: Incidence and Time to Onset of CMV DNAemia (≧100 Copies/mL)

Incidence and time to onset of CMV DNAemia were assessed in subjects stratified to the modified CMV negative stratum as shown below using the Kaplan-Meier Survival method and overall incidence.

For this analysis, CMV DNAemia was defined as any subject having at least one measurement of ≧100 CMV DNA copies/mL at any time during the treatment period; of note, 100 copies/mL is the limit of detection for the Viracor PCR assay. This analysis provides an assessment of the prophylactic activity of various doses of HDP-CDV in CMV seropositive HSCT recipients.

The incidence and time to onset were calculated using the Kaplan-Meier method for all individual HDP-CDV dose Cohorts compared to pooled placebo, for the treatment period (TABLE 4).

All four of the higher doses in HDP-CDV Cohorts were superior to placebo in preventing CMV breakthrough. The overall rates of CMV DNAemia detectable at any time during treatment ranged from 17.4 to 23.8% across the doses of 100 mg QW and higher, compared to the pooled placebo rate of 49%. As part of this analysis, peak CMV viremia levels were also compared across Cohorts. The median peak viral load for HDP-CDV treated subjects in Cohorts 2, 4, and 4a was 2.3; 2.2; 2.0 log₁₀, respectively (200, 158 and 100 copies/mL, respectively), approximately 1 log₁₀ lower than the peak viral load seen in placebo treated patients (3.2 log₁₀; 1,584 copies/mL).

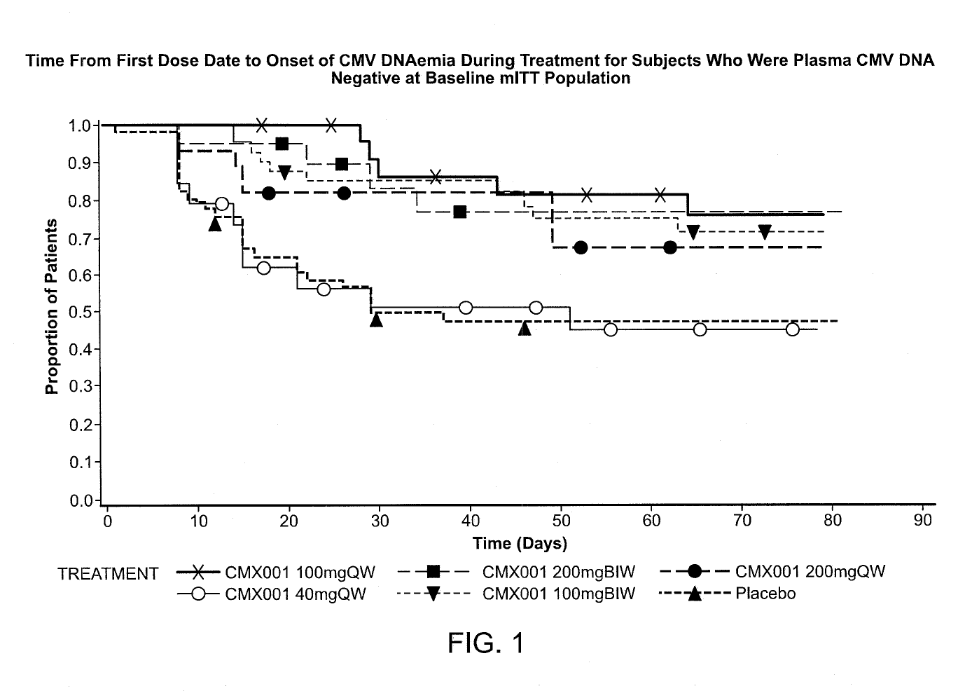

In this analysis of the incidence of CMV viremia, the placebo rate of 49% was more in line with historical results and matched the expected placebo rate for the study. All 4 HDP-CDV doses≧100 mg QW had CMV DNAemia rates≦50% of the placebo rate; these rates of reduction are generally considered to be clinically relevant. (Kharfan-Dabaja M A, et al. A novel therapeutic cytomegalovirus DNA vaccine in allogeneic haemopoietic stem-cell transplantation: a randomised, double-blind, placebo-controlled, phase 2 trial. Lancet Infect Dis. 2012 April; 12(4):290-299). The corresponding Kaplan-Meier survival curve is shown in FIG. 1.

As seen in FIG. 1, the survival curves for placebo and 40 mg QW were indistinguishable while those for the higher doses were similar. Of note, the survival curves for the two Cohorts with the same total weekly dose (i.e., 200 mg QW and 100 mg BIW) were nearly identical. Thus for this analysis, in subjects who were CMV DNAemia negative at baseline, there was no apparent difference in the occurrence of detectable CMV viremia between once versus BIW dosing.

Time to onset and median survival time were also assessed for each treatment group over the entire study period (i.e., including the follow-up period posttreatment) in an effort to investigate the durability of the antiviral activity of HDP-CDV posttreatment, as shown in TABLE 12.

TABLE 12
Time From First Dose to Onset of CMV DNAemia (≧100 copies/mL) During the Whole Study Period for
Subjects Who Were Plasma CMV DNA Negative at Baseline (Modified CMV Negative Stratum, mITT Population)

| | | Cohort 1 | Cohort 2 | Cohort 3 | Cohort 4 | Cohort 4A |
|---|---|---|---|---|---|---|
| | Pooled | HDP-CDV | HDP-CDV | HDP-CDV | HDP-CDV | HDP-CDV |
| | Placebo | 40 mg QW | 100 mg QW | 200 mg QW | 200 mg BIW | 100 mg BIW |
| | N = 59 | N = 25 | N = 27 | N = 39 | N = 30 | N = 50 |
| CMV DNA Negative at Baseline | 49 | 19 | 23 | 30 | 23 | 42 |
| Time to CMV DNAemia (Days)^{[1]} | | | | | | |
| N^{[2]} | 27 | 11 | 8 | 11 | 9 | 14 |
| Mean | 18.3 | 24.5 | 53.4 | 36.3 | 49.0 | 42.8 |
| Median | 15.0 | 15.0 | 38.5 | 36.0 | 42.0 | 32.5 |
| Median Survival Time (Days) | 29.0 | 51.0 | N/A | N/A | 130 | N/A |
| Log-Rank p-value^{[3]} | N/A | 0.864 | 0.033 | 0.094 | 0.164 | 0.020 |

^{[1]}Censored values are not included.
^{[2]}Number of Subjects who had CMV DNAemia by the end of study; those who did not develop DNAemia were considered censored at the end of study for this analysis.
^{[3]}Results based on Kaplan-Meier (KM) method.

The CMV DNAemia rates for Cohorts receiving 100 mg QW, 200 mg QW and 100 mg BIW were approximately the same (˜35%) compared to the placebo DNAemia rate of 55% and the mean and median times to first onset of CMV DNAemia were longer than for placebo. Of the HDP-CDV treatment groups, both the 100 mg QW and 100 mg BIW Cohorts were significantly better than placebo in this analysis (p=0.033 and 0.020, respectively). Moreover, subjects who became CMV viremic during the safety follow-up phase of the study responded to antiviral therapy with alternative agents (e.g., GCV or foscarnet) as discussed in the virology section. Taken together, these results suggest that rebound viremia and/or disease following the end of treatment was not a significant issue in this study.

Taken in combination, the results from the incidence and “time-to” analyses (TABLE 4 and TABLE 12) demonstrate that treatment with HDP-CDV at doses≧100 mg QW were significantly superior to placebo in preventing the emergence of CMV DNAemia during both the treatment and posttreatment follow-up periods and support further assessment of HDP-CDV treatment as prophylaxis against CMV infection.

### Secondary Objectives and Endpoints

The mITT population was also used for analyses of additional secondary efficacy endpoints. The percentage of subjects with a given endpoint was compared between HDP-CDV and placebo groups, similar to the analytic approach for the primary endpoint. Where applicable, peak CMV viremia values were summarized as log₁₀ transformed values of the number of copies/mL. In addition, the Kaplan-Meier (KM) method was used to compare treatment differences in time to onset of CMV DNAemia and the log-rank test was used to assess treatment differences. The number of subjects who had the event or were censored and the median time to onset were noted, and the survival curves plotted by treatment. Similarly, Time to Peak CMV Viremia was also analyzed using the KM method and log-rank test.

### Secondary Efficacy Objectives

Secondary Objective 1:

In subjects who were plasma CMV DNA negative at baseline (CMV modified stratum: negative), to compare the incidence and time to onset of CMV DNAemia between HDP-CDV treated and placebo subjects and between HDP-CDV treatment groups during the treatment and subsequent 8 week follow-up period.

In subjects who were plasma CMV DNA positive at baseline (CMV modified stratum: positive), to compare the change from baseline in CMV DNAemia between HDP-CDV treated and placebo subjects and between HDP-CDV treatment groups during the treatment period and subsequent 8 week follow-up period.

To compare the incidence and time to onset of CMV disease between HDP-CDV treated and placebo subjects and between HDP-CDV treatment groups during the treatment and subsequent 8 week follow-up period.

Secondary Objective 2:

CMV DNAemia Change from Baseline

CMV DNAemia change from baseline was calculated for all subjects who were CMV DNA positive at Baseline. An overall trend in reduction of viral load was seen, especially at the higher doses of HDP-CDV. However, small sample sizes, inconsistency of Baseline viral loads between Cohorts, and inconsistency in site treatment practices resulted in an inability to effectively assess the antiviral activity of HDP-CDV in CMV positive subjects using this analysis, although a trend towards greater activity of BIW doses was discernible.

Secondary Objective 3:

Time to Onset of CMV Disease

Time to onset of CMV disease could not be estimated, due to the low rate of occurrence of disease as reflected by the low number of subjects who discontinued due to CMV disease. As shown in TABLE 13 only nine subjects discontinued from the study due to CMV disease, two in the placebo group and seven among those who received HDP-CDV (3 on 40 mg QW, 3 on 100 mg QW, and 1 on 100 mg BIW). Overall, 2/59 (3.4%) of placebo recipients developed CMV disease versus 4/146 (2.7%) of subjects who received HDP-CDV doses of 100 mg QW or higher, doses that were associated with antiviral activity. When HDP-CDV doses of 200 mg per week or higher are considered, 1/119 (0.8%) of the subjects developed CMV disease.

Reasons for subject discontinuations are further described in TABLE 13.

TABLE 13
Subject Discontinuations by Cohort (1 through 4A) during the
Active Treatment Period (through Week 11), (Subject n (%))

| | Cohort | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| | N = 25 | N = 27 | N = 39 | N = 30 | N = 50 | N = 59 |
| Adverse event | 3 (12.0%) | 3 (11.1%) | 7 (17.9%) | 13 (43.3%) | 8 (16.0%) | 7 (11.9%) |
| Death | 1 (4.0%) | 0 | 3 (7.7%) | 3 (10%) | 4 (8.0%) | 0* |
| Emergence of CMV disease | 3 (12.0%) | 3 (11.1%) | 0 | 0 | 1 (2.0%) | 2 (3.4%) |
| Initiation of alternate | 8 (32.0%) | 4 (14.8%) | 7 (17.9%) | 5 (16.7%) | 6 (12.0%) | 16 (27.1%) |
| CMV therapy | | | | | | |
| Excluded medication, not | 1 (4.0%) | 0 | 1 (2.6%) | 0 | 0 | 1 (1.7%) |
| for CMV | | | | | | |
| Lost to follow-up | 0 | 2 (7.4%) | 0 | 0 | 0 | 0 |
| Physician decision | 0 | 0 | 2 (5.1%) | 1 (3.3%) | 1 (2.0%) | 1 (1.7%) |
| Withdrawal of consent | 1 (4.0%) | 1 (3.7%) | 5 (12.8%) | 1 (3.3%) | 4 (8.0%) | 0 |
| Other | 1 (4.0%) | 0 | 0 | 1 (3.3%) | 0 | 2 (3.4%) |

*Placebo-treated subjects were discontinued for other causes (e.g., initiation of excluded medications to treat CMV) and subsequently died. See next section for complete account of all deaths in the study.

To explore the incidence and time to CMV events, a Kaplan-Meier “time-to” analysis was conducted using CMV disease and/or discontinuation due to initiation of alternative anti-CMV therapy. This endpoint assessed the emergence of CMV disease or CMV DNAemia requiring initiation of antiviral therapy, comparing HDP-CDV- and placebo-treated subjects. As shown in TABLE 7, a total of 55 subjects, distributed across all treatment groups, were discontinued due to CMV events.

The results demonstrate that, 14% of the subject receiving 100 mg BIW required antiviral treatment and/or had CMV disease compared to 30.5% of the placebo subjects. In the pooled HDP-CDV treated subjects 37/171 (21.6%) required initiation of anti-CMV therapy versus 30.5% in the placebo group. Doses of 200 mg QW, 200 mg BIW, and 100 mg BIW were numerically similar with respect to this composite endpoint.

### Additional Post Hoc Analyses

Since increases in CMV DNAemia levels are a prelude to serious CMV infection and disease, site standard practices usually identified CMV viral load at which treatment must be initiated. The level at which antiviral therapy was initiated is 1,000 copies/mL. In addition to the analyses specified in the HDP-CDV-201 protocol and SAP, additional investigations were conducted to further explore the antiviral activity of HDP-CDV in this context.

The first series of evaluations compared HDP-CDV- to placebo-treatment for the ability to prevent emergence of clinically relevant CMV events in the mITT population, modified CMV negative stratum. The events comprising the combination endpoint were CMV disease, CMV viremia requiring treatment based on clinical judgment of the treating physician, and/or emergence of CMV viremia≧1000 copies/mL. The results are shown in TABLE 8.

In this analysis, the combined CMV event rates in the two HDP-CDV dose Cohorts of 200 mg QW were comparable: 13.8% for 200 mg QW versus 12.2% for 100 mg BIW. The results from each of these Cohorts were significantly superior to the pooled placebo rate (35.4%).

Both CMV disease and the clinical decision to treat CMV DNAemia are clinically meaningful endpoints. The CMV viremia endpoint of ≧1000 copies/mL may trigger the decision to institute pre-emptive treatment. For this reason, a separate analysis was performed. Specifically, subjects in Study HDP-CDV-201 were monitored throughout treatment up to and including the posttreatment week 1 follow-up visit for CMV DNAemia levels≧1000 copies/mL. For this analysis and for each Cohort, HDP-CDV treated subjects in the mITT population were compared to placebo treated subjects from the same Cohort and the pooled placebo group. The CMV DNAemia rates per Cohort are shown in TABLE 5.

When all combined HDP-CDV doses were compared to pooled placebo, there was significant difference in CMV DNAemia failure rates, defined as at least one DNAemia value≧1000 copies/mL during the treatment period (17.0% versus 42.4%, p<0.001). When individual dose Cohorts were analyzed, the HDP-CDV 200 mg QW and both BIW doses were also significantly superior to placebo in preventing CMV DNA levels from reaching 1000 copies/mL during treatment; DNAemia rates were 18%, 7%, and 8% compared to the placebo rate of 42% (p=0.015, <0.001, and <0.001 respectively). The three highest HDP-CDV dose Cohorts (200 mg QW, 200 mg BIW, and 100 mg BIW) combined together also met the exploratory endpoint compared to placebo (11% versus 34%, p=0.001).

Post hoc analyses using the ≧1000 copies/mL endpoint were conducted for each modified CMV strata. Results for the modified CMV positive stratum are shown in TABLE 14.

TABLE 14
Rates of CMV DNAemia ≧1,000 copies/mL During the Treatment
Period (mITT Population) Post Hoc Analysis (CMV DNAemia levels
to at least 1000 copies/mL) CMV Modified Strata: Positive

| | | DNAemia | | Difference^{[1]} | | |
|---|---|---|---|---|---|---|
| | | | Rate | Result | | P- |
| Treatment Group | N | n | (%) | (%) | 95% C.I. | value^{[2]} |

Primary Analysis

| | | | | | | |
|---|---|---|---|---|---|---|
| All HDP-CDV | 38 | 21 | 55.3 | −28.1 | −54.4, −1.7 | 0.100 |
| 40 mg QW | 7 | 6 | 85.7 | 2.4 | −31.0, 35.8 | 1.000 |
| 100 mg QW | 4 | 4 | 100.0 | 16.7 | −4.4, 37.8 | 1.000 |
| 200 mg QW | 10 | 5 | 50.0 | −33.3 | −70.8, 4.1 | 0.172 |
| 200 mg BIW | 8 | 2 | 25.0 | −58.3 | −95, −21.7 | 0.019 |
| 100 mg BIW | 9 | 4 | 44.4 | −38.9 | −77.6, −0.2 | 0.159 |
| Pooled Placebo | 12 | 10 | 83.3 | | | |

^{[1]}Difference is HDP-CDV minus placebo. C.I. = Confidence intervals calculated using binomial distribution.
^{[2]}Fisher's exact test relative to placebo.

In the small number of subjects with viremia (≧100 copies/mL) at baseline, the comparison of DNAemia rates (≧1,000 copies/mL at any time during treatment) for all HDP-CDV doses combined was numerically improved from that for the pooled placebo (55.3% versus 83.3%; p=0.1). In addition, the HDP-CDV doses of 200 mg BIW (DNAemia rate of 25%) and 100 mg BIW (DNAemia rate of 44.4%) showed a trend towards greater activity of twice weekly dosing compared to the pooled placebo (DNAemia rate of 83%) in preventing the rise of CMV DNAemia to the clinically significant level. Finally, results from Cohorts with a total dose of 200 mg weekly (i.e., Cohorts 200 mg QW and 100 mg BIW) were numerically similar: 5 out of 10 and 4 out of 9 failures, respectively.

For the modified CMV positive stratum, formal statistical analysis of the change in viral load over time was not possible, given the small group sizes; variability in initial viral loads; and the institution of preemptive CMV therapy in several subjects across the groups. In order to visually present the individual data, smooth-line scatter plots are provided below for the placebo, 200 mg QW and 100 mg BIW subjects, respectively.

The majority of placebo treated subjects who were CMV positive at baseline went on to develop high viral titers and ultimately required preemptive treatment (8/11). While both 200 mg QW and 100 mg BIW had fewer, but similar numbers of subjects initially reaching 1,000 copies/mL, these line plots suggest that 100 mg BIW was more effective in suppressing the low level viral titers, avoiding the potential need for additional anti-CMV intervention.

When the rates of CMV DNAemia≧1,000 copies/mL during the treatment period were evaluated for subjects in the modified CMV negative stratum, the pooled HDP-CDV treatment groups were highly significantly different compared to pooled placebo (p<0.001). All HDP-CDV dose Cohorts except for 40 mg QW were significantly superior to pooled placebo in preventing an increase in CMV DNAemia levels to ≧1000 copies/mL. (See TABLE 6).

Of note, there were no instances of CMV DNAemia≧1000 copies/mL in the BIW treatment groups. In this analysis, HDP-CDV was effective in preventing emergence of CMV DNAemia to levels≧1000 copies/mL.

In order to visually present the individual subject data, smooth-line scatter plots are provided below for the placebo, 200 mg QW and 100 mg BIW subjects in the modified CMV negative stratum.

These scatter plots provide evidence of improved efficacy of BIW dosing. While the placebo-treated subjects experienced clinically meaningful viremia, prompting consideration of preemptive therapy with ganciclovir or valganciclovir, few subjects in the 200 mg QW group achieved DNAemia>1,000 copies/mL. Of note, subjects treated with 100 mg BIW had the lowest frequency and severity of breakthrough viremia, with no one approaching a DNAemia value that would have triggered preemptive treatment.

### Virology Assessments of Possible Resistance

In this study, 171 subjects received active drug and 59 subjects who received placebo. Approximately 25% (43/171) of the subjects receiving HDP-CDV met the primary endpoint overall; 13 of these were in Cohort 1 (40 mg HDP-CDV QW). Subjects who became CMV viremic at the end of treatment or during the safety follow-up phase of the study responded to antiviral therapy with alternative agents (e.g., GCV or foscarnet). Specifically, of the 46 subjects receiving HDP-CDV and for whom follow-up data were available, all but 1 experienced a decrease in CMV DNAemia following the switch to alternate CMV therapy. The subject who didn't respond had received one dose of 40 mg HDP-CDV prior to discontinuing HDP-CDV. Similarly, 20 of the 21 placebo subjects with available follow-up data responded to respond to subsequent GCV therapy.

Plasma samples obtained during the active treatment period from HDP-CDV treated subjects with virologic failure were analyzed. Samples from Cohort 1 were excluded from resistance testing as the 40 mg QW dose of HDP-CDV did not have antiviral activity in Study HDP-CDV-201. Plasma samples were evaluated for genotypic changes in comparison to the AD169 reference or baseline genotype (when available). CMV plasma DNAemia (viremia) was determined by a quantitative PCR assay performed by Viracor-IBT Labs (5500 Cytomegalovirus (CMV) Real-time qPCR). The genotypic assay to assess viral resistance was performed by Viracor-IBT Labs (5600 Cytomegalovirus (CMV) Antiviral Resistance) and involved conventional PCR followed by DNA sequencing of the UL54 and UL97 genes. Sequencing spanned the regions that are known to contain the sites of resistance mutations, including the regions in UL54 expected to be the target of an alternative substrate inhibitor such as the active antiviral formed from HDP-CDV (CDV-PP).

All changes in UL54 from the regions amplified were evaluated. No UL54 mutations that have been proven to confer drug resistance were detected in HDP-CDV-treated subjects. No resistance associated mutations were detected in UL97 in HDP-CDV-treated subjects, as expected. Three subjects harbored CMV which carried mutations in UL54 that have been previously reported in a drug-resistant clinical isolate. (Smith I L, et al. High-Level Resistance of Cytomegalovirus to Ganciclovir Is Associated with Alterations in both the UL97 and DNA Polymerase Genes. J. Infect. Dis. 1997; 176:69-77). This mutation, R1052C, has been reported in a clinical isolate with a CDV resistant phenotype, although the single CMV isolate containing R1052C with published phenotype data also had other mutations in UL54 and UL97.

Of the three subjects who received HDP-CDV and had a detectable CMV variant carrying R1052C, one (receiving 100 mg BIW) had the variant detected at baseline. This subject did not have a virologic response to HDP-CDV but had a complete virologic response to subsequent vGCV. The other two subjects (one receiving 100 mg QW and the other 200 mg QW) had undetectable CMV plasma viremia at baseline, so it was not possible to determine their baseline CMV genotype. Both subjects had the R1052C mutation detected concomitant with viral breakthrough. One of these subjects subsequently initiated CDV therapy while the other initiated vGCV therapy. The subject receiving CDV did not respond, but the subject receiving vGCV had a complete virologic response.

These data suggested R1052C was a candidate to confer resistance to HDP-CDV and CDV. The role of R1052C in HDP-CDV resistance is being assessed by recombinant phenotyping; preliminary results indicate there is no change in CDV resistance from wild-type conferred by this single mutation in isolation.

Overall no UL97 or UL54 mutations proven to confer drug resistance were detected in subjects enrolled on active drug in study this study. The UL54 mutation R1052C was detected in 3 subjects in this study. This mutation was previously reported in a multi-drug resistant CMV isolate that also contained other mutations in UL97 and UL54 known to confer drug resistance. The HDP-CDV phenotype of R1052C is under investigation; preliminary results do not show resistance to CDV.

### Efficacy by Cohort

The results presented above demonstrated that HDP-CDV doses of 100 mg QW or higher demonstrated antiviral activity and resulted in significant benefit over placebo in several analyses. The results for the primary, secondary, and exploratory endpoints are condensed together into TABLE 15. In several analyses, the HDP-CDV 100 mg BIW Cohort demonstrated statistically and clinically significant differences compared to placebo, although the impact of increased group size could not be excluded. While both 200 mg QW and 100 mg BIW appeared similar in many analyses, visual inspection of smoothed-line scatter plots suggest that BIW dosing may be more effective in suppressing CMV DNAemia in subjects who are either CMV negative or have low level CMV viremia at the start of treatment.

TABLE 15
HDP-CDV-201: Summary of Antiviral Activity Results

| | Results | | | | |
|---|---|---|---|---|---|
| Protocol Specified Analyses | | % CMV Event Rate | | | Reference |
| Population | Dose | HDP-CDV | Placebo | p-value | TABLE |

Primary Efficacy Analysis: CMV DNAemia >200 copies/mL at end of treatment OR
diagnosis of CMV disease during treatment

| | | | | | |
|---|---|---|---|---|---|
| mTT | All HDP-CDV (N = 171) | 43 (25.1%) | 22 (37.3%) | 0.041^{a} | 14.2.1 |
| | 100 mg BIW (N = 50) | 5 (10.0)% | | 0.001^{a} | |
| CMV modified strata: negative | All HDP-CDV (N = 133) | 21 (15.8%) | 14 (29.8%) | 0.052 | 14.2.1.4 |
| | 100 mg BIW (N = 41) | 2 (4.9%) | | 0.002 | |
| | Combined (100 mg QW, 200 mg | 8 (9.3%) | 10 (31.3%) | 0.007 | |
| | BIW, 100 mg BIW) (N = 86) | | | | |
| aGVHD modified strata: negative | All HDP-CDV (N = 162) | 41 (25.3%) | 17 (32.1%) | NS | 14.2.1.3 |
| | 100 mg BIW (N = 46) | 4 (8.7%) | | 0.006 | |

Secondary Efficacy Analysis: Incidence of and Time to CMV DNAemia >100 copies/mL at any time

| | | | | | |
|---|---|---|---|---|---|
| mITT (Incidence of CMV DNAemia | 100 mg QW | 5 (21.7%) | 23 (48.9%) | 0.039 | 14.3.3.2.1 |
| During Treatment) (p-value derived using | 200 mg QW | 7 (24.1%) | | 0.052 | |
| Fisher's exact test relative to placebo) | 200 mg BIW/QW | 4 (18.2%) | | 0.018 | |
| Modified CMV negative stratum | 100 mg BIW | 9 (22.5%) | | 0.014 | |
| mITT (Time From First Dose to Onset of | 100 mg QW | 5 (21.7%) | 24 (49.0%) | 0.011 | 14.2.4.1 |
| CMV DNAemia During Treatment) | 200 mg QW | 7 (23.3%) | | 0.045 | |
| (p-value is log-rank p-value based on | 200 mg BIW/QW | 4 (17.4%) | | 0.020 | |
| Kaplan-Meier method) | 100 mg BIW | 10 (23.8%) | | 0.009 | |
| Modified CMV negative stratum | | | | | |
| mITT (Time From First Dose to Onset of | 100 mg QW | 8 (34.8%) | 27 (55.1%) | 0.033 | |
| CMV DNAemia During Whole Study) | 100 mg BIW | 14 (33.3%) | | 0.020 | |
| (p-value is log-rank p-value based on | | | | | |
| Kaplan-Meier method) | | | | | |
| Modified CMV negative stratum | | | | | |

Exploratory Endpoint: Incidence of CMV Disease, Initiation of Excluded CMV
Medications or DNAemia ≧1,000 copies/mL at any time during treatment

| | | | | | |
|---|---|---|---|---|---|
| mITT (modified CMV negative stratum) | 200 mg QW | 4 (13.8%) | 18 (38.3%) | 0.036 | 14.3.3.2.5 |
| (p-value derived using Fisher's exact test | 200 mg BIW/QW | 3 (13.6%) | | 0.050 | |
| relative to placebo) | 100 mg BIW | 5 (12.2%) | | 0.007 | |

Exploratory Endpoint: Incidence of CMV DNAemia ≧1,000 copies/mL at any time during treatment

| | | | | | |
|---|---|---|---|---|---|
| mITT (modified CMV negative stratum) | All HDP-CDV | 8 (6.0%) | 15 (31.9%) | <0.001 | 14.2.4.1 |
| (p-value derived using Fisher's exact test | 100 mg QW | 2 (8.7%) | | 0.040 | |
| relative to placebo) | 200 mg QW | 2 (6.9%) | | 0.012 | |
| | 200 mg BIW/QW | 0 | | 0.002 | |
| | 100 mg BIW | 0 | | <0.001 | |

^{a}Adjusted for CMV modified strata

### Summary of the Results

With respect to the primary endpoint (incidence of CMV Disease at any time during treatment or CMV DNAemia at the end of treatment), the pooled HDP-CDV dose levels achieved a statistically significant reduction versus placebo when the analysis is adjusted for the presence or absence of CMV DNAemia prior to dosing. In the largest Cohort of the Study (Cohort 4A, 100 mg BIW), there was a statistically significant reduction versus placebo in the proportion of subjects who developed CMV disease or CMV progression. Sensitivity analyses within the randomization strata (CMV status at baseline, presence of GVHD requiring treatment) showed similar directions as the mITT analysis. With the exception of the 40 mg QW dose, all other HDP-CDV doses and dose regimens demonstrated antiviral activity. Depending upon the analysis, activity increased with dose and/or dose frequency. HDP-CDV doses of 100 mg QW or higher showed antiviral activity as measured by the most sensitive biomarker of infection (i.e., the incidence of CMV DNAemia by PCR either at 100 copies/mL or at 1,000 copies/mL at any time during treatment). When individual subject data from the 200 mg QW, 100 mg BIW and placebo groups are examined (CMV DNAemia copies/ml over time), the 100 mg BIW dose regimen resulted in lower frequency and/or lower overall levels of CMV DNAemia when visually compared to either placebo or 200 mg QW. These observations suggest that BIW dosing may have an impact on CMV suppression over and above the total weekly administered dose. There was a trend towards lower use of antivirals for CMV preemptive therapy in subjects who received doses of HDP-CDV of 100 mg QW and higher, versus placebo and HDP-CDV 40 mg QW.

Higher doses of HDP-CDV (200 mg QW, 100 mg BIW and 200 mg BIW) were superior to placebo in reducing the proportion of subjects reaching the composite endpoint of initiation of anti-CMV therapy or CMV disease or CMV dsDNAemia>1,000 copies/mL. There was a trend in subjects who received HDP-CDV 100 mg QW versus placebo in preventing this outcome and the 40 mg QW dose was inactive, providing evidence for a dose response.

No UL97 or UL54 mutations proven to confer drug resistance were detected in subjects enrolled on active drug in study HDP-CDV-201.

In subjects who were plasma CMV DNA negative at baseline, the time to onset of CMV DNAemia was delayed in subjects who received 100 mg QW, 200 mg BIW, and 100 mg BIW HDP-CDV compared to placebo.

Insufficient numbers of subjects who developed frank CMV disease, AdV disease or EBV disease were observed to allow efficacy conclusions to be drawn.

BIW dosing of HDP-CDV appears to be effective in both preventing CMV reactivation and controlling preexisting CMV infection, while both QW and BIW dosing regimen had activity in the prophylaxis of CMV infection or disease. In several of the individual Cohort analyses versus placebo, the 100 mg BIW dose achieved numerically and statistically superior results.

### Example 2

### Preemptive Therapy (PrT) of HCT Patients

Because of the importance of CMV to the transplant population, a number of clinical trials have assessed the effectiveness of anti-CMV agents administered for prophylaxis (i.e., administration to all at risk subjects posttransplantation) and/or preemptive therapy (PrT) (i.e., initiation of treatment based on the detection of viral replication during regular monitoring) in post-HCT subjects. The main advantage of PrT is that it exposes fewer patients to potentially toxic drugs, while prophylactic treatment requires no or less monitoring of viral burden to determine when to initiate treatment. Preemptive therapy, in contrast to prophylaxis, has been associated with emergence of drug-resistant CMV isolates.

Randomized clinical trials of ganciclovir (CYTOVENE®, GCV) prophylaxis have shown a significant reduction in early CMV disease, but without any survival benefit because of the associated increase in the occurrence of invasive fungal and bacterial infections and late onset CMV disease. In contrast, PrT with GCV showed both a reduction in disease and a survival benefit, although PrT is also associated with an increased risk of bacterial and fungal infections. While the current standard of care (STOC) in the HCT population relies on PrT using GCV, or its prodrug, valganciclovir (VALCYTE®, vGCV), as first-line therapy to reduce the severity of CMV disease, the use of both is limited by high rates of neutropenia, with concomitant increased risk of bacterial and fungal infections, as well as anemia and thrombocytopenia requiring transfusion. The second-line anti-CMV drugs, foscarnet (FOSCAVIR®, FOS) and intravenously administered (IV) cidofovir (CDV, VISTIDE®) are also associated with significant toxicities; FOS is associated with electrolyte abnormalities and nephrotoxicity and IV CDV with both neutropenia and nephrotoxicity. New anti-CMV drugs, with little or no significant myelotoxicity, which may be administered prophylactically to allogeneic HCT recipients, may shift the current emphasis away from PrT to CMV prophylaxis.

In a multi-center, randomized, double-blind, placebo-controlled, dose-escalation study designed to evaluate HDP-CDV for the prevention and control of CMV infection in 230 R+HCT recipients, HDP-CDV, at total weekly doses of 100 to 200 mg, was shown to be active and tolerated as a CMV prophylactic agent. At the lowest dose of 40 mg/week, HDP-CDV was essentially inactive with regard to anti-CMV activity, and it had a tolerability profile that was not appreciably different from placebo, while at the highest dose of 400 mg/week, tolerability was unacceptable, with a cluster of GI-related serious adverse events (SAEs) necessitating dose reduction. Subjects who developed CMV infection or disease at any time during the up to 11-week treatment period (through approximately 100 days posttransplant) were discontinued from study treatment (i.e., HDP-CDV or placebo) and followed for a period of 4 weeks, while subjects who completed the treatment period were followed for a period of 8 weeks. After study treatment was discontinued, subjects were treated with PrT for CMV infection/disease.

Among the study population (N=230 subjects), subjects who received STOC for PrT, defined as receiving at least one of GCV, vGCV, FOS or IV CDV for the explicit treatment of CMV infection/disease during the follow-up (“FU”) period, were identified.

The FU period was defined as the last day of study treatment through the last day on study, i.e., completion of the +4-week or +8-week posttreatment visit, as applicable.

Baseline measures for clinical laboratory values were defined as the last value on or before the last day of study treatment. The maximal decrease in ANC was determined using the minimum value reported after the last day of study treatment. The maximal increase in serum creatinine was determined using the maximum value reported after the last day of study treatment. For both parameters, only subjects with ≧1 on-treatment and ≧1 FU value were included in the analysis.

AEs were counted if the onset day was after the last day of study treatment. Transfusion was defined as receipt of whole blood product, packed or concentrated red blood cells (RBC), or platelets at any time during the FU period. Receipt of granulocyte colony-stimulating factor (G-CSF) included receipt of filgrastim (NEUPOGEN®) or pegfilgrastim (NEULASTA®) at any time during the FU period. P-values were determined using Fisher's exact test for binary data and an independent t-test (pooled) for continuous data.

Analysis Populations:

Seventy (30.4%) subjects received PrT for the treatment of CMV infection or disease. Of these, 67 (95.7%) subjects were treated within the 100-day posttransplant period and 56 (80.0%) subjects were treated within 9 days of stopping study treatment (i.e., HDP-CDV or placebo).

Preemptive Therapy:

The majority of the subjects (68/70, 97.1%) received GCV, vGCV, or both, 10 (14.3%) subjects received FOS, and 4 (5.7%) subjects received IV CDV. Fifty-two (74.3%) subjects received 1 medication, 16 (22.9%) subjects received 2 medications, and 2 (2.9%) subjects received 3 medications.

Demographics/Baseline Characteristics:

The demographic/baseline characteristics of the two groups are summarized in TABLE 16:

TABLE 16

| | |
|---|---|
| Treated with | Not Treated with |
| Anti-CMV STOC | Anti-CMV STOC |
| (n = 70) | (n = 160) |

Age, years

| | | |
|---|---|---|
| Mean | 50.5 | 50.7 |
| Median | 51.5 | 50.5 |
| Min, Max | 25, 69 | 21, 71 |

Gender, n (%)

| | | |
|---|---|---|
| Female | 28 (40.0) | 70 (43.8) |
| Male | 42 (60.0) | 90 (56.3) |

Race, n (%)

| | | |
|---|---|---|
| Asian | 2 (2.9) | 10 (6.3) |
| Black | 3 (4.3) | 4 (2.5) |
| White | 62 (88.6) | 145 (90.6) |
| Other | 3 (4.3) | 1 (0.6) |

Weight (kg)

| | | |
|---|---|---|
| Mean | 79.0 | 77.2 |
| Median | 78.6 | 77.9 |
| Min, Max | 40.8, 146.9 | 40.6, 131.9 |
| Unrelated donor, n (%) | 47 (67.1) | 77 (48.1) |
| Adult mismatch, n (%) | 15 (21.4) | 18 (11.3) |
| Myeloablative | 52 (74.3) | 95 (59.4) |
| Conditioning, n (%) | | |

The demographics of the two groups were comparable. As might be expected, the group requiring PrT intervention included a higher proportion of subjects with risk factors for CMV reactivation (i.e., unrelated and/or mismatched donor and receipt of a myeloablative conditioning regimen).

### Duration of Follow-Up:

The duration of FU between the two groups is summarized in TABLE 17.

TABLE 17

| | |
|---|---|
| | Not Treated |
| | with Anti-CMV |
| Treated with Anti-CMV STOC | STOC |
| (n = 70) | (n = 160) |

Duration of Follow-up (Days)

| | | |
|---|---|---|
| Mean (SD) | 30.9 (11.8) | 45.4 (19.4) |
| Median | 28 | 53 |
| Q1, Q3 | 27, 33 | 31, 57 |
| Min, Max | 3, 63 | 1, 144 |

No. of Subjects Followed for Specified Period (n, [%]):

| | | |
|---|---|---|
| ≧2 weeks | 66 (94.3%) | 147 (91.9%) |
| ≧4 weeks | 52 (74.3%) | 136 (85.0%) |
| ≧6 weeks | 9 (12.9%) | 104 (65.0%) |
| ≧8 weeks | 5 (7.1%) | 63 (39.4%) |

Subjects who completed study treatment were followed for up to twice as long as subjects who discontinued study treatment prematurely (i.e., 8 weeks vs. 4 weeks) and, therefore, have more time to experience and report potentially adverse findings.

Clinical Laboratory Values of Interest:

ANC and serum creatinine values during the FU period is summarized in TABLE 18.

TABLE 18

| | | |
|---|---|---|
| Treated with | Not Treated | |
| Anti-CMV | with Anti- | |
| STOC | CMV STOC | |
| (n = 70) | (n = 160) | P-value |

Absolute Neutrophil Count (G/L)

| | | | |
|---|---|---|---|
| Maximal ANC Decrease: | | | |
| N | 68 | 149 | |
| Mean (SD) | 2.0 (2.9) | 0.7 (2.0) | |
| Median | 1.4 | 0.5 | 0.0002 |
| Q1, Q3 | 0.3, 3.4 | −0.2, 1.5 | |
| Min, Max | −6.5, 11.4 | −4.4, 8.1 | |
| No. of Subjects with: | | | |
| Any decrease in ANC, n (%) | 56 (82.4%) | 100 (67.1%) | 0.02 |
| >2 G/L decrease in ANC, n (%) | 28 (41.2%) | 27 (18.1%) | 0.0006 |
| ANC <0.5 G/L, n (%) | 3 (4.4%) | 8 (5.4%) | 1.0 |
| ANC <1 G/L, n (%) | 15 (22.1%) | 30 (20.1%) | 0.72 |
| ANC <1.5 G/L, n (%) | 25 (36.8%) | 51 (34.2%) | 0.76 |

Serum Creatinine (μmol/L)

| | | | |
|---|---|---|---|
| Maximal Creatinine Increase: | | | |
| N | 68 | 150 | |
| Mean (SD) | 6.9 (32.2) | 3.3 (37.1) | |
| Median | 4.5 | 8.0 | 0.50 |
| Q1, Q3 | −9.0, 26.0 | −9.0, 17.0 | |
| Min, Max | −53.0, 142.0 | −283.0, 142.0 | |
| No. of subjects with: | | | |
| >20% increase in creatinine, | 23 (33.8%) | 36 (24.0%) | 0.14 |
| n (%) | | | |

There were statistically significant differences in the decreased ANC values reported between the two groups, which were greater in the subjects receiving anti-CMV STOC. Mean increases in serum creatinine values were also higher in subjects receiving anti-CMV STOC, but did not achieve statistical significance.

Relevant Adverse Events:

The incidence of relevant life-threatening or fatal AEs between the two groups is summarized in TABLE 19.

TABLE 19

| | Treated with | Not Treated |
|---|---|---|
| | Anti-CMV | with Anti-CMV |
| Primary System Organ Class | STOC | STOC |
| Preferred Term | (n = 70) | (n = 160) |
| Subjects Reporting ≧1 AE, | 57 (81.4%) | 97 (60.6%) |
| n (%) | | |
| Subjects Experiencing ≧1 | 12 (17.1%) | 14 (8.8%) |
| Life-threatening or Fatal AE, | | |
| n (%) | | |

Blood and Lymphatic System Disorders, n (%)

| | | |
|---|---|---|
| Any AE | 4 (5.7%) | 2 (1.3%) |
| Lymphopenia | 1 (1.4%) | 0 |
| Neutropenia | 0 | 1 (0.6%) |
| Pancytopenia | 2 (2.9%) | 0 |
| Thrombocytopenia | 2 (2.9%) | 1 (0.6%) |

Infections and Infestations, n (%)

| | | |
|---|---|---|
| Any AE | 3 (4.3%) | 2 (1.3%) |
| Cellulitis | 1 (1.4%) | 0 |
| Escherichia sepsis | 0 | 1 (0.6%) |
| Klebsiella bacteremia | 1 (1.4%) | 1 (0.6%) |
| Pneumonia | 1 (1.4%) | 1 (0.6%) |
| Septic shock | 1 (1.4%) | 0 |

Renal and Urinary Disorders, n (%)

| | | |
|---|---|---|
| Any AE | 2 (2.9%) | 0 |
| Renal failure | 2 (2.9%) | 0 |

The number of subjects reporting relevant AEs that either required or prolonged hospitalization between the two groups is summarized in TABLE 20.

TABLE 20

| | | Not Treated |
|---|---|---|
| | Treated with | with Anti-CMV |
| Primary System Organ Class | Anti-CMV STOC | STOC |
| Preferred Term | (n = 70) | (n = 160) |
| Subjects Reporting ≧1 AE, | 57 (81.4%) | 97 (60.6%) |
| n (%) | | |
| Subjects Reporting ≧1 AE | 22 (31.4%) | 35 (21.9%) |
| Requiring or Prolonging | | |
| Hospitalization, n (%) | | |

Blood and Lymphatic System Disorders, n (%)

| | | |
|---|---|---|
| Any AE | 3 (4.3%) | 3 (1.9%) |
| Anemia | 1 (1.4%) | 1 (0.6%) |
| Febrile neutropenia | 0 | 2 (1.3%) |
| Lymphopenia | 1 (1.4%) | 0 |
| Pancytopenia | 1 (1.4%) | 0 |
| Thrombocytopenia | 1 (1.4%) | 0 |

Infections and Infestations, n (%)

| | | |
|---|---|---|
| Any AE | 12 (17.1%) | 10 (6.3%) |
| Bacteremia | 1 (1.4%) | 1 (0.6%) |
| Cellulitis | 1 (1.4%) | 0 |
| Citrobacter infection | 1 (1.4%) | 0 |
| Clostridium difficile infection | 1 (1.4%) | 0 |
| Epstein-Barr virus infection | 1 (1.4%) | 0 |
| Escherichia sepsis | 0 | 1 (0.6%) |
| Klebsiella bacteremia | 1 (1.4%) | 1 (0.6%) |
| Metapneumovirus infection | 0 | 1 (0.6%) |
| Pneumonia | 1 (1.4%) | 2 (1.3%) |
| Pneumonia, Legionella | 0 | 1 (0.6%) |
| Pseudomonal bacteremia | 1 (1.4%) | 0 |
| Sepsis | 1 (1.4%) | 1 (0.6%) |
| Septic shock | 1 (1.4%) | 0 |
| Serratia bacteremia | 0 | (0.6%) |
| Sinusitis | 1 (1.4%) | 0 |
| Staphylococcal bacteremia | 0 | 1 (0.6%) |
| Staphylococcal infection | 1 (1.4%) | 0 |
| Stenotrophomonas infection | 0 | 1 (0.6%) |
| Streptococcal bacteremia | 1 (1.4%) | 0 |

Renal and Urinary Disorders, n (%)

| | | |
|---|---|---|
| Any AE | 2 (2.9%) | 2 (1.3%) |
| Hematuria | 0 | 2 (1.3%) |
| Renal failure | 2 (2.9%) | 0 |

Overall, there was a greater incidence of AEs reported during the FU period by subjects receiving anti-CMV STOC. This was also reflected in a greater incidence of life-threatening AEs and AEs resulting in hospitalization or prolongation of hospitalization in the subjects receiving anti-CMV STOC.

Granulocyte Colony-Stimulating Factor:

The incidence of G-CSF use and receipt of transfusion between the two groups is summarized in TABLE 21.

TABLE 21

| | Treated with | Not Treated | |
|---|---|---|---|
| | Anti- | with Anti- | |
| | CMV STOC | CMV STOC | |
| | (n = 70) | (n = 160) | P-value |
| Received G-CSF, n (%) | 11/70 (15.7%) | 17/160 (10.6%) | 0.28 |
| Received Transfusion, n (%) | 8/70 (11.4%) | 24/160 (15.0%) | 0.54 |

A higher percentage of subjects receiving anti-CMV STOC required G-CSF, although the difference was not statistically significant. In contrast, a lower percentage of subjects receiving anti-CMV STOC appear to have required blood or blood product transfusion, although the difference was again not statistically significant. However, this observation is explained by the different durations of follow-up in the two groups, with a 2.6% incidence of transfusion per subject-week in the group receiving anti-CMV STOC, as compared to a 2.3% incidence of transfusion per subject-week in the group that did not receive anti-CMV STOC.

The results of the post hoc analysis showed that the incidence of preemptive therapy with anti-CMV STOC in subjects who received placebo or the essentially inactive 40 mg HDP-CDV dose was 32 out of a combined 84 subjects (or 38.1%), as compared to 38 out of a combined 146 subjects (or 26.0%) in the four dose cohorts receiving active doses of ≧100 mg HDP-CDV per week. The rate of preemption in the HDP-CDV cohort with the greatest anti-CMV activity, 100 mg twice-weekly, was 22.0% (11/50). These data suggested that, once treatment is completed, subjects treated with HDP-CDV at active antiviral are less likely to need PrT and other interventions.

### Example 3

### Safety Analyses and Results

The safety and tolerability profile of HDP-CDV, including subject demographics and Baseline characteristics; the AE profile; and laboratory abnormalities of interest were analyzed in the cohorts followed in this investigation. Based on these results, potential adverse drug reactions were identified and were further analyzed.

### Baseline Demographics and Characteristics

Subject demographics and Baseline characteristics by dosing Cohorts are presented in TABLE 22. Overall, the baseline demographics were well balanced between Cohorts and between subjects randomized to HDP-CDV or placebo. The range of subjects' weights was broad from 40.6 to 146.9 kg (means ranging from 75.44 to 79.25 kg across Cohorts). The majority of subjects were white males with an average age of 50 years. The most frequent source of stem cells was peripheral blood. Across Cohorts, approximately 10% to 20% of subjects had acute GVHD at the time of treatment initiation in this Study (ranging from 3 of 30 to 5 of 25 subjects). These subject characteristics in Study HDP-CDV-201 are consistent with those of adult HSCT recipients in the United States. (Gooley T A., et al., Reduced Mortality after Allogeneic Hematopoietic-Cell Transplantation. N Engl J. Med. 2010; 363(22):2091-2101).

The distribution of types of conditioning regimens was not well balanced among groups, with more subjects having received a myeloablative conditioning regimen in Cohorts 3 (31/39, 79%), 4A (37/50, 74%) and placebo (43/59, 73%), as compared to Cohorts 1 (12/25, 48%), 2 (16/27, 59%) and 4 (14/30, 47%). The proportion of subjects transplanted with a graft from an unrelated donor was higher in subjects enrolled in Cohort 3 (26/39, 66%) versus all other Cohorts. The proportion of subjects who received a graft from an adult mismatch donor was higher in subjects enrolled in Cohort 4 and 4A (6/30 or 20% and 7/50 or 14%, respectively) as compared to other Cohorts (≦7%). These imbalances are important to keep in mind as they affect the risk of GVHD and of gastrointestinal AEs in HSCT recipients.

TABLE 22
Baseline Characteristics (Subjects n (%))

| | Cohort | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| | N = 25 | N = 27 | N = 39 | N = 30 | N = 50 | N = 59 |
| Age (years) | | | | | | |
| Mean | 56.3 | 51.7 | 49.5 | 50.4 | 48.8 | 50.3 |
| Median | 58.0 | 52.0 | 51.0 | 50.0 | 49.0 | 50.0 |
| Min, Max | 30, 70 | 32, 69 | 23, 70 | 28, 69 | 22, 71 | 21, 70 |
| Gender | | | | | | |
| Female | 10 (40.0%) | 10 (37.0%) | 17 (43.6%) | 12 (40.0%) | 24 (48.0%) | 25 (42.4%) |
| Male | 15 (60.0%) | 17 (63.0%) | 22 (56.4%) | 18 (60.0%) | 26 (52.0%) | 34 (57.6%) |
| Race | | | | | | |
| Asian | 2 (8.0%) | 2 (7.4%) | 3 (7.7%) | 1 (3.3%) | 2 (4.0%) | 2 (3.4%) |
| Black | 0 | 0 | 0 | 0 | 3 (6.0%) | 4 (6.8%) |
| White | 22 (88.0%) | 24 (88.9%) | 35 (89.7%) | 29 (96.7%) | 44 (88.0%) | 53 (89.8%) |
| Other | 1 (4.0%) | 1 (3.7%) | 1 (2.6%) | 0 | 1 (2.0%) | 0 |
| Weight (kg) | | | | | | |
| Mean | 76.20 | 79.25 | 75.44 | 79.05 | 77.63 | 78.55 |
| Median | 74.84 | 77.70 | 75.70 | 80.55 | 78.36 | 79.40 |
| Min, Max | 45.5, 109.7 | 43.1, 130.8 | 46.2, 127.9 | 50.3, 131.9 | 40.6, 113.4 | 40.8, 146.9 |
| Stem cell source | | | | | | |
| Peripheral blood | 20 (80.0%) | 24 (88.9%) | 28 (71.8%) | 24 (80.0%) | 43 (86.0%) | 48 (81.4%) |
| Bone marrow | 2 (8.0%) | 1 (3.7%) | 7 (17.9%) | 6 (20.0%) | 5 (10.0%) | 7 (11.8%) |
| Cord blood | 3 (12.0%) | 2 (7.4%) | 4 (10.3%) | 0 | 2 (4.0%) | 4 (6.8%) |
| GVHD at Baseline | | | | | | |
| Any GVHD | 5 (20.0%) | 5 (18.5%) | 4 (10.2%) | 3 (10.0%) | 6 (12.0%) | 6 (10.2%) |
| Skin | 4 (16.0%) | 4 (14.8%) | 2 (5.2%) | 3 (10.0%) | 5 (10.0%) | 4 (6.8%) |
| Intestine | 1 (4.0%) | 1 (3.7%) | 3 (7.7%) | 0 | 1 (2.0%) | 4 (6.8%) |
| Liver | 0 | 1 (3.7%) | 0 | 0 | 0 | 0 |
| Donor CMV^{a} —, % | 14 (56.0%) | 18 (66.7%) | 23 (59.0%) | 19 (63.3%) | 28 (56.0%) | 27 (45.8%) |
| Unrelated donor^{a}, % | 12 (48.0%) | 16 (59.3%) | 26 (66.7%) | 17 (56.7%) | 26 (52.0%) | 28 (47.5%) |
| Adult mismatch^{a}, % | 1 (4.0%) | 2 (7.4%) | 2 (5.1%) | 6 (20.0%) | 7 (14.0%) | 3 (5.1%) |
| Myeloablative | 12 (48.0%) | 16 (59.3%) | 31 (79.5%) | 14 (46.7%) | 37 (74.0%) | 43 (72.9%) |
| conditioning^{a}, % | | | | | | |
| T-cell depletion^{a}, % | 0 | 1 (3.7%) | 3 (7.7%) | 0 | 4 (8.0%) | 4 (6.8%) |

^{a}Additional data collected from sites outside of the clinical database but prior to database lock and Investigators unblinding

### Subject Disposition and Duration of Exposure

Subjects completing the treatment period received a full course of study medication; those completing the entire study attended visits through 8 weeks of follow up. Subjects who discontinued treatment may have done so for protocol specified reasons (e.g. development of clinically significant CMV infection requiring preemptive treatment with excluded anti-CMV medications) or for other reasons (e.g., withdrawal, adverse events, loss to follow-up). The disposition of subjects in Study HDP-CDV-201 during the double-blind, placebo-controlled treatment period (through Week 11) is listed by dosing Cohort in TABLE 23.

TABLE 23
Subject Disposition by Cohort (1 through 4A) during the Active
Treatment Period (through Week 11) (Subject n (%))

| | Cohort | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Subjects enrolled (ITT) | 25 | 27 | 39 | 30 | 50 | 59 |
| Subjects completed | 9 (36.0%) | 18 (66.7%) | 18 (46.2%) | 7 (23.3%) | 30 (60.0%) | 32 (54.2%) |
| treatment | | | | | | |
| Subjects who permanently | 16 (64.0%) | 9 (33.3%) | 21 (53.8%) | 23 (76.7%) | 20 (40.0%) | 27 (45.8%) |
| discontinued treatment | | | | | | |
| Subjects completed study | 7 (28.0%) | 14 (51.9%) | 14 (35.9%) | 6 (20.0%) | 25 (50.0%) | 30 (50.8%) |

In this dose escalation study, between 23 and 67% of subjects in each Cohort completed the treatment period. The Cohort with the fewest subjects completing treatment was Cohort 4 (200 mg BIW) and the highest treatment completion rate was in Cohort 4a (100 mg BIW) and Cohort 2 (100 mg QW).

The duration of exposure is displayed in TABLE 24 by Cohort.

TABLE 24
Duration of Exposure by Cohort 1 through 4A (Days)

| | Cohort | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Median | 42 | 70 | 49 | 43 | 63 | 61 |
| Mean | 45 | 60 | 49 | 41 | 52 | 50 |
| Min | 7 | 14 | 7 | 4 | 7 | 4 |
| Max | 77 | 78 | 77 | 77 | 79 | 80 |

The exposure to drug was longer in subjects randomized to HDP-CDV 100 mg QW, 100 mg BIW and placebo as compared to subjects randomized to HDP-CDV 40 mg QW, 200 mg QW and 200 mg BIW.

The Cohort with the fewest discontinuations for adverse events was Cohort 2, at a dose of 100 mg QW. The highest rate of discontinuations for adverse events was in Cohort 4, at a dose of 200 mg BIW. The rates of discontinuation in subjects receiving 40 and 200 mg weekly, 100 mg BIW and placebo were comparable. Discontinuations for CMV disease and/or initiation of alternate CMV therapy were less frequent in Cohorts 3, 4 and 4a.

Discontinuations due to death were infrequent. Note that some subjects who discontinued study drug for various reasons during the treatment period subsequently died of other causes during the follow up period. In all, there was a total of 19 fatalities (8% of patients) across all treatment groups (including placebo), spanning the entire study duration.

Information on AEs leading to treatment discontinuation can be found in TABLE 25.

TABLE 25
Summary of all AEs and GI AEs Leading to Study Drug Withdrawal: Cohorts 1 through 4A

| | Cohort 1 | Cohort 2 | Cohort 3 | Cohort 4 | Cohort 4A HDP- | |
|---|---|---|---|---|---|---|
| | HDP-CDV | HDP-CDV | HDP-CDV | HDP-CDV | CDV | Pooled |
| System Organ Class | 40 mg QW | 100 mg QW | 200 mg QW | 200 mg BIW | 100 mg BIW | Placebo |
| Preferred Term | N = 25 | N = 27 | N = 39 | N = 30 | N = 50 | N = 59 |
| All AEs leading to | 15 (60.0%) | 9 (33.3%) | 15 (38.5%) | 18 (60.0%) | 18 (36.0%) | 27 (45.8%) |
| withdrawal | | | | | | |

Gastrointestinal Disorders

| | | | | | | |
|---|---|---|---|---|---|---|
| Abdominal pain | 0 | 0 | 0 | 1 (3.3%) | 1 (2.0%) | 0 |
| Diarrhoea | 0 | 0 | 1 (2.6%) | 7 (23.3%) | 2 (4.0%) | 0 |
| Dyspepsia | 0 | 0 | 2 (5.1%) | 0 | 0 | 0 |
| Nausea | 0 | 0 | 0 | 1 (3.3%) | 1 (2.0%) | 2 (3.4%) |
| Vomiting | 0 | 1 (3.7%) | 0 | 0 | 1 (2.0%) | 0 |

### Adverse Events Leading to Death

The deaths associated with treatment discontinuations are listed in TABLE 13. (See above). TABLE 26 lists the frequency of AEs with a fatal outcome by Cohorts for the entire study duration. None of the deaths was considered related to HDP-CDV by the Investigators and the proportion of subjects who died during the study was similar between treatment arms and placebo.

TABLE 26
Summary of Fatal AEs by Cohorts

| | | | | | | |
|---|---|---|---|---|---|---|
| | | Cohort 2 | | | | |
| | Cohort 1 | HDP-CDV | Cohort 3 | Cohort 4 | Cohort 4A | |
| | HDP-CDV | 100 mg | HDP-CDV | HDP-CDV | HDP-CDV | |
| System Organ Class | 40 mg QW | QW | 200 mg QW | 200 mg BIW | 100 mg BIW | Pooled Placebo |
| Grade | N = 25 | N = 27 | N = 39 | N = 30 | N = 50 | N = 59 |

All AEs

| | | | | | | |
|---|---|---|---|---|---|---|
| Grade 5 (deaths) | 2 (8.0%) | 0 | 3 (7.7%) | 4 (13.3%) | 5 (10.0%) | 5 (8.5%) |

TABLE 27 presents a summary of the subjects who died during the course of the study C.

TABLE 27
Summary of Subjects who Died During the Study Period

| | | | | | | Time between | | Morbidities |
|---|---|---|---|---|---|---|---|---|
| Subject | | Age | | Cause of | Duration | last dose and | | contributing |
| Number | Dose | (yrs) | Gender | death | of Therapy | death | Risk factors | to death |
| 035-004 | Placebo | 60 | Male | Chronic | 29 days | 21 days | Relapsed CLL | Klebsiella |
| (2034) | | | | lymphocytic | | | | bacteremia, |
| | | | | leukemia, | | | | renal failure |
| | | | | multi-organ | | | | |
| | | | | failure | | | | |
| 037-008 | Placebo | 58 | Male | Klebsiella | 1 days | 25 days | CMV treatment | |
| (4103) | | | | bacteremia, | | | with ganciclovir | |
| | | | | multi-organ | | | | |
| | | | | failure | | | | |
| 038-005 | Placebo | 65 | Male | Gut GVHD | 1 day | 22 days | Preexisting GVHD | |
| (3026) | | | | | | | Relapsed mantle | |
| | | | | | | | cell lymphoma | |
| | | | | | | | CMV colitis treated | |
| | | | | | | | with ganciclovir | |
| 036-067 | Placebo | 49 | Male | Relapse | 51 days | 21 days | Relapsed AML | |
| (4091) | | | | AML | | | | |
| 045-001 | Placebo | 54 | Female | PTLD | 7 days | 50 days | Secondary graft | |
| (2035) | | | | | | | failure | |
| 036-006 | 40 mg | 49 | Male | Relapsed | 48 days | 17 days | Relapsed AML | |
| (1018) | QW | | | AML | | | | |
| 025-005 | 40 mg | 58 | Female | Acute | 8 days | 23 days | Unrelated donor | Sepsis |
| (1003) | QW | | | GVHD | | | Myeloablative | |
| | | | | | | | conditioning | |
| | | | | | | | regimen | |
| 025-026 | 200 mg | 49 | Female | Liver | 71 days | 37 days | Mismatched cord | E. coli sepsis |
| (3030) | QW | | | GVHD | | | blood | |
| | | | | | | | transplantation | |
| | | | | | | | Myeloablative | |
| | | | | | | | conditioning | |
| | | | | | | | regimen | |
| 036-020 | 200 mg | 50 | Female | Cardiac | 71 days | 4 days | Capillary leak | Aspiration |
| (3007) | QW | | | tamponade | | | syndrome | pneumonia |
| | | | | | | | associated with | |
| | | | | | | | dinileukin diftitox | |
| | | | | | | | therapy | |
| | | | | | | | Myelodysplastic | |
| | | | | | | | syndrome relapse | |
| 041-005 | 200 mg | 43 | Female | Relapsed | 57 days | 32 days | Relapsed ALL | |
| (3015) | QW | | | ALL | | | | |
| 036-065 | 100 mg | 49 | Male | Alveolar | 60 days | 38 days | Thrombocytopenia | |
| (4072) | BIW | | | hemorrage | | | due to AML relapse | |
| 026-006 | 100 mg | 59 | Female | Acute | 21 days | 16 days | Pre-existing GVHD | Multifocal |
| (4100) | BIW | | | GVHD | | | Unrelated donor | pneumonia |
| | | | | | | | Myeloablative | Sepsis |
| | | | | | | | conditioning | Multiorgan |
| | | | | | | | regimen | failure |
| 041-013 | 100 mg | 50 | Male | Relapsed | 62 days | 13 days | Gastrointestinal | |
| (4056) | BIW | | | AML | | | relapse | |
| 025-044 | 100 mg | 63 | Female | Gut GVHD | 18 days | 9 days | Myeloablative | VRE |
| (4034) | BIW | | | | | | conditioning | meningitis |
| | | | | | | | regimen | |
| 022-012 | 100 mg | 38 | Female | Renal | 62 days | 32 days | Pre-existing GVHD | Pneumonia |
| (4058) | BIW | | | failure, GI | | | Graft failure | |
| | | | | bleed, CVA | | | | |
| | | | | and graft | | | | |
| | | | | failure | | | | |
| 022-017 | 100 mg | 38 | Female | Relapse of | 24 days | 2 days | Relapse | |
| (4104) | BIW | | | large cell | | | | |
| | | | | lymphoma | | | | |
| 036-041 | 200 mg | 49 | Male | Refractory | 36 days | 7 days | Pre-existing GVHD | Sepsis |
| (4003) | BIW | | | GVHD | | | Mismatched | |
| | | | | | | | unrelated donor | |
| | | | | | | | Myeloablative | |
| | | | | | | | conditioning | |
| | | | | | | | regimen | |
| 007-011 | 200 mg | 49 | Male | Acute | 32 days | 2 days | Second HSCT | MSSA |
| (4014) | BIW | | | GVHD | (GVHD | | | bacteremia |
| | | | | | diagnosed | | | |
| | | | | | after one | | | |
| | | | | | dose of | | | |
| | | | | | Study drug) | | | |
| 031-008 | 200 mg | 39 | Male | GI bleed | 53 days | 2 days | Liver and kidney | Mucosal |
| (4002) | BIW | | | | | | transplant | hemorrhage |
| | | | | | | | Thrombocytopenia | (epistaxis) |
| | | | | | | | | Staph |
| | | | | | | | | bacteremia |

A number of subjects died of disease relapse or transplant-related complications during the study. The duration of exposure to HDP-CDV prior to death varied widely and a number of deaths occurred weeks after the last dose of study drug was administered. Six (6) subjects who received HDP-CDV died of acute GVHD and had significant risk factors for GVHD; 1 subject randomized to placebo died of acute GVHD. Five (5) subjects randomized to HDP-CDV therapy died of hematologic malignancy relapse as compared to 2 subjects randomized to placebo (another subject randomized to placebo had a hematologic relapse, although the cause of death was acute GVHD). One (1) subject randomized to HDP-CDV died of gastrointestinal bleeding in the context of severe thrombocytopenia. One (1) subject randomized to placebo died of post-transplant lymphoproliferative disorder; and 1 placebo-treated subject died of sepsis. One (1) subject randomized to HDP-CDV died of graft failure 32 days after the last dose of HDP-CDV. None of the deaths were considered by the Investigators as related to HDP-CDV.

### Treatment-Emergent Adverse Events

In order to evaluate treatment-emergent adverse events (TEAEs) for common “potential” adverse drug reactions (ADRs), TEAEs reported either by at least 15% of subjects in any given treatment Cohort OR by at least 10% of subjects and at least 2-fold greater frequency in any HDP-CDV group compared to Placebo is presented in TABLE 28.

TABLE 28
Summary of Most Frequent Treatment Emergent Adverse Events (TEAE) Reported for
≧15% in any Cohort OR for ≧10% of Subjects and ≧2x More Frequently in Any HDP-CDV
Group Compared to the Placebo Group) by Treatment Group and System Organ Class (Subject n (%).

| | | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| System Organ Class | 40 mg QW | 100 mg QW | 200 mg QW | 200 mg BIW | 100 mg BIW | Placebo |
| Preferred Term | N = 25 | N = 27 | N = 39 | N = 30 | N = 50 | N = 59 |

Blood and Lymphatic System Disorders

| | | | | | | |
|---|---|---|---|---|---|---|
| Neutropenia | 2 (8.0%) | 4 (14.8%) | 4 (10.3%) | 4 (13.3%) | 10 (20.0%) | 7 (11.9%) |
| Thrombocytopenia | 3 (12.0%) | 3 (11.1%) | 0 | 5 (16.7%) | 7 (14.0%) | 4 (6.8%) |
| Increased leukocytosis | 1 (4.0%) | 0 | 2 (5.1%) | 3 (10.0%) | 2 (4.0%) | 1 (1.7%) |

Gastrointestinal Disorders

| | | | | | | |
|---|---|---|---|---|---|---|
| Abdominal pain | 4 (16.0%) | 2 (7.4%) | 5 (12.8%) | 11 (36.7%) | 13 (26.0%) | 4 (6.8%) |
| Diarrhoea | 3 (12.0%) | 8 (29.6%) | 13 (33.3%) | 21 (70.0%) | 26 (52.0%) | 16 (27.1%) |
| Nausea | 6 (24.0%) | 5 (18.5%) | 11 (28.2%) | 11 (36.7%) | 17 (34.0%) | 12 (20.3%) |
| Vomiting | 2 (8.0%) | 6 (22.2%) | 6 (15.4%) | 8 (26.7%) | 22 (44.0%) | 11 (18.6%) |
| Dysgeusia | 1 (4.0%) | 1 (3.7%) | 2 (5.1%) | 3 (10.0%) | 4 (8.0%) | 2 (3.4%) |
| Abdominal pain, Upper | 1 (4.0%) | 2 (7.4%) | 1 (2.6%) | 3 (10.0%) | 2 (4.0%) | 2 (3.4%) |
| GI Reflux Disease | 2 (8.0%) | 4 (14.8%) | 2 (5.1%) | 2 (6.7%) | 0 | 1 (1.7%) |
| Recurrent flatulence | 0 | 1 (3.7%) | 1 (2.6%) | 3 (10.0%) | 3 (6.0%) | 1 (1.7%) |

General Disorders

| | | | | | | |
|---|---|---|---|---|---|---|
| Fatigue | 1 (4.0%) | 3 (11.1%) | 5 (12.8%) | 0 | 9 (18.0%) | 9 (15.3%) |
| Oedema peripheral | 3 (12.0%) | 5 (18.5%) | 3 (7.7%) | 3 (10.0%) | 8 (16.0%) | 6 (10.2%) |
| Pyrexia | 1 (4.0%) | 1 (3.7%) | 7 (17.9%) | 2 (6.7%) | 10 (20.0%) | 11 (18.6%) |
| Generalized oedema | 1 (4.0%) | 0 | 0 | 3 (10.0%) | 1 (2.0%) | 1 (1.7%) |
| Malnutrition | 0 | 0 | 1 (2.6%) | 3 (10.0%) | 1 (2.0%) | 1 (1.7%) |

Hepatobiliary Disorders

| | | | | | | |
|---|---|---|---|---|---|---|
| Hyperbilirubinaemia | 1 (4.0%) | 0 | 3 (7.7%) | 5 (16.7%) | 3 (6.0%) | 0 |

Immune system Disorders

| | | | | | | |
|---|---|---|---|---|---|---|
| Acute GVHD | 6 (24.0%) | 7 (25.5%) | 13 (33.3%) | 24 (80.0%) | 32 (64.0%) | 15 (25.4%) |

Infections and Infestations

| | | | | | | |
|---|---|---|---|---|---|---|
| Cytomegalovirus viremia | 10 (40.0%) | 4 (14.8%) | 7 (17.9%) | 5 (16.7%) | 6 (12.0%) | 11 (18.6%) |
| Staphylococcal Bacteremia | 0 | 1 (3.7%) | 1 (2.6%) | 1 (3.3%) | 6 (12.0%) | 2 (3.4%) |
| Oral Candidiasis | 0 | 0 | 2 (5.1%) | 1 (3.3%) | 6 (12.0%) | 1 (1.7%) |
| Pneumonia | 1 (4.0%) | 2 (7.4%) | 0 | 1 (3.3%) | 5 (10.0%) | 1 (1.7%) |

Investigations

| | | | | | | |
|---|---|---|---|---|---|---|
| ALT increased | 2 (8.0%) | 1 (3.7%) | 2 (5.1%) | 10 (33.3%) | 9 (18.0%) | 1 (1.7%) |
| AST increased | 1 (4.0%) | 1 (3.7%) | 1 (2.6%) | 7 (23.3%) | 5 (10.0%) | 2 (3.4%) |
| Creatinine increased | 0 | 5 (18.5%) | 3 (7.7%) | 2 (6.7%) | 7 (14.0%) | 3 (5.1%) |
| Liver function test abnormal | 0 | 1 (3.7%) | 2 (5.1%) | 3 (10.0%) | 2 (4.0%) | 2 (3.4) |
| Gamma-glutamyltransferase | 0 | 1 (3.7%) | 0 | 3 (10.0%) | 4 (8.0%) | 1 (1.7%) |
| increased | | | | | | |
| Blood lactate dehydrogenase | 0 | 1 (3.7%) | 0 | 3 (10.0%) | 4 (8.0%) | 0 |
| increased | | | | | | |

Metabolism and Nutrition Disorders

| | | | | | | |
|---|---|---|---|---|---|---|
| Decreased appetite | 4 (16.0%) | 5 (18.5%) | 3 (7.7%) | 5 (16.7%) | 9 (18.0%) | 7 (11.9%) |
| Hyperglycaemia | 1 (4.0%) | 1 (3.7%) | 1 (2.6%) | 8 (26.7%) | 9 (18.0%) | 4 (6.8%) |
| Hypocalcaemia | 1 (4.0%) | 0 | 0 | 3 (10.0%) | 2 (4.0%) | 1 (1.7%) |
| Hypokalaemia | 4 (16.0%) | 2 (7.4%) | 5 (12.8%) | 5 (16.7%) | 8 (16.0%) | 4 (6.8%) |
| Hyponatraemia | 3 (12.0%) | 1 (3.7%) | 7 (17.9%) | 2 (6.7%) | 1 (2.0%) | 3 (5.1%) |
| Hyperkalemia | 1 (4.0%) | 1 (3.7%) | 1 (2.6%) | 3 (10.0%) | 3 (6.0%) | 2 (3.4%) |

Musculoskeletal

| | | | | | | |
|---|---|---|---|---|---|---|
| Arthralgias | 0 | 3 (11.1%) | 1 (2.6%) | 1 (3.3%) | 3 (6.0%) | 3 (5.1%) |
| Back Pain | 0 | 2 (7.4%) | 2 (5.1%) | 3 (10.0%) | 4 (8.0%) | 2 (3.4%) |

Neoplasms

| | | | | | | |
|---|---|---|---|---|---|---|
| Acute Myeloid Leukemia | 3 (12.0%) | 0 | 0 | 0 | 3 (6.0%) | 3 (5.1%) |
| recurrent | | | | | | |

Nervous System

| | | | | | | |
|---|---|---|---|---|---|---|
| Tremor | 3 (12.0%) | 3 (11.1%) | 2 (5.1%) | 2 (6.7%) | 1 (2.0%) | 2 (3.4%) |

Psychiatric Disorders

| | | | | | | |
|---|---|---|---|---|---|---|
| Insomnia | 2 (8.0%) | 2 (7.4%) | 5 (12.8%) | 2 (6.7%) | 9 (18.0%) | 1 (1.7%) |
| Anxiety | 0 | 3 (11.1%) | 3 (7.7%) | 2 (6.7%) | 3 (6.0%) | 3 (5.1%) |

Renal and Urinary Disorders

| | | | | | | |
|---|---|---|---|---|---|---|
| Renal Failure | 1 (4.0%) | 2 (7.4%) | 0 | 0 | 5 (10.0%) | 3 (5.1%) |
| Acute Renal Failure | 2 (8.0%) | 0 | 0 | 3 (10.0%) | 1 (2.0%) | 2 (3.4%) |

There was a high frequency of adverse events in the study population overall, attributable to the underlying condition of the subjects. Subjects entered the study at the post-HSCT peri-engraftment period, a time of significant medical intervention. Overall, more AEs were reported by subjects who received HDP-CDV BIW as compared to subjects who received HDP-CDV QW or placebo.

The most common AEs, reported in 20% or more of at least one Cohort were abdominal pain, diarrhea, nausea, and vomiting from the Gastrointestinal SOC; acute GVHD from the Immune System Disorders SOC; ALT and AST increased from the Investigations SOC; hyperglycemia from the Metabolism and Nutritional Disorders SOC; pyrexia from the General Disorders SOC; neutropenia from the Blood and Lymphatic Disorders SOC; and cytomegalovirus viremia from the Infections and Infestations SOC. Of these events, the gastrointestinal-type events (including increased ALT) and hyperglycemia generally had increased frequency with increasing dose of HDP-CDV. Based upon the overall frequency and distribution of adverse events, gastrointestinal, hepatobiliary, and metabolic events were selected for further evaluation below.

### Adverse Events by Severity

Adverse events were examined by severity to ensure that all safety signals were identified in this study population. Because of the frequency of events in HSCT recipients, particular attention was given to evaluation of Grade 3 (moderate), 4 (severe), and 5 (fatal) adverse events.

TABLE 29 displays, by intensity, the most common TEAEs as consistent with the overview presentation in TABLE 28. Specifically, MedDRA preferred terms are included if at least 15% of subjects in any treatment Cohort experienced the event, or if at least 10% of subjects experienced the event in any HDP-CDV treatment Cohort with at least a 2-fold greater frequency than for placebo treated subjects. Only those preferred terms for which at least one Grade 3 or higher event was experienced in any treatment group are listed. Cytomegalovirus-related AEs are excluded from this analysis.

System Organ Classes demonstrating an apparent increase in the incidence of AEs with increasing HDP-CDV dose include Gastrointestinal, Hepatobiliary, Immune Systems, Investigations, and Metabolism.

TABLE 29
Summary of Most Frequent Treatment Emergent Adverse Events
(TEAE) by Cohort, System Organ Class and Grade
Reported for ≧15% in any Cohort OR ≧10% of Subjects
and ≧2x More Frequently in Any HDP-CDV Group Compared to the
Placebo Group) and having at least 1 Grade 3 event in any treatment group

| | | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| System Organ Class | 40 mg QW | 100 mg QW | 200 mg QW | 200 mg BIW | 100 mg BIW | Placebo |
| Preferred Term | N = 25 | N = 27 | N = 39 | N = 30 | N = 50 | N = 59 |

Blood and Lymphatic System Disorders

| | | | | | | | |
|---|---|---|---|---|---|---|---|
| Neutropenia | Frequency | 2 (8.0%) | 4 (14.8%) | 4 (10.3%) | 4 (13.3%) | 10 (20.0%) | 7 (11.9%) |
| | Grade 2 | 0 | 1 (3.7%) | 1 (2.6%) | 0 | 2 (4.0%) | 2 (3.4%) |
| | Grade 3 | 2 (8.0%) | 2 (7.4%) | 3 (7.7%) | 1 (3.3%) | 5 (10.0%) | 3 (5.1%) |
| | Grade 4 | 0 | 1 (3.7%) | 0 | 2 (6.7%) | 2 (4.0%) | 0 |
| | Grade 5 | 0 | 0 | 0 | 0 | 0 | 0 |
| Thrombocytopenia | Frequency | 3 (12.0%) | 3 (11.1%) | 0 | 5 (16.7%) | 7 (14.0%) | 4 (6.8%) |
| | Grade 2 | 1 (4.0%) | 1 (3.7%) | 0 | 2 (6.7%) | 1 (2.0%) | 1 (1.7%) |
| | Grade 3 | 1 (4.0%) | 1 (3.7%) | 0 | 0 | 1 (2.0%) | 1 (1.7%) |
| | Grade 4 | 1 (4.0%) | 1 (3.7%) | 0 | 3 (10.0%) | 2 (4.0%) | 2 (3.4%) |
| | Grade 5 | 0 | 0 | 0 | 0 | 0 | 0 |
| Leukocytosis | Frequency | 1 (4.0%) | 0 | 2 (5.1%) | 3 (10.0%) | 2 (4.0%) | 1 (1.7%) |
| | Grade 2 | 0 | 0 | 0 | 1 (3.3%) | 0 | 0 |
| | Grade 3 | 0 | 0 | 0 | 0 | 1 (2.0%) | 0 |
| | Grade 4 | 0 | 0 | 0 | 0 | 0 | 0 |
| | Grade 5 | 0 | 0 | 0 | 0 | 0 | 0 |

Gastrointestinal Disorders

| | | | | | | | |
|---|---|---|---|---|---|---|---|
| Abdominal pain | Frequency | 4 (16.0%) | 2 (7.4%) | 5 (12.8%) | 11 (36.7%) | 13 (26.0%) | 4 (6.8%) |
| | Grade 2 | 0 | 0 | 1 (2.6%) | 8 (26.7%) | 1 (2.0%) | 1 (1.7%) |
| | Grade 3 | 1 (4.0%) | 1 (3.7%) | 1 (2.6%) | 0 | 1 (2.0%) | 2 (3.4%) |
| | Grade 4 | 0 | 0 | 0 | 0 | 0 | 0 |
| | Grade 5 | 0 | 0 | 0 | 0 | 0 | 0 |
| Diarrhea | Frequency | 3 (12.0%) | 8 (29.6%) | 13 (33.3%) | 21 (70.0%) | 26 (52.0%) | 16 (27.1%) |
| | Grade 2 | 2 (8.0%) | 3 (11.1%) | 5 (12.8%) | 8 (26.7%) | 6 (12.0%) | 5 (8.5%) |
| | Grade 3 | 0 | 0 | 2 (5.1%) | 11 (36.7%) | 9 (18.0%) | 3 (5.1%) |
| | Grade 4 | 0 | 0 | 0 | 0 | 0 | 0 |
| | Grade 5 | 0 | 0 | 0 | 0 | 0 | 0 |
| Nausea | Frequency | 6 (24.0%) | 5 (18.5%) | 11 (28.2%) | 11 (36.7%) | 17 (34.0%) | 12 (20.3%) |
| | Grade 2 | 2 (8.0%) | 1 (3.7%) | 4 (10.3%) | 6 (20.0%) | 9 (18.0%) | 7 (11.9%) |
| | Grade 3 | 1 (4.0%) | 0 | 1 (2.6%) | 1 (3.3%) | 2 (4.0%) | 1 (1.7%) |
| | Grade 4 | 0 | 0 | 0 | 0 | 0 | 0 |
| | Grade 5 | 0 | 0 | 0 | 0 | 0 | 0 |
| Vomiting | Frequency | 2 (8.0%) | 6 (22.2%) | 6 (15.4%) | 8 (26.7%) | 22 (44.0%) | 11 (18.6%) |
| | Grade 2 | 1 (4.0%) | 2 (7.4%) | 1 (2.6%) | 4 (13.3%) | 8 (16.0%) | 3 (5.1%) |
| | Grade 3 | 0 | 0 | 1 (2.6%) | 0 | 2 (4.0%) | 0 |
| | Grade 4 | 0 | 0 | 0 | 0 | 0 | 0 |
| | Grade 5 | 0 | 0 | 0 | 0 | 0 | 0 |

General Disorders

| | | | | | | | |
|---|---|---|---|---|---|---|---|
| Fatigue | Frequency | 1 (4.0%) | 3 (11.1%) | 5 (12.8%) | 0 | 9 (18.0%) | 9 (15.3%) |
| | Grade 2 | 0 | 0 | 0 | 0 | 2 (4.0%) | 1 (1.7%) |
| | Grade 3 | 0 | 0 | 1 (2.6%) | 0 | 0 | 0 |
| | Grade 4 | 0 | 0 | 0 | 0 | 0 | 0 |
| | Grade 5 | 0 | 0 | 0 | 0 | 0 | 0 |
| Pyrexia | Frequency | 1 (4.0%) | 1 (3.7%) | 7 (17.9%) | 2 (6.7%) | 10 (20.0%) | 11 (18.6%) |
| | Grade 2 | 1 (4.0%) | 0 | 2 (5.1%) | 0 | 4 (8.0%) | 4 (6.8%) |
| | Grade 3 | 0 | 0 | 2 (5.1%) | 0 | 1 (2%) | 4 (6.8%) |
| | Grade 4 | 0 | 0 | 0 | 0 | 0 | 0 |
| | Grade 5 | 0 | 0 | 0 | 0 | 0 | 0 |
| Generalized oedema | Frequency | 1 (4.0%) | 0 | 0 | 3 (10.0%) | 1 (2.0%) | 1 (1.7%) |
| | Grade 2 | 1 (4.0%) | 0 | 0 | 0 | 1 (2.0%) | 1 (1.7%) |
| | Grade 3 | 0 | 0 | 0 | 2 (6.7%) | 0 | 0 |
| | Grade 4 | 0 | 0 | 0 | 0 | 0 | 0 |
| | Grade 5 | 0 | 0 | 0 | 0 | 0 | 0 |

Hepatobiliary Disorders

| | | | | | | | |
|---|---|---|---|---|---|---|---|
| Hyperbilirubinaemia | Frequency | 1 (4.0%) | 0 | 3 (7.7%) | 5 (16.7%) | 3 (6.0%) | 0 |
| | Grade 2 | 0 | 0 | 1 (2.6%) | 1 (3.3%) | 1 (2.0%) | 0 |
| | Grade 3 | 0 | 0 | 0 | 0 | 0 | 0 |
| | Grade 4 | 1 (4.0%) | 0 | 1 (2.6%) | 1 (3.3%) | 0 | 0 |
| | Grade 5 | 0 | 0 | 0 | 0 | 0 | 0 |

Immune system Disorders

| | | | | | | | |
|---|---|---|---|---|---|---|---|
| Acute GVHD | Frequency | 6 (24.0%) | 7 (25.5%) | 13 (33.3%) | 24 (80.0%) | 32 (64.0%) | 15 (25.4%) |
| | Grade 2 | 2 (8.0%) | 4 (14.8%) | 5 (12.8%) | 8 (26.6%) | 11 (22.0%) | 5 (8.5%) |
| | Grade 3 | 1 (4.0%) | 2 (7.3%) | 4 (10.3%) | 10 (30.0%) | 12 (24.0%) | 4 (6.8%) |
| | Grade 4 | | | 2 (5.1%) | 2 (6.7%) | 5 (10.0%) | 2 (3.5%) |
| | Grade 5 | | | 1(2.6%) | 3 (10.0%) | | |

Non CMV-Related Infections and Infestations

| | | | | | | | |
|---|---|---|---|---|---|---|---|
| Staphylococcal Bacteremia | Frequency | 0 | 1 (3.7%) | 1 (2.6%) | 1 (3.3%) | 6 (12.0%) | 2 (3.4%) |
| | Grade 2 | 0 | 0 | 0 | 0 | 2 (4.0%) | 1 (1.7%) |
| | Grade 3 | 0 | 1 (3.7%) | 1 (2.6%) | 1 (3.3%) | 3 (6.0%) | 1 (1.7%) |
| | Grade 4 | 0 | 0 | 0 | 0 | 0 | 0 |
| | Grade 5 | 0 | 0 | 0 | 0 | 0 | 0 |
| Pneumonia | Frequency | 1 (4.0%) | 2 (7.4%) | 0 | 1 (3.3%) | 5 (10.0%) | 1 (1.7%) |
| | Grade 2 | 0 | 0 | 0 | 0 | 1 (2.0%) | 0 |
| | Grade 3 | 0 | 1 (3.7%) | 0 | 1 (3.3%) | 3 (6.0%) | 1 (1.7%) |
| | Grade 4 | 1 (4.0%) | 1 (3.7%) | 0 | 0 | 0 | 0 |
| | Grade 5 | 0 | 0 | 0 | 0 | 1 (2.0%) | 0 |

Investigations

| | | | | | | | |
|---|---|---|---|---|---|---|---|
| ALT increased | Frequency | 2 (8.0%) | 1 (3.7%) | 2 (5.1%) | 10 (33.3%) | 9 (18.0%) | 1 (1.7%) |
| | Grade 2 | 1 (4.0%) | 0 | 1 (2.6%) | 4 (13.3%) | 1 (2.0%) | 0 |
| | Grade 3 | 1 (4.0%) | 1 (3.7%) | 1 (2.6%) | 2 (6.7%) | 4 (8.0%) | 1 (1.7%) |
| | Grade 4 | 0 | 0 | 0 | 0 | 0 | 0 |
| | Grade 5 | 0 | 0 | 0 | 0 | 0 | 0 |
| AST increased | Frequency | 1 (4.0%) | 1 (3.7%) | 1 (2.6%) | 7 (23.3%) | 5 (10.0%) | 2 (3.4%) |
| | Grade 2 | 0 | 1 (3.7%) | 1 (2.6%) | 3 (10.0%) | 1 (2.0%) | 0 |
| | Grade 3 | 1 (4.0%) | 0 | 0 | 0 | 3 (6.0%) | 1 (1.7%) |
| | Grade 4 | 0 | 0 | 0 | 0 | 0 | 0 |
| | Grade 5 | 0 | 0 | 0 | 0 | 0 | 0 |
| LFT^{a} Abnormal | Frequency | 0 | 1 (3.7%) | 2 (5.1%) | 3 (10.0%) | 2 (4.0%) | 2 (3.4%) |
| | Grade 2 | 0 | 0 | 0 | 1 (3.3%) | 1 (2.0%) | 1 (1.7%) |
| | Grade 3 | 0 | 0 | 0 | 1 (3.3%) | 0 | 0 |
| | Grade 4 | 0 | 0 | 0 | 0 | 0 | 1 (1.7%) |
| | Grade 5 | 0 | 0 | 0 | 0 | 0 | 0 |
| GGT^{b} Increased | Frequency | 0 | 1 (3.7%) | 0 | 3 (10.0%) | 4 (8.0%) | 1 (1.7%) |
| | Grade 2 | 0 | 1 (3.7%) | 0 | 0 | 1 (2.0%) | 1 (1.7%) |
| | Grade 3 | 0 | 0 | 0 | 1 (3.3%) | 3 (6.0%) | 0 |
| | Grade 4 | 0 | 0 | 0 | 1 (3.3%) | 0 | 0 |
| | Grade 5 | 0 | 0 | 0 | 0 | 0 | 0 |

Metabolism and Nutrition Disorders

| | | | | | | | |
|---|---|---|---|---|---|---|---|
| Hypocalcaemia | Frequency | 1 (4.0%) | 0 | 0 | 3 (10.0%) | 2 (4.0%) | 1 (1.7%) |
| | Grade 2 | 1 (4.0%) | 0 | 0 | 1 (3.3%) | 1 (2.0%) | 1 (1.7%) |
| | Grade 3 | 0 | 0 | 0 | 1 (3.3%) | 1 (2.0%) | 0 |
| | Grade 4 | 0 | 0 | 0 | 0 | 0 | 0 |
| | Grade 5 | 0 | 0 | 0 | 0 | 0 | 0 |
| Decreased appetite | Frequency | 4 (16.0%) | 5 (18.5%) | 3 (7.7%) | 5 (16.7%) | 9 (18.0%) | 7 (11.9%) |
| | Grade 2 | 1 (4.0%) | 2 (7.4%) | 1 (2.6%) | 4 (13.3%) | 3 (6.0% | 0 |
| | Grade 3 | 1 (4.0%) | 0 | 0 | 0 | 3 (6.0%) | 0 |
| | Grade 4 | 0 | 0 | 0 | 0 | 0 | 0 |
| | Grade 5 | 0 | 0 | 0 | 0 | 0 | 0 |
| Hyperglycaemia | Frequency | 1 (4.0%) | 1 (3.7%) | 1 (2.6%) | 8 (26.7%) | 9 (18.0%) | 4 (6.8%) |
| | Grade 2 | 1 (4.0%) | 0 | 0 | 5 (16.7%) | 4 (8.0%) | 3 (5.1%) |
| | Grade 3 | 0 | 1 (3.7%) | 1 (2.6%) | 2 (6.7%) | 2 (4.0%) | 0 |
| | Grade 4 | 0 | 0 | 0 | 0 | 1 (2.0%) | 0 |
| | Grade 5 | 0 | 0 | 0 | 0 | 0 | 0 |
| Hypokalaemia | Frequency | 4 (16.0%) | 2 (7.4%) | 5 (12.8%) | 5 (16.7%) | 8 (16.0%) | 4 (6.8%) |
| | Grade 2 | 0 | 0 | 2 (5.1%) | 1 (3.3%) | 2 (4.0%) | 1 (1.7%) |
| | Grade 3 | 2 (8.0%) | 0 | 0 | 1 (3.3%) | 2 (4.0%) | 1 (1.7%) |
| | Grade 4 | 0 | 0 | 0 | 0 | 0 | 0 |
| | Grade 5 | 0 | 0 | 0 | 0 | 0 | 0 |
| Hyponatraemia | Frequency | 3 (12.0%) | 1 (3.7%) | 7 (17.9%) | 2 (6.7%) | 1 (2.0%) | 3 (5.1%) |
| | Grade 2 | 1 (4.0%) | 0 | 2 (5.1%) | 0 | 0 | 1 (1.7%) |
| | Grade 3 | 1 (4.0%) | 0 | 2 (5.1%) | 0 | 0 | 0 |
| | Grade 4 | 0 | 0 | 0 | 0 | 0 | 0 |
| | Grade 5 | 0 | 0 | 0 | 0 | 0 | 0 |
| Hyperkalemia | Frequency | 1 (4.0%) | 1 (3.7%) | 1 (2.6%) | 3 (10.0%) | 3 (6.0%) | 2 (3.4%) |
| | Grade 2 | 0 | 0 | 0 | 1 (3.3%) | 2 (4.0%) | 0 |
| | Grade 3 | 0 | 1 (3.7%) | 0 | 1 (3.3%) | 0 | 1 (1.7%) |
| | Grade 4 | 0 | 0 | 0 | 0 | 0 | 0 |
| | Grade 5 | 0 | 0 | 0 | 0 | 0 | 0 |

Musculoskeletal

| | | | | | | | |
|---|---|---|---|---|---|---|---|
| Arthralgias | Frequency | 0 | 3 (11.1%) | 1 (2.6%) | 1 (3.3%) | 3 (6.0%) | 3 (5.1%) |
| | Grade 2 | 0 | 0 | 0 | 1 (3.3%) | 1 (2.0%) | 0 |
| | Grade 3 | 0 | 0 | 0 | 0 | 1 (2.0%) | 1 (1.7%) |
| | Grade 4 | 0 | 0 | 0 | 0 | 0 | 0 |
| | Grade 5 | 0 | 0 | 0 | 0 | 0 | 0 |
| Back Pain | Frequency | 0 | 2 (7.4%) | 2 (5.1%) | 3 (10.0%) | 4 (8.0%) | 2 (3.4%) |
| | Grade 2 | 0 | 0 | 2 (5.1%) | 1 (3.3%) | 3 (6.0%) | 0 |
| | Grade 3 | 0 | 0 | 0 | 0 | 0 | 2 (3.4%) |
| | Grade 4 | 0 | 0 | 0 | 0 | 0 | 0 |
| | Grade 5 | 0 | 0 | 0 | 0 | 0 | 0 |

Neoplasms

| | | | | | | | |
|---|---|---|---|---|---|---|---|
| AML^{c}, Recurrent | Frequency | 3 (12.0%) | 0 | 0 | 0 | 3 (6.0%) | 3 (5.1%) |
| | Grade 2 | 0 | 0 | 0 | 0 | 1 (2.0%) | 1 (1.7%) |
| | Grade 3 | 0 | 0 | 0 | 0 | 0 | 2 (3.4%) |
| | Grade 4 | 1 (4.0%) | 0 | 0 | 0 | 1 (2.0%) | 0 |
| | Grade 5 | 0 | 0 | 0 | 0 | 1 (2.0%) | 0 |

Renal and Urinary Disorders

| | | | | | | | |
|---|---|---|---|---|---|---|---|
| Renal Failure | Frequency | 1 (4.0%) | 2 (7.4%) | 0 | 0 | 5 (10.0%) | 3 (5.1%) |
| | Grade 2 | 0 | 2 (7.4%) | 0 | 0 | 1 (2.0%) | 2 (3.4%) |
| | Grade 3 | 1 (4.0%) | 0 | 0 | 0 | 1 (2.0%) | 0 |
| | Grade 4 | 0 | 0 | 0 | 0 | 1 (2.0%) | 1 (1.7%) |
| | Grade 5 | 0 | 0 | 0 | 0 | 0 | 0 |

^{a}LFT, liver function test
^{b}GGT, gamma glumatyl transferase
^{c}AML, acute myeloid laukemia

When examining AEs of Grade 3-5 intensity, the events that generally show a dose-response relationship with increasing doses of HDP-CDV are diarrhea, diagnosis of acute GVHD, increased ALT, and hyperglycemia. Based on these findings, the following categories of events are considered in sections below: GI disturbances, hepatobiliary events, diagnosis of GVHD, and metabolic disorders. No additional safety signals were identified by restricting the review to events of Grade 4 and 5 intensity.

### Adverse Events Leading to Study Drug Withdrawal

Subjects who withdrew from study medication for any cause in Study HDP-CDV-201 are listed in TABLE 25, along with the gastrointestinal TEAEs that led to the discontinuation of treatment.

The rates of discontinuation for GI AEs were highest in the 200 mg BIW dose group; in other system organ classes, discontinuations of study drug were infrequent and without apparent dose relationship, and therefore are not presented in the text. Neutropenia and decrease in GFR were identified in the protocol as potentially important reasons for discontinuation of HDP-CDV. During the study, no subject discontinued due to decreased renal function or related terms (investigations SOC) and only one subject randomized to 40 mg QW discontinued due to acute renal failure. Zero (0), 1, 2, 0 and 1 subject(s) discontinued due to neutropenia in Cohorts 1, 2, 3, 4, and 4A, respectively, as compared to 1 subject randomized to placebo.

### Laboratory Abnormalities by Severity

Laboratory abnormalities, grouped by parameters of potential interest were assessed. Renal and hematologic abnormalities, while liver function tests were tested and are presented below in the context of hepatobiliary conditions.

### Renal and Hematological Laboratory Abnormalities

HDP-CDV is a lipid conjugate of CDV. Because CDV is known to have adverse effects on renal function and bone marrow, decreased GFR and persistent neutropenia were identified as AEs of special interest. Key renal and hematological (TABLE 30) laboratory parameters were measured and listed below by Study HDP-CDV-201 treatment groups. TABLE 30 includes abnormal levels of serum creatinine, neutrophils, platelets, WBCs, and hemoglobin and includes data at Screening and during the treatment period.

TABLE 30
Proportion of Subjects with Abnormal Levels of Serum Creatinine,
Neutrophils, Platelets, WBC, and Hemoglobin, Cohorts 1 through 4A

| | | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Parameter | N = 25 | N = 27 | N = 39 | N = 30 | N = 50 | N = 59 |

Serum Creatinine (Screening)

| | | | | | | |
|---|---|---|---|---|---|---|
| >1 × ULN-1.5 × ULN | 3 (12.0%) | 5 (18.5%) | 4 (10.8%) | 5 (16.7%) | 11 (23.9%) | 9 (15.5%) |
| >1.5 × ULN-3 × ULN | 0 | 0 | 0 | 0 | 0 | 1 (1.7%) |
| >3 × ULN-6 × ULN | 0 | 0 | 0 | 0 | 0 | 0 |
| >6 × ULN | 0 | 0 | 0 | 0 | 0 | 0 |

Serum Creatinine (any time during therapy)

| | | | | | | |
|---|---|---|---|---|---|---|
| >1 × ULN-1.5 × ULN | 8 (34.8%) | 16 (59.3%) | 16 (42.1%) | 11 (37.9%) | 16 (32.0%) | 24 (42.1%) |
| >1.5 × ULN-3 × ULN | 2 (8.7%) | 5 (18.5%) | 2 (5.3%) | 1 (3.4%) | 10 (20.0%) | 3 (5.3%) |
| >3 × ULN-6 × ULN | 0 | 0 | 0 | 0 | 0 | 0 |
| >6 × ULN | 0 | 0 | 0 | 0 | 0 | 0 |

Neutrophils (Screening)

| | | | | | | |
|---|---|---|---|---|---|---|
| <LLN-1.5 GI/L | 4 (25.0%) | 4 (16.7%) | 2 (6.5%) | 0 | 2 (4.5%) | 7 (13.2%) |
| <1.5-1 GI/L | 1 (6.3%) | 1 (4.2%) | 3 (9.7%) | 2 (7.7%) | 3 (6.8%) | 5 (9.4%) |
| <1.0-0.5 GI/L | 3 (18.8%) | 4 (16.7%) | 5 (16.1%) | 3 (11.5%) | 5 (11.4%) | 6 (11.3%) |
| <0.5 GI/L | 1 (6.3%) | 2 (8.3%) | 6 (19.4%) | 2 (7.7%) | 5 (11.4%) | 6 (11.3%) |

Neutrophils (any time during therapy)

| | | | | | | |
|---|---|---|---|---|---|---|
| <LLN-1.5 GI/L | 4 (17.4%) | 1 (3.7%) | 1 (2.6%) | 3 (10.7%) | 5 (10.0%) | 14 (24.6%) |
| <1.5-1 GI/L | 5 (21.7%) | 9 (33.3%) | 11 (28.9%) | 7 (25.0%) | 10 (20.0%) | 11 (19.3%) |
| <1.0-0.5 GI/L | 2 (8.7%) | 2 (7.4%) | 7 (18.4%) | 4 (14.3%) | 10 (20.0%) | 3 (5.3%) |
| <0.5 GI/L | 0 | 3 (11.1%) | 2 (5.3%) | 0 | 3 (6.0%) | 3 (5.3%) |

Platelets (Screening)

| | | | | | | |
|---|---|---|---|---|---|---|
| <LLN-75 GI/L | 4 (16.7%) | 6 (28.6%) | 6 (17.1%) | 5 (21.7%) | 8 (19.0%) | 9 (17.6%) |
| <75-50 GI/L | 1 (4.2%) | 2 (9.5%) | 4 (11.4%) | 4 (17.4%) | 7 (16.7%) | 9 (17.6%) |
| <50-25 GI/L | 7 (29.2%) | 5 (23.8%) | 13 (37.1%) | 3 (13.0%) | 8 (19.0%) | 12 (23.5%) |
| <25 GI/L | 7 (29.2%) | 3 (14.3%) | 7 (20.0%) | 4 (17.4%) | 7 (16.7%) | 11 (21.6%) |

Platelets (any time during therapy)

| | | | | | | |
|---|---|---|---|---|---|---|
| <LLN-75 GI/L | 6 (26.1%) | 7 (25.9%) | 7 (18.4%) | 6 (21.4%) | 21 (42.0%) | 23 (40.4%) |
| <75-50 GI/L | 4 (17.4%) | 6 (22.2%) | 7 (18.4%) | 2 (7.1%) | 10 (20.0%) | 11 (19.3%) |
| <50-25 GI/L | 9 (39.1%) | 6 (22.2%) | 13 (34.2%) | 8 (28.6%) | 7 (14.0%) | 10 (17.5%) |
| <25 GI/L | 4 (17.4%) | 4 (14.8%) | 7 (18.4%) | 8 (28.6%) | 6 (12.0%) | 8 (14.0%) |

WBC (Screening)

| | | | | | | |
|---|---|---|---|---|---|---|
| <LLN-3 GI/L | 1 (4.2%) | 3 (12.0%) | 5 (13.9%) | 4 (13.8%) | 3 (6.5%) | 12 (20.7%) |
| <3-2 GI/L | 3 (12.5%) | 4 (16.0%) | 4 (11.1%) | 2 (6.9%) | 6 (13.0%) | 6 (10.3%) |
| <2-1 GI/L | 2 (8.3%) | 4 (16.0%) | 6 (16.7%) | 3 (10.3%) | 7 (15.2%) | 6 (10.3%) |
| <1 GI/L | 10 (41.7%) | 3 (12.0%) | 9 (25.0%) | 5 (17.2%) | 5 (10.9%) | 12 (20.7%) |

WBC (any time during therapy)

| | | | | | | |
|---|---|---|---|---|---|---|
| <LLN-3 GI/L | 5 (21.7%) | 4 (14.8%) | 6 (15.8%) | 3 (10.7%) | 11 (22.0%) | 9 (15.8%) |
| <3-2 GI/L | 6 (26.1%) | 6 (22.2%) | 7 (18.4%) | 9 (32.1%) | 12 (24.0%) | 18 (31.6%) |
| <2-1 GI/L | 5 (21.7%) | 4 (14.8%) | 13 (34.2%) | 5 (17.9%) | 8 (16.0%) | 6 (10.5%) |
| <1 GI/L | 0 | 3 (11.1%) | 1 (2.6%) | 2 (7.1%) | 4 (8.0%) | 3 (5.3%) |

Hemoglobin (Screening)

| | | | | | | |
|---|---|---|---|---|---|---|
| <LLN-10 g/dL | 10 (41.7%) | 14 (56.0%) | 19 (52.8%) | 9 (31.0%) | 20 (43.5%) | 24 (41.4%) |
| <10-8 g/dL | 11 (45.8%) | 9 (36.0%) | 13 (36.1%) | 15 (51.7%) | 23 (50.0%) | 22 (37.9%) |
| <8-6.5 g/dL | 0 | 0 | 2 (5.6%) | 0 | 0 | 2 (3.4%) |
| <6.5 g/dL | 0 | 0 | 0 | 0 | 0 | 1 (1.7%) |

Hemoglobin (any time during therapy)

| | | | | | | |
|---|---|---|---|---|---|---|
| <LLN-10 g/dL | 10 (43.5%) | 12 (44.4%) | 13 (34.2%) | 12 (42.9%) | 17 (34.0%) | 27 (47.4%) |
| <10-8 g/dL | 11 (47.8%) | 12 (44.4%) | 19 (50.0%) | 12 (42.9%) | 27 (54.0%) | 19 (33.3%) |
| <8-6.5 g/dL | 1 (4.3%) | 1 (3.7%) | 5 (13.2%) | 2 (7.1%) | 4 (8.0%) | 5 (8.8%) |
| <6.5 g/dL | 0 | 1 (3.7%) | 0 | 0 | 1 (2.0%) | 1 (1.8%) |

Neutropenia and decline in GFR were identified in the protocol as reasons for discontinuation of particular interest. There was no Grade 3 or 4 increases in creatinine in subjects receiving HDP-CDV at any dose. There was a slight excess of subjects with Grade 2 creatinine increases in subjects who received HDP-CDV 100 mg BIW; however there was no apparent increase in the proportion of subjects with Grade 2 creatinine increases with increasing doses of HDP-CDV. A review of individual subject creatinine levels over time indicates that most of the Grade 2 creatinine abnormalities were transient and typically returned to Baseline or to the normal range on the next visit. In addition, more subjects randomized to receive HDP-CDV 100 mg BIW initiated amphotericin B therapy during the study (10%) than subjects who received placebo (2%). One subject in Cohort 2 and no subjects in Cohorts 3 and 4 initiated amphotericin B during the study and 4% in Cohort 1 initiated amphotericin B during the Study.

The incidence of Grade 3 or 4 anemia, thrombocytopenia, and leukopenia was similar between subjects who received HDP-CDV once or BIW and subjects randomized to placebo in the study. While there is an apparent increase in the rate of Grade 3 neutropenia in subjects who received HDP-CDV 100 mg BIW compared to placebo, a review of the individual laboratory results over time indicate that these episodes of neutropenia were generally transient and early after initiation of therapy, with the majority resolved at the next Study Visit. The fact that only one subject (2%) discontinued HDP-CDV due to neutropenia in Cohort 4A (compared to 1.7% of the pooled placebo subjects) is supportive of this interpretation of the laboratory results (no subjects in this Cohort discontinued due to febrile neutropenia).

The proportion of subjects who received immunostimulants or relevant blood products during the treatment period is listed by Cohort in TABLE 31.

TABLE 31
Proportion of Subjects Receiving Immunostimulants or Relevant
Blood Products during the Treatment Period by Cohort

| | | | | | |
|---|---|---|---|---|---|
| Cohort 1 | Cohort 2 | Cohort 3 | Cohort 4 | Cohort 4A | |
| HDP-CDV | HDP-CDV | HDP-CDV | HDP-CDV | HDP-CDV | Pooled |
| 40 mg QW | 100 mg QW | 200 mg QW | 200 mg BIW | 100 mg BIW | Placebo |
| N = 25 | N = 27 | N = 39 | N = 30 | N = 50 | N = 59 |

Immunostimulants

| | | | | | | |
|---|---|---|---|---|---|---|
| Aldesleukin | 0 | 0 | 0 | 0 | 0 | 1 (1.7%) |
| Filgrastim | 4 (16.0%) | 4 (14.8%) | 11 (28.2%) | 5 (16.7%) | 11 (22.0%) | 12 (20.3%) |
| Granulocyte Colony | 0 | 0 | 1 (2.6%) | 1 (3.3%) | 0 | 0 |
| Stimulating Factor | | | | | | |
| Pegfilgrastim | 0 | 0 | 1 (2.6%) | 1 (3.3%) | 2 (4.0%) | 1 (1.7%) |
| Sargramostim | 0 | 0 | 1 (2.6%) | 0 | 0 | 0 |

Blood Substitutes and Perfusion Solutions

| | | | | | | |
|---|---|---|---|---|---|---|
| Blood and Related | 1 (4.0%) | 0 | 0 | 0 | 0 | 0 |
| Products | | | | | | |
| Blood, Whole | 0 | 1 (3.7%) | 0 | 0 | 0 | 0 |
| Platelets | 5 (20.0%) | 3 (11.1%) | 6 (15.4%) | 6 (20.0%) | 6 (12.0%) | 4 (6.8%) |
| Red Blood Cells | 0 | 2 (7.4%) | 3 (7.7%) | 4 (13.3%) | 3 (6.0%) | 3 (5.1%) |
| Red Blood Cells, | 2 (8.0%) | 3 (11.1%) | 6 (15.4%) | 5 (16.7%) | 6 (12.0%) | 8 13.6%) |
| Concentrated | | | | | | |

As can be seen in TABLE 31, there was no increase in the proportion of subjects requiring either red blood cell or platelet transfusions, or treatment with G-CSF in any of the HDP-CDV treated groups, as compared to subjects on placebo.

### Safety Signals Identified

Based on the overall safety profile of various doses of HDP-CDV in Study HDP-CDV-201, the following safety signals were identified: hepatobiliary events gastrointestinal events and events reported as acute GVHD. These events demonstrated a correlation between dose of HDP-CDV to AE frequency and intensity.

### Hepatic Events

Hepatic laboratory abnormalities and adverse events were recorded in this study. The hepatic organ system was identified as of interest because of preclinical animal toxicology studies, in which increases in ALT (with no histopathological correlate) were noted in rats, mice, and monkeys.

With respect to preclinical studies, the effect of HDP-CDV on the liver was evaluated in several GLP compliant toxicology studies, including two 14 day studies in the CD-1 mouse; 3 studies of up to 13 weeks duration in the Sprague Dawley rat; and 4 studies of up to 39 weeks duration in the cynomolgus monkey. Following daily administration of HDP-CDV for 14 days in the CD-1 mouse, mean ALT values were increased about 1.5 to 2-fold at doses of 10 or 20 mg HDP-CDV/kg/day relative to values in control animals. ALT increases of up to 5-fold were observed in individual animals, while values in most animals remained normal. There were no other dose-related changes in hepatic endpoints in mice including AST, GGT, total bilirubin, liver weights, macroscopic observations, or liver histopathology.

Cynomolgus monkeys given HDP-CDV daily for 14 days exhibited dose-related increases in ALT which were quickly reversible after cessation of dosing. Following 13 weeks of twice-weekly administration of HDP-CDV in monkeys, ALT and AST increased in treated monkeys relative to controls. However, the change was not dose-dependent, but rather the same regardless of dose in all treatment groups. The change persisted at subsequent sampling intervals during Week 26 and Week 39 but, similarly, did not progress, instead remaining about the same for the full duration of treatment. The ALT elevations reversed quickly upon cessation of dosing. Monkeys exhibited no dose-related changes in GGT, total bilirubin, organ weights, macroscopic observations, or histopathology.

Minor increases (i.e., about 2-5 fold) in ALT were observed in both rodent and non-rodent species in toxicology studies of HDP-CDV and indicated a potential hepatic effect of HDP-CDV. The changes appeared with highest frequency in monkeys, followed by mice and then rats. There was no temporal correlation with ALT increases, rather the increase, if it occurred, was apparent at the first sampling interval after initiation of dosing with the degree of elevation remaining similar at subsequent sampling intervals and quickly reversing after cessation of dosing. Likewise, the ALT elevations did not correlate with dose but were generally of equal magnitude in all treated animals. These trends are most obvious in data from the 39 week study of HDP-CDV in cynomolgus monkeys. There were no gross or microscopic hepatic changes that correlated with the ALT increases, hence the elevations were not judged to be adverse in any study.

For subjects in Study HDP-CDV-201, increases in ALT, AST, alkaline phosphatase, and total bilirubin at Screening and during therapy by Cohort are summarized in TABLE 32.

TABLE 32
Study HDP-CDV-201: Proportion of Subjects with Abnormal
Increases from Baseline in ALT, AST, Alkaline Phosphatase and Total Bilirubin Levels, Cohorts
1 through 4A

| | | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Parameter | N = 25 | N = 27 | N = 39 | N = 30 | N = 50 | N = 59 |

ALT (Screening)

| | | | | | | |
|---|---|---|---|---|---|---|
| >3 × ULN-5 × ULN | 0 | 0 | 0 | 2 (6.7%) | 1 (2.2%) | 2 (3.5%) |
| >5 × ULN-10 × ULN | 0 | 0 | 0 | 2 (6.7%) | 1 (2.2%) | 0 |
| >10 × ULN-20 × ULN | 0 | 0 | 0 | 0 | 1 (2.2%) | 0 |
| >20 × ULN | 0 | 0 | 0 | 0 | 0 | 0 |

ALT (any time during therapy)

| | | | | | | |
|---|---|---|---|---|---|---|
| >3 × ULN-5 × ULN | 2 (8.7%) | 1 (3.7%) | 4 (10.5%) | 4 (13.8%) | 10 (20.0%) | 4 (7.0%) |
| >5 × ULN-10 × ULN | 1 (4.3%) | 0 | 5 (13.2%) | 6 (20.7%) | 4 (8.0%) | 4 (7.0%) |
| >10 × ULN-20 × ULN | 0 | 1 (3.7%) | 0 | 2 (6.9%) | 1 (2.0%) | 1(1.8%) |
| >20 × ULN | 0 | 0 | 1 (2.6%) | 0 | 1 (2.0%) | 0 |

AST (Screening)

| | | | | | | |
|---|---|---|---|---|---|---|
| >3 × ULN-5 × ULN | 0 | 0 | 0 | 1 (3.3%) | 1 (2.2%) | 1 (1.8%) |
| >5 × ULN-10 × ULN | 0 | 0 | 0 | 0 | 1 (2.2%) | 0 |
| >10 × ULN-20 × ULN | 0 | 0 | 0 | 0 | 1 (2.2%) | 0 |
| >20 × ULN | 0 | 0 | 0 | 0 | 0 | 0 |

AST (any time during therapy)

| | | | | | | |
|---|---|---|---|---|---|---|
| >3 × ULN-5 × ULN | 1 (4.3%) | 1 (3.7%) | 5 (13.2%) | 6 (20.7%) | 2 (4.0%) | 1 (1.8%) |
| >5 × ULN-10 × ULN | 0 | 0 | 1 (2.6%) | 0 | 3 (6.0%) | 1 (1.8%) |
| >10 × ULN-20 × ULN | 0 | 0 | 0 | 0 | 0 | 0 |
| >20 × ULN | 0 | 0 | 0 | 0 | 0 | 0 |

Total Bilirubin (Screening)

| | | | | | | |
|---|---|---|---|---|---|---|
| >1-1.5 × ULN | 1 (4.0%) | 1 (3.8%) | 0 | 1 (3.3%) | 3 (6.5%) | 3 (5.2%) |
| >1.5-3 × ULN | 3 (12.0%) | 1 (3.8%) | 0 | 0 | 2 (4.3%) | 1 (1.7%) |
| >3-10 × ULN | 0 | 0 | 0 | 0 | 0 | 0 |
| >10 × ULN | 0 | 0 | 0 | 0 | 0 | 0 |

Total Bilirubin (any time during therapy)

| | | | | | | |
|---|---|---|---|---|---|---|
| >1-1.5 × ULN | 3 (13.0%) | 0 | 1 (2.6%) | 3 (10.3%) | 5 (10.0%) | 0 |
| >1.5-3 × ULN | 1 (4.3%) | 1 (3.7%) | 3 (7.9%) | 1 (3.4%) | 3 (6.0%) | 0 |
| >3-10 × ULN | 0 | 0 | 0 | 1 (3.4%) | 1 (2.0%) | 0 |
| >10 × ULN | 0 | 0 | 0 | 0 | 0 | 0 |

Alkaline Phosphatase (Screening)

| | | | | | | |
|---|---|---|---|---|---|---|
| >ULN-2.5 × ULN | 6 (24.0%) | 6 (22.2%) | 5 (13.5%) | 7 (23.3%) | 8 (17.4%) | 11 (19.3%) |
| >2.5-5.0 × ULN | 0 | 0 | 0 | 0 | 0 | 0 |
| >5.0-20.0 × ULN | 0 | 0 | 0 | 0 | 0 | 0 |
| >20.0 × ULN | 0 | 0 | 0 | 0 | 0 | 0 |

Alkaline Phosphatase (any time during therapy)

| | | | | | | |
|---|---|---|---|---|---|---|
| >ULN-2.5 × ULN | 6 (26.1%) | 7 (25.9%) | 14 (36.8%) | 11 (37.9%) | 11 (22.0%) | 16 (28.1%) |
| >2.5-5.0 × ULN | 0 | 1 (3.7%) | 0 | 0 | 3 (6.0%) | 3 (5.3%) |
| >5.0-20.0 × ULN | 0 | 1 (3.7%) | 0 | 0 | 0 | 0 |
| >20.0 × ULN | 0 | 0 | 0 | 0 | 0 | 0 |

Overall, there is an apparent dose relationship in increases in ALT with increasing doses of HDP-CDV. The frequency and intensity of these increases were numerically higher at total doses that were at least 200 mg QW (Cohorts 4 and 4A).

At doses of HDP-CDV of 200 mg BIW, there was an increase in the proportion of subjects (8 of 30 subjects or 27.6%) with Grade 3 or higher ALT (>5x×ULN) as compared to placebo or other doses. At doses of HDP-CDV of 100 mg BIW, only an increase in the proportion of subjects with moderate increase in ALT (3 to 5×ULN) was observed to be in excess of the rate in subjects receiving placebo and other doses. At doses of HDP-CDV of 200 mg QW, there was an apparent increase in the proportion of subjects with Grade 3 or higher ALT (6 of 39 subjects or 15.8%) when compared to placebo-treated subjects (5 of 59 subjects or 8.8%) and lower doses, but the actual numbers are small. Increases in AST (3 to 5×ULN) were more frequent in subjects who received HDP-CDV at 200 mg QW and 200 mg BIW as compared to other dosing Cohorts and placebo recipients.

A number of subjects entered the study with elevated liver enzymes. The fold change from Baseline in ALT, AST and total bilirubin at peak (maximum ALT value), end of treatment (follow-up Week 1) and end of Study (follow-up Week 4) by Cohort is shown TABLE 33.

TABLE 33
Study HDP-CDV-201: Relative Increase from Baseline in serum ALT Levels

| | Cohort 1 | Cohort 2 | Cohort 3 | Cohort 4 | Cohort 4A | |
|---|---|---|---|---|---|---|
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Serum ALT level | N = 25 | N = 27 | N = 39 | N = 30 | N = 50 | N = 59 |
| Peak Median fold increase | 1.80 | 1.53 | 3.13 | 3.55 | 2.86 | 1.67 |
| from Baseline | | | | | | |
| Median fold increase from | 1.50 | 1.50 | 2.52 | 1.87 | 1.52 | 1.06 |
| Baseline at Follow-up Week 1 | | | | | | |
| Median fold increase from | 1.00 | 0.96 | 1.06 | 1.34 | 1.11 | 1.08 |
| Baseline at Follow-up Week 4 | | | | | | |

The data demonstrated that on average the increase in ALT from Baseline ranged from 1.53 to 3.55 fold at the time of maximum ALT increase. The values at the end of treatment with HDP-CDV were lower than at the time of peak and returned to Baseline levels after discontinuation of treatment. ALT values increased between Week 2 and 4 and remained stable during the remainder of HDP-CDV therapy. This pattern of ALT increase is similar to that observed in animal models. The frequency of these increases, dose and time relationship suggest a drug-related pharmacological effect, rather than any clinically meaningful toxicity.

A small proportion of subjects receiving HDP-CDV demonstrated increases in bilirubin levels (TABLE 32). There was no apparent dose response in the frequency or intensity of these bilirubin increases. Of note, more subjects (27%) in Cohort 4 (200 mg BIW) received total parenteral nutrition (TPN) during the course of the study than in Cohorts 4A and 3 (10%) or in Cohort 1 or 2, as well as subjects who received placebo (≦5%). Administration of TPN may have contributed in some of the cases of bilirubin increases.

During the study, 6 subjects experienced a concomitant increase in ALT>×3ULN and in total bilirubin>2×ULN. Additional details on these 6 subjects are listed in TABLE 34.

TABLE 34
Study HDP-CDV-201: Summary of Overlap between
Bilirubin, ALT, and AST Increases

| Subject | | Demographics | Bilirubin | ALT | AST | Alk Phos^{a} | | |
|---|---|---|---|---|---|---|---|---|
| Number | Dose | Sex/Age | (μmol/L) | (U/L) | (U/L) | (U/L) | Visit | Notes |
| 1015 | placebo | Male/46 | 65 | 147 | Normal | Normal | Post Treatment | |
| | | | | | | | Wk 4 | |
| 2018 | placebo | Male/44 | 352 | 277 | 149 | 634 | Post Treatment | Entered study |
| | | | | | | | Wk 4 | with elevated |
| | | | | | | | | alk phos on |
| | | | | | | | | fluconazole |
| 3044 | 200 QW | Male/51 | 50 | 227 | Normal | 174 | Post Treatment | On Bactrim and |
| | | | | | | Previously | Wk 4 | Fluconazole |
| | | | | | | elevated | | |
| 3001 | 200 QW | Female/51 | 89 | 375 | 164 | 337 | Post Treatment | On Tacro and |
| | | | | | | elevated | Wk 1 | Voriconazole |
| 4008 | 200 BIW | Male/28 | 48 | 137 | Normal | 117 | Posttreatment | On |
| | | | | | | Previously | Wk 1 | voriconazole, |
| | | | | | | elevated | | Tacro, Bactrim |
| 4090 | 100 BIW | Female/29 | 44 | 445 | 195 | Normal | Treatment Wk 8 | Voriconazole |
| | | | Normal | 792 | 312 | Normal | Treatment Wk 10 | discontinued at |
| | | | | | | | | Week 8 |

^{a}Alk phos = alkaline phosphatase

Two subjects were randomized to placebo treatment. Two subjects were randomized to HDP-CDV 200 mg QW and 1 subject was assigned to HDP-CDV 200 mg BIW. In these 3 subjects receiving HDP-CDV, the abnormalities were noted after the end of study drug administration; all 3 subjects were receiving azoles as a concomitant medication and had elevated alkaline phosphatase levels, indicating a possible cholestatic origin for the increased bilirubin.

The subject randomized to HDP-CDV 100 mg BIW (4090) experienced increases in bilirubin and ALT during HDP-CDV dosing (Week 8); the subject was also receiving voriconazole, which was stopped due to the liver enzyme abnormalities. By Week 10, the subject's total bilirubin had returned to normal levels.

In summary, there is no evidence in this study that HDP-CDV is associated with liver enzyme abnormalities compatible with the Hy's law above and beyond what was observed in the placebo recipients. Hy's law is a prognostic indicator that a pure drug-induced liver injury (DILI) leading to jaundice, without a hepatic transplant, has a case fatality rate of 10% to 50%.

Hepatobiliary AEs are summarized in TABLE 35, listed by Cohort and Preferred Term. Overall, very few hepatobiliary events were reported and most of them were consistent with bilirubin increases. These events were mild or moderate in intensity with the exception of 3 episodes of hyperbilirubinemia (one episode each in subjects who received HDP-CDV 40 mg QW, 200 mg QW and 200 mg BIW). One episode of liver injury in each of Cohorts 3 and 4 was considered severe; a third episode in Cohort 3 was considered mild (subject 3025, whose event resolved after discontinuation of isoniazid). One episode of hyperbilirubinemia in a subject randomized to HDP-CDV 100 mg BIW was considered related to study drug by the investigator. The episode of severe liver injury in subject 4008 who received HDP-CDV 200 mg BIW was considered related to HDP-CDV, led to discontinuation of HDP-CDV and resolved after treatment discontinuation. The other events were considered unrelated to HDP-CDV.

TABLE 35
Summary of Hepatobiliary Clinical AEs

| | Cohort 1 | Cohort 2 | Cohort 3 | Cohort 4 HDP- | Cohort 4A | |
|---|---|---|---|---|---|---|
| | HDP-CDV | HDP-CDV | HDP-CDV | CDV | HDP-CDV | Pooled |
| System Organ Class | 40 mg QW | 100 mg QW | 200 mg QW | 200 mg BIW | 100 mg BIW | Placebo |
| Preferred Term | N = 25 | N = 27 | N = 39 | N = 30 | N = 50 | N = 59 |
| Cholelithiasis | 1 (4.0%) | 0 | 0 | 0 | 0 | 0 |
| Hepatomegaly | 0 | 0 | 0 | 1 (3.3%) | 0 | 0 |
| Hyperbilirubinaemia | 1 (4.0%) | 0 | 3 (7.7%) | 5 (16.7%) | 3 (6.0%) | 0 |
| Jaundice | 0 | 0 | 1 (2.6%) | 1 (3.3%) | 0 | 0 |
| Liver injury | 0 | 0 | 2 (5.1%) | 1 (3.3%) | 0 | 0 |

Additional information on these hepatobiliary AEs are displayed in TABLE 36.

TABLE 36
Subjects with Hepatobiliary AEs (by Treatment Cohort)

| | | | | | | |
|---|---|---|---|---|---|---|
| Subject | | | | | | |
| Number | AE Term | Outcome | Grade | Relationship | Action Taken | Comment^{a} |

Cohort 1

| | | | | | | |
|---|---|---|---|---|---|---|
| 1003 | worsening | not recovered/not | 4 | not related | dose not changed | TPN |
| | hyperbilirubinemia | resolved | | | | |
| 1016 | symptomatic | recovered/resolved | 3 | not related | dose not changed | surgery - |
| | gallstones | | | | | cholecystectomy |

Cohort 3

| | | | | | | |
|---|---|---|---|---|---|---|
| 3007 | acute liver injury | not recovered/not | 3 | not related | dose not changed | TPN |
| | | resolved | | | | |
| 3009 | hyperbilirubinemia | recovered/resolved | 1 | not related | dose not changed | |
| 3025 | acute liver injury | recovered/resolved | 1 | not related | dose not changed | isoniazid |
| | | | | | | discontinued |
| 3030 | jaundiced | not recovered/not | 2 | not related | dose not changed | liver labs |
| | | resolved | | | | |
| | hyperbilirubenemia | not recovered/not | 2 | not related | dose not changed | ultrasound, liver |
| | | resolved | | | | biospy |
| 3044 | gallbladder sludge | not recovered/not | 2 | not related | dose not changed | |
| | | resolved | | | | |
| | hyperbilirubinemia | not recovered/not | 4 | not related | dose not changed | |
| | | resolved | | | | |

Cohort 4

| | | | | | | |
|---|---|---|---|---|---|---|
| 4001 | hyperbilirubenemia | not recovered/not | 2 | not related | not applicable | |
| | | resolved | | | | |
| 4002 | jaundice | not recovered/not | 1 | not related | dose not changed | cyclosporine held |
| | | resolved | | | | |
| 4004 | worsening | recovered/resolved | 1 | not related | dose not changed | |
| | hyperbilirubinemia | | | | | |
| 4008 | acute liver injury | recovered/resolved | 3 | possibly related | drug withdrawn | TPN |
| 4014 | hyperbilirubinemia | not recovered/not | 4 | not related | dose not changed | |
| | | resolved | | | | |
| 4019 | hepatomegaly | recovered/resolved | 1 | not related | dose not changed | |
| 4041 | hyperbilirubinemia | recovered/resolved | 1 | not related | dose not changed | |
| 4042 | hyperbilirubinemia | recovered/resolved | 1 | not related | drug interrupted | abdominal x ray |

Cohort 4a

| | | | | | | |
|---|---|---|---|---|---|---|
| 4058 | cholecystitis | recovered/resolved | 3 | not related | not applicable | surgery |
| 4080 | hyperbilirubinemia | not recovered/not | 1 | not related | not applicable | voriconazole held |
| | | resolved | | | | |
| 4090 | hyperbilirubinemia | recovered/resolved | 2 | possibly related | dose not changed | voriconazole held, |
| | | | | | | TPN |
| 4109 | worsening | not recovered/not | 1 | not related | not applicable | |
| | hyperbilirubinemia | resolved | | | | |

^{a}TPN: total parenteral nutrition

Subject 3030, randomized to HDP-CDV 200 mg QW, was reported with an event of hyperbilirubinemia and one event of jaundice, both were moderate in intensity and considered unrelated to HDP-CDV. Subject 3044 was reported with Grade 4 hyperbilirubinemia in the context of gallbladder sludge both considered unrelated to HDP-CDV. Three events of hyperbilirubinemia occurred after HDP-CDV was stopped for other reasons. Only one event of acute liver injury was considered possibly related to HDP-CDV and resolved after drug withdrawal. Fourteen out of 19 subjects who experienced hepatobiliary events had AEs that were considered mild or moderate in intensity.

### Hepatic Events Summary

Dose proportional, limited increases in ALT were noted 2 to 4 weeks after initiation of HDP-CDV therapy. The ALT increases were typically Grade 2 or lower and 2-4 times Baseline, occurring between Weeks 2 and 4 of dosing, at doses of 200 mg per week or higher.

Few hepatobiliary clinical AEs were reported in association with treatment with HDP-CDV and most were mild or moderate in intensity. No case of drug induced liver injury (DILI) clearly attributable to HDP-CDV was noted during the course of the study. One event of liver injury possibly associated with the administration of HDP-CDV 200 mg BIW was reported, this event resolved after discontinuation of HDP-CDV.

### Diarrhea and Gastrointestinal AEs

TABLE 37 displays the frequency of selected gastrointestinal AEs reported by dosing Cohorts.

TABLE 37
Study HDP-CDV-201 Gastrointestinal AEs by Cohort

| | System Organ Class | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Preferred Term | N = 25 | N = 27 | N = 39 | N = 30 | N = 50 | N = 59 |
| Abdominal discomfort | 0 | 1 (3.7%) | 1 (2.6%) | 0 | 4 (8.0%) | 1 (1.7%) |
| Abdominal distention | 2 (8.0%) | 2 (7.4%) | 0 | 1 (3.3%) | 0 | 3 (5.1%) |
| Abdominal pain | 4 (16.0%) | 2 (7.4%) | 5 (12.8%) | 11 (36.7%) | 13 (26.0%) | 4 (6.8%) |
| Abdominal pain upper | 1 (4.0%) | 2 (7.4%) | 1 (2.6%) | 3 (10.0%) | 2 (4.0%) | 2 (3.4%) |
| Constipation | 1 (4.0%) | 0 | 2 (5.1%) | 3 (10.0%) | 2 (4.0%) | 3 (5.1%) |
| Diarrhoea | 3 (12.0%) | 8 (29.6%) | 13 (33.3%) | 21 (70.0%) | 26 (52.0%) | 16 (27.1%) |
| Diverticulum intestinal | 0 | 0 | 1 (2.6%) | 0 | 0 | 0 |
| haemorragic | | | | | | |
| Dyspepsia | 3 (12.0%) | 2 (7.4%) | 4 (10.3%) | 1 (3.3%) | 4 (8.0%) | 4 (6.8%) |
| Dysphagia | 0 | 1 (3.7%) | 0 | 0 | 0 | 1 (1.7%) |
| Epigastric discomfort | 1 (4.0%) | 1 (3.7%) | 0 | 0 | 0 | 0 |
| Flatulence | 0 | 1 (3.7%) | 1 (2.6%) | 3 (10.0%) | 3 (6.0%) | 1 (1.7%) |
| Gastritis | 0 | 1 (3.7%) | 0 | 0 | 0 | 0 |
| GERD | 2 (8.0%) | 4 (14.8%) | 2 (5.1%) | 2 (6.7%) | 0 | 1 (1.7%) |
| Haematochezia | 1 (4.0%) | 0 | 0 | 0 | 0 | 0 |
| Haemorroids | 1 (4.0%) | 0 | 0 | 2 (6.7%) | 1 (2.0%) | 0 |
| Large intestinal ulcer | 1 (4.0%) | 0 | 0 | 0 | 0 | 0 |
| Nausea | 6 (24.0%) | 5 (18.5%) | 11 (28.2%) | 11 (36.7%) | 17 (34.0%) | 12 (20.3%) |
| Odynophagia | 0 | 0 | 0 | 1 (3.3%) | 0 | 2 (3.4%) |
| Oesophagitis | 1 (4.0%) | 0 | 0 | 0 | 0 | 0 |
| Retching | 0 | 1 (3.7%) | 0 | 0 | 2 (4.0%) | 0 |
| Vomiting | 2 (8.0%) | 6 (22.2%) | 6 (15.4%) | 8 (26.7%) | 22 (44.0%) | 11 (18.6%) |

The most frequent gastrointestinal TEAEs reported in more than 10% of the subjects randomized to HDP-CDV and exceeding twice the reporting frequency in the placebo rate were: abdominal pain (Cohorts 1, 4 and 4A), diarrhea (Cohort 4), GERD (Cohort 2), and vomiting (Cohort 4A). A clear relationship between HDP-CDV dose and overall incidence of such AEs was not generally apparent; an increased frequency of diarrhea and abdominal pain was noted in subjects who received HDP-CDV BIW versus QW and placebo.

TABLE 38 displays the intensity of gastrointestinal AEs reported by Cohorts.

TABLE 38
Intensity of Gastrointestinal AEs by Cohort

| | System Organ Class | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Preferred Term | N = 25 | N = 27 | N = 39 | N = 30 | N = 50 | N = 59 |
| Grade 1 | 9 (36.0%) | 6 (22.2%) | 12 (30.8%) | 3 (10.0%) | 12 (24.0%) | 16 (27.1%) |
| Grade 2 | 6 (24.0%) | 10 (37.0%) | 10 (25.6%) | 13 (43.3%) | 17 (34.0%) | 16 (27.1%) |
| Grade 3 | 2 (8.0%) | 1 (3.7%) | 2 (5.1%) | 10 (33.3%) | 10 (20.0%) | 7 (11.9%) |
| Grade 4 | 0 | 0 | 2 (5.1%) | 0 | 1 (2.0%) | 0 |
| Grade 5 | 0 | 0 | 0 | 1 (3.3%) | 0 | 0 |

Overall, the intensity of gastrointestinal AEs was similar between subjects who received HDP-CDV QW and subjects who received placebo. Subjects who received HDP-CDV 200 mg BIW had higher intensity GI AEs than subjects who received placebo and subjects who received HDP-CDV at lower doses. The increased incidence of severe GI AEs in Cohort 4A versus subjects receiving placebo was driven by an excess of subjects with severe diarrhea (13% excess as compared to placebo).

TABLE 39 displays the gastrointestinal AEs that led to study drug discontinuation in Study HDP-CDV-201 by dosing Cohorts.

TABLE 39
Gastrointestinal AEs Leading to Study Drug Discontinuation

| | System Organ Class | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Preferred Term | N = 25 | N = 27 | N = 39 | N = 30 | N = 50 | N = 59 |
| Abdominal pain | 0 | 0 | 0 | 1 (3.3%) | 1 (2.0%) | 0 |
| Diarrhoea | 0 | 0 | 1 (2.6%) | 7 (23.3%) | 2 (4.0%) | 0 |
| Dyspepsia | 0 | 0 | 2 (5.1%) | 0 | 0 | 0 |
| Nausea | 0 | 0 | 0 | 1 (3.3%) | 1 (2.0%) | 2 (3.4%) |
| Vomiting | 0 | 1 (3.7%) | 0 | 0 | 1 (2.0%) | 0 |
| Total subjects | 0 | 1 (3.7%) | 3 (7.7%) | 9 (30.0%) | 5 (10.0%) | 2 (3.4%) |
| discontinued | | | | | | |

With the exception of subjects randomized to receive HDP-CDV 200 mg BIW in Cohort 4, less than 10% of the subjects randomized to HDP-CDV discontinued study drug due to GI AEs at any other dose and the proportion of subjects discontinuing placebo for similar reason was not apparently different for QW dosing regimens. The contrast between the frequency of GI AEs and the low rate of discontinuation for GI AEs suggest that these AEs either had another etiology that was successfully treated or that such AEs were adequately managed allowing continuation of HDP-CDV.

Based on the safety signal identified in Cohort 4, a program-wide Safety Monitoring and Management Plan (SMMP) was generated that included interruption of study drug for subjects experiencing Grade 3 or higher gastrointestinal AEs. The implementation of this plan appears to have been successful in allowing subjects to continue on study medications. Sixteen of 17 subjects interrupted dosing with HDP-CDV in Cohort 4A due to gastrointestinal symptoms (one subject interrupted due to bacteremia); 13 of these resumed therapy. Two (11.8%) subjects (4082 and 4078) had detectable viremia (100 copies/mL) after 19 and 21 days of HDP-CDV interruption; both resumed study drug and had CMV DNA levels of less than 200 copies/mL at the end of treatment. Therefore, based upon these data, dosing interruption appears to be an appropriate strategy to mitigate the consequences of gastrointestinal AEs during HDP-CDV dosing, without significant loss of antiviral activity and allowing completion of the intended therapy duration.

### Analyses of Gastrointestinal Events

Data analyzed by Cohort indicate that QW doses of HDP-CDV appeared sufficiently well tolerated up to 200 mg QW and suitable for further evaluation as CMV prophylaxis in high risk subjects post-HSCT. With BIW dosing, 100 mg BIW and 200 mg BIW were associated with increasing gastrointestinal adverse effects, predominantly diarrhea, which was at times difficult for clinicians to distinguish from gut aGVHD. Although less frequent, vomiting and hepatobiliary events also were noted as potentially dose related adverse drug effects at these higher dosing regimens.

In general, the 200 mg QW dose appeared to have been better tolerated than 100 mg BIW, when frequency and severity of GI AEs were considered. With respect to QW dosing comparisons, overall, there appeared to be slightly more gastrointestinal and hepatobiliary events reported in subjects who received 200 mg QW versus subjects who received 100 mg QW. Based upon anecdotal reports by Investigators that subjects weighing less might have experienced more gastrointestinal AEs, the potential correlation between tolerability and subject size was evaluated.

A common metric for unit based dosing of medications is by weight (mg/kg). When the distribution of weights was examined for all subjects participating in the study, the median was 78 kg and the lower 25^{th} percentile weight was approximately 65 kg, which translates to a cut-off of ˜3 mg/kg for a 200 mg total weekly dose. Based upon this approach, a cut-off of 3 mg/kg was used to assess the frequency of diarrhea and associated GI AEs reported for the subjects in Cohorts 3 or 4a (TABLE 40).

TABLE 40
Summary of Common GI-Related Treatment Emergent Adverse Events for Cohort 3 and 4a, Overall and Based on 3 mg/kg Dose
TEAEs based on System Organ Class and Grade Reported for ≧15% in any Cohort OR ≧10% of Subjects and ≧2x
More Frequently in Any HDP-CDV Group Compared to the Placebo Group) and Having at Least 1 Grade 3 Event in any
Treatment Group; Comparisons for Cohorts 3 & 4a and Grade 2-5

| | System Organ Class | | | | | | |
|---|---|---|---|---|---|---|---|
| | | Cohort 3 | Cohort 3 | Cohort 4A | Cohort 4A | | |
| | Cohort 3 | HDP-CDV | HDP-CDV | HDP-CDV | HDP-CDV | Cohort 4A | |
| | HDP-CDV | 200 QW ≦3 | 200 QW >3 | 100 mg BIW ≦3 | 100 mg BIW >3 | HDP-CDV | Pooled |
| | 200 mg QW | mg/kg | mg/kg | mg/kg | mg/k | 100 mg BIW | Placebo |
| Preferred Term | N = 39 | N = 25 | N = 14 | N = 40 | N = 10 | N = 50 | N = 59 |
| Abdominal pain | 5 (12.8%) | 3 (12%) | 2 (14.3%) | 9 (22.5%) | 4 (40%) | 13 (26.0%) | 4 (6.8%) |
| Overall Frequency | | | | | | | |
| Grade 2 | 1 (2.6%) | 1 (4.0%) | 0 | 1 (2.5%) | 0 | 1 (2.0%) | 1 (1.7%) |
| Grade 3 | 1 (2.6%) | 0 | 1 (7.1%) | 1 (2.5%) | 0 | 1 (2.0%) | 2 (3.4%) |
| Grade 4 | 0 | 0 | 0 | 0 | 0 | 0 | 0 |
| Grade 5 | 0 | 0 | 0 | 0 | 0 | 0 | 0 |
| Diarrhea | 13 (33.3%) | 5 (20%) | 8 (57.1%) | 20 (50%) | 6 (60%) | 26 (52.0%) | 16 (27.1%) |
| Overall Frequency | | | | | | | |
| Grade 2 | 5 (12.8%) | 1 (4.0%) | 4 (28.6%) | 3 (7.5%) | 3 (30%) | 6 (12.0%) | 5 (8.5%) |
| Grade 3 | 2 (5.1%) | 1 (4.0%) | 1 (7.1%) | 6 (15%) | 3 (30%) | 9 (18.0%) | 3 (5.1%) |
| Grade 4 | 0 | 0 | 0 | 0 | 0 | 0 | 0 |
| Grade 5 | 0 | 0 | 0 | 0 | 0 | 0 | 0 |
| Nausea Overall | 11 (28.2%) | 8 (32%) | 3 (21.4%) | 13 (32.5%) | 4 (40%) | 17 (34.0%) | 12 (20.3%) |
| Overall Frequency | | | | | | | |
| Grade 2 | 4 (10.3%) | 3 (12%) | 1 (7.1%) | 6 (15%) | 3 (30%) | 9 (18.0%) | 7 (11.9%) |
| Grade 3 | 1 (2.6%) | 1 (4.0%) | 0 | 1 (2.5%) | 1 (10%) | 2 (4.0%) | 1 (1.7%) |
| Grade 4 | 0 | 0 | 0 | 0 | 0 | 0 | 0 |
| Grade 5 | 0 | 0 | 0 | 0 | 0 | 0 | 0 |
| Vomiting | 6 (15.4%) | 4 (16%) | 2 (14.3%) | 16 (40%) | 6 (60%) | 22 (44.0%) | 11 (18.6%) |
| Overall Frequency | | | | | | | |
| Grade 2 | 1 (2.6%) | 1 (4.0%) | 0 | 4 (10%) | 4 (40.0%) | 8 (16.0%) | 3 (5.1%) |
| Grade 3 | 1 (2.6%) | 1 (4.0%) | 0 | 1 (2.5%) | 1 (10.0%) | 2 (4.0%) | 0 |
| Grade 4 | 0 | 0 | 0 | 0 | 0 | 0 | 0 |
| Grade 5 | 0 | 0 | 0 | 0 | 0 | 0 | 0 |
| Hypokalaemia | 5 (12.8%) | 1 (4.0%) | 4 (28.6%) | 5 (12.5%) | 3 (30%) | 8 (16.0%) | 4 (6.8%) |
| Grade 2 | 2 (5.1%) | 1 (4.0%) | 1 (7.1%) | 1 (2.5%) | 1 (10%) | 2 (4.0%) | 1 (1.7%) |
| Grade 3 | 0 | 0 | 0 | 0 | 2 (20.0%) | 2 (4.0%) | 1 (1.7%) |
| Grade 4 | 0 | 0 | 0 | 0 | 0 | 0 | 0 |
| Grade 5 | 0 | 0 | 0 | 0 | 0 | 0 | 0 |

While the data should be interpreted with caution based on the relatively small sample sizes and an incidence of diarrhea of 27% in the pooled placebo arm, this analysis suggests a trend with increasing incidence of gastrointestinal AEs with increasing weight-adjusted weekly dose of HDP-CDV. In particular, there appears to be a higher frequency and/or grade of diarrhea, abdominal pain, and vomiting in subjects for whom a 200 mg weekly dose exceeded 3 mg/kg based on their individual weights.

A similar approach was applied to evaluate increases in serum ALT values as shown in TABLE 41, which lists the proportion of subjects with various levels of ALT increase at Screening and anytime during HDP-CDV therapy at 200 mg per week, expressed as weight-adjusted doses.

TABLE 41
Summary of Abnormal Alanine Aminotransferase (ALT) Test
Results by Visit, Safety Population, Cohorts 3 and 4A,

| | | Cohort 3 | | | Cohort 4A | | |
|---|---|---|---|---|---|---|---|
| | | HDP-CDV (mg/kg/wk) | | | HDP-CDV (mg/kg/wk) | | |
| | Abnormal | Placebo | ≦3.0 | >3.0 | Placebo | ≦3.0 | >3.0 |
| Visit | Criteria | N = 14 | N = 25 | N = 14 | N = 17 | N = 40 | N = 10 |

| | | | | | | |
|---|---|---|---|---|---|---|
| Screening | | | | | | |
| n | 14 | 24 | 13 | 17 | 36 | 10 |
| >3-5x ULN | 1 (7.1%) | 0 | 0 | 0 | 1 (2.8%) | 0 |
| >5-10x ULN | 0 | 0 | 0 | 0 | 1 (2.8%) | 0 |
| >10-20x ULN | 0 | 0 | 0 | 0 | 1 (2.8%) | 0 |
| >20x ULN | 0 | 0 | 0 | 0 | 0 | 0 |
| Any Time Between Week 2 and Week 10 | | | | | | |
| n | 14 | 24 | 14 | 15 | 40 | 10 |
| >3-5x ULN | 2 (14.3%) | 3 (12.5%) | 1 (7.1%) | 1 (6.7%) | 6 (15.0%) | 4 (40.0%) |
| >5-10x ULN | 1 (7.1%) | 5 (20.8%) | 0 | 2 (13.3%) | 3 (7.5%) | 1 (10.0%) |
| >10-20x ULN | 0 | 0 | 0 | 1 (6.7%) | 1 (2.5%) | 0 |
| >20x ULN | 0 | 1 (4.2%) | 0 | 0 | 0 | 1 (10.0%) |

While there appeared to be a weight-based association with the frequency and severity of diarrhea AEs, there does not appear to be an association with the frequency or severity of ALT elevations, when the 200 mg weekly dose was expressed on a per weight basis, using the 3.0 mg/kg cut off.

### Gastrointestinal AEs Summary

Diarrhea has been identified as a dose limiting toxicity for HDP-CDV. An increased incidence of gastrointestinal AEs (particularly diarrhea and abdominal pain) was observed in subjects randomized to HDP-CDV 200 mg QW or to HDP-CDV administered BIW as compared to placebo. Following introduction of the safety monitoring and management plan during enrollment of Cohort 4, very few subjects (≦10%) discontinued study drug because of GI AEs. The introduction of dose interruption in managing GI AEs, particularly diarrhea, appears to have allowed study completion in a higher proportion of subjects (10 of 53 in Cohort 4 and 30 of 50 in Cohort 4a). In addition, there is some evidence that GI AEs may be managed by using weight based cut-offs. For this reason, dosing in future studies may incorporate a dose adjustment for subjects weighing<65 kg at entry.

### Graft Versus Host Disease

Occurrences of graft versus host disease and in particular the overlap between GVHD of the intestine and gastrointestinal AEs were investigated. Here, presented are: first the incidence of GVHD reported as an AE in the electronic Case Report Forms (eCRF); second displays of the data from the weekly assessment of stage (by organ) and grade of GVHD that was entered by the site for subjects with suspected GVHD during the Study.

Because of the increased frequency of reports of acute GVHD in the BIW Cohorts of Study HDP-CDV-201, an in-depth analysis of reported GVHD was conducted. This analysis includes data from the following sources: Adverse events as reported in the eCRF, including GVHD and GI events; Weekly GVHD assessments (by stage, organ, and grade) as reported by the investigators on the GVHD module of the eCRF. The analyses include the following: Examination of the incidence of GVHD AEs and the overlap of GVHD of the intestine and GI AEs; Assessment of the impact of the incident identification of diarrhea as an event associated with the use of HDP-CDV; Assessment of the impact of implementation of the Safety Monitoring and Management Plan for GI events; A logistic regression analysis of the frequency of gastrointestinal events, GVHD and bilirubin increases (used to Stage GVHD of the liver) versus HDP-CDV dose.

During the conduct of Cohort 4, at a dose of 200 mg BIW, an increased rate of profuse watery diarrhea was observed among study subjects; diarrhea was identified as the dose-limiting toxicity in this patient population. In subjects reported to have GVHD, it was anecdotally observed that diarrhea was out of proportion to the severity of the observed histopathology. The dose of study drug received by subjects continuing in Cohort 4 was reduced to 200 mg QW and a Safety Monitoring and Management Plan (SMMP, which included options of drug interruption or dose reduction), was introduced for gastrointestinal adverse events.

Interpretation of data on GVHD is, therefore, complex for the following reasons: the dose-escalation study design; the way data reporting and collection in the eCRF evolved over the course of the study in response to new information; and the introduction of the SMMP during the conduct of Cohort 4. In particular, the SMMP directed the Investigators to evaluate diarrhea “by measurement of stool volumes per 24 hours, and evaluation for pathogens, GVHD, and other causes,” which led to a possible reporting bias.

### GVHD Reported as an AE

Serious adverse events were reported preferentially as diagnoses rather than signs or symptoms. Therefore, when subjects developed serious diarrhea, the investigator was instructed to assign a cause. GVHD is primarily a clinical diagnosis, and diarrhea in the post-HSCT setting is often considered to be GVHD unless another cause is apparent. Even with a negative GI biopsy, patients are often treated empirically because intestinal involvement can be spotty, and missed due to sampling of unaffected areas.

Adverse events, both serious and nonserious, reported as GVHD are presented in TABLE 42. For nonserious AEs, no documentation in the clinical database is available to assess the diagnostic procedures that led to an event being characterized as GVHD. Therefore, the empirical diagnosis of GVHD cannot be confirmed or refuted for nonserious events. Also, investigators were not consistent in reporting GVHD as GVHD specific (e.g., “acute GVHD of the intestine”) or broad (“GVHD”) and may have reported multiple organ-specific events (e.g., GVHD of skin and GVHD of gut) for a given subject. To facilitate interpretation of the data, and prior to the start of Cohort 4 enrollment, sites were instructed to enter only one event of acute GVHD on the AE page, regardless of organ involvement and to provide detail regarding the organ(s) involved on the GVHD page of the eCRF.

TABLE 42
Graft versus Host Disease Reported as AEs^{a}

| | System Organ Class | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Preferred Term | N = 25 | N = 27 | N = 39 | N = 30 | N = 50 | N = 59 |
| Acute GVHD | 0 | 0 | 4 (10.3%) | 24 (80.0%) | 32 (64.0%) | 8 (13.6%) |
| Acute GVHD in intestine | 2 (8.0%) | 5 (18.5%) | 6 (15.4%) | 0 | 0 | 3 (5.1%) |
| Acute GVHD in liver | 0 | 0 | 2 (5.1%) | 0 | 0 | 0 |
| Acute GVHD in skin | 4 (16.0%) | 3 (11.1%) | 7 (17.9%) | 0 | 0 | 5 (8.5%) |
| Chronic GVHD | 0 | 0 | 1 (2.6%) | 1 (3.3%) | 2 (4.0%) | 0 |
| GVHD | 2 (8.0%) | 2 (7.4%) | 1 (2.6%) | 0 | 1 (2.0%) | 2 (3.4%) |

^{a}Reporting conventions were changed during Cohort 3 at which time GVHD was described as acute vs. chronic rather than organ-specific

Overall, the data show an increased frequency of reporting of acute GVHD with increasing doses of HDP-CDV, particularly when HDP-CDV was administered BIW.

### GVHD Leading to Treatment Discontinuation

Few subjects were discontinued from Study HDP-CDV-201 for AEs of GVHD. TABLE 43 displays the proportion of subjects by Cohorts who discontinued therapy due to GVHD.

TABLE 43
Events of GVHD Leading to Study Drug Discontinuation

| | System Organ Class | | | | | |
|---|---|---|---|---|---|---|
| | Cohort 1 | Cohort 2 | Cohort 3 | Cohort 4 | Cohort 4A | |
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Preferred Term | N = 25 | N = 27 | N = 39 | N = 30 | N = 50 | N = 59 |
| Acute graft versus | 0 | 0 | 0 | 0 | 3 (6.0%) | 0 |
| host disease | | | | | | |
| Acute GVHD in intestine | 0 | 0 | 0 | 0 | 0 | 1 (1.7%) |
| Acute GVHD in skin | 0 | 0 | 0 | 0 | 0 | 1 (1.7%) |

Five (5) subjects discontinued study drug because of AEs categorized as GVHD. Three (3) events, categorized as acute GVHD, led to discontinuation of subjects in Cohort 4a, following the implementation of the SMMP. Two (2) subjects who received placebo discontinued study treatment, 1 each for acute GVHD of the intestine and acute GVHD of the skin, respectively. Overall, very few subjects discontinued study drug due to events identified as GVHD, in part because investigators reported diarrhea as the reason for discontinuation. This disparity suggests that the investigators thought that the diarrhea intensity was disproportionate to the GVHD syndrome in these subjects.

### Weekly Assessment of GVHD Stage and Grade

For all subjects, the sites were instructed to record the presence or absence of GVHD, and stage and grade of GVHD, if present, at each visit on a separate eCRF page. This data is considered to be more objective and standardized than the reporting of GVHD as an AE. Stage and grade of GVHD were evaluated per the NIH consensus guidelines (Przepiorka D, et al. Consensus conference on acute GVHD grading. Bone Marrow Transplant. 1995; 15(6): 825-828), which focus on the intensity of symptoms or laboratory abnormalities associated with GVHD (for example, the staging of GVHD of the intestine is based on the volume of diarrhea). TABLE 44 shows the stage and grade of GVHD overall and by organ compared to the incidence of AEs of diarrhea by Cohort.

TABLE 44
Stage and Grade of GVHD compared to AEs of Diarrhea by Grade (maximum
intensity reported by treatment group in Cohorts 1 through 4A)

| | | GVHD Stage | | | | GVHD Grade | |
|---|---|---|---|---|---|---|---|
| | Overall GVHD | Skin | Intestine | Liver | Subjects Reporting | | Mean |
| Dose Group | Incidence | 1/2/3/4 | 1/2/3/4 | 1/2/3/4 | Diarrhea as an AE | 1/2/3/4 | (Median) |
| Pooled | 21/59 (35.6%) | 7/6/2/2 | 6/2/1/0 | 0/0/0/0 | 16 (27.1%) | 11/6/3/1 | 1.7 (1) |
| placebo | | | | | | | |
| n = 59 | | | | | | | |
| HDP-CDV | 11/25 (44%) | 3/4/1/0 | 4/1/0/0 | 0/1/0/0 | 3 (12.0%) | 7/4/0/0 | 1.4 (1) |
| 40 mg QW | | | | | | | |
| n = 25 | | | | | | | |
| HDP-CDV | 16/27 (59.3%) | 8/5/0/0 | 6/1/1/0 | 1/0/0/0 | 8 (29.6%) | 7/8/1/0 | 1.6 (2) |
| 100 mg QW | | | | | | | |
| n = 27 | | | | | | | |
| HDP-CDV | 18/39 (46.2%) | 10/2/2/0 | 7/4/2/0 | 0/1/0/0 | 13 (33.3%) | 10/5/3/0 | 1.6 (1) |
| 200 mg QW | | | | | | | |
| n = 39 | | | | | | | |
| HDP-CDV | 31/50 (62%) | 5/4/4/0 | 11/10/5/2 | 3/1/1/0 | 26 (52.0%) | 4/14/12/1 | 2.3 (2) |
| 100 mg BIW | | | | | | | |
| n = 50 | | | | | | | |
| HDP-CDV | 27/30 (90%) | 6/3/4/0 | 4/8/9/3 | 5/1/2/0 | 21 (70.0%) | 5/5/15/2 | 2.5 (3) |
| 200 mg BIW | | | | | | | |
| n = 30 | | | | | | | |

The overall frequency of GVHD ranged from 44 to 59% in the QW dosing groups, compared to the pooled placebo rate of 35.6%; the frequency of GVHD in the HDP-CDV 100 mg BIW Cohort was 62%, driven by GVHD of the intestine. An increased frequency (90%) and severity of events diagnosed as GVHD was apparent in subjects who received 200 mg HDP-CDV BIW. This increased frequency and severity was entirely driven by events diagnosed as GVHD of the intestine.

Unlike events in QW Cohorts (1-3, 40 mg to 200 mg), the frequency of GVHD of the intestine was very similar to the frequency of diarrhea in the BIW Cohorts (4 and 4a, 200 and 100 mg BIW): 80% incidence of GVHD of the intestine versus 70% incidence diarrhea and 56% incidence of GVHD of the intestine versus 52% incidence of diarrhea Cohorts 4 (HDP-CDV 200 mg BIW) and 4A (100 mg BIW), respectively. This finding suggests an overlap between diarrhea associated with the use of HDP-CDV and reporting of diarrhea as GVHD of the intestine.

In subjects who received HDP-CDV QW at doses up to 200 mg, there was no apparent increase in the frequency or severity of GVHD as compared to placebo and no apparent correlation of frequency or intensity of GVHD with HDP-CDV dose.

### Analyses of GVHD by Organ System Involvement

The incidence, maximum grade or stage, and organ involved in subjects enrolled are summarized in TABLE 45.

TABLE 45
Analysis of GVHD by Organ and Severity

| | Cohort 1 | Cohort 2 | Cohort 3 | Cohort 4 | Cohort 4A | |
|---|---|---|---|---|---|---|
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| Dose group | N = 25 | N = 27 | N = 39 | N = 30 | N = 50 | N = 59 |
| No GVHD, (%) | 14 (56.0%) | 10 (37.0%) | 20 (51.3%) | 3 (10.0%) | 18 (36.0%) | 38 (64.4%) |
| Acute GVHD, any grade | 11 (44.0%) | 17 (63.0%) | 19 (48.7%) | 27 (90.0%) | 32 (64.0%) | 21 (35.6%) |
| Highest grade of GVHD | | | | | | |
| during study, through | | | | | | |
| posttreatment Week 1 | | | | | | |
| Grade 1 | 5 (20.0%) | 6 (22.2%) | 6 (15.4%) | 2 (6.7%) | 3 (6.0%) | 9 (15.3%) |
| Grade 2 | 5 (20.0%) | 10 (37.0%) | 9 (23.1%) | 8 (26.7%) | 12 (24.0%) | 8 (13.6%) |
| Grade 3 | 0 | 1 (3.7%) | 4 (10.3%) | 15 (50.0%) | 16 (32.0%) | 3 (5.1%) |
| Grade 4 | 1 (4.0%) | 0 | 0 | 2 (6.7%) | 1 (2.0%) | 1 (1.7%) |
| Acute GVHD and GI | | | | | | |
| involvement, highest | | | | | | |
| stage | | | | | | |
| Any stage | 5 (20.0%) | 10 (37.0%) | 13 (33.3%) | 24 (80.0%) | 28 (56.0%) | 9 (15.3%) |
| Stage ≧2 | 1 (4.0%) | 2 (7.4%) | 7 (17.9%) | 20 (66.7%) | 18 (36.0%) | 4 (6.8%) |
| Acute GVHD and skin | | | | | | |
| involvement, highest | | | | | | |
| stage | | | | | | |
| Any stage | 8 (32.0%) | 13 (48.2%) | 15 (38.5%) | 14 (46.7%) | 14 (28.0%) | 17 (28.8%) |
| Stage ≧2 | 5 (20.0%) | 7 (25.9%) | 5 (12.8%) | 8 (26.7%) | 8 (16.0%) | 10 (16.9%) |
| Acute GVHD and liver | | | | | | |
| involvement, highest | | | | | | |
| stage | | | | | | |
| Any stage | 1 (4.0%) | 1 (3.7%) | 1 (2.6%) | 8 (26.7%) | 6 (12.0%) | 0 |
| Stage ≧2 | 1 (4.0%) | 0 | 1 (2.6%) | 3 (10.0%) | 1 (2.0%) | 0 |

These data demonstrated that there is no increase in incidence or severity of GVHD of the skin related to the administration of HDP-CDV and no dose proportionality in the incidence or severity of GVHD of the skin. These results support that HDP-CDV is not associated with the triggering or exacerbation of systemic GVHD.

### Logistic Regression Comparing Frequency of GI AEs and GVHD Versus HDP-CDV Dose

To exhaustively explore the relationship between HDP-CDV, dose and GVHD, a logistic regression was conducted, comparing the frequency of gastrointestinal events (diarrhea, abdominal pain and vomiting of graded severity); the frequency of GVHD of the skin and intestine; and the frequency of bilirubin increases (as a surrogate for the stage of GVHD of the liver), to HDP-CDV dose. The results are presented as odds ratio of occurrence associated with HDP-CDV compared to pooled placebo. The results of the logistic regression are presented in TABLE 46.

TABLE 46
Logistic Regression of GVHD or GI Symptoms^{a} versus HDP-CDV Dose
Cohorts

| | Overall | | | |
|---|---|---|---|---|
| | Treatment | | | |
| Endpoint | p-value | Dose | Odds Ratio (95% CI) | |
| Diarrhea Grade 2 | <0.0001 | 40 mg QW | 0.554 | (0.109, 2.817) |
| or higher | | 100 mg QW | 0.797 | (0.194, 3.273) |
| | | 200 mg QW | 1.395 | (0.461, 4.217) |
| | | 100 mg BIW | 3.000 | (1.157, 7.782) |
| | | 200 mg BIW | 11.011 | (3.845, 31.533) |
| Diarrhea Grade 3 | 0.0111 | 40 mg QW | <0.001 | (<0.001, >999) |
| or higher | | 100 mg QW | <0.001 | (<0.001, >999) |
| | | 200 mg QW | 0.743 | (0.129, 4.268) |
| | | 100 mg BIW | 3.018 | (0.869, 10.485) |
| | | 200 mg BIW | 7.961 | (2.263, 27.998) |
| Abdominal Pain | 0.1305 | 40 mg QW | 1.196 | (0.205, 6.990) |
| Grade 2 or higher | | 100 mg QW | 1.100 | (0.189, 6.406) |
| | | 200 mg QW | 1.146 | (0.242, 5.425) |
| | | 100 mg BIW | 0.573 | (0.100, 3.267) |
| | | 200 mg BIW | 4.185 | (1.116, 15.687) |
| Abdominal Pain | 0.9965 | 40 mg QW | 1.188 | (0.103, 13.725) |
| Grade 3 or higher | | 100 mg QW | 1.096 | (0.095, 12.637) |
| | | 200 mg QW | 0.750 | (0.066, 8.564) |
| | | 100 mg BIW | 0.582 | (0.051, 6.611) |
| | | 200 mg BIW | <0.001 | (<0.001, >999) |
| Vomiting Grade 2 | 0.2835 | 40 mg QW | <0.001 | (<0.001, >999) |
| or higher | | 100 mg QW | 1.493 | (0.235, 9.500) |
| | | 200 mg QW | 1.009 | (0.161, 6.333) |
| | | 100 mg BIW | 4.098 | (1.044, 16.082) |
| | | 200 mg BIW | 2.872 | (0.599, 13.770) |
| GVHD Stage 2 or | <0.0001 | 40 mg QW | 0.573 | (0.061, 5.399) |
| higher (intestine) | | 100 mg QW | 1.100 | (0.189, 6.406) |
| | | 200 mg QW | 2.500 | (0.657, 9.516) |
| | | 100 mg BIW | 7.734 | (2.406, 24.864) |
| | | 200 mg BIW | 27.500 | (7.743, 97.674) |
| GVHD Grade 3 | 0.0002 | 40 mg QW | 2.417 | (0.145, 40.234) |
| or higher | | 100 mg QW | 2.231 | (0.134, 37.058) |
| (intestine) | | 200 mg QW | 3.135 | (0.274, 35.812) |
| | | 100 mg BIW | 9.442 | (1.120, 79.618) |
| | | 200 mg BIW | 38.667 | (4.700, 318.110) |
| GVHD G2 or | 0.7634 | 40 mg QW | 1.058 | (0.293, 3.819) |
| higher (Skin) | | 100 mg QW | 0.694 | (0.172, 2.800) |
| | | 200 mg QW | 0.635 | (0.181, 2.226) |
| | | 100 mg BIW | 1.058 | (0.375, 2.985) |
| | | 200 mg BIW | 1.691 | (0.560, 5.101) |
| GVHD G3 or | 0.8927 | 40 mg QW | <0.001 | (<0.001, >999) |
| higher (Skin) | | 100 mg QW | <0.001 | (<0.001, >999) |
| | | 200 mg QW | 0.743 | (0.129, 4.268) |
| | | 100 mg BIW | 1.196 | (0.283, 5.047) |
| | | 200 mg BIW | 2.115 | (0.490, 9.130) |
| Bilirubin >2 ULN | 0.2995 | 40 mg QW | 0.778 | (0.077, 7.861) |
| | | 100 mg QW | 0.718 | (0.071, 7.237) |
| | | 200 mg QW | 1.556 | (0.297, 8.133) |
| | | 100 mg BIW | 3.039 | (0.742, 12.442) |
| | | 200 mg BIW | 3.734 | (0.827, 16.850) |

^{a}Gastrointestinal symptoms defined as diarrhea, abdominal pain, and vomiting

The results of this analysis show a strong correlation between diarrhea (but not other GI symptoms) and HDP-CDV dose, as well as a strong correlation between GVHD of the intestine and HDP-CDV dose, but no correlation between HDP-CDV and GVHD of the skin or increase in bilirubin. The correlations between HDP-CDV dose and GVHD of the intestine are driven by the BIW regimens of HDP-CDV, but are not observed (lower bound of the 95% CI of less than 1) with weekly doses of HDP-CDV up to 200 mg.

In order to confirm that the excess reporting of GVHD of the intestine in Cohort 4A was associated with attribution of diarrhea to GVHD, the data available for subjects who experienced an SAE of GVHD was assessed for the likelihood of GVHD in these subjects compared to other potential etiologies. The data suggested that the highest dose of HDP-CDV (400 mg per week, given as 200 mg BIW) was associated with a greater percentage of subjects (63%) reporting SAEs involving the gastrointestinal tract compared to subjects receiving lower doses.

The frequency of SAEs involving the gastrointestinal tract among subjects who received active drug in Cohort 4a (200 mg total dose of HDP-CDV per week as 100 mg BIW) was 42%; the frequency of stage 2-4 gut GVHD in this Cohort was not statistically different from historical controls, but was higher than among concurrent study patients receiving placebo (p=0.0415).

While there were some subjects who developed diarrhea while on study drug and who did not have clear evidence of gut GVHD, the majority of subjects who developed diarrhea had convincing clinical, radiographic, or histological evidence of GVHD. Patients with an intact and functioning ileum and right colon may compensate for a drug that causes diarrhea. In addition, patients whose gut cannot retrieve salt and water in the ileum and right colon because of mucosal injury (the situation with gut GVHD) may not compensate when a drug that causes diarrhea is given. A similar situation was observed when magnesium salts were given orally to replace urinary magnesium losses after transplant. When oral magnesium salts were given in the absence of gut GVHD, there may be little diarrhea; and when the drug was given in a patient with gut GVHD, the diarrhea may be profuse.

This analysis does not take into account potential confounding variables that can lead to increased diarrheal volumes, such as oral magnesium, use of mycophenolate mofetil, infection by non-culturable viruses (e.g., Rotavirus, Astrovirus, Norovirus), and dietary allowances (e.g., lactose, sucrose, maltose). Nor was the analysis adjusted for factors that may have led to worse gut GVHD (e.g., HLA mismatches, inadequate GVHD prophylaxis, and delayed treatment for GVHD). I get the sense from reading the narratives describing gastrointestinal SAEs that some centers delay treatment for GVHD for longer than others.”

The results of his analysis are presented in TABLE 47 and TABLE 48.

TABLE 47
Frequency of GI AEs by Study Cohort, their Cause(s), and Relatedness to Study Drug.

| | C1 Cohort | C2 Cohort | C3 Cohort | C4a Cohort | C4 Cohort | Pooled placebo |
|---|---|---|---|---|---|---|
| | (40 mg QW) | (100 mg QW) | (200 mg QW) | (100 mg BIW) | (200 mg BIW) | subjects |
| Number of patients | 25 | 27 | 39 | 50 | 30 | 59 |
| Number of patients | 3 (12%) | 3 (11%) | 9 (23%) | 21 (42%) | 19 (63%) | 4 (7%) |
| with SAE that | | | | | | |
| included diarrhea or | | | | | | |
| gut dysfunction (%) | | | | | | |
| Assessment of cause | | | | | | |
| of gut AE (n): | | | | | | |
| GVHD | 2 | 2 | 6 | 15 | 16 | 3 |
| GVHD + infection | 0 | 1 | 0 | 2 | 0 | 1 |
| Infection | 1 | 0 | 1 | 1 | 1 | 0 |
| Other | 0 | 0 | 2 | 0 | 1 | 0 |
| Unknown | 0 | 0 | 0 | 3 | 1 | 0 |
| Assignment of | | | | | | |
| relatedness to study | | | | | | |
| drug (n): | | | | | | |
| Not related | 3 | 3 | 9 | 16 | 7 | 4 |
| Probably not related | 0 | 0 | 0 | 1 | 1 | 0 |
| Possibly related | 0 | 0 | 0 | 3 | 7 | 0 |
| Probably related | 0 | 0 | 0 | 0 | 1 | 0 |
| Not enough data | 0 | 0 | 0 | 1 | 3 | 0 |
| GBM^{a} assignment-- | | | | | | |
| peak GVHD gastro- | | | | | | |
| intestinal stage (n): | | | | | | |
| Stage 1 | 0 | 0 | 2 | 9 | 2 | 2 |
| Stage 2 | 1 | 3 | 4 | 4 | 10 | 1 |
| Stage 3 | 1 | 0 | 0 | 2 | 4 | 1 |
| Stage 4 | 0 | 0 | 0 | 2 | 0 | 0 |

TABLE 48
Comparison of the Frequency of More Severe Intestinal GVHD Cohorts vs. Placebo Subjects v. Historical Allograft Controls

| | | | | | | | Historical |
|---|---|---|---|---|---|---|---|
| | | | | | | Pooled | allograft controls |
| | C1 Cohort | C2 Cohort | C3 Cohort | C4a Cohort | C4 Cohort | placebo | from FHCRC 2003- |
| | (40 mg QW) | (100 mg QW) | (200 mg QW) | (100 mg BIW) | (200 mg BIW) | subjects | 2007 (Gooley 2010) |
| Frequency of | 1/25 (4%) | 3/27 (11%) | 4/39 (10%) | 4/50 (8%) | 10/30 ^{a, b} (33%) | 1/59 (2%) | 46/1148 (4%) |
| Stage 2 | | | | | | | |
| gut GVHD | | | | | | | |
| Frequency of | 2/25 (8%) | 3/27 (11%) | 4/39 (10%) | 8/50 ^{c} (16%) | 14/30 ^{a, b} (33%) | 2/59 (3%) | 119/1148 (10%) |
| Stages 2-4 | | | | | | | |
| gut GVHD | | | | | | | |
| Frequency of | 1/25 (4%) | 0 | 0 | 4/50 (8%) | 4/30 ^{d} (13%) | 1/59 (2%) | 73/1148 (6%) |
| Stages 3-4 | | | | | | | |
| gut GVHD | | | | | | | |

^{a} Significantly different from historical controls, p < 0.0001 (Fishers Exact Test)
^{b} Significantly different from pooled placebo subjects, p < 0.0001 (Fishers Exact Test)
^{c} Significantly different from pooled placebo subjects, p = 0.0415 (Fishers Exact Test)

Overall, there is no apparent causal relationship between HDP-CDV administration and emergence of GVHD; however gastrointestinal symptoms, in particular diarrhea, occurring during treatment with HDP-CDV are frequently reported as GVHD and appear to have been managed as such.

### Corticosteroid Treatment

First line therapy for GVHD depends on the severity of the presenting symptoms. Dietary intake and concomitant medications are routinely assessed for contribution to low grade and/or intermittent diarrhea. Initial therapy of suspected GVHD, if mild, may consist of corticosteroids, for e.g., budesonide and beclomethasone oral therapy. For more severe symptoms, indicative of higher grade GVHD and/or a more definitive diagnosis, systemic steroids are typically administered. If the diagnosis is tentative, pulse steroids may be used. If the diagnosis is more certain, systemic steroids are administered for a longer, tapering course of therapy. The proportion of subjects who received therapy with systemic or oral steroids in is presented TABLE 49.

TABLE 49
Proportion of Subjects treated with Systemic or Oral Steroids by Cohort

| | Cohort 1 | Cohort 2 | Cohort 3 | Cohort 4 | Cohort 4A | |
|---|---|---|---|---|---|---|
| | HDP-CDV | HDP-CDV | HDP-CDV | HDP-CDV | HDP-CDV | Pooled |
| | 40 mg QW | 100 mg QW | 200 mg QW | 200 mg BIW | 100 mg BIW | Placebo |
| | N = 25 | N = 27 | N = 39 | N = 30 | N = 50 | N = 59 |
| Systemic Corticosteroids | 10 (40.0%) | 13 (48.1%) | 23 (59.0%) | 25 (83.3%) | 36 (72.0%) | 27 (45.8%) |
| Beclometasone Dipropionate | 0 | 2 (7.4%) | 3 (7.7%) | 5 (16.7%) | 5 (10.0%) | 3 (5.1%) |
| Budesonide | 2 (8.0%) | 3 (11.1%) | 9 (23.1%) | 12 (40.0%) | 15 (30.0%) | 6 (10.2%) |
| Dexamethasone | 2 (8.0%) | 1 (3.7%) | 0 | 1 (3.3%) | 2 (4.0%) | 2 (3.4%) |
| Dexamethasone Phosphate | 0 | 0 | 0 | 0 | 1 (2.0%) | 0 |
| Methylprednosolone | 1 (4.0%) | 2 (7.4%) | 5 (12.8%) | 14 (46.7%) | 16 (32.0%) | 7 (11.9%) |
| Methylprednosolone Acetate | 0 | 0 | 0 | 0 | 1 (2.0%) | 0 |
| Methylprednosolone Sodium | 1 (4.0%) | 0 | 0 | 6 (20.0%) | 4 (8.0%) | 7 (11.9%) |
| Succinate | | | | | | |
| Prednisone | 8 (32.0%) | 11 (40.7%) | 18 (46.2%) | 21 (70.0%) | 30 (60%) | 16 (27.1%) |

After initiation of study treatment 46% of the placebo subjects received systemic or oral corticosteroids, similar to subjects who received HDP-CDV 40 mg and 100 mg QW. Comparatively, 59%, 83% and 72% of the subjects who received HDP-CDV 200 mg QW, 200 mg BIW and 100 mg BIW, respectively, required treatment with steroids during the study. This increased use of corticosteroids likely contributed to the higher proportion of subjects that experienced hyperglycemia, hypokalemia, and/or insomnia in Cohorts 4 and 4A.

### Additional Analyses of GVHD

In order to further explore this association, the 200 mg QW and 100 mg BIW Cohorts were further analyzed, based upon a 3 mg/kg cut off. Similar to the analysis for diarrhea, GVHD reported as AEs was analyzed first (TABLE 50).

TABLE 50
Frequency and Severity of GVHD Reported as Adverse Events for Cohort 3 and 4a, Overall and Based on 3 mg/kg

| | System Organ Class | | | | | | |
|---|---|---|---|---|---|---|---|
| | | Cohort 3 | Cohort 3 | Cohort 4A | Cohort 4A | | |
| | Cohort 3 | HDP-CDV | HDP-CDV | HDP-CDV | HDP-CDV | Cohort 4A | |
| | HDP-CDV | 200 QW ≦3 | 200 QW >3 | 100 mg BIW ≦3 | 100 mg BIW >3 | HDP-CDV | Pooled |
| | 200 mg QW | mg/kg | mg/kg | mg/kg | mg/k | 100 mg BIW | Placebo |
| Preferred Term | N = 39 | N = 25 | N = 14 | N = 40 | N = 10 | N = 50 | N = 59 |
| Acute GVHD Overall | 13 (33.3%) | 8 (32%) | 5 (35.7%) | 25 (62.5%) | 7 (70%) | 32 (64.0%) | 15 (25.4%) |
| Overall Frequency | | | | | | | |
| Grade 2 | 4 (10.3%) | 2 (8.0%) | 2 (14.3%) | 8 (20%) | 3 (30%) | 11 (22%) | 5 (8.5%) |
| Grade 3 | 4 (10.3%) | 3 (12.0%) | 1 (7.1%) | 10 (25%) | 2 (20%) | 12 (24%) | 4 (6.8%) |
| Grade 4 | 2 (5.1%) | 1 (4.0%) | 1 (7.1%) | 3 (7.5%) | 2 (20%) | 5 (10%) | 2 (3.5%) |
| Grade 5 | 1 (2.6%) | 1 (4.0%) | 0 | 0 | 0 | 0 | 0 |

Based upon AE reporting, the frequency and severity of GVHD was higher in the 100 mg BIW group. Unlike diarrhea, there did not appear to be an increase in frequency or severity using the 3 mg/kg cut-off.

GVHD events were then analyzed using the data from the eCRF pages, which represented a more systematic cataloging of events (TABLE 51).

TABLE 51
Incidence, Severity and Organ Involvement of GVHD in Cohorts 3 and
4a displayed by Weight Base Dose Groups

| | | Cohort 3 | Cohort 4A | Cohort 4A | |
|---|---|---|---|---|---|
| | | HDP-CDV | HDP-CDV | HDP-CDV | |
| | Cohort 3 HDP-CDV | 200 mg | 100 mg | 100 mg | |
| | 200 mg QW | QW | BIW | BIW | Pooled |
| | ≦3 mg/kg | >3 mg/kg | ≦3 mg/kg | >3 mg/kg | Placebo |
| Dose group | N (%) | N (%) | N (%) | N (%) | N (%) |
| N | 25 | 14 | 40 | 10 | 59 |
| No GVHD, (%) | 15 (60%) | 6 (43%) | 16 (40%) | 3 (30%) | 38 (64%) |
| Acute GVHD, any grade | 10 (40%) | 8 (57%) | 24 (60%) | 7 (70%) | 21 (36%) |
| Highest grade of GVHD | | | | | |
| during study, through | | | | | |
| posttreatment Week 1 | | | | | |
| Grade 1 | 5 (20%) | 5 (36%) | 4 (10%) | 0 | 9 (15%) |
| Grade 2 | 4 (16%) | 1 (7%) | 10 (25%) | 4 (40%) | 8 (14%) |
| Grade 3 | 1 (4%) | 2 (14%) | 10 (25%) | 2 (20%) | 3 (5%) |
| Grade 4 | 0 | 0 | 0 | 1 (10%) | 1 (2%) |
| Acute GVHD and GI | | | | | |
| involvement, highest stage | | | | | |
| Any stage | 7 (28%) | 6 (43%) | 21 (53%) | 7 (70%) | 9 (15%) |
| Stage ≧2 | 3 (12%) | 3 (21%) | 14 (35%) | 3 (30%) | 4 (7%) |
| Acute GVHD and skin | | | | | |
| involvement, highest stage | | | | | |
| Any stage | 8 (32%) | 6 (43%) | 12 (30%) | 1 (10%) | 17 (29%) |
| Stage ≧2 | 3 (12%) | 1 (7%) | 8 (20%) | 0 | 10 (17%) |
| Acute GVHD and liver | | | | | |
| involvement, highest stage | | | | | |
| Any stage | 1 (4%) | 0 | 4 (10%) | 1 (10%) | 0 |
| Stage ≧2 | 1 (4%) | 0 | 1 (2.5%) | 1 (10%) | 0 |

Based upon overall GVHD frequency involving the intestine, there appears to be a modest weight-based association; perhaps because of small total group size for subjects receiving>3 mg/kg, an association with severity is not apparent.

Since diarrhea is part of the clinical definition for GVHD of the intestine, an additional analysis was performed to explore the possibility of a weigh-based association. In this assessment of subjects in Cohorts 3 or 4a, the proportion of subjects who had evidence of significant diarrhea and/or GVHD (i.e., either≧Grade 2 diarrhea OR Grade 2, 3 or 4 GVHD (per eCRF entry) OR both) was tallied for each Cohort, according to the dose each individual received on a per weight basis. This analysis is presented in TABLE 52.

TABLE 52
Incidence of Stage 2 and Higher GVHD of the Intestine AND/OR Grade 2 Diarrhea
by HDP-CDV dose Expressed as mg/kg, Cohorts 3 and 4A versus Pooled Placebo

| | System Organ Class | | | | | | |
|---|---|---|---|---|---|---|---|
| | | Cohort 3 | Cohort 3 | Cohort 4A | Cohort 4A | | |
| | Cohort 3 | HDP-CDV | HDP-CDV | HDP-CDV | HDP-CDV | Cohort 4A | |
| | HDP-CDV | 200 mg QW ≦3 | 200 QW >3 | 100 mg BIW ≦3 | 100 mg BIW >3 | HSP-CDV | Placebo |
| | 20 mg QW | mg/kg | mg/kg | mg/kg | mg/k | 100 mg BIW | Pooled |
| Preferred Term | N = 39 | N = 25 | N = 14 | N = 40 | N = 10 | N = 50 | N = 59 |
| Frequency gut GVHD | 6/39 | 3/25 | 3/14 | 7/40 | 3/10 | 10/50 | 2/59 |
| Stage 2, 3, 4 | 15% | 12% | 21% | 18% | 30% | 20% | 5% |
| Frequency Diarrhea | 7/39 | 2/25 | 5/14 | 9/40 | 6/10 | 15/50 | 8/59 |
| Grade ≧2 | 18% | 8% | 36% | 23% | 60% | 30% | 14% |
| # subjects with | 11/39 | 5/25 | 6/14 | 12/40 | 6/10 | 18/50 | 10/59 |
| Either GVHD ≧3 or | 28% | 25% | 43% | 30% | 60% | 36% | 17% |
| Diarrhea ≧2 or Both | | | | | | | |
| (not double-counted) | | | | | | | |

As shown in this table, when subjects who had either or both GVHD of the intestine (≧grade 3) and diarrhea (≧grade 2) are considered according to their administered weight-adjusted dose, those subjects who had received>3 mg/kg had a higher frequency of clinically significant GI events when compared to those subjects receiving<3 mg/kg within each Cohort. This analysis also supports the concept that reducing the dose for smaller subjects may be of benefit in decreasing the overall incidence of GI AEs.

### GVHD Summary

Data show an increase in AE reporting of GVHD for subjects who received HDP-CDV BIW. This increase in incidence of GVHD is associated with an increased reporting of GVHD of the intestine, both in terms of frequency and severity and is not associated with changes in the frequency and intensity of GVHD of the skin or increases in bilirubin in subjects treated with HDP-CDV versus subjects who received placebo. Taken together with the changes in event reporting and the use of an SMMP instituted during the course of this dose-escalation study, the data suggest that the increased gastrointestinal symptoms occurring during HDP-CDV therapy administered BIW resulted in an over classification of acute GVHD occurrences during treatment.

The increased frequency of clinical GVHD diagnoses in Study HDP-CDV-201 led to more subjects being presumptively treated with corticosteroids, which, in turn, had an impact on the type of metabolic side effects experienced by subjects receiving HDP-CDV.

While not as apparent as with the analysis of diarrhea, there appears to be a modest weight-based association with the overall GVHD frequency involving the intestine; perhaps because of small numbers in the >3 mg/kg subgroups, an association with severity is not seen.

In the proposed study HDP-CDV-301, detailed data on the diagnosis of GVHD is collected and a detailed guidance to the Investigator on the management of persistent diarrhea and/or presumptive GVHD is provided. While two HDP-CDV dosing regimens—QW and BIW—are assessed versus placebo, within each dosing regimen, subjects are assigned to a higher or lower total weekly dose based on weight (i.e., <60 kg versus 60-120 kg). Given this weight adjustment, dose administration to subjects in a fed state whenever possible, and the use of a SMMP, it is anticipated that the incidence of persistent diarrhea are comparable between study treatments (including placebo).

### Post-Hoc Safety Analysis

The current standard of care for the treatment of clinically significant CMV infection post-HSCT is the administration of preemptive antiviral therapy in high risk patients with CMV viremia. Preemptive therapy is typically effective, although drug-related toxicities, e.g., myelotoxicity and nephrotoxicity, limit the use of currently approved antivirals in a significant proportion of patients. Subjects who discontinued study drug for treatment of CMV infection or disease were followed for 4 weeks after initiation of alternate anti-CMV therapy. The results of this review are summarized here: 74 out of 230 subjects (32%) required therapy with antivirals with activity against CMV (ganciclovir, valganciclovir, foscarnet or cidofovir). Of these 74 subjects, 71 had follow-up data available. Following the switch to CMV preemptive therapy, 50 of the 71 subjects (70%) had decreases in neutrophil counts: 11 of 71 (15%) of subjects had a significant drop in neutrophil count (>2G/L decrease from the last value on blinded study medication), an additional 29 of 71 (41%) experienced severe or life-threatening neutropenia (<1 G/L) and another 10 of 71 (14%) had moderate neutropenia (<1.5 G/L). Eighteen (18) of 71 (25%) of subjects experienced an increase in creatinine level of more than 20% after initiation of anti-CMV therapy. Eleven (11) of 71 (15%) of subjects required G-CSF therapy, 5 of 71 (7%) required new RBC transfusions and 2 of 71 (3%) required new platelet transfusions. Ten (10) of 71 (14%) of subjects who initiated therapy with ganciclovir or valganciclovir had to be switched to a second line therapy (foscarnet or cidofovir) due to the toxicity of the initial regimen.

### Overall Safety Summary

Data provide further characterization of the safety and tolerability profile of HDP-CDV when administered for the prevention of CMV infection in high risk (R+) adult subjects post-HSCT. There is no indication of nephrotoxicity or myelotoxicity associated with HDP-CDV, regardless of dose and dosing frequency.

HDP-CDV doses of 40 and 100 mg QW had tolerability profiles similar to placebo in terms of AEs and laboratory abnormalities. In the limited number of subjects who received HDP-CDV at a dose of 200 mg QW in the fasted state, more subjects discontinued study medications than subjects who received 100 mg weekly or subjects on placebo (54% versus 33% and 46%, respectively); however, no clear safety signal was demonstrated for the discontinuations at this dose.

A dose-related increase in ALT was noted in association with HDP-CDV therapy. The ALT increases were typically Grade 2 or lower and 2-4 times baseline values, occurring between Weeks 2 and 4 of dosing, at doses of 200 mg per week or higher. Increases in ALT typically resolved after completion of HDP-CDV therapy and do not appear to be of toxicologic importance, based upon the preclinical and clinical safety profiles to date.

Few clinical hepatobiliary AEs were reported in association with treatment with HDP-CDV and most were mild or moderate in intensity. No case of drug induced liver injury (DILI) clearly attributable to HDP-CDV was noted during the course of the study. One event of liver injury considered by the investigator to be possibly associated with the administration of HDP-CDV at 200 mg BIW was reported; this event resolved after discontinuation of HDP-CDV. Therefore, these abnormalities appear to be manageable through careful monitoring and dosing reduction/interruption guidelines as detailed in the SMMP.

Diarrhea, frequently associated with other gastrointestinal symptoms, and often reported as GVHD, was dose limiting in this study and a dose of 200 mg BIW is not considered tolerable in this subject population. In contrast, with respect to diarrhea, QW HDP-CDV doses in the range studied (40 to 200 mg QW) were sufficiently well tolerated to support evaluation in future clinical trials. When comparing Cohorts 3 and 4a (each involving 200 mg total weekly dosing), the frequency and severity of diarrhea appeared to be somewhat related to weight, using a 3 mg/kg cut off.

Events of diarrhea (often reported as GVHD of the intestine) were more frequent and more severe, compared to placebo, in subjects who received HDP-CDV 100 mg BIW in the fasted state in Cohort 4a. However, these events infrequently led to permanent discontinuation of HDP-CDV, suggesting that a 100 mg BIW dose of HDP-CDV may be sufficiently well tolerated to include in future studies. In this regard, approximately one-third of the subjects in the 100 mg BIW Cohort (17 out of 50) interrupted HDP-CDV due to an adverse event; the majority were able to resume dosing. Therefore diarrhea appears to have been a manageable AE in this patient population.

An increased frequency and severity of apparent GVHD of the intestine, but not of the skin or the liver, was noted in subjects receiving HDP-CDV BIW. Analysis of these findings suggests that this increased reporting of GVHD was prompted by the occurrence of gastrointestinal symptoms commonly ascribed to a presumptive diagnosis of GVHD, but were in fact due to a HDP-CDV-related diarrheal event. This increase in frequency and severity was not noted in the HDP-CDV QW Cohorts, prior to the implementation of the Safety Monitoring and Management Plan, which may have introduced a reporting bias. In the CMV Prophylaxis Study HDP-CDV-301, once a week (QW) and BIW dosing of HDP-CDV are compared. Additional data may be collected and further guidance may be provided to the Investigators in order to better differentiate and manage diarrhea versus GVHD.

The safety profile of HDP-CDV 200 mg per week appears acceptable in the context of the benefit derived from the prevention of CMV reactivation as compared to the safety profile of preemptive therapy which is the current standard of care.

### Example 4

### HDP-CDV-106 Safety and PK of HDP-CDV in Subjects with Impaired Hepatic Function

Study HDP-CDV-106 was a Phase I, open-label, single-dose study that evaluated the effect of moderate to severe hepatic impairment on the safety, tolerability, and pharmacokinetics of HDP-CDV. Subjects with moderate hepatic impairment and matched healthy control subjects with normal hepatic function were enrolled and dosed first in Cohort 1, each receiving a single 200 mg dose of HDP-CDV under fasting conditions. The safety, tolerability and PK data from these subjects was reviewed prior to initiating dosing in subjects with severe hepatic impairment in Cohort 2. The study was conducted at four centers in the USA. Up to approximately 24 male and female subjects were planned to be enrolled in this study, including 8 subjects with moderate hepatic impairment and 8 matched healthy control subjects in Cohort 1 and 8 subjects with severe hepatic impairment in Cohort 2. The severity of hepatic impairment was assessed according to the Child-Pugh-Turcotte (CPT) score. Assessments are as follows: CPT Class B (moderate impairment) has a score of 7 to 9 points; and Class C (severe impairment) has a score of >9 points. For the purposes of this study, Cohort 2 subjects were required to have a CPT score of 10 or 11.

Administration of a single dose of HDP-CDV to subjects with moderate and severe hepatic impairment and to healthy control subjects with normal hepatic function was generally well-tolerated. Although a greater proportion of subjects with moderate or severe hepatic impairment reported AEs, the majority of AEs were mild in intensity, with no reports of severe or serious AEs that were considered related to HDP-CDV administration. Serious AEs were reported for 2 subjects with hepatic impairment during the study, one of whom later died. This subject had severe hepatic impairment, with a history of alcoholism and hepatitis C infection. The SAEs in this subject (esophageal varices hemorrhage, coma and hepatic cirrhosis) were assessed as unrelated to HDP-CDV administration by the investigator and the death occurred 14 days after a single dose of HDP-CDV. The remaining subject with moderate hepatic impairment was hospitalized 14 days after a single dose of HDP-CDV due to urinary tract infection and Escherichia bacteremia, both of which resolved following treatment and were considered unrelated to HDP-CDV administration. As expected, baseline values for all liver enzymes reflected the level of hepatic function for each group of subjects. Modest increases in AST and ALT were observed for subjects with moderate and severe hepatic impairment following administration of HDP-CDV (Day 4). However, these elevations were mild and transient, as AST and ALT levels were in decline by Day 14. Smaller increases in ALT and AST were observed for healthy subjects at Day 4. Unlike intravenously administered cidofovir, there was no evidence of changes in renal function following a single, orally administered dose of 200 mg HDP-CDV.

The overall mean concentration-time profiles for HDP-CDV and CMX021 (cidofovir, CDV) were similar between all study groups. Although mean HDP-CDV Cₘₐₓ values in the severe hepatic impairment group were lower when compared to both the healthy subjects and the moderate hepatic group, and the exposure (AUC_{inf}) to HDP-CDV was greatest in the severe hepatic impairment group, the difference from the healthy control subjects was not statistically significant based on ANOVA calculations. The peak concentrations and exposure (AUC_{inf}) to CDV were not statistically significantly different between healthy and hepatic impairment groups, and the metabolite to parent AUC_{inf} ratios of CDV to HDP-CDV were also consistent between study groups. Severe hepatic impairment resulted in statistically significantly longer Tₘₐₓ values for CDV compared to healthy subjects but not for HDP-CDV. Hepatic impairment did not affect the percentage of HDP-CDV bound to plasma protein. Overall, these data indicate that no HDP-CDV dose adjustment is warranted for subjects with moderate or severe hepatic disease and Study HDP-CDV-201 reflects this conclusion. The HDP-CDV-106 CSR was submitted in SN0260 dated 12APR2012.

### Effect of Renal Impairment on PK of HDP-CDV

Based on an analysis of EIND patients for whom both pre-dose creatinine clearance and single (i.e., first) dose AUCinf data were available, no adjustment of HDP-CDV dosage is required in patients with renal impairment, regardless of severity, with the exception of patients with end-stage renal disease (ESRD) who are not receiving dialysis treatment. ESRD patients not on dialysis have no ability to eliminate the metabolite CDV. As such, specific labeling for ESRD patients not currently on dialysis may not be sought. Furthermore, such subjects are/may be excluded from future studies with HDP-CDV. As noted above, population PK analysis of HDP-CDV and CDV may be conducted in ongoing controlled clinical trials in order to confirm these results that HDP-CDV dose adjustment is not needed in patients with renal impairment.

Study HDP-CDV-112:

Mass Balance and Metabolite Profiling Following ¹⁴C-Radiolabeled HDP-CDV Administration to Healthy Subjects.

Study HDP-CDV-112 was a Phase I, open-label, single-dose study that evaluated the mass balance, metabolite profiles and safety following administration of a single 200 mg dose of HDP-CDV containing ˜100 μCi of ¹⁴C-radiolabeled HDP-CDV to six healthy male subjects. Study drug administration on Day 1 was followed by a minimum 7-day sample collection period, i.e., through the morning of Day 8 (or 168 hours postdose), during which time, serial whole blood and plasma samples, cumulative urine voided, and all stools passed were collected at or over predetermined collection intervals. The overall duration of the sample collection period for some or all matrices was extended for up to an additional 7 days, i.e., up to 14 days total duration, to the morning of Day 15 (or 336 hours post-dose) using LSC radioanalysis to monitor the amount of ¹⁴C-radioactivity present in the whole blood, plasma, urine and stool samples.

Whole Blood and Plasma: After the 168-hour time point, additional blood samples for whole blood and plasma were collected at 24-hour intervals up to the morning of Day 15 OR until either of the following conditions was met: (1) assays indicated that the radioactivity levels in two consecutive samples had decreased to ≦twice the level of background radioactivity, or (2) both the urine and stool collections were discontinued (see below), whichever occurred first.

Urine and Stool:

After the 168-hour time point, all urine voided and all stools passed were collected over successive 24-hour collection intervals up to the morning of Day 15 OR until assays indicate that the radioactivity levels in samples from two consecutive collection intervals were ≦1% of the administered dose and the cumulative ¹⁴C-radioactivity recovered in urine and stool was ≧90% of the administered dose. The criteria for stopping urine and stool collection were assessed independently of each other.

Good recovery of radioactivity was achieved in all subjects (≧90%), with approximately 50% of the radioactivity excreted in urine and 40% excreted in feces. The major HDP-CDV-derived metabolites circulating in plasma were CMX103 (3-hydroxypropyl ester of CDV), CMX064 (4-(3-propoxy)butanoic acid ester of CDV) and CDV. Based on area under the plasma concentration versus time curve (AUCinf) using ng-equivalent concentrations of each metabolite, the AUC of CMX103 and CMX064 comprised approximately 32% and 23%, respectively, of total HDP-CDV-derived radioactivity AUC through 24 h postdose. Exposures to these metabolites have been previously characterized in animals, each having equal or greater exposure in at least one of the primary toxicology species compared to that expected after administration of a 100 mg to 200 mg dose of HDP-CDV. The in vitro pharmacological activity and cytotoxicity of these metabolites is low compared to HDP-CDV.

The major drug-derived metabolites found in urine in order of decreasing percent dose excreted were CMX103 (21%), CDV (10%), CMX064 (10%), and CMX104 (3-propoxy)acetic acid ester of CDV, 3%). The major drug-derived metabolites found in feces in order of decreasing percent dose excreted were CDV (32%) and CMX103 (6%). The final CSR is expected in 2Q2012. These data suggest that adequate toxicology coverage exist for HDP-CDV and its major metabolites in support of the HDP-CDV-301 protocol.

Single oral doses of 200 mg HDP-CDV were well-tolerated. There were no serious or severe adverse events (AEs) and no subject withdrew from the study due to an AE. Two AEs (oropharyngeal pain and dry skin) were reported by a single subject. Both events were assessed as mild in severity and unrelated to HDP-CDV administration.

Study HDP-CDV-108:

Effects of HDP-CDV on ECG Following a Therapeutic and Supratherapeutic Dose in Healthy Subjects.

Study HDP-CDV-108 is randomized, four-way crossover study designed to evaluate the effects of HDP-CDV administered as clinical and supratherapeutic doses on ECG parameters, as compared to placebo and moxifloxacin, in healthy subjects. Fifty-two (52) subjects (including approximately equal numbers of men and women) are randomized to receive each of the following four single-dose treatments according to a double Williams square schema: (1) placebo; (2) 400 mg moxifloxacin; (3) 200 mg HDP-CDV (“clinical dose”), and (4) 350 mg HDP-CDV (“supratherapeutic dose”) with a≧2-week washout interval between treatments. Administration of the placebo and both HDP-CDV doses are double-blind, the moxifloxacin is administered under open-label conditions. A dose of 350 mg HDP-CDV was selected as the supratherapeutic dose because this is the highest dose administered in HDP-CDV clinical trials. ECGs are obtained digitally using a continuous 12 lead digital recorder, starting approximately 1 hour prior to each treatment administration and continuing through approximately 23 hours postdose. Blood samples for PK analysis are obtained over the same period. The primary endpoint of the study is the time-matched change from baseline in QT interval corrected for heart rate (QTc), placebo-adjusted, based on Fridericia's correction (QTcF) method (i.e., delta-delta QTcF). Secondary endpoints include QTc with Bazett correction (QTcB); heart rate; PR interval; QRS interval; uncorrected QT interval; ECG morphology; and correlation between the QTcF change from baseline and plasma concentrations of HDP-CDV and CDV. The moxifloxacin may be used as a positive control to determine the “assay sensitivity” with an expected magnitude change from baseline (placebo-corrected) of 5 to 10 ms using a time-averaged analysis or 10 to 15 ms using a time-matched analysis.

Study HDP-CDV-113:

Effect of HDP-CDV on the Pharmacokinetics of Oral and Intravenous Midazolam a CYP3A4/5 Substrate in Healthy Subjects

HDP-CDV is a moderate inhibitor of CYP3A activity in vitro, indicating that a clinical drug interaction between HDP-CDV and other medications that are eliminated primarily by CYP3A is possible. Study HDP-CDV-113 is an open-label, randomized, two-period crossover study designed to evaluate the effect of HDP-CDV on the PK of the CYP3A substrate, midazolam (MDZ), following coadministration of single doses of HDP-CDV with single oral (PO) and intravenous (IV) doses of MDZ in healthy subjects. Twenty (20) eligible male and female subjects are randomized to receive two 1 mg doses of MDZ administered intravenously on 2 consecutive days and two 2.5 mg doses of MDZ administered orally on 2 consecutive days. The second PO dose of MDZ and the second IV dose of MDZ are each co-administered with single 200 mg doses of HDP-CDV. There may be a washout interval (dose-to-dose) of ≧14 days between the two HDP-CDV doses. The order in which each subject receives the IV and PO MDZ treatments is determined by a randomization schedule based on a Latin square. Blood samples for analysis of plasma concentrations of MDZ and its primary metabolite, 1′-hydroxy midazolam (1-OH MDZ) are collected through 24 hours after each MDZ dose. Comparison of the PK parameters for MDZ and 1-OH MDZ when administered in combination with HDP-CDV versus alone for each route of administration is used to estimate the potential for HDP-CDV to cause drug-drug interactions mediated through CYP3A inhibition and the contribution of HDP-CDV-mediated inhibition in the gut (vs. PO MDZ) and/or liver (vs. IV MDZ).

The HDP-CDV-301 eCRF may include provision for capturing trough levels of immunosuppressants (e.g., tacrolimus and cyclosporine A).

### Effect of HDP-CDV on the Pharmacokinetics of Digoxin a P-Gp Substrate

Based on in vitro results, HDP-CDV may inhibit P-gp, an important membrane transporter involved in ADME of digoxin, a P-gp substrate with a narrow therapeutic range. Due to this potential interaction, digoxin is excluded medication in Study HDP-CDV-301 (see Example 5).

### Effect of Food on HDP-CDV Pharmacokinetics and Antiviral Activity

In Study HDP-CDV-103, when HDP-CDV was given to subjects as a tablet following a high fat meal, peak plasma concentration (Cmax) was reduced by 48% and systemic exposure (AUC0-inf) was reduced by 28%. However, the clinical significance of these findings is currently unknown and anecdotal reports indicate that gastrointestinal symptoms during HDP-CDV therapy may be mitigated by administration of HDP-CDV with food. All subjects in HDP-CDV studies, therefore, were given a dose with food, where possible. In Study HDP-CDV-301, HDP-CDV is dosed with food; there is no restriction on the amount or type of food eaten. Therefore, evidence of the effectiveness of HDP-CDV is established in Study CMX00-301 in subjects who receive HDP-CDV in the fed state.

In addition, a pharmacokinetics sub-study is performed as part of Study HDP-CDV-301. As part of this sub-study, the fed state of HDP-CDV administration is captured in the eCRF and used to assess the effect of food on HDP-CDV PK parameters. Effectiveness if demonstrated in Study HDP-CDV-301, HDP-CDV administration may then be recommended to be in conjunction with food, if possible.

### Effect of CYP Inhibition or Induction on Pharmacokinetics of HDP-CDV

The need for a clinical study to investigate the effect of CYP inhibition or induction on the PK of HDP-CDV is currently being assessed in Studies HDP-CDV-112, HDP-CDV-NCA-051 and HDP-CDV-NCA-049. Based on non-clinical studies to date, HDP-CDV is likely eliminated by multiple CYP and non-CYP-mediated pathways, and therefore, has a low risk of being a victim of a DDI due to inhibition or induction of a single CYP pathway. However, additional clinical (human AME study HDP-CDV-112) and non-clinical (rat AME study HDP-CDV-NCA-051) study results are being evaluated in order to design a definitive CYP reaction phenotyping study in human liver microsomes. Taken together, the results from these studies are used to further quantify the risk for CYP-mediated DDI on HDP-CDV PK.

### Studies in Support of Clinical Pharmacology Plan

Study HDP-CDV-NCA-051:

AME of ¹⁴C-HDP-CDV Following Oral Administration to Bile Duct-Intact and Bile Duct-Cannulated Rats. In order to more fully define the CYP enzymes involved in HDP-CDV elimination, the disposition and metabolism of ¹⁴C-radiolabeled-HDP-CDV was studied in bile-duct cannulated rats either with or without pretreatment with the broad-spectrum CYP inhibitor aminobenzotriazole (ABT). This study has completed the in-life phase and sample analysis is ongoing.

Good recovery of radioactivity was observed in both treatment groups (≧90%), with approximately 30%, 57% and 8% of the administered radioactivity excreted in urine, feces and bile from non-treated animals, respectively, compared with 28%, 55% and 3% of the radioactivity excreted in ABT-treated animals. A complex metabolite profile was observed in bile of non-treated animals comprised of multiple alkyl chain-shortened carboxylic acid metabolites or conjugates of alkyl chain-shortened carboxylic acid metabolites. Parent HDP-CDV comprised a small percentage (approximately 3%) of the chromatographic radioactivity (approximately 0.3% of the dose) in non-treated animals, but increased to approximately 40% of the chromatographic radioactivity (approximately 1% of the dose) in ABT-treated animals, with a concomitant decrease in the peak areas of many of the metabolite peaks. Interpretation of study results is ongoing, with an expected completion in 2Q2012.

Study HDP-CDV-NCA-049:

In Vitro CYP Reaction Phenotyping of HDP-CDV in Human Liver Microsomes and Recombinant Human CYP Enzymes.

Due to the low in vitro turnover of HDP-CDV observed in previous studies using liver microsomes from multiple species, in this study LC/MS/MS detection was used, and formation of the major human HDP-CDV oxidative metabolites (or oxidative precursors of major metabolites), which were identified in the rat ADME study as being produced via CYP-dependent pathways was determined. This study is expected to definitively quantify the major human metabolites arising from specific CYP enzymes and inform the need for a clinical DDI study investigating the effect of inhibition of these CYP enzymes on the PK of HDP-CDV.

Study HDP-CDV-VIR-036:

In Vitro Pharmacological Activity and Cytotoxicity of Select Metabolites. The in vitro pharmacological activity and cytotoxicity of the major human metabolites against ectromelia, a double-stranded DNA virus, was assessed previously and indicated lower pharmacologic and cytotoxic activity of the following HDP-CDV metabolites, CMX103, CMX064 and CDV. Additional in vitro pharmacological and cytotoxic activity determinations are conducted in order to confirm the lack toxicity and of activity of these metabolites against CMV and AdV.

Additional In Vitro CYP and Drug Transporter Interaction Studies with Selected Metabolites

After the human radio-labeled HDP-CDV AME results from Study HDP-CDV-112 have been compiled and reviewed, additional in vitro experiments may be conducted to characterize the interaction of major metabolites with CYP enzymes and drug transporters. If conducted, such studies would likely be completed within a year of the completion of the AME study.

### Rationale for Phase 3 Study Design

A randomized, double-blind, placebo-controlled, parallel-group, dosing regimen comparison study in CMV-seropositive adult patients following hematopoietic stem cell transplantation is proposed. Study HDP-CDV-301 evaluates the safety and efficacy of two HDP-CDV dosing regimens versus placebo, for the prevention of clinically significant CMV infection and disease post-transplant. The following sections outlines key patient selection criteria; the rationale for the dose regimens; the primary and key secondary endpoints; key protocol design choices; and the safety data collection and monitoring plan.

### Rationale for Subject Populations

The subject population selected for Study HDP-CDV-301 is comprised of subjects post-HSCT who are at high risk of developing CMV infection, defined as those who are seropositive for CMV prior to transplantation (i.e., R+). This subject population has been selected as it represents immunocompromised patients who could derive significant benefit from CMV prevention, thereby increasing the benefit to risk ratio for study participants.

Only subjects who are CMV negative by PCR within 5 days prior to randomization to initiate study drug are selected for participation in the study, as subjects who are CMV positive by PCR do not qualify for prevention therapy. Subjects who were CMV negative on screening within the 5 day window, but subsequently are found to have been CMV viremic on the first day of dosing, are continued in the study. Based on the subject population enrolled in the study described above, subjects who are CMV positive and enrolled in the study make up less than 5% of the total population enrolled.

Subjects are allowed to enroll in Study HDP-CDV-301 regardless of source or type of graft, and regardless of conditioning regimen. This population is purposely inclusive in order to reflect the true make-up of at-risk patients, post-HSCT and to allow for data-driven generalizability of the study results. Because myeloablative conditioning regimens are used in 40-50% of the patients prior to HSCT, such conditioning regimen is allowed in the study population; however, since myeloablative conditioning regimen is a known risk factor for GI side effects and severity of GVHD, the type of conditioning regimen is used as a variable for stratification of randomization and all analyses (subjects who received a myeloablative conditioning regimen are included in the “high likelihood” stratum).

### Rationale for Dosing Regimen

A range of HDP-CDV doses has been characterized by the results in Study HDP-CDV-201, having both antiviral activity and acceptable tolerability for up to 11 weeks of treatment in adults following HSCT.

The lower range of doses is bound by HDP-CDV 40 mg QW, which is considered as ineffective, given that the efficacy results in this Cohort were not different from placebo, regardless of the endpoint considered. The highest evaluated dose, HDP-CDV 200 mg BIW was not sufficiently well tolerated for this prophylaxis indication and duration of therapy, due to the incidence of severe diarrhea and events reported as GVHD of the intestine. Other intermediate doses of HDP-CDV (100 mg QW, 200 mg QW and 100 mg BIW) were generally well tolerated and showed evidence of antiviral activity as summarized in TABLE 53.

TABLE 53
Proportion of Subjects Meeting Key Safety, Tolerability or Antiviral
Activity Endpoints, Cohorts 2, 3 and 4A versus Placebo

| Cohort | | | |
|---|---|---|---|
| Cohort 2 HDP- | Cohort 3 HDP- | | |
| CDV | CDV | Cohort 4a | Pooled |
| 100 mg QW | 200 mg QW | 100 mg BIW | Placebo |

Safety

| | | | | |
|---|---|---|---|---|
| Proportion of Subjects | 13/27 (48.1%) | 24/39 (61.6%) | 31/50 (62.0%) | 34/59 (67.7%) |
| with Grade 3 or higher | | | | |
| AEs | | | | |
| Proportion of Subjects | 0 | 2/39 (5.1%) | 9/50 (18.0%) | 3/59 (5.1%) |
| with Grade 3 Diarrhea^{a} | | | | |
| Proportion of Subjects | 0 | 0 | 4/50 (8.0%) | 1/59 (1.7%) |
| with Stage 3 or 4 Gut | | | | |
| GVHD | | | | |

Tolerability

| | | | | |
|---|---|---|---|---|
| Proportion of Subjects | 9/27 (33.3%) | 15/39 (38.5%) | 18/50 (36.0%) | 27/59 (45.8%) |
| with AEs Leading to | | | | |
| Treatment Withdrawal | | | | |
| Proportion of Subjects | 6/27 (22.2%) | 17/39 (43.6%) | 17/50 (34.0%) | 11/59 (18.6%) |
| Who Discontinued Study | | | | |
| for Non CMV Reasons | | | | |

Activity

| | | | | |
|---|---|---|---|---|
| Proportion of Subjects | 6/27 (22.2%) | 12/39 (30.8%) | 5/50^{c} (10.0%) | 22/59 (37.3%) |
| with CMV Disease or | | | | |
| Infection^{b} at the End of | | | | |
| Treatment | | | | |
| mITT | | | | |
| Modified CMV negative | 3/23 (13.0%) | 6/29 (20.7%) | 2/41^{d} (4.9%) | 14/47 (29.8%) |
| stratum | | | | |
| Modified CMV positive | 3/4 (75%) | 6/10 (60.0%) | 3/9 (33.3%) | 8/12 (66.7%) |
| stratum | | | | |
| Proportion of Subjects | 7/27 (25.9%) | 7/39 (17.9%) | 7/50 (14.0%) | 18/59 (30.5%) |
| Initiating Antiviral | | | | |
| Therapy or Developing | | | | |
| CMV disease (mITT) | | | | |
| Proportion of Subjects | 5/23 (21.7%) | 4/29^{g} (13.8%) | 5/41^{h} (12.2%) | 18/47 (38.3%) |
| Developing Clinically | | | | |
| Significant CMV Infection^{e} | | | | |
| (modified CMV negative | | | | |
| stratum)^{f} | | | | |

^{a}No subjects had Grade 4 or 5 diarrhea in any Cohort.
^{b}Defined as >200 copies/mL
^{c}p = 0.002 versus pooled placebo, Fisher's exact test
^{d}p = 0.002 versus pooled placebo, Fisher's exact test
^{e}p = 0.02 versus pooled placebo, Fisher's exact test (modified CMV negative stratum)
^{f}Clinically significant CMV infection is defined as: development of CMV disease or initiation of CMV preemptive therapy or plasma CMV ≧1,000 copies/mL at any time during treatment
^{g}Defined as CMV disease, initiation of CMV therapy or plasma CMV DNA >1,000 copies/mL
^{h}p = 0.036 versus pooled placebo, Fisher's exact test
ⁱp = 0.007 versus pooled placebo, Fisher's exact test

Efficacy analyses suggest that a dose of 100 mg QW may be slightly less effective than higher doses, particularly when considering subjects with detectable CMV viremia at Baseline or when clinically relevant endpoints, including the need to initiate preemptive antiviral therapy, are considered. In addition, HDP-CDV is administered with food in future studies, rather than the fasted state, which may marginally decrease drug exposure (in the food interaction Study HDP-CDV-103, exposure to HDP-CDV was reduced by 28% when the drug was administered with a high fat meal to healthy volunteers). By contrast, a weekly 200 mg dose of HDP-CDV (either 200 mg weekly or 100 mg BIW), in Study HDP-CDV-201, appeared to have good antiviral activity as measured by virologic and clinical endpoints, with evidence of greater activity for the BIW regimen in some analyses. Consequently, a dose higher than 100 mg weekly in Study HDP-CDV-301 is proposed.

When comparing QW versus BIW dosing regimens, more frequent and more severe AEs were reported in subjects who received HDP-CDV 100 mg BIW versus subjects who received 200 mg QW. By contrast, subjects who received HDP-CDV 100 mg BIW were more likely to complete the intended duration of treatment and in spite of drug interruptions and ended up, on average, receiving a higher total dose of HDP-CDV, as compared to subjects randomized to HDP-CDV 200 mg QW.

A direct comparison of safety and tolerability parameters between the 200 mg QW and 100 mg BIW doses should be made with caution for several reasons. First, significant changes were made in study conduct and the extent of safety information provided in the 100 mg BIW group (Cohort 4a). Regarding the occurrence of GI AEs in association with the maximum dose Cohort 4 (200 mg BIW), there was heightened monitoring of subjects in Cohort 4A (100 mg BIW) including increased application of Safety Monitoring and Management Plan guidelines, which recommended drug interruptions to manage GI AEs. In addition, the baseline subject characteristics of the 200 mg QW and the 100 mg BIW Cohorts were unbalanced for factors associated with a risk of GVHD and GI side effects, with higher risk subjects participating in Cohort 4A; this difference existed between the 2 Cohorts, as well as in comparison to the pooled placebo and other HDP-CDV dose Cohorts.

To examine whether the safety profile of HDP-CDV administered as 200 mg QW or 100 mg BIW in the Cohorts 3 and 4a was dependent on weight, the safety and tolerability by subgroups was investigated, defined as subjects having received either≦3 mg/kg or >3 mg/kg in Cohorts 3 (200 mg QW) and 4A (100 mg BIW), (The cut-off was chosen as the approximate weekly dose expressed in mg/kg for a 65 kg subject receiving 200 mg, 65 kg representing the approximate lower 20^{th} percentile weight of subjects participating in the study). Individual GI AEs were assessed in prior sections, supporting a dose adjustment for lower weight individuals. An assessment of diarrhea and GVHD by severity is presented in TABLE 54.

TABLE 54
Incidence of Stage 2 and Higher GVHD of the Intestine AND/OR Grade
2 Diarrhea by HDP-CDV dose Expressed as mg/kg, Cohorts 3 and 4A versus Pooled Placebo

| | Cohort 3 | Cohort 3 | Cohort 4A | Cohort 4A | |
|---|---|---|---|---|---|
| | HDP-CDV | HDP-CDV | HDP-CDV | HDP-CDV | |
| | 200 QW | 200 QW | 100 mg BIW | 100 mg BIW | Placebo |
| System Organ Class | ≦3 mg/kg | >3 mg/kg | ≦3 mg/kg | >3 mg/k | Pooled |
| Preferred Term | N = 25 | N = 14 | N = 40 | N = 10 | N = 59 |
| Frequency gut GVHD Stage 2, 3, 4 | 3/25 | 2/14 | 7/40 | 3/10 | 2/59 |
| | 12% | 14% | 18% | 30% | 5% |
| Frequency Diarrhea Grade ≧2 | 2/25 | 5/14 | 9/40 | 6/10 | 8/59 |
| | 8% | 42% | 23% | 60% | 14% |
| # subjects with Either GVHD ≧3 | 5/25 | 6/14 | 12/40 | 6/10 | 10/59 |
| or Diarrhea ≧2 or Both (not | 25% | 43% | 30% | 60% | 17% |
| double-counted) | | | | | |

These data show that administration of a smaller total weekly dose of HDP-CDV to lower weight individuals has the potential to reduce the overall incidence and severity of diarrhea and/or events reported as GVHD of the intestine in subjects receiving HDP-CDV QW. This rationale may be incorporated in the proposed dosing regimens.

### Proposed Dose and Dosing Regimens

Based on the overall safety and activity data, a comparison of 2 dosing regimens of HDP-CDV-200 mg QW and 75 mg BIW-versus placebo in the proposed double-blind, parallel group, placebo-controlled Phase 3 Study HDP-CDV-301 is planned, which evaluates the efficacy and safety of HDP-CDV for use in the prevention of clinically significant CMV infection in CMV seropositive adult HSCT recipients.

Randomization in the study may be 1:1:1 and stratified by weight and risk factors for GVHD and CMV infection (i.e., high likelihood versus lower likelihood). All HDP-CDV doses is administered with food and subjects are managed according to the safety monitoring and management plan, which allows either dose interruption, dose reduction (for the QW arm), or change in dosing frequency (for the BIW arm).

The proposed HDP-CDV dosing regimens are summarized in TABLE 55. While they are listed as once or BIW, it should be noted that all subjects receive tablets for BIW administration—the “QW” arm receives matching placebo for the second dose each week in order to maintain the overall double-blinding of the study.

TABLE 55
Proposed HDP-CDV Dosing Regimens in Study HDP-CDV-301

| | | |
|---|---|---|
| Subject weight | QW arm | BIW arm |

Initial dose

| | | |
|---|---|---|
| Between 65 and 120 kg | 200 mg QW/Placebo QW | 75 mg BIW |
| <65 kg | 150 mg QW/Placebo QW | No change |

Subject requiring dose modification per SMMP

| | | |
|---|---|---|
| Between 65 and 120 kg | 150 mg QW/Placebo QW | 150 mg QW/ |
| | | Placebo QW |
| <65 kg | 150 mg QW/Placebo QW | No change |

### Rationale for QW Regimen

A 200 mg QW dose of HDP-CDV has been chosen for further development in Phase 3. The rationale for choosing this dosing regimen is that 200 mg QW demonstrated antiviral activity across several analyses in the study described above (Study HDP-CDV-201). While overall sufficiently well tolerated, this dose showed a trend toward an increased risk of GI AEs in comparison to lower doses and placebo; the GI AEs were more apparent in subjects for whom the weekly dose exceeded 3 mg/kg as shown in the prior section. Therefore, subjects who have a body weight less than 65 kg and are randomized to the QW arm receive a lower unit dose (150 mg QW) or matching placebo, in order to provide such patients with a potentially more tolerable dose at the outset and to reduce the need for dose interruptions during the course of treatment.

The 200 mg QW dosing regimen was chosen in order to provide a favorable benefit risk profile for subjects by: Demonstrating antiviral activity since the total weekly dose is within the range of weekly doses demonstrating such activity in Study HDP-CDV-201 (i.e., 100 to 200 mg weekly). Providing at the outset for a decreased rate of GI side effects, since HDP-CDV is administered with food and exposures are limited for smaller subjects weighing<65 kg. Leading to a decreased frequency of higher grade GI events and a higher completion rate during the course of the study, through use of the Safety Monitoring and Management Plan, which allows for dose interruption and if necessary dose reduction.

### Example 5

### DOSAGE: Rationale for BIW Dose and Regimen

A 75 mg BIW dose of HDP-CDV has also been chosen for further evaluation in Phase 3.

The rationale for choosing this dosing regimen is to further explore the potential for enhanced efficacy that may be associated with BIW dosing in the prevention of CMV and other dsDNA infections. Overall, the 100 mg BIW dose in Study HDP-CDV-201 tended to show increased efficacy versus QW doses in the primary endpoint analyses, which may have been related in part to: the larger group size; longer time on drug; or better coverage with the BIW dosing interval.

In the more objective secondary virologic analyses, the results were similar to those of the 200 mg QW group across a range of endpoints. However, the 100 mg BIW dosing regimen achieved improved antiviral activity in subjects with detectable CMV DNAemia at Baseline. In addition, regardless of CMV DNAemia status at Baseline, when individual subject results were examined, the 100 mg BIW dose appeared to achieve greater suppression of CMV reactivation and DNAemia in comparison to the 200 mg QW dose. This greater suppression was in spite of an increased frequency of treatment with systemic steroids in Cohort 4A, which, in theory, should have decreased the potential for HDP-CDV antiviral activity in that group. Because of the increased antiviral activity across multiple endpoints, further exploration of a BIW dosing regimen is warranted.

While the 100 mg BIW dose was tolerable and led to a high rate of treatment completion in Study HDP-CDV-201, in comparison to 200 mg QW, 100 mg BIW was associated with an increased rate of GI AEs. Treatment was continued in most patients, resulting in a 60% completion rate, indicating that these AEs were manageable with the use of dose interruptions. However, in the proposed Study HDP-CDV-301, the BIW dosing scheme proposes to explicitly test without interruption, if possible. Therefore, in order to limit the need for such interruptions, 75 mg BIW in Study HDP-CDV-301 is proposed.

A twice-weekly 75 mg dose of HDP-CDV has been chosen in order to: Potentially provide additional antiviral activity based upon the BIW dosing paradigm, while maintaining a total weekly dose that is within the range of weekly doses of HDP-CDV with demonstrated antiviral activity in Study HDP-CDV-201 (i.e., 100 to 200 mg weekly). Provide a decreased rate of GI side effects, particularly when HDP-CDV is administered with food, because all subjects receive less than 3 mg/kg per week and begin treatment at a 25% lower total dose than that studied in Cohort 4A. Maximize the possibility of limiting dose interruptions and/or subsequent reversion to QW dosing, maintaining BIW dosing whenever possible.

### Rationale for Comparator

Currently, there is an unmet medical need for a prophylactic drug for CMV infection in HSCT recipients. No drug is approved or recommended by ASBMT guidelines (Tomblyn M, et al. Guidelines for Preventing Infectious Complications among Hematopoietic Cell Transplantation Recipients: A Global Perspective. Biol Blood Marrow Transplant 2009; 15:1143-1238) for the indication under study. Therefore, use of a placebo as a control in Study HDP-CDV-301 is proposed. This comparison (prevention of clinically significant CMV infection or disease with HDP-CDV versus placebo) may represent the primary analysis in the study. Additional analyses may compare the safety and benefits of CMV prevention with HDP-CDV with those of preemptive therapy initiated based on a positive CMV assay (DNA or antigenemia). Previous prophylaxis studies with GCV were unsuccessful because of an increase in secondary infections following GCV treatment and limited case studies with foscarnet were not expanded due to toxicity; for those reasons, preemptive therapy is the current practice. Therefore, a placebo controlled trial is acceptable since placebo recipients receive standard of care, with preemptive therapy initiated on the basis of regular CMV DNAemia monitoring.

### Rationale for Primary End Point

In Study HDP-CDV-201, additional data on 43 subjects who discontinued due to CMV viremia/disease were collected. 11 subjects were discontinued due to confirmed CMV disease. 17 subjects were discontinued due to increasing CMV viremia without any other specific signs and/or symptoms of CMV infection. 5 subjects had aGVHD and/or upper GI symptoms that the sites reported as contributory to the decision to discontinue the subject and treat with alternative anti-CMV therapy. 4 subjects (3 from one site) had an event of fever which contributed to the decision to discontinue the subject. CMV end-organ disease is a reasonably well-defined event; however, the goal of CMV monitoring and pre-emptive therapy is to prevent CMV disease. CMV “syndrome” is well defined for SOT recipients, but there is no analogous syndrome or combination of clinical signs and symptoms for HSCT recipients.

Given the enormous heterogeneity of the HSCT recipient population, including differences in underlying disease, conditioning regimen, stem cell source, and donor:recipient match, there is not any one clinical event used to guide treatment decisions. Rather, CMV DNA in plasma detected by PCR is the key driver of treatment initiation, depending on the risk of CMV progression. The investigators emphasized that clinical judgment based on CMV viremia and the patient's unique medical history is used to determine when a patient is treated for CMV. The investigators also noted that standard practices differ in terms of levels of viremia which trigger initiation of treatment for a particular subject population (e.g., subjects exposed to myeloablative conditioning regimens or subjects with a T-cell depleted graft).

For initiation of antiviral therapy, 1000 copies/mL of viral DNA was a generally accepted threshold in subjects at low risk of subsequently developing end organ CMV disease. Consequently, the proposed composite primary endpoint for this study was the proportion of subjects who develop clinically significant CMV infection, disease, or the initiation of pre-emptive anti-CMV treatment within the time period of initiation of dosing and Day 100 post-transplant. This composite endpoint was defined as involving at least one of the following outcomes: CMV end-organ disease and/or Initiation of anti-CMV specific therapy based on the treating physician's judgment of the patient's clinical status and documented CMV DNAemia, and/or CMV DNAemia>1,000 copies/mL (conducted at the central laboratory, confirmed by a second increasing value of at least 2,000 copies/mL).

In comparison to placebo, there may be a dose of HDP-CDV that reduces the percentage of patients developing clinically significant CMV events as defined by at least 50% within the time period up to one week posttreatment (i.e., Day 98 after transplantation). The diagnosis of CMV disease is adjudicated accordingly.

The aim of this composite endpoint is to capture all clinical failures of the prophylactic treatment regimen, including development of CMV disease; the clinical decision to initiate preemptive therapy (and its associated toxicity); and progressing CMV PCR positivity that predisposes subjects to the onset of CMV disease. A key secondary endpoint is the proportion of subjects developing these CMV events at any time during study participation (i.e., including during the treatment-free follow-up, up to Week 21 post-transplant), in order to assess the impact of later onset CMV events posttreatment.

The composite endpoint is analyzed to determine the dose of HDP-CDV that is both safe and effective in the prevention of clinically significant CMV events in adults at high risk post-HSCT. Sensitivity analyses are conducted on the components of this endpoint to confirm that the direction of the treatment effect is consistent with the primary outcome.

CMV end organ disease is the most serious outcome of CMV infection and is characterized by significant morbidity and mortality. For this study, CMV disease diagnosis may be based on the criteria defined by Ljungman, et al. The Definitions of CMV Infection and Disease in Transplant Recipients. CID 2002; 34:1094-1097.

Ljungman provides definitions and guidelines for diagnosis of CMV pneumonia, gastrointestinal disease, hepatitis, CNS disease, retinitis, nephritis, cystitis, myocarditis, pancreatitis, and CMV-associated graft failure. Subjects developing CMV disease during the study are followed until Week 21 after HSCT.

Initiation of preemptive therapy by the Investigator is an important medical event since such therapies are associated with significant toxicities (e.g., neutropenia, cytopenia, nephrotoxicity) and denote the failure of the prophylaxis approach. Subjects initiating anti-CMV preemptive therapy during the study are followed until Week 21 after transplantation.

The decision to initiate preemptive anti-CMV treatment for any given HSCT recipient is based upon the Investigator's assessment of the degree of individual's risk of developing CMV disease. Numerous risk factors contribute to this decision including level of viremia, progressive viremia and factors promoting rapid progression to CMV disease (corticosteroid or ATG therapy, cord blood transplantation, T-cell depletion, donor seronegativity against CMV, etc.). For each individual patient, the clinical decision to initiate preemptive treatment signifies that the risk of developing CMV disease exceeds the potential side effects associated with commercially available preemptive therapy.

To illustrate these toxicities, during the course of Study CMX01-201, 74 subjects required therapy with antivirals with activity against CMV (ganciclovir, valganciclovir, foscavir or cidofovir). Of these, 71 subjects had follow-up data available. Following the switch to CMV preemptive therapy, 14 subjects (20%) had no apparent significant toxicity based on need for transfusion, growth factors, alteration in their neutrophil count or increase in creatinine levels. However, 41% experienced severe or life-threatening neutropenia (<1 G/L decrease from the last value on blinded study medication); 15% had a significant decrease in their neutrophil count (>2G/L); and 14% had moderate neutropenia (<1.5 G/L). Twenty-five percent (25%) of the subjects experienced an increase in creatinine level of more than 20% after initiation of anti-CMV therapy. In addition, 15% of the subjects required G-CSF therapy, 7% required new RBC transfusion and 3% required new platelet transfusions. Fourteen percent (14%) of the subjects who initiated preemptive therapy with ganciclovir or valganciclovir had to be switched to a second line therapy (foscavir or cidofovir), due to the toxicity of the initial regimen.

Because of the significant toxicities associated with currently available anti-CMV medications, initiation of preemptive treatment may be considered as a clinically important event suitable for inclusion in the proposed primary endpoint.

Subjects enrolled in Study HDP-CDV-301 who develop confirmed CMV DNAemia with PCR values of >1,000 copies/mL are also considered prophylaxis failures. Subjects are required to discontinue study medication and are managed per the local standard of care. In the absence of a clinical decision to initiate preemptive treatment based upon the initial CMV measurement in association with other high risk factors, PCR assays are performed by the central laboratory and confirmed by a subsequent measurement of >1,000 copies/mL, if medically acceptable. (If not medically acceptable, the site may be asked to collect a second sample prior to initiation of standard preemptive therapy but need not wait for the results to initiate treatment; in this case, the subject may be viewed as prophylaxis failure because of the need for preemptive treatment).

Progressive CMV viremia>1,000 copies/mL, rising on confirmation 5 to 7 days later, is proposed as a clinically meaningful endpoint requiring withdrawal of study drug (or placebo), since it indicates lack of antiviral response. The affected subjects are followed in the study until Week 21 post-transplant.

### Rationales for Key Secondary Endpoints

Treatment Emergent Resistance:

Generation of drug resistant virus may be a byproduct of the use of antiviral therapies and is a consideration whenever an antiviral drug is prescribed. Therefore, one key secondary endpoint is to compare the incidence of viral breakthrough and the emergence of HDP-CDV-resistant CMV isolates, between HDP-CDV- and placebo-treated subjects and between each HDP-CDV dose group and placebo. Subjects experiencing virologic failure are evaluated for genotypic changes in the CMV UL54 and UL97 genes; mutations identified are characterized for phenotypic resistance as appropriate.

Emergence of Viremia During the Intended Prevention Period (i.e., Up to One Week after the Last Planned Dose of Study Drug):

In order to select the most active dosing regimen of HDP-CDV, the incidence of CMV viremia at any time on study (defined as CMV DNAemia>1000 copies/mL, followed by analysis of ≧200 copies/mL, if necessary) is used as a measure of antiviral activity in order to differentiate between doses with similar efficacy and safety, if necessary.

Non-Relapse Mortality:

In order to characterize the positive outcome of prophylaxis therapy with HDP-CDV versus standard of care, non-relapse mortality are compared between subjects randomized to receive HDP-CDV versus placebo.

### Rationale for Duration of Dosing and Follow-Up

Based on the available literature and current standard of care, the risk for CMV reactivation post-HSCT is highest between transplantation and Day 100 post-transplant. In addition, many patients are returned to the care of their primary oncologist (often in another location) by Day 100 post-transplant. In order to cover this risk period, subjects in the study may receive study drug from engraftment through Week 13 post-transplant, returning for the “End of Treatment” Follow-up Week 1 visit during Week 14 post-transplant. This allows for all treatment visits and the first safety follow-up visit to occur at the study site before the patient is discharged back to their “hometown” physician/oncologist. Depending on the time of engraftment, the total duration of therapy for individual subjects may vary between 9 to 1 weeks and may be similar to that of Study HDP-CDV-201.

After the end of the planned duration of randomized therapy, subjects are followed for 8 weeks to monitor for CMV rebound in order to assess the risk of relapse after cessation of preventative therapy. Results from Study HDP-CDV-201 demonstrated that this duration of follow-up is sufficient, since less than 10% of the subjects had emergence of viremia between the end of treatment and then end of the treatment-free follow-up (8 weeks follow-up).

Subjects who discontinue study medication for any reason (except death, withdrawal of consent or loss to follow-up) are continued to be followed in the study until they complete the Week 21 post-transplant visit.

Additional long term follow-up is planned for subjects who complete the study as part of a long-term registry. Participants in the registry are contacted every 6 months to assess survival and hematologic malignancy relapse.

### Safety Data Collection and the Safety Monitoring and Management Plan

Based on the safety findings and analyses from Study HDP-CDV-201, HDP-CDV doses not exceeding 3 mg/kg/week are used administered with food, in order to minimize gastrointestinal AEs.

The components of the SMMP include the following: Diagnosis of Cause(S) of Gastrointestinal Signs and Symptoms in HSCT Patients; Etiologies of Diarrhea; Etiologies of Upper Gastrointestinal Symptoms; Etiologies of Hepatobiliary Symptoms and Laboratory Abnormalities; Management of Subjects with Gastrointestinal AEs in HDP-CDV Clinical Studies. For subjects with Grade 1 GI-related AEs. For subjects with Grade 2 GI-related AEs. For subjects with Grade 3 or higher GI-related AEs; Management of Subjects with Incident Serum Elevations in Liver Enzymes; Safety Oversight; Adverse Events of Special Interest.

Therefore, in addition to overall surveillance for adverse events, subjects enrolled in Study HDP-CDV-301 and other future HDP-CDV studies are monitored for liver enzyme elevations, gastrointestinal signs and symptoms and reports of GVHD. Study HDP-CDV-201 indicated that these events are seen in association with the use of HDP-CDV. The safety management plan, along with data-driven dose selection and the planned administration of HDP-CDV with food are expected to minimize the rate of drug-related adverse events in study HDP-CDV-301.

Additional information on such events are captured in the eCRF for any future studies including, but not limited to, gastrointestinal symptoms and GVHD. Gastrointestinal assessments include the following: symptoms at each visit; measurements of stool volumes (if possible for inpatients) or number of stools; grading and staging of GVHD, if present, assessed for each organ of involvement; results of any diagnostic investigations; central reading of biopsy slides (blinded to treatment assignment for biopsies of gut and/or skin); measurements of immunosuppressant blood concentrations; and collection of treatment and response details. In addition, details of skin findings are collected when skin GVHD is reported.

### Example 6

HDP-CDV-301 is a randomized, placebo-controlled, double-blind, parallel groups dosing regimen comparison study in adult HSCT recipients for evaluating the safety and efficacy of two HDP-CDV dosing regimen versus placebo for the prevention of clinically significant CMV infection. Study HDP-CDV-301 evaluates the safety and efficacy of two HDP-CDV dosing regimens versus placebo, for the prevention of clinically significant CMV infection and disease post-transplant in HSCT recipients.

To date more than 700 subjects have received HDP-CDV. Findings from the Phase 1 and 2 studies are summarized in TABLE 56. In the Phase 1 study, healthy volunteer were provided with single and multiple dose tolerability and preliminary pharmacokinetics (PK) of doses up to 1 mg/kg 6 days for 3 doses.

Data from 210 Patients Treated with HDP-CDV Under Emergency INDs (EINDs)

TABLE 56
HDP-CDV Studies in Support of the Initiation of Study HDP-CDV-301
for Prevention of CMV Infection.

| | | | | | |
|---|---|---|---|---|---|
| | | | | | Actual |
| | | | | | Enrollment or |
| Study | Subject | Control | Primary End | Duration of | Estimated |
| Number | Population | Group | Point(s) | Dosing/Follow-up | Sample Size |

Clinical Pharmacology Studies (Completed)

| | | | | | |
|---|---|---|---|---|---|
| HDP- | Healthy | Placebo | Safety/tolerability, | Single ascending | Completed |
| CDV-102 | subjects | | PK | doses (SAD; 0.025-2.0 mg/kg) | study |
| | Age: 18-55 yrs. | | | and | Overall: |
| | | | | multiple ascending | 84 subjects |
| | | | | doses (MAD; 0.1-1.0 mg/kg, | (56 HDP-CDV, |
| | | | | q6 days | 28 placebo) |
| | | | | for 3 doses) | SAD: |
| | | | | | 54 subjects |
| | | | | | (36 HDP-CDV, |
| | | | | | 18 placebo) |
| | | | | | MAD: |
| | | | | | 30 subjects |
| | | | | | (20 HDP-CDV, |
| | | | | | 10 placebo) |
| HDP- | Healthy | NA | Comparative | Three single 40 mg | Completed |
| CDV-103 | subjects | | bioavailability, | doses at ≧14 day | study, |
| | Age: 18-55 yrs. | | food effect, PK, | intervals | 24 subjects |
| | | | safety/tolerability | | enrolled |

Clinical Pharmacology Studies (Ongoing and Planned)

| | | | | | |
|---|---|---|---|---|---|
| HDP- | Subjects with | NA | Safety/tolerability, | Single 200 mg dose | Completed |
| CDV-106 | moderate and | | PK | | study, |
| | severe | | | | 25 subjects |
| | hepatic | | | | (8 with normal |
| | impairment | | | | hepatic |
| | and healthy | | | | function, 9 with |
| | control | | | | moderate |
| | subjects with | | | | hepatic |
| | normal renal | | | | impairment, |
| | function | | | | 8 with severe |
| | Age: 18-65 yrs. | | | | hepatic |
| | | | | | impairment |
| | | | | | enrolled) |
| HDP- | Healthy male | NA | Absorption, | Single 200 mg dose | Enrollment |
| CDV-112 | subjects | | metabolism and | | completed, |
| | Age: 18-55 yrs. | | excretion, | | 6 subjects |
| | | | elimination | | |
| | | | (AME), PK, | | |
| | | | safety/tolerability | | |
| HDP- | Healthy | Placebo, | Thorough QT | Single doses of 200 mg | Planned for |
| CDV-108 | subjects | moxifloxacin | (TQT) study, | and 350 mg | 56 subjects |
| | Age: 18-45 yrs. | | safety/tolerability, | (=supra-therapeutic | |
| | | | PK | dose) HDP-CDV | |
| | | | | and one 400 mg | |
| | | | | dose of | |
| | | | | moxifloxacin | |
| HDP- | Healthy | NA | Drug-drug | Two single 200 mg | Planned for |
| CDV-113 | subjects | | interaction | HDP-CDV doses, 2 | 20 subjects |
| | Age: 18-55 yrs. | | between HDP- | IV (1 mg) doses of | |
| | | | CDV and | MDZ and two per | |
| | | | midazolam | os (PO) (2.5 mg) | |
| | | | (MDZ), PK, | doses of MDZ | |
| | | | safety/tolerability | | |

Completed Studies in Transplant Recipients

| | | | | | |
|---|---|---|---|---|---|
| HDP- | Adult HSCT | Placebo | Safety endpoints | Dose escalation, | Enrollment |
| CDV-201 | recipients | | include clinical | drug versus | completed |
| Viral | CMV | | assessments and | placebo. | Overall: |
| target: | seropositive | | laboratory values, | Up to 11 weeks | 239 subjects: |
| CMV | (R+) at the | | adverse events | treatment in one of | Cohort 1: 40 |
| | time of | | (AEs) (and serious | five Cohorts: | (30A:10P) |
| | transplant | | adverse events | Cohort 1: 40 mg | Cohort 2: 39 |
| | | | [SAEs]), changes | QW | (29A:10P) |
| | | | from baseline in | Cohort 2: 100 mg | Cohort 3: 53 |
| | | | laboratory values, | QW | (39A:14P) |
| | | | vital signs, | Cohort 3: 200 mg | Cohort 4: 40 |
| | | | electrocardiograms | QW | (30A:10P) |
| | | | (ECGs) and renal | Cohort 4: 200 mg | Cohort 4A: 67 |
| | | | function | BIW | (50A:17P) |
| | | | Efficacy endpoint | Cohort 4A: | |
| | | | is CMV DNAemia | 100 mg BIW, | |
| | | | >200 copies/mL at | 3 active (A): | |
| | | | the conclusion of | 1 placebo (P) | |
| | | | treatment or | randomization/ | |
| | | | diagnosis of CMV | 8 weeks | |
| | | | disease during the | posttreatment | |
| | | | treatment period | follow-up | |

Ongoing and Planned Controlled Studies in Transplant Recipients

| | | | | | |
|---|---|---|---|---|---|
| HDP- | Pediatric and | Placebo | Safety, prevention | From 6 to 12 | Planned |
| CDV-202 | adult | | of AdV disease or | weeks therapy with | enrollment, |
| Viral | subjects, | | confirmed increase | HDP-CDV QW or | 48 subjects |
| target: | post-HSCT | | in plasma AdV | BIW (max. | (32 active, |
| AdV | with | | DNA requiring | 200 mg/week in | 16 placebo); |
| | asymptomatic | | alternative therapy | adults or max. | actual |
| | AdV viremia | | | 4 mg/kg/week in | enrollment, |
| | | | | children)/4 weeks | 17 subjects as |
| | | | | treatment-free | of 13Apr2012 |
| | | | | follow-up | |

Ongoing Uncontrolled Studies in Immunocompromised Subjects

| | | | | | |
|---|---|---|---|---|---|
| HDP- | Adults and | NA | Safety, antiviral | Up to 6 months | Enrollment |
| CDV-350 | pediatric | | activity, PK | therapy with once | completed |
| Viral | subjects with | | | or BIW dosing | Planned |
| target: | severe and | | | with HDP-CDV | enrollment |
| various | life- | | | (max. | 200 subjects; |
| dsDNA | threatening | | | 200 mg/week for | actual |
| viruses | dsDNA | | | most adults, max. | enrollment |
| (including | disease or | | | 4 mg/kg/week for | 207 subjects |
| CMV and | infection | | | most | |
| AdV) | | | | children)/4 weeks | |
| | | | | treatment-free | |
| | | | | follow-up | |
| | | | | Treatment may be | |
| | | | | extended for | |
| | | | | individual patients | |
| | | | | after 6 months at | |
| | | | | Agency's | |
| | | | | discretion on a | |
| | | | | case-by-case basis. | |
| HDP- | Subset of | NA | Safety, antiviral | Up to 6 months | Planned |
| CDV-350 | subjects | | activity, PK | therapy with once | enrollment |
| CDV-PP | participating | | | or BIW dosing | 30 subjects; |
| PK | in HDP- | | | with HDP-CDV | actual |
| Substudy | CDV-350 at | | | (max. | enrollment |
| Viral | selected | | | 200 mg/week for | 8 subjects |
| target: | investigative | | | most adults, max. | |
| various | sites is | | | 4 mg/kg/week for | |
| dsDNA | enrolled into | | | most | |
| viruses | this Substudy | | | children)/4 weeks | |
| (including | which | | | treatment-free | |
| CMV and | measures | | | follow-up | |
| AdV) | intracellular | | | Treatment may be | |
| | levels of | | | extended for | |
| | CDV-PP in | | | individual patients | |
| | peripheral | | | after 6 months at | |
| | blood | | | Agency's | |
| | mononuclear | | | discretion on a | |
| | cells. | | | case-by-case basis. | |
| | Subjects must | | | | |
| | weigh | | | | |
| | ≧60 kg. | | | | |
| EINDs | Adults and | NA | Safety, antiviral | Up to 6 months | Enrollment |
| Viral | pediatric | | activity, PK | dosing | completed |
| target: | subjects with | | | Treatment may be | (possible |
| various | severe and | | | extended for | exceptions) |
| dsDNA | life- | | | individual patients | Planned |
| viruses | threatening | | | after 6 months at | enrollment, up |
| (including | dsDNA | | | Agency's | to 250 subjects; |
| CMV and | disease or | | | discretion on a | actual |
| AdV) | infection | | | case-by-case basis. | enrollment, 214 |
| | | | | | subjects as of |
| | | | | | Feb. 13, 2012 |

Clinical pharmacology studies included in this study were: (1) Absorption-metabolism-excretion (AME) study; (2) hepatic impairment study; (3) thorough QTc study; (4) drug-drug interaction study between HDP-CDV and intra-venous (IV) and oral midazolam; and (5) additional DDI studies based on in vitro data as well as results of the AME study.

Protocol for HDP-CDV-301:

HDP-CDV-301 Protocol Synopsis

A protocol concept sheet is presented in TABLE 57.

TABLE 57
HDP-CDV-301 Protocol Synopsis

Name of Investigational Product: HDP-CDV
Name of Active Ingredient: HDP-CDV (Phosphoric acid, [[(S)-2-(4-amino-2-oxo-1(2H)-
pyrimidinyl)-1-(hydroxymethyl)ethoxy]methyl]mono[3-(hexadecyloxy)propyl] ester)
Title of Study: A Randomized, Double-Blind, Placebo-Controlled, Parallel-Group, Multicenter, Phase
3 Study of the Safety, Tolerability, and Efficacy of two Dosing Regimens of HDP-CDV for the
Prevention of Cytomegalovirus Infection in R+ Hematopoietic Stem Cell Transplant Recipients
Study period: est. 4^{th} quarter 2012-4^{th} quarter 2014 Phase of development: 3
Description of Study Drug: HDP-CDV is an orally administered lipid conjugate of the synthetic
nucleotide analog cidofovir (CDV). The conjugate is absorbed in the small intestine, circulates as
HDP-CDV in the periphery, and is delivered to target organs throughout the body. Inside the cell,
HDP-CDV is cleaved by intracellular enzymes to release CDV, which is converted to the active
antiviral agent, CDV-diphosphate, by intracellular anabolic kinases.
Overall Objective: The overall objective of the study is to assess the safety and efficacy of 2 dosing
regimens of HDP-CDV compared to placebo in the prevention of clinically significant CMV infection
in CMV seropositive (R+) hematopoietic stem cell transplant (HSCT) recipients who are CMV DNA
negative from transplant up until screening (no more than 5 days prior to dosing).
Primary Efficacy Endpoint: The primary efficacy endpoint is development of clinically significant
CMV infection measured (for the primary analysis) by Week 14 post-transplant (Post Treatment Week
1). Clinically significant CMV infection is defined as the occurrence of any one of the following three
outcomes:
1) CMV end-organ disease and/or
2) Initiation of anti-CMV specific therapy based on the treating physician's judgment of the
patient's clinical status and documented CMV DNAemia, and/or
3) CMV DNAemia >1,000 copies/mL (conducted at the central laboratory, confirmed by a
second increasing value of at least 2,000 copies/mL)
Subjects who meet any of the above 3 criteria while receiving blinded study drug must discontinue
treatment with study drug. Following discontinuation from randomized study treatment, all subjects
are managed at the discretion of the Investigator according to site standard of care but are continued to
be followed in the study until Week 21 post-transplant.
Secondary Efficacy Objectives:
1) For subjects confirmed to be CMV-negative on the first day of dosing, to compare
the antiviral activity of the two HDP-CDV regimens based on the incidence of CMV
DNAemia >1,000 copies/mL and ≧200 copies/mL occurring by Week 14 post-transplant (Post
Treatment Week 1) and by Week 21 post-transplant (End of Study visit).
2) To compare the incidence of clinically significant CMV infection as defined
above between the 2 HDP-CDV dose regimens and placebo at the End of Study (Week 21
post-transplant)
3) To compare the emergence of clinical and laboratory events associated with dsDNA viral
infections (other than CMV) between HDP-CDV- and placebo-treated subjects and between each
HDP-CDV treatment group and placebo treated subjects.
4) To compare the incidence of initiation of CMV pre-emptive therapy between HDP-CDV-
and placebo-treated subjects by Week 14 post-transplant (Post Treatment Week 1) and by Week 21
post-transplant (End of Study visit).
5) To compare the incidence of CMV end-organ disease (as adjudicated by the Endpoint
Adjudication Committee) between HDP-CDV- and placebo-treated subjects by Week 14 post-
transplant (Post Treatment Week 1) and by Week 21 post-transplant (End of Study visit).
Secondary Efficacy Endpoints
1&2) CMV DNAemia >1,000 copies/mL and ≧200 copies/mL.
3) Initiation of alternate anti-CMV therapy (e.g., GCV, vGCV, foscarnet, cidofovir)
4) CMV end-organ disease confirmed according to the definitions/procedures described by
Ljungman et al (Ljungman 2002)
5) Emergence of confirmed disease or laboratory abnormalities caused by another dsDNA
virus including, for example, AdV, HSV1, HSV2, VZV, EBV and HHV6.
Overall Safety Objective:
The overall safety objective of the study is to characterize the safety and tolerability of prophylaxis
with two dosing regimens of HDP-CDV followed by standard of care compared to placebo, followed
by standard of care in allogeneic R+ HSCT recipients beginning postengraftment. The assessment of
safety and tolerability are based on treatment burden, overall AE and laboratory abnormalities, AEs of
special interest, mortality, and resistance emergence, as described below
Individual Safety Objectives:
Treatment burden (from Baseline through Week 21 post-transplant) compared between HDP-CDV
dosing regimens and placebo and between HDP-CDV dosing regimens:
1) To compare total duration of hospitalization, use of transfusions, hematopoietic growth
factors and anti-infective medications between each HDP-CDV dose regimen and placebo, during the
treatment period and during the entire study duration (between Baseline and Week 21 post-transplant).
2) To compare the incidence and severity of secondary infections (bacterial, fungal or viral,
excluding CMV and other dsDNA viruses) between HDP-CDV- and placebo-treated subjects.
3) To compare the incidence of renal dialysis or renal impairment requiring dose adjustments
of concomitant medications
4) To compare the incidence of invasive GI procedures (endoscopies, biopsies and related
procedures).
Overall safety:
1) To compare the incidence of treatment emergent adverse events between HDP-CDV- and
placebo-treated subjects, and between HDP-CDV dose regimens with particular attention to Grade 3 to
5 treatment emergent AEs.
2) To compare subject drop-out rates and/or time to discontinuation from blinded study
medication and from study for all non-CMV related events between HDP-CDV-versus placebo treated
subjects, and between HDP-CDV dose regimens.
GI events of special interest:
1) To compare between HDP-CDV dose regimens and versus placebo the rates of blinded
treatment interruptions and/or dose reductions, occurring to manage tolerability events.
2) To compare the incidence, severity and progression of episodes of diarrhea for GI events
between HDP-CDV-versus placebo-treated subjects, and between HDP-CDV dose regimens.
3) To compare the incidence, severity and progression of acute GVHD, and GI GVHD
(specifically of the intestine) between HDP-CDV-versus placebo-treated subjects, and between HDP-
CDV dose regimens.
4) To compare the incidence, severity and progression of liver related laboratory
abnormalities between HDP-CDV-versus placebo-treated subjects, and between HDP-CDV dose
regimens.
5) To assess the effect of HDP-CDV treatment on a mg/kg basis compared to placebo on the
overall incidence of adverse events and, specifically, GI events and liver related abnormalities.
Other Safety Outcomes:
6) To compare rates of non-relapse mortality (NRM) and transplant-related mortality (TRM)
occurring on-treatment and posttreatment between HDP-CDV- and placebo-treated subjects and
between HDP-CDV dose regimens and placebo.
7) To compare the incidence of emergence of HDP-CDV-resistant CMV isolates between
HDP-CDV- and placebo-treated subjects and between each HDP-CDV dose regimens and placebo.
Safety Parameters:
1&2) Adverse events and serious adverse events, hospitalization, concomitant
therapies and procedures are collected throughout treatment and posttreatment follow-up periods.
3) Reason for discontinuation from study treatment and from the study during posttreatment
follow-up are recorded for each subject and categorized to assess similarities across treatment groups.
4) Diarrhea is assessed on a separate eCRF page designed to capture individual events.
Frequency, estimated volumes (as available from in-patients), results of any diagnostic procedures and
CTCAE grade is recorded in addition to specific dates of worsening and/or improvement. Treatment
interruptions/dose reductions (as described in the Safety Monitoring and Management Plan (SMMP))
are also recorded. Correlation to GVHD is assessed by the Investigator and a blinded adjudication
committee.
5) GVHD is assessed weekly throughout treatment on a separate CRF page. Organ stage and
overall grade are recorded at each visit and more frequently if GVHD events persist or require
additional, unscheduled visits. Medications administered for GVHD is specifically recorded including
indication (prophylaxis versus treatment), dose, duration of therapy and dose adjustments.
6) Analytes predictive of liver abnormalities including ALT, AST, total bilirubin, direct
bilirubin, albumin and alkaline phosphatase are monitored through weekly blood draws analyzed at the
central laboratory. Toxicity grades, shift in grades and changes from baseline are captured.
7) GI AEs and liver laboratory abnormalities are monitored as noted above and categorized
based on mg/kg dose of HDP-CDV or placebo.
8) AEs with an outcome of “fatal” is captured on the eCRF and investigated/described as part
of the SAE monitoring plan.
9) Emergence of antiviral resistant CMV as defined by clinical definition and/or retrospective
genotypic and phenotypic analysis (definitions of virologic failures are followed the virology analysis
plan).
10) All documented infections collected on the AE page with causal agent identified.
Dosing Regimen and Treatment Groups: The activity of 2 dosing regimens of HDP-CDV is assessed
compared to placebo and to each other. Subjects are randomized in a 1:1:1 ratio to one of three
treatment arms:
75 mg HDP-CDV BIW (BIW),
200 mg HDP-CDV QW (QW) for subjects weighing >65 kg or 150 mg QW for subjects
weighing ≦65 kg,
Placebo.
Randomization is stratified by weight (>65 kg versus ≦65 kg) and likelihood of CMV infection/disease
(highly likely versus less likely).
All site staff, subjects, and study personnel are blinded to both frequency and treatment assignment;
hence, all subjects take study medication and/or placebo two times each week. Doses should be
administered at an interval of 3 days followed by an interval of 4 days (e.g., Monday and Thursday, or
Tuesday and Friday).
Study Design:
Screening and Randomization: Study HDP-CDV-301 is a randomized, multicenter, double-blind,
parallel-group, placebo-controlled study of 2 dose regimens of HDP-CDV versus matching placebo.
All subjects are consented for participation in the study before or after transplant. Subjects who have
provided informed consent are screened following transplant, prior to dosing. As soon as possible
following engraftment and as close as possible to the scheduled time of first dose (defined as
enrollment), subjects should be randomized using the automated IVRS/IWRS system. CMV DNAemia
must be assessed by the central laboratory within 5 days of enrollment; only subjects who are CMV
DNA negative up until and at screening are eligible for randomization.
Subjects who meet all eligibility criteria is randomized 1:1:1 to HDP-CDV dose regimen QW, HDP-
CDV dose regimen BIW, or matching placebo as shown above. During randomization, subjects are
stratified based on the likelihood for progression to clinically significant CMV infection/disease
(highly likely versus less likely) and weight (≧65 kg versus <65 kg). Subjects who received a matched,
non T-cell depleted graft, non-myeloablative conditioning, are aGVHD negative, and who have not
received anti-thymocyte globulin (ATG) or systemic steroids are stratified in the “lower likelihood”
group. All others are stratified in the “higher likelihood” group.
Protocol Defined Treatment and Follow-Up Period: HSCT recipients who are CMV seropositive
pretransplant are considered at risk of CMV infection and managed under the care of their transplant
team for approximately 100 days following transplant. Therefore, the protocol defined treatment
period for this study was established to match this risk period. Dosing for all subjects is initiated no
later than Day 30 post-transplant and no later than 7 days post engraftment, and continues through
Week 13 post-transplant. Subjects take blinded study medication twice per week with food. During
the protocol specified treatment period (through Week 13 post-transplant), subjects return to the clinic
QW for safety and virology assessments, regardless of whether or not they are still receiving blinded
study medication. All subjects return to the clinic for the posttreatment Week 1 follow-up visit during
Week 14 post-transplant (i.e., coincident with the end of the 100 day monitoring period). Subjects are
then followed for 8 weeks posttreatment according to the time and events schedule. As noted, all
subjects who discontinue treatment early are continued to be followed in the study (i.e., weekly until
Week 13 post-transplant and then at Weeks 14, 17 and 21 post-transplant) according to the Time and
Events Schedule.
If any subject develops CMV disease or infection requiring alternate treatment or CMV DNAemia
>1,000 copies/mL (as measured by the central laboratory, confirmed by a second increasing value of at
least 2,000 copies/mL) during the treatment phase, that subject must discontinue study drug. Subjects
should have a confirmatory plasma sample drawn for assessment of CMV viremia prior to initiating
alternate CMV therapy, returning to the clinic for an unscheduled visit if necessary.
Number of Patients (Planned): Eligible subjects with be randomized to each HDP-CDV dose
regimen or placebo at a ratio of 1:1:1. Approximately 180 subjects are enrolled into each HDP-CDV-
treatment arm and 180 subjects into the placebo arm of the study; a total of approximately 540 subjects
are enrolled. Randomization is stratified by weight and likelihood for CMV infection (higher
likelihood versus lower likelihood).
Diagnosis and Main Criteria for Inclusion: Adult, allogeneic stem cell transplant recipients who
were CMV seropositive (R+) at the time of transplant and are CMV DNAemia negative from
transplant to the screening visit (no more than 5 days prior to dosing) are eligible for enrollment.
Key Inclusion Criteria Include:
1. Allogeneic HSCT recipients who were CMV seropositive before transplantation (i.e., R+
patients) but who are CMV DNAemia negative from transplant up until screening (no more than 5 days
prior to dosing).
2. Age ≧18 years. Males must be able and willing to use adequate contraceptive methods
throughout the treatment and follow-up phases of the study. Females must be postmenopausal,
surgically sterile or, for those female subjects of reproductive potential with a non-sterile male partner,
willing to agree to use 2 acceptable methods of birth control throughout the study, with at least one
being a barrier method.
3. Recipients who are up to and including 30 days post qualifying HSCT, but no more than 7
days following evidence of engraftment, defined by one of the following:
a) Absolute neutrophil count (ANC) increasing for 3 consecutive days with a count ≧500 cells/mm³
by the third day OR
b) Two (2) consecutive days with an ANC ≧500 cells/mm³,
[Note: For sites where standard site practice is to monitor white blood count (WBC) early after
transplant as opposed to ANC, engraftment is defined as WBC increasing for 3 consecutive days with
an ANC ≧500 cell/mm³ on the third day. For non-myeloablative or reduced-intensity transplants (i.e.,
mini-transplants) where ANC does not fall below 500 cells/mm³, the site definition of engraftment
should be used.]
4. Able to ingest and absorb oral medication (in the judgment of the investigator and based on
lack of significant GI events).
5. Willing and able to understand and provide written informed consent.
6. To the best of his or her knowledge, willing and able to participate in all required study
activities for the entire duration of the study.
Key Exclusion Criteria Include:
1. Patients who test positive for CMV DNAemia any time between transplant, screening and
the Baseline visit.
2. Patients weighing ≧120 kg.
3. Patients with hypersensitivity (not renal dysfunction or eye disorder) to cidofovir or HDP-
CDV or excipients.
4. Recipients who received any of the following:
a) Ganciclovir (GCV), vGCV, or foscarnet anytime post-transplant,
b) CDV within 30 days prior to enrollment,
c) Any other anti-CMV therapy following transplantation (including Cytogam ®¹) within 7
days prior to enrollment,
d) Any CMV vaccine,
e) Any investigational drug within 14 days prior to enrollment without the prior written
consent of the medical monitor. [Note: an investigational drug is defined as one that is not approved
by the FDA for any indication.]
f) Prior treatment with HDP-CDV.
5. Patients receiving high dose acyclovir (ACV) (>2000 mg total oral daily dose or >5 mg/kg
IV three times daily) or vACV (Valtrex; >3000 mg total daily dose) at the time of dosing.
6. Concomitant therapy with digoxin at the time of dosing.
7. Patients with active CMV disease diagnosed within 6 months prior to enrollment.
8. Patients who are HIV positive; patients with active HCV or HBV infection as evidenced by
plasma levels of HCV RNA or HBV DNA, respectively. [Note: Historical measurements taken
immediately prior to transplant may be used to satisfy this criterion.]
9. HSCT recipients who, other than the qualifying HSCT, received another allogeneic HSCT
within the past 2 years.
10. Patients with renal insufficiency as evidenced by GFR <30 mL/min.
11. Patients with hepatic abnormalities as evidenced by ALT or AST >5 x ULN or direct
bilirubin >5 x ULN.
12. Patients with active solid tumor malignancies with the exception of basal cell carcinoma or
the condition necessitating the stem cell transplant.
13. Patients with Stage 2 or higher GVHD of the GI tract; patients with any GI disease that
would, in the judgment of the investigator, preclude the patient from taking or absorbing oral
medication (e.g., clinically active Crohn's disease, ischemic colitis, moderate or severe ulcerative
colitis, small bowel resection, ileus, or any condition expected to require abdominal surgery during the
course of study participation).
14. Any other condition including abnormal laboratory values that would in the judgment of
the investigator put the subject at increased risk for participating in the trial, or interferes with the
conduct of the trial.
Investigational Product, Dosage and Mode of Administration: HDP-CDV tablets and matching
placebo is supplied for this study. Study drug is dosed orally (i.e., PO) and should be stored at room
temperature with excursions allowed as discussed in the Investigator's Brochure.
Duration of Treatment: Treatment begins at engraftment but no later than 30 days post-transplant and
continues through Week 13 post-transplant; subjects receive between 9 and 11 weeks of dosing of
study medication. All subjects are followed until Week 21 post-transplant.
Ongoing Safety Monitoring and Safety Reviews: Subjects return to the clinic weekly for safety and
efficacy assessments as described in the Time and Events Schedule. Subjects are managed according
to the Safety Monitoring and Management Plan (SMMP).
Subjects are discontinued from the randomized study treatment if either of the 2 the following events
occur:
The observation of clinically relevant signs/symptoms of liver injury and ALT or AST >3x
ULN (and 2x baseline), including increased fatigue, nausea, vomiting, right upper quadrant pain or
tenderness, fever, rash or eosinophilia (>5%); OR
Pregnancy
Study drug is interrupted and subjects managed according to the SMMP if any of the following events
occur:
Persistent diarrhea, grade 2 for 3 consecutive days or higher, if no other etiology has been
identified; OR
Confirmed increase in liver function tests (ALT or AST) ≧5x upper limit of normal (ULN)
for at least 2 weeks and >2x baseline; or >8x ULN and 2x baseline; or >3x ULN and having a total
bilirubin >2x ULN or INR >1.5 ULN, if no other etiology has been identified; OR
Stage 3 or higher GVHD of the intestine unresponsive to standard of care therapy
Statistical Considerations and methods:
General Considerations: Statistical analyses are reported using summary tables, figure, and data
listings. Continuous variables are summarized using the mean, standard deviation, median, quartiles,
minimum, and maximum. Categorical variables are summarized by numbers and percentages of
subjects in corresponding categories. All raw data obtained from the case report form (CRF) and any
derived data are included in data listings.
Unless otherwise stated, all statistical tests are two-sided. A p-value of <0.05 is considered statistically
significant. Values for missing data are imputed. Every effort is made to avoid missing data. A
missing value equal failure approach is applied for the purpose of the primary efficacy analyses. A
sensitivity analysis using last observation carried forward for missing efficacy variables may be
conducted. All efficacy endpoints are analyzed by treatment group and also by treatment group and
randomization strata.
Analysis Sets: The intent to treat (ITT) analysis set includes all subjects who took at least one dose of
study drug. This analysis set is used to summarize all baseline characteristics, efficacy and safety
outcomes based on the randomized treatment. The modified ITT (mITT) analysis set includes all
subjects from the ITT analysis set who have at least one efficacy evaluation following baseline. This
analysis set is used to summarize baseline characteristics and efficacy endpoints based on the treatment
actually received, including cases where the subject is treated contrary to the randomization schedule.
The per protocol (PP) analysis set includes all subjects in the mITT analysis set who complete the
study and do not have any major protocol deviations. This analysis set is used to summarize baseline
characteristics and efficacy endpoints based on the treatment actually received, including cases where
the subject is treated contrary to the randomization schedule. The SAFETY analysis set includes all
subjects who took at least one dose of study medication. This analysis set is used to summarize all
baseline characteristics and safety outcomes based on the treatment actually received, including cases
where the subject is treated contrary to the randomization schedule.
Sample Size: Approximately 540 subjects are enrolled into this study. Sample size calculations were
based on the primary study endpoint: the development of clinically significant CMV infection.
Qualified subjects are randomized to receive one of two HDP-CDV dosing regimens or placebo. The
randomization ratio is 1:1:1 among the three treatment arms. Randomization is stratified by weight and
likelihood for CMV infection (high versus low likelihood).
Power statement in support of primary endpoint:
Prior data indicate that the proportion of placebo subjects developing clinically significant CMV
infection is 0.30. A clinically meaningful relative risk of meeting the primary endpoint for HDP-CDV
subjects relative to placebo is 0.5 (i.e., 50% reduction). A two group continuity corrected χ² test with a
0.050 two-sided significance level have 85% power to detect the difference between a HDP-CDV
proportion of 0.15 and a Placebo proportion of 0.30 when the sample sizes are 150 in each treatment
group (a total sample size of 450 subjects allocated 1:1:1 among three treatment groups). Assuming a
20% dropout rate in the study, 180 subjects are enrolled in each study arm, for a total of 540 subjects.
Power statements in support of comparing the 2 HDP-CDV dose regimens:
The 2 HDP-CDV dose regimens are first compared based on the primary endpoint (i.e., clinically
significant CMV infection). For the purpose of comparing the 2 HDP-CDV dose regimens, when the
sample size is 180 per group, a two group continuity corrected χ² test with a 0.050 two-sided
significance level have 76% power to detect the difference between a Group 1 proportion, p₁, of 0.080
and a Group 2 proportion, p₂, of 0.180 (odds ratio of 2.524).
If no difference between the 2 HDP-CDV dose regimens is observed based on the primary endpoint,
then the 2 groups are compared based on virologic endpoints, first using CMV DNAemia in plasma
>1000 copies/mL, then CMV DNAemia in plasma ≧200 copies/mL. If one of the dose regimens is
superior to the other using any of the 3 analyses, then this regimen is considered as having greater
antiviral activity. When the sample size is 180 subjects per group and based on prior data indicating
that the failure rate among one of the HDP-CDV dose regimen is 0.1. If the true relative risk of failure
for subjects receiving the other HDP-CDV dose regimen relative to the first dose regimen is 2, we are
able to reject the null hypothesis that this relative risk equals 1 with a probability (power) of 0.759. The
Type I error probability associated with this test of this null hypothesis is 0.05. We are using an
uncorrected chi-squared statistic to evaluate this null hypothesis.
Baseline and Demographic Data: Distributions of demographic variables (e.g., age, sex) and
baseline variables are presented by treatment group to assess group homogeneity. Variables are
summarized using descriptive statistics appropriate for the data type (i.e., continuous, categorical).
Efficacy Endpoints
Primary Endpoint: The primary efficacy endpoint is a composite endpoint for the development of
clinically significant CMV infection. For the primary analysis, the primary endpoint are measured
during the protocol specified treatment period (through Week 14 post-transplant +/−4 days). The
proportion of subjects meeting this endpoint is compared between HDP-CDV treatment arms and
placebo using either a continuity-corrected χ² test or a Fisher's exact test in a stepwise fashion: first, the
more active HDP-CDV regimen (defined as the regimen with the lowest rate of subjects meeting the
primary endpoint) are compared to placebo; if the difference between this dosing regimen and placebo
is statistically significant, then the second dosing regimen is compared to placebo; if the two HDP-
CDV dosing regimen are statistically significantly different from placebo, then the two dosing
regimens groups are compared to each other. Additional sensitivity analyses pooling HDP-CDV
dosing regimens based on total weekly dose may be conducted. Subjects for whom the primary
endpoint is missing are considered failure. An approach such as Benjamini-Hochberg is used to
preserve alpha (i.e., Type I error) and control the false discovery rate (FDR) in this multiple testing
situation. A continuity-corrected χ² test is used to assess the association between the active HDP-CDV
dosing regimens and the primary endpoint.
Secondary Endpoints: Dichotomous secondary endpoints are analyzed using the same methods as
those used for the primary endpoint. Continuous endpoints are analyzed using normal-theory based
methods or non-parametric methods (e.g., van Elteren test).
Safety Endpoints: Safety endpoints include occurrence of adverse events (particularly GI events),
occurrence of GVHD, withdrawal due to an adverse event, treatment burden, dose interruption or
reduction due to AEs, clinically significant laboratory abnormalities, and change from baseline in
laboratory values (e.g., clinical chemistry, hematology).
Dichotomous safety endpoints are analyzed using the same methods as those used for the primary
endpoint. Continuous endpoints (e.g., change from baseline in laboratory values) are analyzed using
normal-theory based methods or non-parametric methods (e.g., van Elteren test).
Note: A PK substudy is implemented at selected sites. Details of the substudy are agreed upon with
the Agency prior to initiation of the substudy.

TABLE 58
Time and Events Schedule

| | Screening | | | | | | | | | | | | | | |
|---|---|---|---|---|---|---|---|---|---|---|---|---|---|---|---|
| | Post- | Treatment Week | | | | | | | | | | | Safety Follow-up Week | | |
| Assessment | transplant | FDD | | | | | | | | | | | EOT | | EOS |
| Study Week Assessment | to −1 days | Wk 1 | 2 | 3 | 4 | 5 | 6 | 7 | 8 | 9 | 10 | 11 | Wk 1 | 4 | Wk 8 |
| ICF/Inc./Exc. | X | X | | | | | | | | | | | | | |
| Medical History | X | | | | | | | | | | | | | | |
| Physical Exam | X | | | | X | | | | X | | | | X | | X |
| Vital signs/weight/Height | X | X | | | X | | | | X | | | | X | | |
| Pregnancy test | X | X | | | | | | | | | | | X | | X |
| Plasma for CMV PCR | X | X | X | X | X | X | X | X | X | X | X | X | X | X | X |
| (DNAemia) | | | | | | | | | | | | | | | |
| Plasma for storage for | X | X | X | X | X | X | X | X | X | X | X | X | X | X | X |
| resistance analysis | | | | | | | | | | | | | | | |
| Plasma for storage for | X | X | X | X | X | X | X | X | X | X | X | X | X | X | X |
| analysis of other dsDNA | | | | | | | | | | | | | | | |
| viruses | | | | | | | | | | | | | | | |
| Hematology | X | X | X | X | X | X | X | X | X | X | X | X | X | X | X |
| Chemistry | X | X | X | X | X | X | X | X | X | X | X | X | X | X | X |
| GVHD Assessment | X | X | X | X | X | X | X | X | X | X | X | X | X | X | X |
| Adverse events/ConMeds/ | X | X | X | X | X | X | X | X | X | X | X | X | X | X | X |
| procedures | | | | | | | | | | | | | | | |

### Example 7

### HDP-CDV-350 Study Design and Observations

Study HDP-CDV-350 was an open-label, expanded access study of HDP-CDV for the treatment of serious or immediately life-threatening diseases or conditions caused by dsDNA viruses in patients for whom no comparable or satisfactory alternative therapy was available. The virologic responses of 16 allogeneic HCT recipients enrolled and treated with HDP-CDV due to refractory CMV infection or disease and/or intolerance to available anti-CMV medications were evaluated.

All subjects completed a screening assessment (including obtaining written informed consent and, where applicable, assent) prior to initiating treatment with HDP-CDV. Key eligibility criteria were as follows: (1) immediately life-threatening or serious disease or condition caused by infection with a dsDNA virus; (2) life-expectancy of ≧2 weeks and commitment to continuation of supportive care for ≧4 weeks; (3) ability to ingest and absorb oral medicines, and (4) no comparable or satisfactory therapeutic alternative available, including being ineligible for controlled clinical studies evaluating HDP-CDV.

HDP-CDV was given orally twice weekly (BIW); 100 mg BIW for adults or 2 mg/kg BIW for pediatrics. CMV viral load (VL) was measured at baseline (BL), every day during treatment (“tx”) and at 1 week and 1 month post-treatment (“post-tx”). Eligible subjects received either a weight-based or fixed dose of HDP-CDV. Pediatric subjects (≦12 years old) received a 4 mg/kg total weekly dose (not-to-exceed 200 mg), as either 2 mg/kg twice-weekly (BIW) or 4 mg/kg once-weekly (QW). Adults and adolescent (13-17 years old) subjects received a 200 mg (not to exceed 4 mg/kg) total weekly dose, as either 100 mg BIW or 200 mg QW. BIW doses were administered at alternating 3- and 4-day intervals and QW doses on the same day each week.

Subjects were treated for an initial period of up to 3 months until either resolution or stabilization of their clinical disease or for 4 weeks following resolution of viral DNAemia, depending on the disease under treatment, whichever was longer. Treatment is extended for up to an additional 3 months, for a total duration of 6 months treatment, depending on the subject's clinical response.

Subjects underwent extensive safety monitoring, including routine safety laboratory assessments, physical examination, and vital sign measurements at regular intervals. In addition, subjects were monitored for viral DNA burden in plasma (DNAemia) and other affected compartments (e.g., urine, stool, CSF, etc.). Assessments were performed weekly for the first month, every 1 or 2 weeks for the next 2 months, and monthly thereafter for as long as treatment was continued. Subjects were monitored for adverse events (AEs) and for selected concomitant medication throughout the entire study, including the 1 month posttreatment follow-up period.

Mean age of the subjects was 49.3 years (range 7.6-66.8), and ten subjects (63%) among them were male. Underlying diseases were: acute leukemia in six subjects, multiple myeloma in three subjects, MDS in three subjects, lymphoma in two subjects, other diseases in two subjects. Fourteen had myeloablative HCT (88%), ten mismatched unrelated donor (63%), twelve T-cell depleted graft (75%), four cord blood (25%). At BL, all had received≧1 anti-CMV antiviral. Four (25%) had resistant CMV at BL (three were resistant to GCV, one was resistant to FOS+GCV). Four (25%) had CMV disease at BL (one pneumonitis, two GI, one encephalitis). Median exposure was 10 doses HDP-CDV (4-48), over a median 45 days (range 10-184) starting a median 82 days (36-2555) post HCT. Mean log₁₀ CMV VL at BL was 3.5 (2.3-5.5). Maximal reduction in CMV VL from BL was mean log₁₀ 1.13 (−0.30-2.60). Thirteen (81%) had >1 log₁₀ reduction or ≦LOQ (2 log₁₀) during tx. Seven subjects died (3 GvHD, one among these seven subjects developed CMV disease during tx; three subjects developed transplant-related toxicity; and one subject developed CMV pneumonitis). Three subjects discontinued tx due to AEs (two due to increased LFTs, one due to diarrhea). AEs were reversible after discontinuation. Tx is completed in four and is ongoing in two subjects.

### Demographic/Baseline Characteristics

The demographic and baseline characteristics are summarized in TABLE 59 and TABLE 60, respectively.

TABLE 59
Summary of Subject Demographics

| Age (yr) | |
|---|---|
| Mean | 49.3 |
| Median | 52.8 |
| Range | 7.6 to 66.8 |
| Sex (n [%]) | |
| Male | 10 (63%) |
| Female | 6 (38%) |
| Race (n [%]) | |
| White/Caucasian | 12 (75%) |
| Asian | 2 (13%) |
| Unknown | 2 (13%) |

TABLE 60
Summary of Baseline Characteristics

| Underlying Disease (n [%]) | |
|---|---|
| Acute leukemia | 6 (38%) |
| Multiple myeloma | 3 (19%) |
| Myelodysplastic syndrome | 3 (19%) |
| Lymphoma | 2 (13%) |
| Other | 2 (13%) |
| Transplant Details (n [%]) | |
| Myeloablative conditioning regimen | 14 (88%) |
| Mismatched, unrelated donor | 10 (63%) |
| T-cell depleted graft | 12 (75%) |
| Cord blood graft | 4 (25%) |
| Baseline CMV DNAemia (log₁₀ copies/mL) | |
| Mean | 3.48 |
| Median | 3.54 |
| Range | 2.30 to 5.51 |
| CMV Disease Present at Baseline (n [%]) | |
| Gastrointestinal (GI) disease | 2 (13%) |
| Encephalitis | 1 (6%) |
| Pneumonitis | 1 (6%) |
| CMV Resistant Isolate at Baseline (n [%]) | |
| Ganciclovir | 3 (19%) |
| Ganciclovir plus foscarnet | 1 (6%) |

### Duration of HDP-CDV Therapy

The median duration of HDP-CDV treatment was 12 doses (range: 4 to 50 doses) over a median 67 days (range: 10 to 184 days) and starting a median 82 days (range: 36 to 2,555 days) post-HCT.

### Subject Disposition

The final study disposition for all 16 subjects is summarized in TABLE 61.

TABLE 61
Subject Disposition

| Completed: | |
|---|---|
| HDP-CDV treatment | 4 (25%) |
| Study (i.e., including 1 month posttreatment follow-up period) | 8 (50%) |
| Reasons for Premature HDP-CDV Treatment Discontinuation: | |
| Death | 4 (25%) |
| Adverse event | 3 (19%) |
| Futility of care decision | 2 (13%) |
| Other | 2 (13%)ⁱ |
| Protocol noncompliance | 1 (6%) |
| Reason for Premature Study Discontinuation: | |
| Death | 7 (44%) |
| Futility of care decision | 1 (6%) |

ⁱBoth subjects completed the maximum 6 months HDP-CDV treatment without resolution of CMV infection

Overall, 7 subjects died; 3 from complications due to graft versus host disease (GVHD), including 1 subject who developed CMV disease (CMV end-organ infection—GI tract) during treatment, 2 from other transplant-related toxicities, and 2 from worsening of their underlying CMV disease (1 CMV pneumonitis and 1 CMV colitis/esophagitis).

### Adverse Events

Given that subjects had to have a serious or life-threatening disease and/or to have failed other available treatment options in order to qualify for this study, there was an expected high frequency of AEs in the study population overall. With the exception of diarrhea, with or without associated GI signs and symptoms, the vast majority of AEs were attributed to the underlying conditions of the subjects and not to HDP-CDV administration.

Treatment-related AEs (i.e., assessed as possibly or probably related to HDP-CDV) are summarized in TABLE 62.

TABLE 62
Summary of Treatment-related Adverse Events

| System Organ Class | No. of Subjects |
|---|---|
| Preferred Term | (%) |
| Blood and Lymphatic System Disorders | |
| Neutropenia | 1 (6%) |
| Gastrointestinal Disorders | |
| Colitis | 1 (6%) |
| Diarrhea | 12 (75%) |
| Flatulence | 1 (6%) |
| Nausea | 2 (13%) |
| Investigations | |
| Liver function tests abnormal | 3 (19%) |
| Metabolism and Nutrition Disorders | |
| Decreased appetite | 1 (6%) |

Of the 3 subjects who explicitly discontinued HDP-CDV treatment due to AEs, 2 subjects had elevated liver function tests (LFTs) and 1 subject had diarrhea. AEs were generally reversible after HDP-CDV discontinuation.

### Virologic Response

The maximal post-baseline change and the last on-treatment change from baseline CMV DNAemia for the 16 subjects are summarized in TABLE 63.

TABLE 63
Maximal Post-Baseline and Last On-Treatment Change in
CMV DNAemia

| | |
|---|---|
| Mean (range) maximal postbaseline change in CMV | −1.13 |
| DNAemia (log₁₀ copies/mL): | (+0.30, −2.60) |
| Proportion of subjects with ≧1 log₁₀ postbaseline | 13/16 |
| reduction in CMV | |
| DNAemia or postbaseline reduction to ≦ LLOQ^{a:} | (81.3%) |
| Mean (range) last on-treatment change from baseline | −0.63 |
| CMV DNAemia (log₁₀ copies/mL): | (+2.76, −1.76) |
| Proportion of subjects with ≧1 log₁₀ last on-treatment | 10/16 |
| reduction or last on-treatment reduction to ≦ LLOQ: | (62.5%) |

Error! Reference source not found.
Measurements performed by Viracor-IBT using the CMV quantitative polymerase chain reaction (qPCR) plasma 5500 assay (range: 100 to 1 × 10¹⁰ copies/mL)
LLOQ = lower limit of quantitation (100 copies/mL)

The virological response to HDP-CDV treatment, as mean change from baseline CMV DNAemia over time, is summarized in TABLE 64.

TABLE 64
Change from Baseline CMV DNAemia by Study Visit

| | | CMV DNAemia^{a}(log₁₀ copies/mL) | | |
|---|---|---|---|---|
| Study Visit | N | Mean | Minimum | Maximum |
| Baseline | 16 | 3.48 | 2.30 | 5.51 |
| Change from Baseline at: | | | | |
| Week 2 | 16 | −0.22 | +0.54 | −0.82 |
| Week 3 | 15 | −0.30 | +0.96 | −1.36 |
| Week 4 | 15 | −0.52 | +1.17 | −1.76 |
| Week 5 | 14 | −0.63 | +0.71 | −2.60 |
| Week 6 | 6 | −0.66 | +1.10 | −1.76 |
| Week 7 | 10 | −0.16 | +1.81 | −1.60 |
| Week 8 | 3 | +0.22 | +1.86 | −1.49 |
| Week 9 | 8 | −0.02 | +1.98 | −1.01 |
| Week 10 | 2 | +0.11 | +1.70 | −1.49 |
| Week 11 | 9 | +0.06 | +2.27 | −1.49 |
| Month 3 | 7 | +0.16 | +2.76 | −1.35 |
| Month 4 | 4 | −0.89 | +1.10 | −1.65 |
| Month 5 | 3 | −0.84 | +0.52 | −1.65 |
| Month 6 | 3 | −0.80 | +0.52 | −1.65 |
| +1 Week Posttreatment | 9 | −0.63 | +0.48 | −1.65 |
| +1 Month Posttreatment | 8 | −1.00 | +0.06 | −2.06 |

^{a}Measurements performed by Viracor-IBT using the CMV qPCR plasma 5500 assay (range: 100 to 1 × 10¹⁰ copies/mL)

The virologic responses of 8 subjects with no CMV disease whose last on-treatment CMV DNAemia value was ≦1,000 copies/mL were evaluated. The ≦1,000 copies/mL viral titer is a generally accepted threshold value above which preemptive therapy is initiated in subjects who are approaching or beyond the initial 100-day posttransplant period of greatest risk of CMV reactivation. The CMV DNAemia values for most subjects were reduced to the Lower Limit of Quantitation (“LLOQ”) of the CMV qPCR assay (100 copies/mL or 2 log₁₀ copies/mL), including 1 subject with preexisting GCV resistance (GCV-R) mutations at H520Q and A594P.

The virologic response and outcome for the 4 subjects with CMV disease at baseline, as well as for the 1 subject who developed CMV disease (end organ infection—GI tract) on-treatment were evaluated. All 5 subjects had an extremely poor prognosis, with 4 deaths and 1 subject discontinuing treatment due to futility of care. A transient virologic response was observed in 1 subject with a preexisting GCV-R mutation at L595F who developed CMV disease on-treatment, possibly indicating the development of resistance. In contrast, the subject with a preexisting GCV+FOS resistance mutation at V781I and CMV colitis at baseline achieved CMV DNAemia values of <LLOQ, before eventually succumbing to GVHD.

The response of 3 subjects who had no pre-existing CMV disease and a last CMV DNAemia value on-treatment of >1,000 copies/mL were also evaluated. Of these subjects, 2 subjects had their HDP-CDV treatment prematurely discontinued due to AEs (1 elevated LFTs and 1 diarrhea). The third subject completed the maximum 6-month period of HDP-CDV treatment. This subject's CMV DNAemia was declining at the end of treatment (last on-treatment value=1,000 copies/mL) and was ≦LLOQ at the +1 month posttreatment FU visit.

Overall, HDP-CDV was generally well-tolerated and was associated with a substantial reduction of CMV viral load in this highly immune suppressed and complex subject population with infections refractory to previous therapies. While subjects with preexisting CMV disease had a poor prognosis, preexisting resistance to anti-CMV therapies (mainly ganciclovir) did not preclude an antiviral response and potential therapeutic benefit.

TABLE 65
List of Abbreviations

| Abbreviation or | |
|---|---|
| Specialist Term | Explanation |
| AdV | Adenovirus |
| ACV | Acyclovir |
| ABT | Aminobenzotriazole |
| ADME | Absorption, distribution, metabolism, and excretion |
| AE | Adverse event (or experience) |
| aGVHD | Acute graft versus host disease |
| ALL | Acute lymphoblastic leukemia |
| AML | Acute myeloid leukemia |
| ALT | Alanine aminotransferase (or transaminase) |
| AME | Absorption, metabolism, and excretion |
| AST | Aspartate aminotransferase (or transaminase) |
| AUC | Area under curve (for parameter measurement over time) |
| AUCinf | Area under the plasma concentration-time curve from |
| | 0-time extrapolated to infinity (inf) |
| BIW | Twice weekly |
| CDV | Cidofovir |
| CDV-PP | Cidofovir diphosphate |
| Cmax | Maximum observed plasma concentration |
| CMV | Cytomegalovirus |
| CPT | Child-Pugh-Turcotte |
| CRF | Case report form |
| CYP | Cytochrome P450 |
| DNA | Deoxyribonucleic acid |
| DNAemia | CMV DNA detected in plasma samples by PCR testing |
| dsDNA | Double-stranded DNA |
| DSMB | Data safety monitoring board |
| EBV | Epstein-Barr virus |
| ECG | Electrocardiogram |
| eCRF | Electronic case report form |
| EIND | Emergency Investigational New Drug |
| ESRD | End stage renal disease |
| FDA | (US) Food and Drug Administration |
| FDD | First Dose Day |
| FU | Follow-up |
| G-CSF | Granulocyte-colony stimulating factor |
| GCV | Ganciclovir |
| GERD | Gastroesophageal reflux disease |
| GI | Gastrointestinal |
| GVHD | Graft versus host disease |
| HC | Healthy control |
| HSCT | Hematopoietic stem cell transplantation |
| HHV-6 | Human herpesvirus-6 |
| HSV-1 | Herpes simplex virus type-1 |
| HSV-2 | Herpes simplex virus 2 |
| IND | Investigational New Drug Application |
| ITT | Intent-to-treat |
| IV | Intravenously |
| KM | Kaplan Meier |
| LLN | Lower limit of normal |
| MAD | Multiple ascending doses |
| MDZ | Midazolam |
| MedDRA | Medical Dictionary for Regulatory Activities |
| MHI | Moderate hepatic impairment |
| mITT | Modified intent-to-treat |
| PCR | Polymerase chain reaction |
| PK | Pharmacokinetic(s) |
| PP | Per protocol |
| PO | Orally |
| PTLD | Posttransplant lymphoproliferative disorder |
| QT | Interval from the beginning of the QRS complex to the |
| | end of the T wave on ECG |
| QTc | QT interval corrected |
| QTcB | QT interval corrected using Bazett's formula |
| QTcF | QT interval corrected using Fridericia's formula |
| QW | Once weekly |
| R+ | CMV seropositive recipient status |
| RBC | Red blood cell |
| SAD | Single ascending dose |
| SAE | Serious adverse event (or experience) |
| SAP | Statistical Analysis Plan |
| SHI | Severe hepatic impairment |
| SMMP | Safety monitoring and management plan |
| SOC | System Organ Class |
| STOC | Standard of Care |
| TEAE | Treatment-emergent adverse event |
| Tmax | Time of maximum plasma concentration |
| TPN | Total parenteral nutrition |
| ULN | Upper limit of normal |
| vACV | Valaciclovir |
| vGCV | Valganciclovir |
| VZV | Varicella zoster virus |

### EQUIVALENTS

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of treatment, prevention, or delaying on-set of cytomegalovirus (CMV) infection or a CMV infection associated disease or disorder, the method comprising orally administering to a subject a pharmaceutical composition comprising a therapeutically effective dose of a compound selected from: 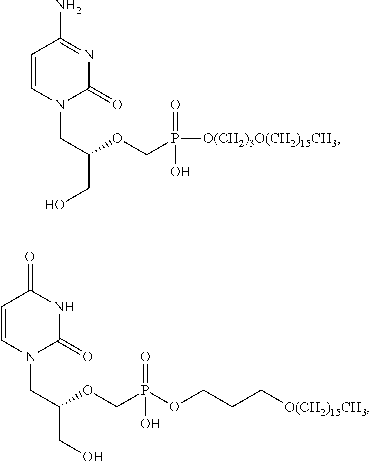
and a pharmaceutically acceptable salt thereof, wherein said subject is a post-hematopoietic stem cell transplant (HSCT) subject and is CMV seropositive before transplantation.

2. The method according to claim 1, wherein said subject is treated once a week (QW) with about 200 mg or twice a week (BIW) with about 100 mg of said compound.

3. The method according to claim 2, wherein said subject is treated twice a week (BIW) with about 100 mg of said compound.

4. The method according to claim 1, wherein said subject is treated once a week (QW) with about 150 mg or about 200 mg, or twice a week (BIW) with about 75 mg or about 100 mg of said compound.

5. The method according to claim 1, wherein said HSCT subject received an allogeneic stem cell transplant.

6. A method of prophylactic treatment, prevention, or delaying on-set of cytomegalovirus (CMV) infection or a CMV infection associated disease or disorder, the method comprising orally administering to a subject a pharmaceutical composition comprising a therapeutically effective dose of a compound selected from: 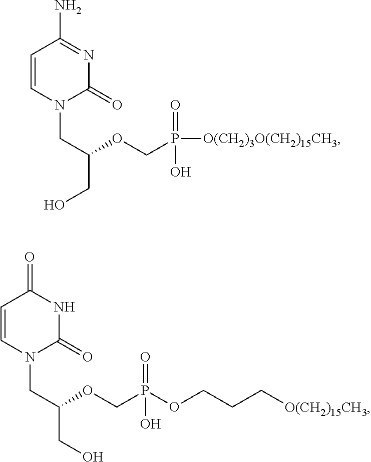
and a pharmaceutically acceptable salt thereof, wherein said subject is a post-hematopoietic stem cell transplant (HSCT) subject and is CMV seronegative before transplantation.

7. The method according to claim 6, wherein said subject is treated once a week (QW) with about 200 mg or twice a week (BIW) with about 100 mg of said compound.

8. The method according to claim 7, wherein said subject is treated once a week (QW) with about 200 mg of said compound.

9. The method according to claim 6, wherein said subject is treated once a week (QW) with about 150 mg or about 200 mg, or twice a week (BIW) with about 75 mg or about 100 mg of said compound.

10. The method according to claim 6, wherein said HSCT subject received an allogeneic stem cell transplant.

11. A method of treatment, prevention, or delaying on-set of cytomegalovirus (CMV) infection or a CMV infection associated disease or disorder, the method comprising orally administering to a subject a pharmaceutical composition comprising a therapeutically effective dose of a compound selected from: 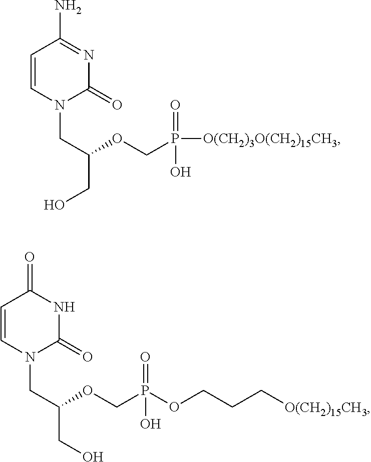
and a pharmaceutically acceptable salt thereof, in combination with one or more of a compound or composition selected from the group consisting of an immunosuppressant and an antiviral agent; wherein said subject is a post-hematopoietic stem cell transplant (HSCT) subject and is CMV seronegative before transplantation.

12. The method of claim 11, wherein said pharmaceutical composition is administered in combination with one or more compounds or compositions selected from the group consisting of: midazolam, cyclosporin A, tacrolimus, one or more azoles, ganciclovir, valganciclovir, foscavir, one or more second-line anti-CMV drugs, filgrastim, pegfilgrastim, one or more corticosteroids, beclomethasone, and one or more broad-spectrum CYP inhibitor aminobenzotriazoles.

13. The method of claim 12, wherein said one or more corticosteroids comprise budesonide.

14. The method of claim 12, wherein said one or more second-line anti-CMV drugs are selected from the group consisting of cidofovir, foscarnet, and intravenously administered (IV) cidofovir.
